(19) United States
(12) Patent Application Publication
(10) Pub. No.: US 2022/0288150 A1
(43) Pub. Date: Sep. 15, 2022

(54) PLANT MESSENGER PACKS ENCAPSULATING POLYPEPTIDES AND USES THEREOF

(71) Applicant: Flagship Pioneering Innovations VI, LLC, Cambridge, MA (US)

(72) Inventors: Maria Helena Christine VAN ROOIJEN, Cambridge, MA (US); John Patrick CASEY, JR., Boston, MA (US); Nataliya Vladimirovna NUKOLOVA, Cambridge, MA (US); Simon SCHWIZER, Boston, MA (US); Daniel Garcia CABANILLAS, Boston, MA (US)

(21) Appl. No.: 17/383,617

(22) Filed: Jul. 23, 2021

Related U.S. Application Data
(63) Continuation of application No. PCT/US2020/028007, filed on Apr. 13, 2020.
(60) Provisional application No. 62/848,482, filed on May 15, 2019.
(60) Provisional application No. 62/833,685, filed on Apr. 13, 2019.

(51) Int. Cl. A61K 36/752; C07K 14/62; A61K 31/164

(52) U.S. Cl. CPC A61K 36/752; A61K 31/164; C07K 14/62

(57) ABSTRACT

Disclosed herein are plant messenger packs (PMPs) encapsulating one or more exogenous polypeptides. Also disclosed are methods of producing a PMP comprising an exogenous polypeptide.

### BACKGROUND

Polypeptides (e.g., proteins or peptides) are used in therapies (e.g., for the treatment of a disease or condition), for diagnostic purposes, and as pathogen control agents. However, current methods of delivering polypeptides to cells may be limited by the mechanism of delivery, e.g., the efficiency of delivery of the polypeptide to a cell. Therefore, there is a need in the art for methods and compositions for the delivery of polypeptides to cells.

### SUMMARY OF THE INVENTION

In one aspect, the invention features a plant messenger pack (PMP) comprising one or more exogenous polypeptides, wherein the one or more exogenous polypeptides are mammalian therapeutic agents and are encapsulated by the PMP, and wherein the exogenous polypeptides are not pathogen control agents.

In some aspects, the mammalian therapeutic agent is an enzyme. In some aspects, the enzyme is a recombination enzyme or an editing enzyme.

In some aspects, the mammalian therapeutic agent is an antibody or an antibody fragment.

In some aspects, the mammalian therapeutic agent is an Fc fusion protein.

In some aspects, the mammalian therapeutic agent is a hormone. In some aspects, the mammalian therapeutic agent is insulin.

In some aspects, the mammalian therapeutic agent is a peptide.

In some aspects, the mammalian therapeutic agent is a receptor agonist or a receptor antagonist.

In some aspects, the mammalian therapeutic agent is an antibody of Table 1, a peptide of Table 2, an enzyme of Table 3, or a protein of Table 4.

In some aspects, the mammalian therapeutic agent has a size of less than 100 kD.

In some aspects, the mammalian therapeutic agent has a size of less than 50 kD.

In some aspects, the mammalian therapeutic agent has an overall charge that is neutral. In some aspects, the mammalian therapeutic agent has been modified to have a charge that is neutral. In some aspects, the mammalian therapeutic agent has an overall charge that is positive. In some aspects, the mammalian therapeutic agent has an overall charge that is negative.

In some aspects, the exogenous polypeptide is released from the PMP in a target cell with which the PMP is contacted. In some aspects, the exogenous polypeptide exerts activity in the cytoplasm of the target cell. In some aspects, the exogenous polypeptide is translocated to the nucleus of the target cell.

In some aspects, the exogenous polypeptide exerts activity in the nucleus of the target cell.

In some aspects, uptake by a cell of the exogenous polypeptide encapsulated by the PMP is increased relative to uptake of the exogenous polypeptide not encapsulated by a PMP.

In some aspects, the effectiveness of the exogenous polypeptide encapsulated by the PMP is increased relative to the effectiveness of the exogenous polypeptide not encapsulated by a PMP.

In some aspects, the exogenous polypeptide comprises at least 50 amino acid residues.

In some aspects, the exogenous polypeptide is at least 5 kD in size.

In some aspects, the PMP comprises a purified plant extracellular vesicle (EV), or a segment or extract thereof. In some aspects, the EV or segment or extract thereof is obtained from a citrus fruit, e.g., a grapefruit or a lemon.

In another aspect, the invention features a composition comprising a plurality of the PMPs of any of the above aspects.

In some aspects, the PMPs in the composition are at a concentration effective to increase the fitness of a mammal.

In some aspects, the exogenous polypeptide is at a concentration of at least 0.01, 0.1, 0.2, 0.3, 0.4, 0.5, or 1 μg polypeptide/mL.

In some aspects, at least 15% of PMPs in the plurality of PMPs encapsulate the exogenous polypeptide. In some aspects, at least 50% of PMPs in the plurality of PMPs encapsulate the exogenous polypeptide. In some aspects, at least 95% of PMPs in the plurality of PMPs encapsulate the exogenous polypeptide.

In some aspects, the composition is formulated for administration to a mammal. In some aspects, the composition is formulated for administration to a mammalian cell.

In some aspects, the composition further comprises a pharmaceutically acceptable vehicle, carrier, or excipient.

In some aspects, the composition is stable for at least one day at room temperature, and/or stable for at least one week at 4° C. In some aspects, the PMPs are stable for at least 24 hours, 48 hours, seven days, or 30 days at 4° C. In some aspects, the PMPs are further stable at a temperature of at least 20° C., 24° C., or 37° C.

In another aspect, the disclosure features a composition comprising a plurality of PMPs, wherein each of the PMPs is a plant EV, or a segment or extract thereof, wherein each of the plurality of PMPs encapsulate an exogenous polypeptide, wherein the exogenous polypeptide is a mammalian therapeutic agent, the exogenous polypeptide is not a pathogen control agent, and the composition is formulated for delivery to an animal.

In another aspect, the disclosure features a pharmaceutical composition comprising a composition according to any one of the above aspects and a pharmaceutically acceptable vehicle, carrier, or excipient.

In another aspect, the disclosure features a method of producing a PMP comprising an exogenous polypeptide, wherein the exogenous polypeptide is a mammalian therapeutic agent, and wherein the exogenous polypeptide is not a pathogen control agent, the method comprising (a) providing a solution comprising the exogenous polypeptide; and (b) loading the PMP with the exogenous polypeptide, wherein the loading causes the exogenous polypeptide to be encapsulated by the PMP.

In some aspects, the exogenous polypeptide is soluble in the solution.

In some aspects, the loading comprises one or more of sonication, electroporation, and lipid extrusion. In some aspects, the loading comprises sonication and lipid extrusion. In some aspects, the loading comprises lipid extrusion. In some aspects, PMP lipids are isolated prior to lipid extrusion. In some aspects, the isolated PMP lipids comprise glycosylinositol phosphorylceramides (GIPCs).

In another aspect, the disclosure features a method for delivering a polypeptide to a mammalian cell, the method comprising (a) providing a PMP comprising one or more exogenous polypeptides, wherein the one or more exogenous polypeptides are mammalian therapeutic agents and are encapsulated by the PMP, and wherein the exogenous polypeptides are not pathogen control agents; and (b) contacting the cell with the PMP, wherein the contacting is performed with an amount and for a time sufficient to allow uptake of the PMP by the cell. In some aspects, the cell is a cell in a subject.

In another aspect, the disclosure features a PMP, composition, pharmaceutical composition, or method of any of the above aspects, wherein the mammal is a human.

In another aspect, the disclosure features a method for treating diabetes, the method comprising administering to a subject in need thereof an effective amount of a composition comprising a plurality of PMPs, wherein one or more exogenous polypeptides are encapsulated by the PMP. In some aspects, the administration of the plurality of PMPs lowers the blood sugar of the subject. In some aspects, the exogenous polypeptide is insulin.

In another aspect, the disclosure features a PMP, composition, pharmaceutical composition, or method of any of the above aspects, wherein the PMP is not significantly degraded by gastric fluids, e.g., is not significantly degraded by fasted gastric fluids.

In a further aspect, the disclosure features a plant messenger pack (PMP) comprising one or more exogenous polypeptides, wherein the one or more exogenous polypeptides are encapsulated by the PMP.

In some aspects, the exogenous polypeptide is a therapeutic agent. In some aspects, the therapeutic agent is insulin.

In some aspects, the exogenous polypeptide is an enzyme. In some aspects, the enzyme is a recombination enzyme or an editing enzyme.

In some aspects, the exogenous peptide is a pathogen control agent.

In some aspects, the exogenous polypeptide is released from the PMP in a target cell with which the PMP is contacted. In some aspects, the exogenous polypeptide exerts activity in the cytoplasm of the target cell. In some aspects, the exogenous polypeptide is translocated to the nucleus of the target cell. In some aspects, the exogenous polypeptide exerts activity in the nucleus of the target cell.

In some aspects, uptake by a cell of the exogenous polypeptide encapsulated by the PMP is increased relative to uptake of the exogenous polypeptide not encapsulated by a PMP.

In some aspects, the effectiveness of the exogenous polypeptide encapsulated by the PMP is increased relative to the effectiveness of the exogenous polypeptide not encapsulated by a PMP.

In some aspects, the exogenous polypeptide comprises at least 50 amino acid residues. In some aspects, the exogenous polypeptide is at least 5 kD in size.

In some aspects, the exogenous polypeptide comprises fewer than 50 amino acid residues.

In some aspects, the PMP comprises a purified plant extracellular vesicle (EV), or a segment or extract thereof. In some aspects, the EV or segment or extract thereof is obtained from a citrus fruit. In some aspects, the citrus fruit is a grapefruit or a lemon.

In another aspect, the disclosure features a composition comprising a plurality of the PMPs of any of the above aspects.

In some aspects, the PMPs in the composition are at a concentration effective to increase the fitness of an organism. In some aspects, the PMPs in the composition are at a concentration effective to decrease the fitness of an organism.

In some aspects, the exogenous polypeptide is at a concentration of at least 0.01, 0.1, 0.2, 0.3, 0.4, 0.5, or 1 μg polypeptide/mL.

In some aspects, at least 15% of PMPs in the plurality of PMPs encapsulate the exogenous polypeptide. In some aspects, at least 50% of PMPs in the plurality of PMPs encapsulate the exogenous polypeptide. In some aspects, at least 95% of PMPs in the plurality of PMPs encapsulate the exogenous polypeptide.

In some aspects, the composition is formulated for administration to an animal. In some aspects, the composition is formulated for administration to an animal cell. In some aspects, the composition further comprises a pharmaceutically acceptable vehicle, carrier, or excipient.

In some aspects, the composition is formulated for administration to a plant. In some aspects, the composition is formulated for administration to a bacterium. In some aspects, the composition is formulated for administration to a fungus.

In some aspects, the composition is stable for at least one day at room temperature, and/or stable for at least one week at 4° C. In some aspects, the PMPs are stable for at least 24 hours, 48 hours, seven days, or 30 days at 4° C. In some aspects, the PMPs are further stable at a temperature of at least 20° C., 24° C., or 37° C.

In another aspect, the disclosure features a composition comprising a plurality of PMPs, wherein each of the PMPs is a plant EV, or a segment or extract thereof, wherein each of the plurality of PMPs encapsulate an exogenous polypeptide, and wherein the composition is formulated for delivery to an animal.

In another aspect, the disclosure features a pharmaceutical composition comprising a composition according to claim 1 and a pharmaceutically acceptable vehicle, carrier, or excipient.

In another aspect, the disclosure features a method of producing a PMP comprising an exogenous polypeptide, the method comprising (a) providing a solution comprising the exogenous polypeptide; and (b) loading the PMP with the exogenous polypeptide, wherein the loading causes the exogenous polypeptide to be encapsulated by the PMP.

In some aspects, the exogenous polypeptide is soluble in the solution.

In some aspects, the loading comprises one or more of sonication, electroporation, and lipid extrusion. In some aspects, the loading comprises sonication and lipid extrusion.

In some aspects, loading comprises lipid extrusion. In some aspects, PMP lipids are isolated prior to lipid extrusion. In some aspects, the isolated PMP lipids comprise glycosylinositol phosphorylceramides (GIPCs).

In another aspect, the disclosure features a method for delivering a polypeptide to a cell, the method comprising (a) providing a PMP comprising one or more exogenous polypeptides, wherein the one or more exogenous polypeptides are encapsulated by the PMP; and (b) contacting the cell with the PMP, wherein the contacting is performed with an amount and for a time sufficient to allow uptake of the PMP by the cell.

In some aspects, the cell is an animal cell. In some aspects, the cell is a cell in a subject.

In another aspect, the disclosure features a method for treating diabetes, the method comprising administering to a subject in need thereof an effective amount of a composition comprising a plurality of PMPs, wherein one or more exogenous polypeptides are encapsulated by the PMP. In some aspects, the administration of the plurality of PMPs lowers the blood sugar of the subject. In some aspects, the exogenous polypeptide is insulin.

### BRIEF DESCRIPTION OF THE DRAWINGS

FIG. 1A is a scatter plot and a bar graph showing PMP final concentration (PMPs/mL) and PMP size (in nm) in combined PMP-containing size exclusion chromatography (SEC) fractions following filter sterilization.

FIG. 1B is a graph showing PMP protein concentration (in μg/mL) in individual eluted fractions from SEC, as measured using a bicinchoninic acid assay (BCA assay). PMPs are eluted in fractions 4-6.

FIG. 2A is a schematic diagram showing the use of the Cre reporter system with plant messenger packs (PMPs) loaded with Cre recombinase. Human embryonic kidney 293 cells (HEK293 cells) comprising a Cre reporter transgene express GFP in the absence of the Cre protein (Unrecombined reporter⁺ cell), and express RFP in the presence of the Cre protein (Recombined reporter⁺ cell). The Cre protein is delivered to the cell in a PMP (+Cre-PMP).

FIG. 2B is a set of micrographs showing expression of fluorescent proteins in HEK293 cells that have been treated with Cre recombinase (Cre) and grapefruit (GF) PMPs that have not been electroporated; GFP PMPs only; CRE only; or Cre-loaded grapefruit PMPs. The top row shows fluorescence of GFP. The middle row shows fluorescence of RFP. RFP is expressed only in cells that have received Cre-loaded GF PMPs. The bottom row shows an overlay of the GFP and RFP fluorescent signals and a brightfield channel.

FIG. 3 is a schematic diagram showing an assay for the stability of loaded PMPs provided by oral delivery. (i) shows a PMP loaded with a human insulin polypeptide and comprising the covalent membrane dye DL800 IR or Alexa488. (ii) shows an in vitro assay for stability of PMPs and insulin exposed to mimetics of gastrointestinal (GI) juice. (iii) shows an in vivo assay for stability of PMPs and insulin provided by oral delivery (PMP gavage) to a streptzotocin-induced diabetes model mouse. Blood glucose levels, blood human insulin levels, immune profile, and biodistribution of DL800-labeled PMPs are measured.

FIG. 4 is a schematic diagram showing an assay for in vivo delivery by PMPs of Cre recombinase to a mouse having a luciferase Cre reporter construct (Lox-STOP-Lox-LUC). When Cre recombinase is delivered to a cell or tissue, recombination occurs and luciferase is expressed. Biodistribution of Cre recombinase by PMPs is measured by assessing luciferase expression in mouse tissues.

FIG. 5A is a schematic diagram showing a protocol for grapefruit PMP production using a destructive juicing step involving the use of a blender, followed by ultracentrifugation and sucrose gradient purification. Images are included of the grapefruit juice after centrifugation at 1000×g for 10 min and the sucrose gradient band pattern after ultracentrifugation at 150,000×g for 2 hours.

FIG. 5B is a plot of the PMP particle distribution measured by the Spectradyne NCS1.

FIG. 6 is a schematic diagram showing a protocol for grapefruit PMP production using a mild juicing step involving use of a mesh filter, followed by ultracentrifugation and sucrose gradient purification. Images are included of the grapefruit juice after centrifugation at 1000×g for 10 min and the sucrose gradient band pattern after ultracentrifugation at 150,000×g for 2 hours.

FIG. 7A is a schematic diagram showing a protocol for grapefruit PMP production using ultracentrifugation, followed by size exclusion chromatography (SEC) to isolate the PMP-containing fractions. The eluted SEC fractions are analyzed for particle concentration (NanoFCM), median particle size (NanoFCM), and protein concentration (BCA).

FIG. 7B is a graph showing particle concentration per mL in eluted size exclusion chromatography (SEC) fractions (NanoFCM). The fractions containing the majority of PMPs (“PMP fraction”) are indicated with an arrow. PMPs are eluted in fractions 2-4.

FIG. 7C is a set of graphs and a table showing particle size in nm for selected SEC fractions, as measured using NanoFCM. The graphs show PMP size distribution in fractions 1, 3, 5, and 8.

FIG. 7D is a graph showing protein concentration in μg/mL in SEC fractions, as measured using a BCA assay. The fraction containing the majority of PMPs (“PMP fraction”) is labeled, and an arrow indicates a fraction containing contaminants.

FIG. 8A is a schematic diagram showing a protocol for scaled PMP production from 1 liter of grapefruit juice (˜7 grapefruits) using a juice press, followed by differential centrifugation to remove large debris, 100× concentration of the juice using TFF, and size exclusion chromatography (SEC) to isolate the PMP containing fractions. The SEC elution fractions are analyzed for particle concentration (NanoFCM), median particle size (NanoFCM) and protein concentration (BCA).

FIG. 8B is a pair of graphs showing protein concentration (BCA assay, top panel) and particle concentration (NanoFCM, bottom panel) of SEC eluate volume (ml) from a scaled starting material of 1000 ml of grapefruit juice, showing a high amount of contaminants in the late SEC elution volumes.

FIG. 8C is a graph showing that incubation of the crude grapefruit PMP fraction with a final concentration of 50 mM EDTA, pH 7.15 followed by overnight dialysis using a 300 kDa membrane, successfully removed contaminants present in the late SEC elution fractions, as shown by absorbance at 280 nm. There was no difference in the dialysis buffers used (PBS without calcium/magnesium pH 7.4, MES pH 6, Tris pH 8.6).

FIG. 8D is a graph showing that incubation of the crude grapefruit PMP fraction with a final concentration of 50 mM EDTA, pH 7.15, followed by overnight dialysis using a 300 kDa membrane, successfully removed contaminants present in the late elution fractions after SEC, as shown by BCA protein analysis, which, besides detecting protein, is sensitive to the presence of sugars and pectins.

There was no difference in the dialysis buffers used (PBS without calcium/magnesium pH 7.4, MES pH 6, Tris pH 8.6).

FIG. 9A is a graph showing particle concentration (particles/ml) in eluted BMS plant cell culture SEC fractions, as measured by nano-flow cytometry (NanoFCM). PMPs were eluted in SEC fractions 4-6.

FIG. 9B is a graph showing absorbance at 280 nm (A.U.) in eluted BMS SEC fractions, measured on a SpectraMax® spectrophotometer. PMPs were eluted in fractions 4-6; fractions 9-13 contained contaminants.

FIG. 9C is a graph showing protein concentration (μg/ml) in eluted BMS SEC fractions, as determined by BCA analysis. PMPs were eluted in fractions 4-6; fractions 9-13 contained contaminants.

FIG. 9D is a scatter plot showing particles in the combined BMS PMP-containing SEC fractions as measured by nano-flow cytometry (NanoFCM). PMP concentration (particles/ml) was determined using a bead standard according to NanoFCM's instructions.

FIG. 9E is a graph showing the size distribution of BMS PMPs (nm) for the gated particles (background subtracted) of FIG. 6D. Median PMP size (nm) was determined using Exo bead standards according to NanoFCM's instructions.

FIG. 10 is a graph showing the luminescence (R.L.U., relative luminescence unit) of Pseudomonas aeruginosa bacteria that were treated with Ultrapure water (negative control), 3 ng free luciferase protein (protein only control) or with an effective luciferase protein dose of 3 ng by luciferase protein-loaded PMPs (PMP-Luc) in duplicate samples for 2 hrs at RT. Luciferase protein in the supernatant and pelleted bacteria was measured by luminescence using the ONE-Glo™ luciferase assay kit (Promega) and measured on a SpectraMax® spectrophotometer.

FIG. 11A is a Western blot showing insulin protein from insulin-loaded reconstructed PMPs recPMPs) that have been treated with a 1% Triton™ X-100 solution (Triton; Tx), a Proteinase K (ProtK) solution, a Tx solution followed by a ProtK solution, or a ProtK solution followed by a Tx solution. An untreated control is also shown.

FIG. 11B is a Western blot showing insulin protein from insulin-loaded recPMPs from lemon PMP lipids after incubation in simulated gastrointestinal fluids or a phosphate buffered saline (PBS) control at 37° C. PBS, pH 7.4, Fasted gastric fluid (Gastric Fasted), pH 1.6, 1 hour incubation; fasted intestinal fluid (Intestine Fasted), pH 6.4, 4 hour incubation; fed intestinal fluid (Intestine Fed), pH 5.8, 4 hour incubation.

### DETAILED DESCRIPTION OF THE INVENTION

### I. Definitions

As used herein, the term “encapsulate” or “encapsulated” refers to an enclosure of a moiety (e.g., an exogenous polypeptide as defined herein) within an enclosed lipid membrane structure, e.g., a lipid bilayer. The lipid membrane structure may be, e.g., a plant messenger pack (PMP) or a plant extracellular vesicle (EV), or may be obtained from or derived from a plant EV. An encapsulated moiety (e.g., an encapsulated exogenous polypeptide) is enclosed by the lipid membrane structure, e.g., such an encapsulated moiety is located in the lumen of the enclosed lipid membrane structure (e.g., the lumen of a PMP). The encapsulated moiety (e.g., the encapsulated polypeptide) may, in some instances, interact or associate with the inner face of the lipid membrane structure. The exogenous polypeptide may, in some instances, be intercalated with the lipid membrane structure. In some instances, the exogenous polypeptide has an extraluminal portion.

As used herein, the term “exogenous polypeptide” refers to a polypeptide (as is defined herein) that is encapsulated by a PMP (e.g., a PMP derived from a plant extracellular vesicle) that does not naturally occur in a plant lipid vesicle (e.g., does not naturally occur in a plant extracellular vesicle) or that is encapsulated in a PMP in an amount not found in a naturally occurring plant extracellular vesicle. The exogenous polypeptide may, in some instances, naturally occur in the plant from which the PMP is derived. In other instances, the exogenous polypeptide does not naturally occur in the plant from which the PMP is derived. The exogenous polypeptide may be artificially expressed in the plant from which the PMP is derived, e.g., may be a heterologous polypeptide. The exogenous polypeptide may be derived from another organism. In some aspects, the exogenous polypeptide is loaded into the PMP, e.g., using one or more of sonication, electroporation, lipid extraction, and lipid extrusion. The exogenous polypeptide may be, e.g., a therapeutic agent, an enzyme (e.g., a recombination enzyme or an editing enzyme), or a pathogen control agent.

As used herein, “delivering” or “contacting” refers to providing or applying a PMP composition (e.g., a PMP composition comprising an exogenous protein or peptide) to an organism, e.g., an animal, a plant, a fungus, or a bacterium. Delivery to an animal may be, e.g., oral delivery (e.g., delivery by feeding or by gavage) or systemic delivery (e.g., delivery by injection). The PMP composition may be delivered to the digestive tract, e.g., the stomach, the small intestine, or the large intestine. The PMP composition may be stable in the digestive tract.

As used herein, the term “animal” refers to humans, livestock, farm animals, invertebrates (e.g., insects), or mammalian veterinary animals (e.g., including for example, dogs, cats, horses, rabbits, zoo animals, cows, pigs, sheep, chickens, and non-human primates).

As used herein “decreasing the fitness of a pathogen” refers to any disruption to pathogen physiology as a consequence of administration of a PMP composition described herein, including, but not limited to, any one or more of the following desired effects: (1) decreasing a population of a pathogen by about 10%, 20%, 30%, 40%, 50%, 60%, 70%, 80%, 90%, 95%, 99%, 100% or more; (2) decreasing the reproductive rate of a pathogen by about 10%, 20%, 30%, 40%, 50%, 60%, 70%, 80%, 90%, 95%, 99%, 100% or more; (3) decreasing the mobility of a pathogen by about 10%, 20%, 30%, 40%, 50%, 60%, 70%, 80%, 90%, 95%, 99%, 100% or more; (4) decreasing the body weight or mass of a pathogen by about 10%, 20%, 30%, 40%, 50%, 60%, 70%, 80%, 90%, 95%, 99%, 100% or more; (5) decreasing the metabolic rate or activity of a pathogen by about 10%, 20%, 30%, 40%, 50%, 60%, 70%, 80%, 90%, 95%, 99%, 100% or more; or (6) decreasing pathogen transmission (e.g., vertical or horizontal transmission of a pathogen from one insect to another) by a pathogen by about 10%, 20%, 30%, 40%, 50%, 60%, 70%, 80%, 90%, 95%, 99%, 100% or more. A decrease in pathogen fitness can be determined, e.g., in comparison to an untreated pathogen.

As used herein “decreasing the fitness of a vector” refers to any disruption to vector physiology, or any activity carried out by said vector, as a consequence of administration of a vector control composition described herein, including, but not limited to, any one or more of the following desired effects: (1) decreasing a population of a vector by about 10%, 20%, 30%, 40%, 50%, 60%, 70%, 80%, 90%, 95%, 99%, 100% or more; (2) decreasing the reproductive rate of a vector (e.g., insect, e.g., mosquito, tick, mite, louse) by about 10%, 20%, 30%, 40%, 50%, 60%, 70%, 80%, 90%, 95%, 99%, 100% or more; (3) decreasing the mobility of a vector (e.g., insect, e.g., mosquito, tick, mite, louse) by about 10%, 20%, 30%, 40%, 50%, 60%, 70%, 80%, 90%, 95%, 99%, 100% or more; (4) decreasing the body weight of a vector (e.g., insect, e.g., mosquito, tick, mite, louse) by about 10%, 20%, 30%, 40%, 50%, 60%, 70%, 80%, 90%, 95%, 99%, 100% or more; (5) increasing the metabolic rate or activity of a vector (e.g., insect, e.g., mosquito, tick, mite, louse) by about 10%, 20%, 30%, 40%, 50%, 60%, 70%, 80%, 90%, 95%, 99%, 100% or more; (6) decreasing vector-vector pathogen transmission (e.g., vertical or horizontal transmission of a vector from one insect to another) by a vector (e.g., insect, e.g., mosquito, tick, mite, louse) by about 10%, 20%, 30%, 40%, 50%, 60%, 70%, 80%, 90%, 95%, 99%, 100% or more; (7) decreasing vector-animal pathogen transmission by about 10%, 20%, 30%, 40%, 50%, 60%, 70%, 80%, 90%, 95%, 99%, 100% or more; (8) decreasing vector (e.g., insect, e.g., mosquito, tick, mite, louse) lifespan by about 10%, 20%, 30%, 40%, 50%, 60%, 70%, 80%, 90%, 95%, 99%, 100% or more; (9) increasing vector (e.g., insect, e.g., mosquito, tick, mite, louse) susceptibility to pesticides (e.g., insecticides) by about 10%, 20%, 30%, 40%, 50%, 60%, 70%, 80%, 90%, 95%, 99%, 100% or more; or (10) decreasing vector competence by a vector (e.g., insect, e.g., mosquito, tick, mite, louse) by about 10%, 20%, 30%, 40%, 50%, 60%, 70%, 80%, 90%, 95%, 99%, 100% or more. A decrease in vector fitness can be determined, e.g., in comparison to an untreated vector.

As used herein, the term “formulated for delivery to an animal” refers to a PMP composition that includes a pharmaceutically acceptable carrier.

As used herein, the term “formulated for delivery to a pathogen” refers to a PMP composition that includes a pharmaceutically acceptable or agriculturally acceptable carrier.

As used herein, the term “formulated for delivery to a vector” refers to a PMP composition that includes an agriculturally acceptable carrier.

As used herein, the term “infection” refers to the presence or colonization of a pathogen in an animal (e.g., in one or more parts of the animal), on an animal (e.g., on one or more parts of the animal), or in the habitat surrounding an animal, particularly where the infection decreases the fitness of the animal, e.g., by causing a disease, disease symptoms, or an immune (e.g., inflammatory) response.

As used herein the term “pathogen” refers to an organism, such as a microorganism or an invertebrate, which causes disease or disease symptoms in an animal by, e.g., (i) directly infecting the animal, (ii) by producing agents that causes disease or disease symptoms in an animal (e.g., bacteria that produce pathogenic toxins and the like), and/or (iii) that elicit an immune (e.g., inflammatory response) in animals (e.g., biting insects, e.g., bedbugs). As used herein, pathogens include, but are not limited to bacteria, protozoa, parasites, fungi, nematodes, insects, viroids and viruses, or any combination thereof, wherein each pathogen is capable, either by itself or in concert with another pathogen, of eliciting disease or symptoms in humans.

As used herein, the term polypeptide,” “peptide,” or “protein” encompasses any chain of naturally or non-naturally occurring amino acids (either D- or L-amino acids), regardless of length (e.g., at least 2, 3, 4, 5, 6, 7, 10, 12, 14, 16, 18, 20, 25, 30, 40, 50, 100, 150, 200, 250, 300, 350, 400, 450, 500, 550, 600, 650, 700, 750, 800, 850, 900, 950, 1000, or more than 1000 amino acids), the presence or absence of post-translational modifications (e.g., glycosylation or phosphorylation), or the presence of, e.g., one or more non-amino acyl groups (for example, sugar, lipid, etc.) covalently linked to the polpeptide, and includes, for example, natural polypeptides, synthetic or recombinant polypeptides, hybrid molecules, peptoids, or peptidomimetics. The polypeptide may be, e.g. at least 0.1, at least 1, at least 5, at least 10, at least 15, at least 20, at least 30, at least 40, at least 50, or more than 50 kD in size. The polypeptide may be a full-length protein. Alternatively, the polypeptide may comprise one or more domains of a protein.

As used herein, the term “antibody” encompasses an immunoglobulin, whether natural or partly or wholly synthetically produced, and fragments thereof, capable of specifically binding to an antigen. The term also covers any protein having a binding domain which is homologous to an immunoglobulin binding domain. These proteins can be derived from natural sources, or partly or wholly synthetically produced. “Antibody” further includes a polypeptide comprising a framework region from an immunoglobulin gene or fragments thereof that specifically binds and recognizes an antigen. Use of the term “antibody” is meant to include whole antibodies, polyclonal, monoclonal and recombinant antibodies, fragments thereof, and further includes single-chain antibodies (nanobodies); humanized antibodies; murine antibodies; chimeric, mouse-human, mouse-primate, primate-human monoclonal antibodies, anti-idiotype antibodies, antibody fragments, such as, e.g., scFv, (scFv)2, Fab, Fab′, and F(abr′)2, F(ab1)2, Fv, dAb, and Fd fragments, diabodies, and antibody-related polypeptides. “Antibody” further includes bispecific antibodies and multispecific antibodies.

The term “antigen binding fragment”, as used herein, refers to fragments of an intact immunoglobulin, and any part of a polypeptide including antigen binding regions having the ability to specifically bind to the antigen. For example, the antigen binding fragment may be a F(ab′)2 fragment, a Fab′ fragment, a Fab fragment, a Fv fragment, or a scFv fragment, but is not limited thereto. A Fab fragment has one antigen binding site and contains the variable regions of a light chain and a heavy chain, the constant region of the light chain, and the first constant region CH₁ of the heavy chain. A Fab′ fragment differs from a Fab fragment in that the Fab′ fragment additionally includes the hinge region of the heavy chain, including at least one cysteine residue at the C-terminal of the heavy chain CH₁ region, The F(ab′)2 fragment is produced whereby cysteine residues of the Fab′ fragment are joined by a disulfide bond at the hinge region. A Fv fragment is the minimal antibody fragment having only heavy chain variable regions and light chain variable regions, and a recombinant technique for producing the Fv fragment is well known in the art. Two-chain Fv fragments may have a structure in which heavy chain variable regions are linked to light chain variable regions by a non-covalent bond. Single-chain Fv (scFv) fragments generally may have a dimer structure as in the two-chain Fv fragments in which heavy chain variable regions are covalently bound to light chain variable regions via a peptide linker or heavy and light chain variable regions are directly inked to each other at the C-terminal thereof. The antigen binding fragment may be obtained using a protease (for example, a whole antibody is digested with papain to obtain Fab fragments, and is digested with pepsin to obtain F(ab′)2 fragments), and may be prepared by a genetic recombinant technique. A dAb fragment consists of a VH domain.

Single-chain antibody molecules may comprise a polymer with a number of individual molecules, for example, dimer, trimer or other polymers.

As used herein, the term “heterologous” refers to an agent (e.g., a polypeptide) that is either (1) exogenous to the plant (e.g., originating from a source that is not the plant or plant part from which the PMP is produced) (e.g., an agent which is added to the PMP using loading approaches described herein) or (2) endogenous to the plant cell or tissue from which the PMP is produced, but present in the PMP (e.g., added to the PMP using loading approaches described herein, genetic engineering, as well as in vitro or in vivo approaches) at a concentration that is higher than that found in nature (e.g., higher than a concentration found in a naturally-occurring plant extracellular vesicle).

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “plant” refers to whole plants, plant organs, plant tissues, seeds, plant cells, seeds, and progeny of the same. Plant cells include, without limitation, cells from seeds, suspension cultures, embryos, meristematic regions, callus tissue, leaves, roots, shoots, gametophytes, sporophytes, pollen, and microspores. Plant parts include differentiated and undifferentiated tissues including, but not limited to the following: roots, stems, shoots, leaves, pollen, seeds, fruit, harvested produce, tumor tissue, and various forms of cells and culture (e.g., single cells, protoplasts, embryos, and callus tissue). The plant tissue may be in a plant or in a plant organ, tissue, or cell culture. In addition, a plant may be genetically engineered to produce a heterologous protein or RNA.

As used herein, the term “plant extracellular vesicle”, “plant EV”, or “EV” refers to an enclosed lipid-bilayer structure naturally occurring in a plant. Optionally, the plant EV includes one or more plant EV markers. As used herein, the term “plant EV marker” refers to a component that is naturally associated with a plant, such as a plant protein, a plant nucleic acid, a plant small molecule, a plant lipid, or a combination thereof, including but not limited to any of the plant EV markers listed in the Appendix. In some instances, the plant EV marker is an identifying marker of a plant EV but is not a pesticidal agent. In some instances, the plant EV marker is an identifying marker of a plant EV and also a pesticidal agent (e.g., either associated with or encapsulated by the plurality of PMPs, or not directly associated with or encapsulated by the plurality of PMPs).

As used herein, the term “plant messenger pack” or “PMP” refers to a lipid structure (e.g., a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter that is derived from (e.g., enriched, isolated or purified from) a plant source or segment, portion, or extract thereof, including lipid or non-lipid components (e.g., peptides, nucleic acids, or small molecules) associated therewith and that has been enriched, isolated or purified from a plant, a plant part, or a plant cell, the enrichment or isolation removing one or more contaminants or undesired components from the source plant. PMPs may be highly purified preparations of naturally occurring EVs. Preferably, at least 1% of contaminants or undesired components from the source plant are removed (e.g., at least 2%, 5%, 10%, 15%, 20%, 25%, 30%, 40%, 45%, 50%, 55%, 60%, 70%, 80%, 90%, 95%, 96%, 98%, 99%, or 100%) of one or more contaminants or undesired components from the source plant, e.g., plant cell wall components; pectin; plant organelles (e.g., mitochondria; plastids such as chloroplasts, leucoplasts or amyloplasts; and nuclei); plant chromatin (e.g., a plant chromosome); or plant molecular aggregates (e.g., protein aggregates, protein-nucleic acid aggregates, lipoprotein aggregates, or lipido-proteic structures). Preferably, a PMP is at least 30% pure (e.g., at least 40% pure, at least 50% pure, at least 60% pure, at least 70% pure, at least 80% pure, at least 90% pure, at least 99% pure, or 100% pure) relative to the one or more contaminants or undesired components from the source plant as measured by weight (w/w), spectral imaging (% transmittance), or conductivity (S/m).

In some instances, the PMP is a lipid extracted PMP (LPMP). As used herein, the terms “lipid extracted PMP” and “LPMP” refer to a PMP that has been derived from a lipid structure (e.g., a lipid bilayer, unilamellar, multilamellar structure; e.g., a vesicular lipid structure) derived from (e.g., enriched, isolated or purified from) a plant source, wherein the lipid structure is disrupted (e.g., disrupted by lipid extraction) and reassembled or reconstituted in a liquid phase (e.g., a liquid phase containing a cargo) using standard methods, e.g., reconstituted by a method comprising lipid film hydration and/or solvent injection, to produce the LPMP, as is described herein. The method may, if desired, further comprise sonication, freeze/thaw treatment, and/or lipid extrusion, e.g., to reduce the size of the reconstituted PMPs. A PMP (e.g., a LPMP) may comprise between 10% and 100% lipids derived from the lipid structure from the plant source, e.g., may contain at least 10%, at least 20%, at least 30%, at least 40%, at least 50%, at least 60%, at least 70%, at least 80%, at least 90%, at least 95%, or 100% lipids derived from the lipid structure from the plant source. A PMP (e.g., a LPMP) may comprise all or a fraction of the lipid species present in the lipid structure from the plant source, e.g., it may contain at least 10%, at least 20%, at least 30%, at least 40%, at least 50%, at least 60%, at least 70%, at least 80%, at least 90%, or 100% of the lipid species present in the lipid structure from the plant source. A PMP (e.g., a LPMP) may comprise none, a fraction, or all of the protein species present in the lipid structure from the plant source, e.g., may contain 0%, less than 1%, less than 5%, less than 10%, less than 15%, less than 20%, less than 30%, less than 40%, less than 50%, less than 60%, less than 70%, less than 80%, less than 90%, less than 100%, 01100% of the protein species present in the lipid structure from the plant source. In some instances, the lipid bilayer of the PMP (e.g., LPMP) does not contain proteins. In some instances, the lipid structure of the PMP (e.g., LPMP) contains a reduced amount of proteins relative to the lipid structure from the plant source.

PMPs (e.g., LPMPs) may optionally include exogenous lipids, e.g., lipids that are either (1) exogenous to the plant (e.g., originating from a source that is not the plant or plant part from which the PMP is produced) (e.g., added the PMP using methods described herein) or (2) endogenous to the plant cell or tissue from which the PMP is produced, but present in the PMP (e.g., added to the PMP using methods described herein, genetic engineering, in vitro or in vivo approaches) at a concentration that is higher than that found in nature (e.g., higher than a concentration found in a naturally-occurring plant extracellular vesicle). The lipid composition of the PMP may include 0%, less than 1%, or at least 1%, 2%, 5%, 10%, 15%, 20%, 25%, 30%, 40%, 50%, 60%, 70%, 80%, 90%, 95%, or more than 95% exogenous lipid. Exemplary exogenous lipids include cationic lipids, ionizable lipids, zwitterionic lipids, and lipidoids.

PMPs may optionally include additional agents, such as polypeptides, therapeutic agents, polynucleotides, or small molecules. The PMPs can carry or associate with additional agents (e.g., polypeptides) in a variety of ways to enable delivery of the agent to a target plant, e.g., by encapsulating the agent, incorporation of the agent in the lipid bilayer structure, or association of the agent (e.g., by conjugation) with the surface of the lipid bilayer structure. Heterologous functional agents can be incorporated into the PMPs either in vivo (e.g., in planta) or in vitro (e.g., in tissue culture, in cell culture, or synthetically incorporated).

As used herein, the term “pure” refers to a PMP preparation in which at least a portion (e.g., at least 20%, 25%, 30%, 40%, 45%, 50%, 55%, 60%, 70%, 80%, 90%, 95%, 96%, 98%, 99%, or 100%) of plant cell wall components, plant organelles (e.g., mitochondria, chloroplasts, and nuclei), or plant molecule aggregates (protein aggregates, protein-nucleic acid aggregates, lipoprotein aggregates, or lipido-proteic structures) have been removed relative to the initial sample isolated from a plant, or part thereof.

As used herein, the term “repellent” refers to an agent, composition, or substance therein, that deters pathogen vectors (e.g., insects, e.g., mosquitos, ticks, mites, or lice) from approaching or remaining on an animal. A repellent may, for example, decrease the number of pathogen vectors on or in the vicinity of an animal, but may not necessarily kill or decreasing the fitness of the pathogen vector.

As used herein, the term “treatment” refers to administering a pharmaceutical composition to an animal or a plant for prophylactic and/or therapeutic purposes. To “prevent an infection” refers to prophylactic treatment of an animal or a plant that does not yet have a disease or condition, but which is susceptible to, or otherwise at risk of, a particular disease or condition. To “treat an infection” refers to administering treatment to an animal or a plant already suffering from a disease to improve or stabilize the animal's condition.

As used herein, the term “treat an infection” refers to administering treatment to an individual (e.g., a plant or an animal) already having a disease to improve or stabilize the individual's condition. This may involve reducing colonization of a pathogen in, on, or around an animal or a plant by one or more pathogens (e.g., by about 1%, 2%, 5%, 10%, 20%, 30%, 40%, 50%, 60%, 70%, 80%, 90%, or 100%) relative to a starting amount and/or allow benefit to the individual (e.g., reducing colonization in an amount sufficient to resolve symptoms). In such instances, a treated infection may manifest as a decrease in symptoms (e.g., by about 1%, 2%, 5%, 10%, 20%, 30%, 40%, 50%, 60%, 70%, 80%, 90%, or 100%). In some instances, a treated infection is effective to increase the likelihood of survival of an individual (e.g., an increase in likelihood of survival by about 1%, 2%, 5%, 10%, 20%, 30%, 40%, 50%, 60%, 70%, 80%, 90%, or 100%) or increase the overall survival of a population (e.g., an increase in likelihood of survival by about 1%, 2%, 5%, 10%, 20%, 30%, 40%, 50%, 60%, 70%, 80%, 90%, or 100%). For example, the compositions and methods may be effective to “substantially eliminate” an infection, which refers to a decrease in the infection in an amount sufficient to sustainably resolve symptoms (e.g., for at least 1, 2, 3, 4, 5, 6, 7, 8, 9, 10, 11, or 12 months) in the animal or plant.

As used herein, the term “prevent an infection” refers to preventing an increase in colonization in, on, or around an animal or plant by one or more pathogens (e.g., by about 1%, 2%, 5%, 10%, 20%, 30%, 40%, 50%, 60%, 70%, 80%, 90%, 100%, or more than 100% relative to an untreated animal or plant) in an amount sufficient to maintain an initial pathogen population (e.g., approximately the amount found in a healthy individual), prevent the onset of an infection, and/or prevent symptoms or conditions associated with infection. For example, an individual (e.g., an animal, e.g., a human) may receive prophylaxis treatment to prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised individuals (e.g., individuals with cancer, with HIV/AIDS, or taking immunosuppressive agents), or in individuals undergoing long term antibiotic therapy.

As used herein, the term “stable PMP composition” (e.g., a composition including loaded or non-loaded PMPs) refers to a PMP composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of the initial number of PMPs (e.g., PMPs per mL of solution) relative to the number of PMPs in the PMP composition (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)); or retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity relative to the initial activity of the PMP (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

In some aspects, the stable PMP continues to encapsulate or remains associated with an exogenous polypeptide with which the PMP has been loaded, e.g., continues to encapsulate or remains associated with an exogenous polypeptide for at least 24 hours, at least 48 hours, at least 1 week, at least 2 weeks, at least 3 weeks, at least 4 weeks, at least 30 days, at least 60 days, at least 90 days, or 90 or more days.

As used herein, the term “vector” refers to an insect that can carry or transmit an animal pathogen from a reservoir to an animal. Exemplary vectors include insects, such as those with piercing-sucking mouthparts, as found in Hemiptera and some Hymenoptera and Diptera such as mosquitoes, bees, wasps, midges, lice, tsetse fly, fleas and ants, as well as members of the Arachnidae such as ticks and mites.

As used herein, the term “juice sac” or “juice vesicle” refers to a juice-containing membrane-bound component of the endocarp (carpel) of a hesperidium, e.g., a citrus fruit. In some aspects, the juice sacs are separated from other portions of the fruit, e.g., the rind (exocarp or flavedo), the inner rind (mesocarp, albedo, or pith), the central column (placenta), the segment walls, or the seeds. In some aspects, the juice sacs are juice sacs of a grapefruit, a lemon, a lime, or an orange.

### II. PMPs Comprising an Encapsulated Polypeptide and Compositions Thereof

The present invention includes plant messenger packs (PMPs) and compositions including a plurality of plant messenger packs (PMP). A PMP is a lipid (e.g., lipid bilayer, unilamellar, or multilamellar structure) structure that includes a plant EV, or segment, portion, or extract (e.g., lipid extract) thereof. Plant EVs refer to an enclosed lipid-bilayer structure that naturally occurs in a plant and that is about 5-2000 nm in diameter. Plant EVs can originate from a variety of plant biogenesis pathways. In nature, plant EVs can be found in the intracellular and extracellular compartments of plants, such as the plant apoplast, the compartment located outside the plasma membrane and formed by a continuum of cell walls and the extracellular space. Alternatively, PMPs can be enriched plant EVs found in cell culture media upon secretion from plant cells. Plant EVs can be isolated from plants (e.g., from the apoplastic fluid or from extracellular media), thereby producing PMPs, by a variety of methods, further described herein.

The PMPs and PMP compositions described herein include PMPs comprising an exogenous polypeptide, e.g., an exogenous polypeptide described in Section III herein. The exogenous polypeptide may be, e.g., a therapeutic agent, a pathogen control agent (e.g., an agent having antipathogen activity (e.g., antibacterial, antifungal, antinematicidal, antiparasitic, or antiviral activity)), or an enzyme (e.g., a recombination enzyme or an editing enzyme.

The plurality of PMPs in a PMP composition may be loaded with the exogenous polypeptide such that at least 5%, at least 10%, at least 15%, at least 25%, at least 50%, at least 75%, at least 90%, or at least 95% of PMPs in the plurality of PMPs encapsulate the exogenous polypeptide.

PMPs can include plant EVs, or segments, portions, or extracts, thereof, in which the plant EVs are about 5-2000 nm in diameter. For example, the PMP can include a plant EV, or segment, portion, or extract thereof, that has a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1250 nm, about 1250-1500 nm, about 1500-1750 nm, or about 1750-2000 nm. In some instances, the PMP includes a plant EV, or segment, portion, or extract thereof, that has a mean diameter of about 5-950 nm, about 5-900 nm, about 5-850 nm, about 5-800 nm, about 5-750 nm, about 5-700 nm, about 5-650 nm, about 5-600 nm, about 5-550 nm, about 5-500 nm, about 5-450 nm, about 5-400 nm, about 5-350 nm, about 5-300 nm, about 5-250 nm, about 5-200 nm, about 5-150 nm, about 5-100 nm, about 5-50 nm, or about 5-25 nm. In certain instances, the plant EV, or segment, portion, or extract thereof, has a mean diameter of about 50-200 nm. In certain instances, the plant EV, or segment, portion, or extract thereof, has a mean diameter of about 50-300 nm. In certain instances, the plant EV, or segment, portion, or extract thereof, has a mean diameter of about 200-500 nm. In certain instances, the plant EV, or segment, portion, or extract thereof, has a mean diameter of about 30-150 nm.

In some instances, the PMP may include a plant EV, or segment, portion, or extract thereof, that has a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, or at least 1000 nm. In some instances, the PMP includes a plant EV, or segment, portion, or extract thereof, that has a mean diameter less than 1000 nm, less than 950 nm, less than 900 nm, less than 850 nm, less than 800 nm, less than 750 nm, less than 700 nm, less than 650 nm, less than 600 nm, less than 550 nm, less than 500 nm, less than 450 nm, less than 400 nm, less than 350 nm, less than 300 nm, less than 250 nm, less than 200 nm, less than 150 nm, less than 100 nm, or less than 50 nm. A variety of methods (e.g., a dynamic light scattering method) standard in the art can be used to measure the particle diameter of the plant EVs, or segment, portion, or extract thereof.

In some instances, the PMP may include a plant EV, or segment, portion, or extract thereof, that has a mean surface area of 77 nm² to 3.2×10⁶ nm² (e.g., 77-100 nm², 100-1000 nm², 1000-1×10⁴ nm², 1×10⁴-1×10⁵ nm², 1×10⁵-1×10⁶ nm², or 1×10⁶-3.2×10⁶ nm²). In some instances, the PMP may include a plant EV, or segment, portion, or extract thereof, that has a mean volume of 65 nm³ to 5.3×10⁸ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-5.3×10⁸ nm³). In some instances, the PMP may include a plant EV, or segment, portion, or extract thereof, that has a mean surface area of at least 77 nm², (e.g., at least 77 nm², at least 100 nm², at least 1000 nm², at least 1×10⁴ nm², at least 1×10⁵ nm², at least 1×10⁶ nm², or at least 2×10⁶ nm²). In some instances, the PMP may include a plant EV, or segment, portion, or extract thereof, that has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 2×10⁸ nm³, at least 3×10⁸ nm³, at least 4×10⁸ nm³, or at least 5×10⁸ nm³.

In some instances, the PMP can have the same size as the plant EV or segment, extract, or portion thereof. Alternatively, the PMP may have a different size than the initial plant EV from which the PMP is produced. For example, the PMP may have a diameter of about 5-2000 nm in diameter. For example, the PMP can have a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1200 nm, about 1200-1400 nm, about 1400-1600 nm, about 1600-1800 nm, or about 1800-2000 nm. In some instances, the PMP may have a mean diameter of at least 5 nm, at least 50 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1000 nm, at least 1200 nm, at least 1400 nm, at least 1600 nm, at least 1800 nm, or about 2000 nm. A variety of methods (e.g., a dynamic light scattering method) standard in the art can be used to measure the particle diameter of the PMPs. In some instances, the size of the PMP is determined following loading of heterologous functional agents, or following other modifications to the PMPs.

In some instances, the PMP may have a mean surface area of 77 nm² to 1.3×10⁷ nm² (e.g., 77-100 nm², 100-1000 nm², 1000-1×10⁴ nm², 1×10⁴-1×10⁵ nm², 1×10⁵-1×10⁶ nm², or 1×10⁶-1.3×10⁷ nm²). In some instances, the PMP may have a mean volume of 65 nm³ to 4.2×10⁹ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-1×10⁹ nm³, or 1×10⁹-4.2×10⁹ nm³). In some instances, the PMP has a mean surface area of at least 77 nm², (e.g., at least 77 nm², at least 100 nm², at least 1000 nm², at least 1×10⁴ nm², at least 1×10⁵ nm², at least 1×10⁶ nm², or at least 1×10⁷ nm²). In some instances, the PMP has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 1×10⁹ nm³, at least 2×10⁹ nm³, at least 3×10⁹ nm³, or at least 4×10⁹ nm³).

In some instances, the PMP may include an intact plant EV. Alternatively, the PMP may include a segment, portion, or extract of the full surface area of the vesicle (e.g., a segment, portion, or extract including less than 100% (e.g., less than 90%, less than 80%, less than 70%, less than 60%, less than 50%, less than 40%, less than 30%, less than 20%, less than 10%, less than 10%, less than 5%, or less than 1%) of the full surface area of the vesicle) of a plant EV. The segment, portion, or extract may be any shape, such as a circumferential segment, spherical segment (e.g., hemisphere), curvilinear segment, linear segment, or flat segment. In instances where the segment is a spherical segment of the vesicle, the spherical segment may represent one that arises from the splitting of a spherical vesicle along a pair of parallel lines, or one that arises from the splitting of a spherical vesicle along a pair of non-parallel lines. Accordingly, the plurality of PMPs can include a plurality of intact plant EVs, a plurality of plant EV segments, portions, or extracts, or a mixture of intact and segments of plant EVs. One skilled in the art will appreciate that the ratio of intact to segmented plant EVs will depend on the particular isolation method used. For example, grinding or blending a plant, or part thereof, may produce PMPs that contain a higher percentage of plant EV segments, portions, or extracts than a non-destructive extraction method, such as vacuum-infiltration.

In instances where, the PMP includes a segment, portion, or extract of a plant EV, the EV segment, portion, or extract may have a mean surface area less than that of an intact vesicle, e.g., a mean surface area less than 77 nm², 100 nm², 1000 nm², 1×10⁴ nm², 1×10⁵ nm², 1×10⁶ nm², or 3.2×10⁶ nm²). In some instances, the EV segment, portion, or extract has a surface area of less than 70 nm², 60 nm², 50 nm², 40 nm², 30 nm², 20 nm², or 10 nm²). In some instances, the PMP may include a plant EV, or segment, portion, or extract thereof, that has a mean volume less than that of an intact vesicle, e.g., a mean volume of less than 65 nm³, 100 nm³, 1000 nm³, 1×10⁴ nm³, 1×10⁵ nm³, 1×10⁶ nm³, 1×10⁷ nm³, 1×10⁸ nm³, or 5.3×10⁸ nm³).

In instances where the PMP includes an extract of a plant EV, e.g., in instances where the PMP includes lipids extracted (e.g., with chloroform) from a plant EV, the PMP may include at least 1%, 2%, 5%, 10%, 20%, 30%, 40%, 50%, 60%, 70%, 80%, 90%, 95%, 99%, or more than 99% of lipids extracted (e.g., with chloroform) from a plant EV. The PMPs in the plurality may include plant EV segments and/or plant EV-extracted lipids or a mixture thereof.

Further outlined herein are details regarding methods of producing PMPs, plant EV markers that can be associated with PMPs, and formulations for compositions including PMPs.

### A. Production Methods

PMPs may be produced from plant EVs, or a segment, portion or extract (e.g., lipid extract) thereof, that occur naturally in plants, or parts thereof, including plant tissues or plant cells. An exemplary method for producing PMPs includes (a) providing an initial sample from a plant, or a part thereof, wherein the plant or part thereof comprises EVs; and (b) isolating a crude PMP fraction from the initial sample, wherein the crude PMP fraction has a decreased level of at least one contaminant or undesired component from the plant or part thereof relative to the level in the initial sample. The method can further include an additional step (c) comprising purifying the crude PMP fraction, thereby producing a plurality of pure PMPs, wherein the plurality of pure PMPs have a decreased level of at least one contaminant or undesired component from the plant or part thereof relative to the level in the crude EV fraction. Each production step is discussed in further detail, below. Exemplary methods regarding the isolation and purification of PMPs is found, for example, in Rutter and Innes, Plant Physiol. 173(1): 728-741, 2017; Rutter et al, Bio. Protoc. 7(17): e2533, 2017; Regente et al, J of Exp. Biol. 68(20): 5485-5496, 2017; Mu et al, Mol. Nutr. Food Res., 58, 1561-1573, 2014, and Regente et al, FEBS Letters. 583: 3363-3366, 2009, each of which is herein incorporated by reference.

For example, a plurality of PMPs may be isolated from a plant by a process which includes the steps of: (a) providing an initial sample from a plant, or a part thereof, wherein the plant or part thereof comprises EVs; (b) isolating a crude PMP fraction from the initial sample, wherein the crude PMP fraction has a decreased level of at least one contaminant or undesired component from the plant or part thereof relative to the level in the initial sample (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%); and (c) purifying the crude PMP fraction, thereby producing a plurality of pure PMPs, wherein the plurality of pure PMPs have a decreased level of at least one contaminant or undesired component from the plant or part thereof relative to the level in the crude EV fraction (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%).

The PMPs provided herein can include a plant EV, or segment, portion, or extract thereof, isolated from a variety of plants. PMPs may be isolated from any genera of plants (vascular or nonvascular), including but not limited to angiosperms (monocotyledonous and dicotyledonous plants), gymnosperms, ferns, selaginellas, horsetails, psilophytes, lycophytes, algae (e.g., unicellular or multicellular, e.g., archaeplastida), or bryophytes. In certain instances, PMPs can be produced from a vascular plant, for example monocotyledons or dicotyledons or gymnosperms. For example, PMPs can be produced from alfalfa, apple, Arabidopsis, banana, barley, canola, castor bean, chicory, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat or vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes, kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, or wheat.

PMPs may be produced from a whole plant (e.g., a whole rosettes or seedlings) or alternatively from one or more plant parts (e.g., leaf, seed, root, fruit, vegetable, pollen, phloem sap, or xylem sap). For example, PMPs can be produced from shoot vegetative organs/structures (e.g., leaves, stems, or tubers), roots, flowers and floral organs/structures (e.g., pollen, bracts, sepals, petals, stamens, carpels, anthers, or ovules), seed (including embryo, endosperm, or seed coat), fruit (the mature ovary), sap (e.g., phloem or xylem sap), plant tissue (e.g., vascular tissue, ground tissue, tumor tissue, or the like), and cells (e.g., single cells, protoplasts, embryos, callus tissue, guard cells, egg cells, or the like), or progeny of same. For instance, the isolation step may involve (a) providing a plant, or a part thereof, wherein the plant part is an Arabidopsis leaf. The plant may be at any stage of development. For example, the PMP can be produced from seedlings, e.g., 1 week, 2 week, 3 week, 4 week, 5 week, 6 week, 7 week, or 8 week old seedlings (e.g., Arabidopsis seedlings). Other exemplary PMPs can include PMPs produced from roots (e.g., ginger roots), fruit juice (e.g., grapefruit juice), vegetables (e.g., broccoli), pollen (e.g., olive pollen), phloem sap (e.g., Arabidopsis phloem sap), or xylem sap (e.g., tomato plant xylem sap). In some aspects, the PMP is produced from a citrus fruit, e.g., a grapefruit or a lemon.

PMPs can be produced from a plant, or part thereof, by a variety of methods. Any method that allows release of the EV-containing apoplastic fraction of a plant, or an otherwise extracellular fraction that contains PMPs comprising secreted EVs (e.g., cell culture media) is suitable in the present methods. EVs can be separated from the plant or plant part by either destructive (e.g., grinding or blending of a plant, or any plant part) or non-destructive (washing or vacuum infiltration of a plant or any plant part) methods. For instance, the plant, or part thereof, can be vacuum-infiltrated, ground, blended, or a combination thereof to isolate EVs from the plant or plant part, thereby producing PMPs. For instance, the isolating step may involve (b) isolating a crude PMP fraction from the initial sample (e.g., a plant, a plant part, or a sample derived from a plant or a plant part), wherein the crude PMP fraction has a decreased level of at least one contaminant or undesired component from the plant or part thereof relative to the level in the initial sample; wherein the isolating step involves vacuum infiltrating the plant (e.g., with a vesicle isolation buffer) to release and collect the apoplastic fraction. Alternatively, the isolating step may involve (b) grinding or blending the plant to release the EVs, thereby producing PMPs.

Upon isolating the plant EVs, thereby producing PMPs, the PMPs can be separated or collected into a crude PMP fraction (e.g., an apoplastic fraction). For instance, the separating step may involve separating the plurality of PMPs into a crude PMP fraction using centrifugation (e.g., differential centrifugation or ultracentrifugation) and/or filtration to separate the PMP-containing fraction from large contaminants, including plant tissue debris, plant cells, or plant cell organelles (e.g., nuclei or chloroplast). As such, the crude PMP fraction will have a decreased number of large contaminants, including, for example, plant tissue debris, plant cells, or plant cell organelles (e.g., nuclei, mitochondria or chloroplast), as compared to the initial sample from the source plant or plant part.

The crude PMP fraction can be further purified by additional purification methods to produce a plurality of pure PMPs. For example, the crude PMP fraction can be separated from other plant components by ultracentrifugation, e.g., using a density gradient (iodixanol or sucrose), size-exclusion, and/or use of other approaches to remove aggregated components (e.g., precipitation or size-exclusion chromatography). The resulting pure PMPs may have a decreased level of contaminants or undesired components from the source plant (e.g., one or more non-PMP components, such as protein aggregates, nucleic acid aggregates, protein-nucleic acid aggregates, free lipoproteins, lipido-proteic structures), nuclei, cell wall components, cell organelles, or a combination thereof) relative to one or more fractions generated during the earlier separation steps, or relative to a pre-established threshold level, e.g., a commercial release specification. For example, the pure PMPs may have a decreased level (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) of plant organelles or cell wall components relative to the level in the initial sample. In some instances, the pure PMPs are substantially free (e.g., have undetectable levels) of one or more non-PMP components, such as protein aggregates, nucleic acid aggregates, protein-nucleic acid aggregates, free lipoproteins, lipido-proteic structures), nuclei, cell wall components, cell organelles, or a combination thereof. Further examples of the releasing and separation steps can be found in Example 1. The PMPs may be at a concentration of, e.g., 1×10⁹, 5×10⁹, 1×10¹⁰, 5×10¹⁰, 5×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, or more than 1×10¹³ PMPs/mL.

For example, protein aggregates may be removed from isolated PMPs. For example, the isolated PMP solution can be taken through a range of pHs (e.g., as measured using a pH probe) to precipitate out protein aggregates in solution. The pH can be adjusted to, e.g., pH 3, pH 5, pH 7, pH 9, or pH 11 with the addition of, e.g., sodium hydroxide or hydrochloric acid. Once the solution is at the specified pH, it can be filtered to remove particulates. Alternatively, the isolated PMP solution can be flocculated using the addition of charged polymers, such as Polymin-P or Praestol 2640. Briefly, Polymin-P or Praestol 2640 is added to the solution and mixed with an impeller. The solution can then be filtered to remove particulates. Alternatively, aggregates can be solubilized by increasing salt concentration. For example NaCl can be added to the isolated PMP solution until it is at, e.g., 1 mol/L. The solution can then be filtered to isolate the PMPs. Alternatively, aggregates are solubilized by increasing the temperature. For example, the isolated PMPs can be heated under mixing until the solution has reached a uniform temperature of, e.g., 50° C. for 5 minutes. The PMP mixture can then be filtered to isolate the PMPs. Alternatively, soluble contaminants from PMP solutions can be separated by size-exclusion chromatography column according to standard procedures, where PMPs elute in the first fractions, whereas proteins and ribonucleoproteins and some lipoproteins are eluted later. The efficiency of protein aggregate removal can be determined by measuring and comparing the protein concentration before and after removal of protein aggregates via BCA/Bradford protein quantification. In some aspects, protein aggregates are removed before the exogenous polypeptide is encapsulated by the PMP. In other aspects, protein aggregates are removed after the exogenous polypeptide is encapsulated by the PMP.

Any of the production methods described herein can be supplemented with any quantitative or qualitative methods known in the art to characterize or identify the PMPs at any step of the production process. PMPs may be characterized by a variety of analysis methods to estimate PMP yield, PMP concentration, PMP purity, PMP composition, or PMP sizes. PMPs can be evaluated by a number of methods known in the art that enable visualization, quantitation, or qualitative characterization (e.g., identification of the composition) of the PMPs, such as microscopy (e.g., transmission electron microscopy), dynamic light scattering, nanoparticle tracking, spectroscopy (e.g., Fourier transform infrared analysis), or mass spectrometry (protein and lipid analysis). In certain instances, methods (e.g., mass spectroscopy) may be used to identify plant EV markers present on the PMP, such as markers disclosed in the Appendix. To aid in analysis and characterization, of the PMP fraction, the PMPs can additionally be labelled or stained. For example, the PMPs can be stained with 3,3′-dihexyloxacarbocyanine iodide (DIOC₆), a fluorescent lipophilic dye, PKH67 (Sigma Aldrich); Alexa Fluor® 488 (Thermo Fisher Scientific), or DyLight™ 800 (Thermo Fisher). In the absence of sophisticated forms of nanoparticle tracking, this relatively simple approach quantifies the total membrane content and can be used to indirectly measure the concentration of PMPs (Rutter and Innes, Plant Physiol. 173(1): 728-741, 2017; Rutter et al, Bio. Protoc. 7(17): e2533, 2017). For more precise measurements, and to assess the size distributions of PMPs, nanoparticle tracking, nano flow cytometry, or Tunable Resistive Pulse Sensing can be used.

During the production process, the PMPs can optionally be prepared such that the PMPs are at an increased concentration (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) relative to the EV level in a control or initial sample. The isolated PMPs may make up about 0.1% to about 100% of the PMP composition, such as any one of about 0.01% to about 100%, about 1% to about 99.9%, about 0.1% to about 10%, about 1% to about 25%, about 10% to about 50%, about 50% to about 99%, about. In some instances, the composition includes at least any of 0.1%, 0.5%, 1%, 5%, 10%, 20%, 30%, 40%, 50%, 60%, 70%, 80%, 90%, 95%, or more PMPs, e.g., as measured by wt/vol, percent PMP protein composition, and/or percent lipid composition (e.g., by measuring fluorescently labelled lipids); See, e.g., Example 3). In some instances, the concentrated agents are used as commercial products, e.g., the final user may use diluted agents, which have a substantially lower concentration of active ingredient. In some embodiments, the composition is formulated as a PMP concentrate formulation, e.g., an ultra-low-volume concentrate formulation. In some aspects, the PMPs in the composition are at a concentration effective to increase the fitness of an organism, e.g., a plant, an animal, an insect, a bacterium, or a fungus. In other aspects, the PMPs in the composition are at a concentration effective to decrease the fitness of an organism, e.g., a plant, an animal, an insect, a bacterium, or a fungus.

As illustrated by Example 1, PMPs can be produced from a variety of plants, or parts thereof (e.g., the leaf apoplast, seed apoplast, root, fruit, vegetable, pollen, phloem, or xylem sap). For example, PMPs can be released from the apoplastic fraction of a plant, such as the apoplast of a leaf (e.g., apoplast Arabidopsis thaliana leaves) or the apoplast of seeds (e.g., apoplast of sunflower seeds). Other exemplary PMPs are produced from roots (e.g., ginger roots), fruit juice (e.g., grapefruit juice), vegetables (e.g., broccoli), pollen (e.g., olive pollen), phloem sap (e.g., Arabidopsis phloem sap), xylem sap (e.g., tomato plant xylem sap), or cell culture supernatant (e.g. BY2 tobacco cell culture supernatant). This example further demonstrates the production of PMPs from these various plant sources.

As illustrated by Example 2, PMPs can be produced and purified by a variety of methods, for example, by using a density gradient (iodixanol or sucrose) in conjunction with ultracentrifugation and/or methods to remove aggregated contaminants, e.g., precipitation or size-exclusion chromatography. For instance, Example 2 illustrates purification of PMPs that have been obtained via the separation steps outlined in Example 1. Further, PMPs can be characterized in accordance with the methods illustrated in Example 3.

In some instances, the PMPs of the present compositions and methods can be isolated from a plant, or part thereof, and used without further modification to the PMP. In other instances, the PMP can be modified prior to use, as outlined further herein.

### B. Plant EV-Markers

The PMPs of the present compositions and methods may have a range of markers that identify the PMP as being produced from a plant EV, and/or including a segment, portion, or extract thereof. As used herein, the term “plant EV-marker” refers to a component that is naturally associated with a plant and incorporated into or onto the plant EV in planta, such as a plant protein, a plant nucleic acid, a plant small molecule, a plant lipid, or a combination thereof. Examples of plant EV-markers can be found, for example, in Rutter and Innes, Plant Physiol. 173(1): 728-741, 2017; Raimondo et al., Oncotarget. 6(23): 19514, 2015; Ju et al., Mol. Therapy. 21(7):1345-1357, 2013; Wang et al., Molecular Therapy. 22(3): 522-534, 2014; and Regente et al, J of Exp. Biol. 68(20): 5485-5496, 2017; each of which is incorporated herein by reference. Additional examples of plant EV-markers are listed in the Appendix, and are further outlined herein.

The plant EV marker can include a plant lipid. Examples of plant lipid markers that may be found in the PMP include phytosterol, campesterol, β-sitosterol, stigmasterol, avenasterol, glycosyl inositol phosphoryl ceramides (GIPCs), glycolipids (e.g., monogalactosyldiacylglycerol (MGDG) or digalactosyldiacylglycerol (DGDG)), or a combination thereof. For instance, the PMP may include GIPCs, which represent the main sphingolipid class in plants and are one of the most abundant membrane lipids in plants. Other plant EV markers may include lipids that accumulate in plants in response to abiotic or biotic stressors (e.g., bacterial or fungal infection), such as phosphatidic acid (PA) or phosphatidylinositol-4-phosphate (P14P).

Alternatively, the plant EV marker may include a plant protein. In some instances, the protein plant EV marker may be an antimicrobial protein naturally produced by plants, including defense proteins that plants secrete in response to abiotic or biotic stressors (e.g., bacterial or fungal infection). Plant pathogen defense proteins include soluble N-ethylmalemide-sensitive factor association protein receptor protein (SNARE) proteins (e.g., Syntaxin-121 (SYP121; GenBank Accession No.: NP_187788.1 or NP_974288.1), Penetration1 (PEN1; GenBank Accession No: NP_567462.1)) or ABC transporter Penetration3 (PEN3; GenBank Accession No: NP_191283.2). Other examples of plant EV markers includes proteins that facilitate the long-distance transport of RNA in plants, including phloem proteins (e.g., Phloem protein2-A1 (PP2-A1), GenBank Accession No: NP_193719.1), calcium-dependent lipid-binding proteins, or lectins (e.g., Jacalin-related lectins, e.g., Helianthus annuus jacalin (Helja; GenBank: AHZ86978.1). For example, the RNA binding protein may be Glycine-Rich RNA Binding Protein-7 (GRP7; GenBank Accession Number: NP_179760.1). Additionally, proteins that regulate plasmodesmata function can in some instances be found in plant EVs, including proteins such as Synap-Totgamin A A (GenBank Accession No: NP_565495.1). In some instances, the plant EV marker can include a protein involved in lipid metabolism, such as phospholipase C or phospholipase D. In some instances, the plant protein EV marker is a cellular trafficking protein in plants. In certain instances where the plant EV marker is a protein, the protein marker may lack a signal peptide that is typically associated with secreted proteins. Unconventional secretory proteins seem to share several common features like (i) lack of a leader sequence, (ii) absence of PTMs specific for ER or Golgi apparatus, and/or (iii) secretion not affected by brefeldin A which blocks the classical ER/Golgi-dependent secretion pathway. One skilled in the art can use a variety of tools freely accessible to the public (e.g., SecretomeP Database; SUBA3 (SUBcellular localization database for Arabidopsis proteins)) to evaluate a protein for a signal sequence, or lack thereof.

In instances where the plant EV marker is a protein, the protein may have an amino acid sequence having at least 35%, 40%, 45%, 50%, 55%, 60%, 65%, 70%, 75%, 80%, 85%, 90%, 95%, 98%, 99%, or 100% sequence identity to a plant EV marker, such as any of the plant EV markers listed in the Appendix. For example, the protein may have an amino acid sequence having at least 35%, 40%, 45%, 50%, 55%, 60%, 65%, 70%, 75%, 80%, 85%, 90%, 95%, 98%, 99%, or 100% sequence identity to PEN1 from Arabidopsis thaliana (GenBank Accession Number: NP_567462.1).

In some instances, the plant EV marker includes a nucleic acid encoded in plants, e.g., a plant RNA, a plant DNA, or a plant PNA. For example, the PMP may include dsRNA, mRNA, a viral RNA, a microRNA (miRNA), or a small interfering RNA (siRNA) encoded by a plant. In some instances, the nucleic acid may be one that is associated with a protein that facilitates the long-distance transport of RNA in plants, as discussed herein. In some instances, the nucleic acid plant EV marker may be one involved in host-induced gene silencing (HIGS), which is the process by which plants silence foreign transcripts of plant pests (e.g., pathogens such as fungi). For example, the nucleic acid may be one that silences bacterial or fungal genes. In some instances, the nucleic acid may be a microRNA, such as miR159 or miR166, which target genes in a fungal pathogen (e.g., Verticillium dahliae). In some instances, the protein may be one involved in carrying plant defense compounds, such as proteins involved in glucosinolate (GSL) transport and metabolism, including Glucosinolate Transporter-1-1 (GTR1; GenBank Accession No: NP_566896.2), Glucosinolate Transporter-2 (GTR2; NP_201074.1), orEpithiospecific Modifier 1 (ESM1; NP_188037.1).

In instances where the plant EV marker is a nucleic acid, the nucleic acid may have a nucleotide sequence having at least 35%, 40%, 45%, 50%, 55%, 60%, 65%, 70%, 75%, 80%, 85%, 90%, 95%, 98%, 99%, or 100% sequence identity to a plant EV marker, e.g., such as those encoding the plant EV markers listed in the Appendix. For example, the nucleic acid may have a polynucleotide sequence having at least 35%, 40%, 45%, 50%, 55%, 60%, 65%, 70%, 75%, 80%, 85%, 90%, 95%, 98%, 99%, or 100% sequence identity to miR159 or miR166.

In some instances, the plant EV marker includes a compound produced by plants. For example, the compound may be a defense compound produced in response to abiotic or biotic stressors, such as secondary metabolites. One such secondary metabolite that be found in PMPs are glucosinolates (GSLs), which are nitrogen and sulfur-containing secondary metabolites found mainly in Brassicaceae plants. Other secondary metabolites may include allelochemicals.

In some instances, the PMP may also be identified as being produced from a plant EV based on the lack of certain markers (e.g., lipids, polypeptides, or polynucleotides) that are not typically produced by plants, but are generally associated with other organisms (e.g., markers of animal EVs, bacterial EVs, or fungal EVs). For example, in some instances, the PMP lacks lipids typically found in animal EVs, bacterial EVs, or fungal EVs. In some instances, the PMP lacks lipids typical of animal EVs (e.g., sphingomyelin). In some instances, the PMP does not contain lipids typical of bacterial EVs or bacterial membranes (e.g., LPS). In some instances, the PMP lacks lipids typical of fungal membranes (e.g., ergosterol).

Plant EV markers can be identified using any approaches known in the art that enable identification of small molecules (e.g., mass spectroscopy, mass spectrometry), lipds (e.g., mass spectroscopy, mass spectrometry), proteins (e.g., mass spectroscopy, immunoblotting), or nucleic acids (e.g., PCR analysis). In some instances, a PMP composition described herein includes a detectable amount, e.g., a pre-determined threshold amount, of a plant EV marker described herein.

### C. Pharmaceutical Formulations

Included herein are PMP compositions that can be formulated into pharmaceutical compositions, e.g., for administration to an animal, such as a human. The pharmaceutical composition may be administered to an animal with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The single dose may be in a unit dose form as needed.

A PMP composition may be formulated for e.g., oral administration, intravenous administration (e.g., injection or infusion), or subcutaneous administration to an animal (e.g., a human). For injectable formulations, various effective pharmaceutical carriers are known in the art (See, e.g., Remington: The Science and Practice of Pharmacy, 22^{nd} ed., (2012) and ASHP Handbook on Injectable Drugs, 18th ed., (2014)).

Pharmaceutically acceptable carriers and excipients in the present compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. The compositions may be formulated according to conventional pharmaceutical practice. The concentration of the compound in the formulation will vary depending upon a number of factors, including the dosage of the active agent (e.g., the exogenous polypeptide encapsulated by the PMP) to be administered, and the route of administration.

For oral administration to an animal, the PMP composition can be prepared in the form of an oral formulation. Formulations for oral use can include tablets, caplets, capsules, syrups, or oral liquid dosage forms containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided in unit dosage form as chewable tablets, non-chewable tablets, caplets, capsules (e.g., as hard gelatin capsules wherein the active ingredient is mixed with an inert solid diluent, or as soft gelatin capsules wherein the active ingredient is mixed with water or an oil medium). The compositions disclosed herein may also further include an immediate-release, extended release or delayed-release formulation.

For parenteral administration to an animal, the PMP compositions may be formulated in the form of liquid solutions or suspensions and administered by a parenteral route (e.g., topical, subcutaneous, intravenous, or intramuscular). The pharmaceutical composition can be formulated for injection or infusion. Pharmaceutical compositions for parenteral administration can be formulated using a sterile solution or any pharmaceutically acceptable liquid as a vehicle. Pharmaceutically acceptable vehicles include, but are not limited to, sterile water, physiological saline, or cell culture media (e.g., Dulbecco's Modified Eagle Medium (DMEM), α-Modified Eagles Medium (α-MEM), F-12 medium). Formulation methods are known in the art, see e.g., Gibson (ed.) Pharmaceutical Preformulation and Formulation (2nd ed.) Taylor & Francis Group, CRC Press (2009).

### D. Agricultural Formulations

Included herein are PMP compositions that can be formulated into agricultural compositions, e.g., for administration to pathogen or pathogen vector (e.g., an insect). The agricultural composition may be administered to a pathogen or pathogen vector (e.g., an insect) with an agriculturally acceptable diluent, carrier, and/or excipient. Further examples of agricultural formulations useful in the present compositions and methods are further outlined herein.

To allow ease of application, handling, transportation, storage, and activity, the active agent, here PMPs, can be formulated with other substances. PMPs can be formulated into, for example, baits, concentrated emulsions, dusts, emulsifiable concentrates, fumigants, gels, granules, microencapsulations, seed treatments, suspension concentrates, suspoemulsions, tablets, water soluble liquids, water dispersible granules or dry flowables, wettable powders, and ultra-low volume solutions. For further information on formulation types see “Catalogue of Pesticide Formulation Types and International Coding System” Technical Monograph n° 2, 5th Edition by CropLife International (2002).

Active agents (e.g., PMPs comprising an exogenous polypeptide) can be applied most often as aqueous suspensions or emulsions prepared from concentrated formulations of such agents. Such water-soluble, water-suspendable, or emulsifiable formulations are either solids, usually known as wettable powders, or water dispersible granules, or liquids usually known as emulsifiable concentrates, or aqueous suspensions. Wettable powders, which may be compacted to form water dispersible granules, comprise an intimate mixture of the pesticide, a carrier, and surfactants. The carrier is usually selected from among the attapulgite clays, the montmorillonite clays, the diatomaceous earths, or the purified silicates. Effective surfactants, including from about 0.5% to about 10% of the wettable powder, are found among sulfonated lignins, condensed naphthalenesulfonates, naphthalenesulfonates, alkylbenzenesulfonates, alkyl sulfates, and non-ionic surfactants such as ethylene oxide adducts of alkyl phenols.

Emulsifiable concentrates can comprise a suitable concentration of PMPs, such as from about 50 to about 500 grams per liter of liquid dissolved in a carrier that is either a water miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are selected from conventional anionic and non-ionic surfactants.

Aqueous suspensions comprise suspensions of water-insoluble pesticides dispersed in an aqueous carrier at a concentration in the range from about 5% to about 50% by weight. Suspensions are prepared by finely grinding the pesticide and vigorously mixing it into a carrier comprised of water and surfactants. Ingredients, such as inorganic salts and synthetic or natural gums may also be added, to increase the density and viscosity of the aqueous carrier.

PMPs may also be applied as granular compositions that are particularly useful for applications to the soil. Granular compositions usually contain from about 0.5% to about 10% by weight of the pesticide, dispersed in a carrier that includes clay or a similar substance. Such compositions are usually prepared by dissolving the formulation in a suitable solvent and applying it to a granular carrier which has been pre-formed to the appropriate particle size, in the range of from about 0.5 to about 3 mm. Such compositions may also be formulated by making a dough or paste of the carrier and compound and crushing and drying to obtain the desired granular particle size.

Dusts containing the present PMP formulation are prepared by intimately mixing PMPs in powdered form with a suitable dusty agricultural carrier, such as kaolin clay, ground volcanic rock, and the like. Dusts can suitably contain from about 1% to about 10% of the packets. They can be applied as a seed dressing or as a foliage application with a dust blower machine.

It is equally practical to apply the present formulation in the form of a solution in an appropriate organic solvent, usually petroleum oil, such as the spray oils, which are widely used in agricultural chemistry.

PMPs can also be applied in the form of an aerosol composition. In such compositions the packets are dissolved or dispersed in a carrier, which is a pressure-generating propellant mixture. The aerosol composition is packaged in a container from which the mixture is dispensed through an atomizing valve.

Another embodiment is an oil-in-water emulsion, wherein the emulsion includes oily globules which are each provided with a lamellar liquid crystal coating and are dispersed in an aqueous phase, wherein each oily globule includes at least one compound which is agriculturally active, and is individually coated with a monolamellar or oligolamellar layer including: (1) at least one non-ionic lipophilic surface-active agent, (2) at least one non-ionic hydrophilic surface-active agent and (3) at least one ionic surface-active agent, wherein the globules having a mean particle diameter of less than 800 nanometers. Further information on the embodiment is disclosed in U.S. patent publication 20070027034 published Feb. 1, 2007. For ease of use, this embodiment will be referred to as “OIWE.”

Additionally, generally, when the molecules disclosed above are used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulfate; sodium dioctyl sulfosuccinate; alkyl phenol ethoxylates; and aliphatic alcohol ethoxylates.

A dispersing agent is a substance which adsorbs onto the surface of particles and helps to preserve the state of dispersion of the particles and prevents them from reaggregating. Dispersing agents are added to agrochemical formulations to facilitate dispersion and suspension during manufacture, and to ensure the particles redisperse into water in a spray tank. They are widely used in wettable powders, suspension concentrates and water-dispersible granules. Surfactants that are used as dispersing agents have the ability to adsorb strongly onto a particle surface and provide a charged or steric barrier to reaggregation of particles. The most commonly used surfactants are anionic, non-ionic, or mixtures of the two types. For wettable powder formulations, the most common dispersing agents are sodium lignosulfonates. For suspension concentrates, very good adsorption and stabilization are obtained using polyelectrolytes, such as sodium naphthalene sulfonate formaldehyde condensates. Tristyrylphenol ethoxylate phosphate esters are also used. Non-ionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces. Examples of dispersing agents used in agrochemical formulations are: sodium lignosulfonates; sodium naphthalene sulfonate formaldehyde condensates; tristyrylphenol ethoxylate phosphate esters; aliphatic alcohol ethoxylates; alkyl ethoxylates; EO-PO (ethylene oxide-propylene oxide) block copolymers; and graft copolymers.

An emulsifying agent is a substance which stabilizes a suspension of droplets of one liquid phase in another liquid phase. Without the emulsifying agent the two liquids would separate into two immiscible liquid phases. The most commonly used emulsifier blends contain alkylphenol or aliphatic alcohol with twelve or more ethylene oxide units and the oil-soluble calcium salt of dodecylbenzenesulfonic acid. A range of hydrophile-lipophile balance (“HLB”) values from 8 to 18 will normally provide good stable emulsions. Emulsion stability can sometimes be improved by the addition of a small amount of an EO-PO block copolymer surfactant.

A solubilizing agent is a surfactant which will form micelles in water at concentrations above the critical micelle concentration. The micelles are then able to dissolve or solubilize water-insoluble materials inside the hydrophobic part of the micelle. The types of surfactants usually used for solubilization are non-ionics, sorbitan monooleates, sorbitan monooleate ethoxylates, and methyl oleate esters.

Surfactants are sometimes used, either alone or with other additives such as mineral or vegetable oils as adjuvants to spray-tank mixes to improve the biological performance of the pesticide on the target. The types of surfactants used for bioenhancement depend generally on the nature and mode of action of the pesticide. However, they are often non-ionics such as: alkyl ethoxylates; linear aliphatic alcohol ethoxylates; aliphatic amine ethoxylates.

A carrier or diluent in an agricultural formulation is a material added to the pesticide to give a product of the required strength. Carriers are usually materials with high absorptive capacities, while diluents are usually materials with low absorptive capacities. Carriers and diluents are used in the formulation of dusts, wettable powders, granules, and water-dispersible granules.

Organic solvents are used mainly in the formulation of emulsifiable concentrates, oil-in-water emulsions, suspoemulsions, and ultra low volume formulations, and to a lesser extent, granular formulations. Sometimes mixtures of solvents are used. The first main groups of solvents are aliphatic paraffinic oils such as kerosene or refined paraffins. The second main group (and the most common) includes the aromatic solvents such as xylene and higher molecular weight fractions of C9 and C10 aromatic solvents. Chlorinated hydrocarbons are useful as cosolvents to prevent crystallization of pesticides when the formulation is emulsified into water. Alcohols are sometimes used as cosolvents to increase solvent power. Other solvents may include vegetable oils, seed oils, and esters of vegetable and seed oils.

Thickeners or gelling agents are used mainly in the formulation of suspension concentrates, emulsions, and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. Examples of these types of materials, include, but are not limited to, montmorillonite, bentonite, magnesium aluminum silicate, and attapulgite. Water-soluble polysaccharides have been used as thickening-gelling agents for many years. The types of polysaccharides most commonly used are natural extracts of seeds and seaweeds or are synthetic derivatives of cellulose. Examples of these types of materials include, but are not limited to, guar gum; locust bean gum; carrageenam; alginates; methyl cellulose; sodium carboxymethyl cellulose (SCMC); hydroxyethyl cellulose (HEC). Other types of anti-settling agents are based on modified starches, polyacrylates, polyvinyl alcohol, and polyethylene oxide. Another good anti-settling agent is xanthan gum.

Microorganisms can cause spoilage of formulated products. Therefore preservation agents are used to eliminate or reduce their effect. Examples of such agents include, but are not limited to: propionic acid and its sodium salt; sorbic acid and its sodium or potassium salts; benzoic acid and its sodium salt; p-hydroxybenzoic acid sodium salt; methyl p-hydroxybenzoate; and 1,2-benzisothiazolin-3-one (BIT).

The presence of surfactants often causes water-based formulations to foam during mixing operations in production and in application through a spray tank. In order to reduce the tendency to foam, anti-foam agents are often added either during the production stage or before filling into bottles. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

“Green” agents (e.g., adjuvants, surfactants, solvents) can reduce the overall environmental footprint of crop protection formulations. Green agents are biodegradable and generally derived from natural and/or sustainable sources, e.g., plant and animal sources. Specific examples are: vegetable oils, seed oils, and esters thereof, also alkoxylated alkyl polyglucosides.

In some instances, PMPs can be freeze-dried or lyophilized. See U.S. Pat. No. 4,311,712. The PMPs can later be reconstituted on contact with water or another liquid. Other components can be added to the lyophilized or reconstituted liposomes, for example, other antipathogen agents, pesticidal agents, repellent agents, agriculturally acceptable carriers, or other materials in accordance with the formulations described herein.

Other optional features of the composition include carriers or delivery vehicles that protect the PMP composition against UV and/or acidic conditions. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

The composition may additionally be formulated with an attractant (e.g., a chemoattractant) that attracts a pest, such as a pathogen vector (e.g., an insect), to the vicinity of the composition. Attractants include pheromones, a chemical that is secreted by an animal, especially a pest, or chemoattractants which influences the behavior or development of others of the same species. Other attractants include sugar and protein hydrolysate syrups, yeasts, and rotting meat. Attractants also can be combined with an active ingredient and sprayed onto foliage or other items in the treatment area. Various attractants are known which influence a pest's behavior as a pest's search for food, oviposition, or mating sites, or mates. Attractants useful in the methods and compositions described herein include, for example, eugenol, phenethyl propionate, ethyl dimethylisobutyl-cyclopropane carboxylate, propyl benszodioxancarboxylate, cis-7,8-epoxy-2-methyloctadecane, trans-8,trans-0-dodecadienol, cis-9-tetradecenal (with cis-11-hexadecenal), trans-11-tetradecenal, cis-11-hexadecenal, (Z)-11,12-hexadecadienal, cis-7-dodecenyl acetate, cis-8-dodecenyul acetate, cis-9-dodecenyl acetate, cis-9-tetradecenyl acetate, cis-11-tetradecenyl acetate, trans-11-tetradecenyl acetate (with cis-11), cis-9,trans-11-tetradecadienyl acetate (with cis-9,trans-12), cis-9,trans-1 2-tetradecadienyl acetate, cis-7,cis-11-hexadecadienyl acetate (with cis-7,trans-11), cis-3,cis-13-octadecadienyl acetate, trans-3,cis-13-octadecadienyl acetate, anethole and isoamyl salicylate.

For further information on agricultural formulations, see “Chemistry and Technology of Agrochemical Formulations” edited by D. A. Knowles, copyright 1998 by Kluwer Academic Publishers. Also see “Insecticides in Agriculture and Environment-Retrospects and Prospects” by A. S. Perry, I. Yamamoto, I. Ishaaya, and R. Perry, copyright 1998 by Springer-Verlag.

### III. Exogenous Polypeptides

The present invention includes plant messenger packs (PMPs) and PMP compositions wherein the PMP encapsulates an exogenous polypeptide. The exogenous polypeptide may be enclosed within the PMP, e.g., located inside the lipid membrane structure, e.g., separated from the surrounding material or solution by both leaflets of a lipid bilayer. In some aspects, the encapsulated exogenous polypeptide may interact or associate with the inner lipid membrane of the PMP. In some aspects, the encapsulated exogenous polypeptide may interact or associate with the outer lipid membrane of the PMP. The exogenous polypeptide may, in some instances, be intercalated with the lipid membrane structure. In some instances, the exogenous polypeptide has an extraluminal portion. In some instances, the exogenous polypeptide is conjugated to the outer surface of the lipid membrane structure, e.g., using click chemistry.

The exogenous polypeptide may be a polypeptide that does not naturally occur in a plant EV. Alternatively, the exogenous polypeptide may be a polypeptide that naturally occurs in a plant EV, but that is encapsulated in a PMP in an amount not found in a naturally occurring plant extracellular vesicle. The exogenous polypeptide may, in some instances, naturally occur in the plant from which the PMP is derived. In other instances, the exogenous polypeptide does not naturally occur in the plant from which the PMP is derived. The exogenous polypeptide may be artificially expressed in the plant from which the PMP is derived, e.g., may be a heterologous polypeptide. The exogenous polypeptide may be derived from another organism. In some aspects, the exogenous polypeptide is loaded into the PMP, e.g., using one or more of sonication, electroporation, lipid extraction, and lipid extrusion.

Polypeptides included herein may include naturally occurring polypeptides or recombinantly produced variants. In some instances, the polypeptide may be a functional fragments or variants thereof (e.g., an enzymatically active fragment or variant thereof). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a polypeptide of interest.

The polypeptides described herein may be formulated in a composition for any of the uses described herein. The compositions disclosed herein may include any number or type (e.g., classes) of polypeptides, such as at least about any one of 1 polypeptide, 2, 3, 4, 5, 10, 15, 20, or more polypeptides. A suitable concentration of each polypeptide in the composition depends on factors such as efficacy, stability of the polypeptide, number of distinct polypeptides in the composition, the formulation, and methods of application of the composition. In some instances, each polypeptide in a liquid composition is from about 0.1 ng/mL to about 100 mg/mL. In some instances, each polypeptide in a solid composition is from about 0.1 ng/g to about 100 mg/g.

Methods of making a polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Methods for producing a polypeptide involve expression in plant cells, although recombinant proteins can also be produced using insect cells, yeast, bacteria, mammalian cells, or other cells under the control of appropriate promoters. Mammalian expression vectors may comprise nontranscribed elements such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture a recombinant polypeptide agent. Examples of mammalian expression systems include CHO cells, COS cells, HeLA and BHK cell lines. Processes of host cell culture for production of protein therapeutics are described in, e.g., Zhou and Kantardjieff (Eds.), Mammalian Cell Cultures for Biologics Manufacturing (Advances in Biochemical Engineering/Biotechnology), Springer (2014). Purification of proteins is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012). Alternatively, the polypeptide may be a chemically synthesized polypeptide.

In some instances, the PMP includes an antibody or antigen binding fragment thereof. For example, an agent described herein may be an antibody that blocks or potentiates activity and/or function of a component of the pathogen. The antibody may act as an antagonist or agonist of a polypeptide (e.g., enzyme or cell receptor) in the pathogen. The making and use of antibodies against a target antigen in a pathogen is known in the art. See, for example, Zhiqiang An (Ed.), Therapeutic Monoclonal Antibodies: From Bench to Clinic, 1st Edition, Wiley, 2009 and also Greenfield (Ed.), Antibodies: A Laboratory Manual, 2nd Edition, Cold Spring Harbor Laboratory Press, 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

The exogenous polypeptide may be released from the PMP in the target cell. In some aspects, the exogenous polypeptide exerts activity in the cytoplasm of the target cell or in the nucleus of the target cell. The exogenous polypeptide may be translocated to the nucleus of the target cell.

In some aspects, uptake by a cell of the exogenous polypeptide encapsulated by the PMP is increased relative to uptake of the exogenous polypeptide not encapsulated by a PMP.

In some aspects, the effectiveness of the exogenous polypeptide encapsulated by the PMP is increased relative to the effectiveness of the exogenous polypeptide not encapsulated by a PMP.

### A. Therapeutic Agents

The exogenous polypeptide may be a therapeutic agent, e.g., an agent used for the prevention or treatment of a condition or a disease. In some aspects, the disease is a cancer, an autoimmine condition, or a metabolic disorder.

In some examples, the therapeutic agent is a peptide (e.g., a naturally occurring peptide, a recombinant peptide, or a synthetic peptide) or a protein (e.g., a naturally occurring protein, a recombinant protein, or a synthetic protein). In some examples, the protein is a fusion protein.

In some examples, the polypeptide is endogenous to the organism (e.g., mammal) to which the PMP is delivered. In other examples, the polypeptide is not endogenous to the organism.

In some examples, the therapeutic agent is an antibody (e.g., a monoclonal antibody, e.g., a monospecific, bispecific, or multispecific monoclonal antibody) or an antigen-binding fragment thereof (e.g., an scFv, (scFv)2, Fab, Fab′, and F(ab′)2, F(ab1)2, Fv, dAb, and Fd fragment, or a diabody), a nanobody, a conjugated antibody, or an antibody-related polypeptide.

In some examples, the therapeutic agent is an antimicrobial, antibacterial, antifungal, antinematicidal, antiparasitic, or antiviral polypeptide.

In some examples, the therapeutic agent is an allergenic, an allergen, or an antigen.

In some examples, the therapeutic agent is a vaccine (e.g., a conjugate vaccine, an inactivated vaccine, or a live attenuated vaccine),

In some examples, the therapeutic agent is an enzyme, e.g., a metabolic recombinase, a helicase, an integrase, a RNAse, a DNAse, an ubiquitination protein. In some examples, the enzyme is a recombinant enzyme.

In some examples, the therapeutic agent is a gene editing protein, e.g., a component of a CRISPR-Cas system, TALEN, or zinc finger.

In some examples, the therapeutic agent is any one of a cytokine, a hormone, a signaling ligand, a transcription factor, a receptor, a receptor antagonist, a receptor agonist, a blocking or neutralizing polypeptide, a riboprotein, or a chaperone.

In some examples, the therapeutic agent is a pore-forming protein, a cell-penetrating peptide, a cell-penetrating peptide inhibitor, or a proteolysis targeting chimera (PROTAC).

In some examples, the therapeutic agent is any one of an aptamer, a blood derivative, a cell therapy, or an immunotherapy (e.g., a cellular immunotherapy.

In some aspects, the therapeutic agent is a protein or peptide therapeutic with enzymatic activity, regulatory activity, or targeting activity, e.g., a protein or peptide with activity that affects one or more of endocrine and growth regulation, metabolic enzyme deficiencies, hematopoiesis, hemostasis and thrombosis; gastrointestinal-tract disorders; pulmonary disorders; immunodeficiencies and/or immunoregulation; fertility; aging (e.g., anti-aging activity); autophagy regulation; epigenetic regulation; oncology; or infectious diseases (e.g., anti-microbial peptides, anti-fungals, or anti-virals).

In some aspects, the therapeutic agent is a protein vaccine, e.g., a vaccine for use in protecting against a deleterious foreign agent, treating an autoimmune disease, or treating cancer (e.g., a neoantigen).

In some examples, the polypeptide is globular, fibrous, or disordered.

In some examples, the polypeptide has a size of less than 1, less than 2, less than 5, less than 10, less than 15, less than 20, less than 30, less than 40, less than 50, less than 60, less than 70, less than 80, less than 90, or less than 100 kD, e.g., has a size of 1-50 kD (e.g., 1-10, 10-20, 20-30, 30-40, or 40-50 kD) or 50-100 kD (e.g., 50-60, 60-70, 70-80, 80-90, or 90-100 kD).

In some examples, the polypeptide has an overall charge that is positive, negative, or neutral. The polypeptide may be modified such that the overall charge is altered, e.g., modified by adding one or more charged amino acids, for example, one or more (for example, 1-10 or 5-10) positively or negatively charged amino acids, such as an arginine tail (e.g., 5-10 arginine residues) to the N-terminus or C-terminus of the polypeptide.

In some aspects, the disease is diabetes, e.g., diabetes mellitus, e.g., Type 1 diabetes mellitus. In some aspects, diabetes is treated by administering to a patient an effective amount of a composition comprising a plurality of PMPs, wherein one or more exogenous polypeptides are encapsulated by the PMP. In some aspects, the administration of the plurality of PMPs lowers the blood sugar of the subject. In some aspects, the therapeutic agent is insulin.

In some examples, the therapeutic agent is an antibody shown in Table 1, a peptide shown in Table 2, an enzyme shown in Table 3, or a protein shown in Table 4.

TABLE 1
Antibodies

| Broad class | Molecule Type | Drug Name |
|---|---|---|
| Antibody | Monoclonal Antibody | 1D-09C3 |
| Antibody | Monoclonal Antibody Conjugated | 212 Pb-TCMC-Trastuzumab |
| Antibody | Monoclonal Antibody | 2141 V-11 |
| Antibody | Monoclonal Antibody | 3BNC-117 |
| Antibody | Monoclonal Antibody | 3BNC-117LS |
| Antibody | Monoclonal Antibody | 8H-9 |
| Antibody | Monoclonal Antibody Conjugated | A-166 |
| Antibody | Bispecific Monoclonal Antibody | A-337 |
| Antibody | Monoclonal Antibody | AB-011 |
| Antibody | Monoclonal Antibody | AB-022 |
| Antibody | Monoclonal Antibody | AB-023 |
| Antibody | Monoclonal Antibody | AB-154 |
| Antibody | Monoclonal Antibody | abagovomab |
| Antibody | Monoclonal Antibody Conjugated | ABBV-011 |
| Antibody | Monoclonal Antibody | ABBV-0805 |
| Antibody | Monoclonal Antibody Conjugated | ABBV-085 |
| Antibody | Monoclonal Antibody | ABBV-151 |
| Antibody | Monoclonal Antibody Conjugated | ABBV-155 |
| Antibody | Bispecific Monoclonal Antibody | ABBV-184 |
| Antibody | Monoclonal Antibody Conjugated | ABBV-321 |
| Antibody | Monoclonal Antibody Conjugated | ABBV-3373 |
| Antibody | Monoclonal Antibody | ABBV-368 |
| Antibody | Monoclonal Antibody | ABBV-927 |
| Antibody | Monoclonal Antibody | abciximab |
| Antibody | Monoclonal Antibody | abelacimab [INN] |
| Antibody | Monoclonal Antibody Conjugated | AbGn-107 |
| Antibody | Monoclonal Antibody | AbGn-168H |
| Antibody | Monoclonal Antibody | abituzumab |
| Antibody | Monoclonal Antibody | ACT-017 |
| Antibody | Monoclonal Antibody Conjugated | Actimab-A |
| Antibody | Monoclonal Antibody Conjugated | Actimab-M |
| Antibody | Cellular Immunotherapy; Gene | ACTR-087 + SEA-BCMA |
| | Therapy; Monoclonal Antibody | |
| Antibody | Cellular Immunotherapy; Gene | ACTR-707 |
| | Therapy; Monoclonal Antibody | |
| Antibody | Monoclonal Antibody | adalimumab |
| Antibody | Monoclonal Antibody | adalimumab biosimilar |
| Antibody | Monoclonal Antibody; Small | adavosertib + durvalumab |
| | Molecule | |
| Antibody | Monoclonal Antibody Conjugated | ADCT-602 |
| Antibody | Antibody | adder [Vipera bents] antivenom |
| Antibody | Monoclonal Antibody | ADG-106 |
| Antibody | Monoclonal Antibody | ADG-116 |
| Antibody | Monoclonal Antibody | adrecizumab |
| Antibody | Monoclonal Antibody | aducanumab |
| Antibody | Monoclonal Antibody | Aerucin |
| Antibody | Bispecific Monoclonal Antibody | AFM-13 |
| Antibody | Monoclonal Antibody | AGEN-1181 |
| Antibody | Monoclonal Antibody | AGEN-2373 |
| Antibody | Monoclonal Antibody Conjugated | AGS-16C3F |
| Antibody | Monoclonal Antibody | AGS-1C4D4 |
| Antibody | Monoclonal Antibody Conjugated | AGS-62P1 |
| Antibody | Monoclonal Antibody | AHM |
| Antibody | Monoclonal Antibody | AIMab-7195 |
| Antibody | Monoclonal Antibody | AK-002 |
| Antibody | Monoclonal Antibody | AK-101 |
| Antibody | Bispecific Monoclonal Antibody | AK-104 |
| Antibody | Monoclonal Antibody | AK-111 |
| Antibody | Bispecific Monoclonal Antibody | AK-112 |
| Antibody | Monoclonal Antibody | AL-001 |
| Antibody | Monoclonal Antibody | AL-002 |
| Antibody | Monoclonal Antibody | AL-003 |
| Antibody | Monoclonal Antibody | AL-101 |
| Antibody | Monoclonal Antibody | alemtuzumab |
| Antibody | Monoclonal Antibody | alirocumab |
| Antibody | Monoclonal Antibody Conjugated | ALTP-7 |
| Antibody | Bispecific Monoclonal Antibody | ALXN-1720 |
| Antibody | Antibody | AMAG-423 |
| Antibody | Monoclonal Antibody | amatuximab |
| Antibody | Bispecific Monoclonal Antibody | AMG-160 |
| Antibody | Bispecific Monoclonal Antibody | AMG-211 |
| Antibody | Monoclonal Antibody Conjugated | AMG-224 |
| Antibody | Monoclonal Antibody | AMG-301 |
| Antibody | Bispecific Monoclonal Antibody | AMG-330 |
| Antibody | Monoclonal Antibody | AMG-404 |
| Antibody | Bispecific Monoclonal Antibody | AMG-420 |
| Antibody | Bispecific Monoclonal Antibody | AMG-424 |
| Antibody | Bispecific Monoclonal Antibody | AMG-427 |
| Antibody | Bispecific Monoclonal Antibody | AMG-509 |
| Antibody | Monoclonal Antibody | AMG-529 |
| Antibody | Bispecific Monoclonal Antibody | AMG-673 |
| Antibody | Bispecific Monoclonal Antibody | AMG-701 |
| Antibody | Monoclonal Antibody | AMG-714 |
| Antibody | Bispecific Monoclonal Antibody | AMG-757 |
| Antibody | Monoclonal Antibody | AMG-820 |
| Antibody | Bispecific Monoclonal Antibody | AMV-564 |
| Antibody | Monoclonal Antibody | ANB-019 |
| Antibody | Monoclonal Antibody | andecaliximab |
| Antibody | Monoclonal Antibody Conjugated | anetumab ravtansine |
| Antibody | Monoclonal Antibody | anifrolumab |
| Antibody | Antibody | anthrax immune globulin (human) |
| Antibody | Antibody | anti-thymocyte globulin (equine) |
| Antibody | Antibody | anti-thymocyte globulin (rabbit) |
| | | antivenin latrodectus equine |
| Antibody | Antibody | immune F(ab)2 |
| Antibody | Monoclonal Antibody | ANX-005 |
| Antibody | Monoclonal Antibody | ANX-007 |
| Antibody | Monoclonal Antibody | AP-101 |
| Antibody | Monoclonal Antibody | apitegromab |
| Antibody | Monoclonal Antibody | APL-501 |
| Antibody | Monoclonal Antibody | APL-502 |
| Antibody | Bispecific Monoclonal Antibody | APVO-436 |
| Antibody | Monoclonal Antibody | APX-003 |
| Antibody | Monoclonal Antibody | APX-005M |
| Antibody | Monoclonal Antibody | ARGX-109 |
| Antibody | Monoclonal Antibody | ARP-1536 |
| Antibody | Monoclonal Antibody Conjugated | ARX-788 |
| Antibody | Monoclonal Antibody | ascrinvacumab |
| Antibody | Monoclonal Antibody | ASLAN-004 |
| Antibody | Monoclonal Antibody | ASP-1650 |
| Antibody | Monoclonal Antibody | ASP-6294 |
| Antibody | Monoclonal Antibody | ASP-8374 |
| Antibody | Monoclonal Antibody | AT-1501 |
| Antibody | Monoclonal Antibody | atezolizumab |
| Antibody | Monoclonal Antibody | ATI-355 |
| Antibody | Monoclonal Antibody Conjugated | ATL-101 |
| Antibody | Bispecific Monoclonal Antibody | ATOR-1015 |
| Antibody | Monoclonal Antibody | ATOR-1017 |
| Antibody | Monoclonal Antibody | ATRC-101 |
| Antibody | Monoclonal Antibody | Atrosab |
| Antibody | Monoclonal Antibody Conjugated | Aurixim |
| Antibody | Monoclonal Antibody | AV-1 |
| Antibody | Monoclonal Antibody | avelumab |
| Antibody | Monoclonal Antibody Conjugated | AVID-100 |
| Antibody | Monoclonal Antibody Conjugated | AVID-200 |
| Antibody | Monoclonal Antibody | axatilimab |
| Antibody | Monoclonal Antibody | B-001 |
| Antibody | Monoclonal Antibody | balstilimab |
| Antibody | Monoclonal Antibody | basiliximab |
| Antibody | Monoclonal Antibody | BAT-4406 |
| Antibody | Monoclonal Antibody | batoclimab |
| Antibody | Monoclonal Antibody | bavituximab |
| Antibody | Monoclonal Antibody | BAY-1093884 |
| Antibody | Monoclonal Antibody | BAY-1834942 |
| Antibody | Monoclonal Antibody | BAY-1905254 |
| Antibody | Monoclonal Antibody Conjugated | BAY-2287411 |
| Antibody | Monoclonal Antibody Conjugated | BAY-2315497 |
| Antibody | Monoclonal Antibody Conjugated | BB-1701 |
| Antibody | Monoclonal Antibody Conjugated | BC-8SA |
| Antibody | Monoclonal Antibody Conjugated | BC-8Y90 |
| Antibody | Monoclonal Antibody | BCBA-445 |
| Antibody | Monoclonal Antibody | BCD-089 |
| Antibody | Monoclonal Antibody | BCD-096 |
| Antibody | Bispecific Monoclonal Antibody | BCD-121 |
| Antibody | Monoclonal Antibody | BCD-132 |
| Antibody | Monoclonal Antibody | BCD-145 |
| Antibody | Monoclonal Antibody | BCD-217 |
| Antibody | Monoclonal Antibody | begelomab |
| Antibody | Monoclonal Antibody Conjugated | belantamab mafodotin |
| Antibody | Monoclonal Antibody | belimumab |
| Antibody | Monoclonal Antibody | bemarituzumab |
| Antibody | Monoclonal Antibody | benralizumab |
| Antibody | Monoclonal Antibody | bentracimab |
| Antibody | Monoclonal Antibody | bermekimab |
| Antibody | Monoclonal Antibody | bertilimumab |
| Antibody | Monoclonal Antibody Conjugated | Betalutin |
| Antibody | Monoclonal Antibody | bevacizumab |
| Antibody | Monoclonal Antibody | bevacizumab biosimilar |
| Antibody | Monoclonal Antibody | bezlotoxumab |
| Antibody | Monoclonal Antibody | BG-00011 |
| Antibody | Monoclonal Antibody | BGB-149 |
| Antibody | Monoclonal Antibody | BHQ-880 |
| Antibody | Monoclonal Antibody | BI-1206 |
| Antibody | Monoclonal Antibody | BI-201 |
| Antibody | Monoclonal Antibody | BI-505 |
| Antibody | Monoclonal Antibody | BI-655064 |
| Antibody | Monoclonal Antibody | BI-655088 |
| Antibody | Monoclonal Antibody | BI-754091 |
| Antibody | Monoclonal Antibody | BI-754111 |
| Antibody | Monoclonal Antibody | BI-836826 |
| Antibody | Monoclonal Antibody | BI-836858 |
| Antibody | Bispecific Monoclonal Antibody | BI-836880 |
| Antibody | Monoclonal Antibody Conjugated | BIIB-015 |
| Antibody | Monoclonal Antibody | BIIB-059 |
| Antibody | Monoclonal Antibody | BIIB-076 |
| Antibody | Monoclonal Antibody | bimagrumab |
| Antibody | Monoclonal Antibody | bimekizumab |
| Antibody | Monoclonal Antibody | birtamimab |
| Antibody | Bispecific Monoclonal Antibody | Bispecific Monoclonal Antibody to |
| | | Agonize CD3 for Acute Myelocytic |
| | | Leukemia |
| Antibody | Bispecific Monoclonal Antibody | Bispecific Monoclonal Antibody to |
| | | Inhibit HIV 1 Env for HIV Infections |
| Antibody | Bispecific Monoclonal Antibody | Bispecific Monoclonal Antibody to |
| | | Target CD3 and FLT3 for Acute |
| | | Myelocytic Leukemia, Acute |
| | | Lymphocytic Leukemia and |
| | | Myelodysplastic Syndrome |
| Antibody | Bispecific Monoclonal Antibody | Bispecific Monoclonal Antibody to |
| | | Target GD2 and CD3 for Oncology |
| Antibody | Bispecific Monoclonal Antibody | Bispecific Monoclonal Antibody to |
| | | Target PD-L1 and CTLA4 for |
| | | Pancreatic Ductal Adenocarcinoma |
| Antibody | Monoclonal Antibody | BIVV-020 |
| Antibody | Monoclonal Antibody | BIW-8962 |
| Antibody | Antibody | black widow spider [Latrodectus |
| | | mactans] antivenom [equine] |
| Antibody | Monoclonal Antibody | bleselumab |
| Antibody | Bispecific Monoclonal Antibody | blinatumomab |
| Antibody | Monoclonal Antibody Conjugated | BMS-936561 |
| Antibody | Monoclonal Antibody | BMS-986012 |
| Antibody | Monoclonal Antibody Conjugated | BMS-986148 |
| Antibody | Monoclonal Antibody | BMS-986156 |
| Antibody | Monoclonal Antibody | BMS-986178 |
| Antibody | Monoclonal Antibody | BMS-986179 |
| Antibody | Monoclonal Antibody | BMS-986207 |
| Antibody | Monoclonal Antibody | BMS-986218 |
| Antibody | Monoclonal Antibody | BMS-986226 |
| Antibody | Monoclonal Antibody | BMS-986253 |
| Antibody | Monoclonal Antibody | BMS-986258 |
| Antibody | Monoclonal Antibody | BNC-101 |
| Antibody | Monoclonal Antibody | BOS-161721 |
| Antibody | Antibody | botulism immune globulin |
| Antibody | Monoclonal Antibody | brazikumab |
| Antibody | Monoclonal Antibody Conjugated | brentuximab vedotin |
| Antibody | Monoclonal Antibody | BrevaRex MAb-AR20.5 |
| Antibody | Monoclonal Antibody | briakinumab |
| Antibody | Monoclonal Antibody | brodalumab |
| Antibody | Monoclonal Antibody | brolucizumab |
| Antibody | Monoclonal Antibody | BT-063 |
| Antibody | Antibody | BT-084 |
| Antibody | Antibody | BT-086 |
| Antibody | Antibody | BT-595 |
| Antibody | Monoclonal Antibody | BTI-322 |
| Antibody | Bispecific Monoclonal Antibody | BTRC-4017A |
| Antibody | Monoclonal Antibody | budigalimab |
| Antibody | Monoclonal Antibody | burosumab |
| Antibody | Monoclonal Antibody | BVX-20 |
| Antibody | Monoclonal Antibody | cabiralizumab |
| Antibody | Monoclonal Antibody | CAEL-101 |
| Antibody | Monoclonal Antibody | CAL |
| Antibody | Monoclonal Antibody Conjugated | camidanlumab tesirine |
| Antibody | Monoclonal Antibody | camrelizumab |
| Antibody | Monoclonal Antibody | canakinumab |
| Antibody | Monoclonal Antibody Conjugated | cantuzumab mertansine |
| Antibody | Monoclonal Antibody | caplacizumab |
| Antibody | Monoclonal Antibody | carotuximab |
| Antibody | Bispecific Monoclonal Antibody | catumaxomab |
| Antibody | Monoclonal Antibody | CBP-201 |
| Antibody | Bispecific Monoclonal Antibody | CC-1 |
| Antibody | Monoclonal Antibody | CC-90002 |
| Antibody | Monoclonal Antibody | CC-90006 |
| Antibody | Bispecific Monoclonal Antibody | CC-93269 |
| Antibody | Monoclonal Antibody Conjugated | CC-99712 |
| Antibody | Monoclonal Antibody Conjugated | CCW-702 |
| Antibody | Monoclonal Antibody | CDX-3379 |
| Antibody | Cellular Immunotherapy; | Cellular Immunotherapy + |
| | Recombinant Protein | edodekin alfa |
| Antibody | Monoclonal Antibody | cemiplimab |
| Antibody | Monoclonal Antibody | cendakimab |
| Antibody | Monoclonal Antibody | CERC-002 |
| Antibody | Monoclonal Antibody | CERC-007 |
| Antibody | Monoclonal Antibody | certolizumab pegol |
| Antibody | Monoclonal Antibody | certolizumab pegol biosimilar |
| Antibody | Monoclonal Antibody | cetrelimab |
| Antibody | Monoclonal Antibody | cetuximab |
| Antibody | Monoclonal Antibody | cetuximab biosimilar |
| Antibody | Monoclonal Antibody Conjugated | cetuximab sarotalocan |
| Antibody | Monoclonal Antibody | CHOH-01 |
| Antibody | Bispecific Monoclonal Antibody | cibisatamab |
| Antibody | Monoclonal Antibody | cinpanemab |
| Antibody | Monoclonal Antibody | CIS-43 |
| Antibody | Monoclonal Antibody | CJM-112 |
| Antibody | Monoclonal Antibody | clazakizumab |
| Antibody | Monoclonal Antibody Conjugated | clivatuzumab tetraxetan |
| Antibody | Monoclonal Antibody | CM-101 |
| Antibody | Monoclonal Antibody | CNTO-6785 |
| Antibody | Monoclonal Antibody | codrituzumab |
| Antibody | Monoclonal Antibody Conjugated | cofetuzumab pelidotin |
| Antibody | Monoclonal Antibody | COM-701 |
| Antibody | Monoclonal Antibody | concizumab |
| Antibody | Monoclonal Antibody | COR-001 |
| Antibody | Antibody | coral snake [Micrurus] (polyvalent) |
| | | immunoglobulin F (ab) 2 + Fab |
| | | immunoglobulin G antivenom |
| Antibody | Monoclonal Antibody | cosibelimab |
| Antibody | Monoclonal Antibody | CPI-006 |
| Antibody | Monoclonal Antibody | crenezumab |
| Antibody | Monoclonal Antibody | crizanlizumab |
| Antibody | Monoclonal Antibody | crovalimab |
| Antibody | Monoclonal Antibody | CS-1001 |
| Antibody | Monoclonal Antibody | CS-1003 |
| Antibody | Monoclonal Antibody | CSL-311 |
| Antibody | Monoclonal Antibody | CSL-324 |
| Antibody | Monoclonal Antibody | CSL-346 |
| Antibody | Monoclonal Antibody | CSL-360 |
| Antibody | Monoclonal Antibody | CTX-471 |
| Antibody | Monoclonal Antibody | cusatuzumab |
| Antibody | Antibody | Cutaquig |
| Antibody | Antibody | Cuvitru |
| Antibody | Monoclonal Antibody | CX-072 |
| Antibody | Monoclonal Antibody Conjugated | CX-2009 |
| Antibody | Monoclonal Antibody Conjugated | CX-2029 |
| Antibody | Monoclonal Antibody | Cyto-111 |
| Antibody | Antibody | cytomegalovirus immune globulin |
| | | (human) |
| Antibody | Monoclonal Antibody; Small | dabrafenib mesylate + |
| | Molecule | panitumumab + trametinib dimethyl |
| | | sulfoxide |
| Antibody | Monoclonal Antibody | daclizumab |
| Antibody | Monoclonal Antibody | dalotuzumab |
| Antibody | Antisense Oligonucleotide; | danvatirsen + durvalumab |
| | Monoclonal Antibody | |
| Antibody | Monoclonal Antibody | dapirolizumab pegol |
| Antibody | Monoclonal Antibody | daratumumab |
| Antibody | Monoclonal Antibody | daxdilimab |
| Antibody | Monoclonal Antibody | DE-098 |
| Antibody | Antibody | death adder [Acanthophis |
| | | antarcticus] antivenom [equine] |
| Antibody | Monoclonal Antibody | demcizumab |
| Antibody | Monoclonal Antibody | denosumab |
| Antibody | Monoclonal Antibody | denosumab biosimilar |
| Antibody | Monoclonal Antibody | depatuxizumab |
| Antibody | Monoclonal Antibody Conjugated | depatuxizumab mafodotin |
| Antibody | Monoclonal Antibody | dezamizumab |
| Antibody | Antibody | digoxin immune Fab (ovine) |
| Antibody | Monoclonal Antibody | dilpacimab |
| Antibody | Monoclonal Antibody | dinutuximab |
| Antibody | Monoclonal Antibody | dinutuximab beta |
| Antibody | Monoclonal Antibody | diridavumab |
| Antibody | Monoclonal Antibody | DKN-01 |
| Antibody | Monoclonal Antibody Conjugated | DNP-001 |
| Antibody | Monoclonal Antibody | DNP-002 |
| Antibody | Monoclonal Antibody | domagrozumab |
| Antibody | Monoclonal Antibody | donanemab |
| Antibody | Monoclonal Antibody | dostarlimab |
| Antibody | Monoclonal Antibody Conjugated | DP-303c |
| Antibody | Monoclonal Antibody Conjugated | DS-1062 |
| Antibody | Monoclonal Antibody Conjugated | DS-7300 |
| Antibody | Monoclonal Antibody | DS-8273 |
| Antibody | Monoclonal Antibody | dupilumab |
| Antibody | Monoclonal Antibody | durvalumab |
| Antibody | Monoclonal Antibody | durvalumab + monalizumab |
| Antibody | Monoclonal Antibody | durvalumab + oleclumab |
| Antibody | Monoclonal Antibody; Small | durvalumab + selumetinib sulfate |
| | Molecule | |
| Antibody | Monoclonal Antibody | durvalumab + tremelimumab |
| Antibody | Monoclonal Antibody | EBI-031 |
| Antibody | Monoclonal Antibody | eculizumab |
| Antibody | Monoclonal Antibody | eculizumab biosimilar |
| Antibody | Monoclonal Antibody | edrecolomab |
| Antibody | Monoclonal Antibody | efalizumab |
| Antibody | Monoclonal Antibody | efgartigimod alfa |
| Antibody | Monoclonal Antibody | efungumab |
| Antibody | Monoclonal Antibody | elezanumab |
| Antibody | Monoclonal Antibody | elgemtumab |
| Antibody | Monoclonal Antibody | elipovimab |
| Antibody | Monoclonal Antibody | elotuzumab |
| Antibody | Monoclonal Antibody | emactuzumab |
| Antibody | Monoclonal Antibody | emapalumab |
| Antibody | Bispecific Monoclonal Antibody | emicizumab |
| Antibody | Monoclonal Antibody | enamptcumab |
| Antibody | Monoclonal Antibody Conjugated | enapotamab vedotin |
| Antibody | Monoclonal Antibody Conjugated | enfortumab vedotin |
| Antibody | Monoclonal Antibody | enoblituzumab |
| Antibody | Monoclonal Antibody | ensituximab |
| Antibody | Bispecific Monoclonal Antibody | epcoritamab |
| Antibody | Monoclonal Antibody | epratuzumab |
| Antibody | Monoclonal Antibody | eptinezumab |
| Antibody | Monoclonal Antibody | erenumab |
| Antibody | Bispecific Monoclonal Antibody | ertumaxomab |
| Antibody | Bispecific Monoclonal Antibody | ERY-974 |
| Antibody | Monoclonal Antibody | etaracizumab |
| Antibody | Monoclonal Antibody | etigilimab |
| Antibody | Monoclonal Antibody | etokimab |
| Antibody | Monoclonal Antibody | etrolizumab |
| Antibody | Monoclonal Antibody | evinacumab |
| Antibody | Monoclonal Antibody | evolocumab |
| Antibody | Monoclonal Antibody; Synthetic | exenatide + ND-017 |
| | Peptide | |
| Antibody | Monoclonal Antibody | F-598 |
| Antibody | Bispecific Monoclonal Antibody | faricimab |
| Antibody | Monoclonal Antibody | farletuzumab |
| Antibody | Monoclonal Antibody | fasinumab |
| Antibody | Monoclonal Antibody | FAZ-053 |
| Antibody | Monoclonal Antibody | FB-704A |
| Antibody | Monoclonal Antibody | FB-825 |
| Antibody | Antibody | FBF-001 |
| Antibody | Antibody | Ferritarg |
| Antibody | Monoclonal Antibody Conjugated | FF-21101 |
| Antibody | Monoclonal Antibody | ficlatuzumab |
| Antibody | Bispecific Monoclonal Antibody | fiotetuzumab |
| Antibody | Monoclonal Antibody | FLYSYN |
| Antibody | Monoclonal Antibody | FM-101 |
| Antibody | Monoclonal Antibody Conjugated | FOR-46 |
| Antibody | Monoclonal Antibody | foralumab |
| Antibody | Monoclonal Antibody | FR-104 |
| Antibody | Monoclonal Antibody | fremanezumab |
| Antibody | Monoclonal Antibody | fresolimumab |
| Antibody | Monoclonal Antibody | FS-102 |
| Antibody | Bispecific Monoclonal Antibody | FS-118 |
| Antibody | Monoclonal Antibody | fulranumab |
| Antibody | Monoclonal Antibody | galcanezumab |
| Antibody | Monoclonal Antibody | ganitumab |
| Antibody | Monoclonal Antibody | gantenerumab |
| Antibody | Monoclonal Antibody | garadacimab |
| Antibody | Monoclonal Antibody | garetosmab |
| Antibody | Monoclonal Antibody | gatipotuzumab |
| Antibody | Monoclonal Antibody | GC-1118A |
| Antibody | Monoclonal Antibody | GEM-103 |
| Antibody | Bispecific Monoclonal Antibody | GEM-333 |
| Antibody | Bispecific Monoclonal Antibody | GEM-3PSCA |
| Antibody | Monoclonal Antibody Conjugated | gemtuzumab ozogamicin |
| Antibody | Bispecific Monoclonal Antibody | GEN-1046 |
| Antibody | Monoclonal Antibody | gevokizumab |
| Antibody | Monoclonal Antibody | gimsilumab |
| Antibody | Monoclonal Antibody | girentuximab |
| Antibody | Monoclonal Antibody Conjugated | glembatumumab vedotin |
| Antibody | Monoclonal Antibody | GLS-010 |
| Antibody | Monoclonal Antibody | GMA-102 |
| Antibody | Monoclonal Antibody | GMA-161 |
| Antibody | Monoclonal Antibody | GMA-301 |
| Antibody | Monoclonal Antibody | golimumab |
| Antibody | Monoclonal Antibody | gosuranemab |
| Antibody | Monoclonal Antibody | GR-1501 |
| Antibody | Bispecific Monoclonal Antibody | gremubamab |
| Antibody | Bispecific Monoclonal Antibody | GS-1423 |
| Antibody | Monoclonal Antibody | GSK-1070806 |
| Antibody | Monoclonal Antibody | GSK-2330811 |
| Antibody | Monoclonal Antibody | GSK-2831781 |
| Antibody | Monoclonal Antibody | GSK-3050002 |
| Antibody | Monoclonal Antibody | GSK-3174998 |
| Antibody | Monoclonal Antibody | GSK-3359609 |
| Antibody | Monoclonal Antibody | GSK-3511294 |
| Antibody | Monoclonal Antibody | GT-103 |
| Antibody | Monoclonal Antibody | guselkumab |
| Antibody | Monoclonal Antibody | GWN-323 |
| Antibody | Monoclonal Antibody | H-11 |
| Antibody | Monoclonal Antibody | HAB-21 |
| Antibody | Monoclonal Antibody | HBM-4003 |
| Antibody | Monoclonal Antibody | HDIT-101 |
| Antibody | Antibody | hepatitis B immune globulin |
| | | (human) |
| Antibody | Antibody | hepatitis C virus immune globulin |
| | | (human) |
| Antibody | Monoclonal Antibody | HLX-06 |
| Antibody | Monoclonal Antibody | HLX-07 |
| Antibody | Monoclonal Antibody | HLX-10 |
| Antibody | Monoclonal Antibody | HLX-20 |
| Antibody | Monoclonal Antibody | HPN-217 |
| Antibody | Monoclonal Antibody | HPN-424 |
| Antibody | Monoclonal Antibody | HPN-536 |
| Antibody | Monoclonal Antibody | HS-006 |
| Antibody | Monoclonal Antibody Conjugated | HTI-1066 |
| Antibody | Monoclonal Antibody | Hu8F4 |
| Antibody | Antibody | human immunoglobulin |
| | | antistaphylococcal |
| Antibody | Monoclonal Antibody | ianalumab |
| Antibody | Monoclonal Antibody | ibalizumab |
| Antibody | Monoclonal Antibody | IBI-101 |
| Antibody | Monoclonal Antibody | IBI-188 |
| Antibody | Monoclonal Antibody | IBI-306 |
| Antibody | Bispecific Monoclonal Antibody | IBI-322 |
| Antibody | Monoclonal Antibody Conjugated | ibritumomab tiuxetan |
| Antibody | Monoclonal Antibody | IC-14 |
| Antibody | Monoclonal Antibody | ICT-01 |
| Antibody | Monoclonal Antibody | idarucizumab |
| Antibody | Monoclonal Antibody | ieramilimab |
| Antibody | Monoclonal Antibody | ifabotuzumab |
| Antibody | Monoclonal Antibody | IFX-1 |
| Antibody | Monoclonal Antibody | IGEM-F |
| Antibody | Bispecific Monoclonal Antibody | IGM-2323 |
| Antibody | Antibody | immune globulin (human) |
| Antibody | Antibody | immune globulin (human) 2 |
| Antibody | Bispecific Monoclonal Antibody | INBRX-105 |
| Antibody | Monoclonal Antibody | INCAGN-1876 |
| Antibody | Monoclonal Antibody | INCAGN-1949 |
| Antibody | Monoclonal Antibody | INCAGN-2385 |
| Antibody | Monoclonal Antibody | inclacumab |
| Antibody | Monoclonal Antibody Conjugated | indatuximab ravtansine |
| Antibody | Monoclonal Antibody Conjugated | indusatumab vedotin |
| Antibody | Monoclonal Antibody | inebilizumab |
| Antibody | Monoclonal Antibody | infliximab |
| Antibody | Monoclonal Antibody | infliximab biobetter |
| Antibody | Monoclonal Antibody | infliximab biosimilar |
| Antibody | Monoclonal Antibody | INM-004 |
| Antibody | Monoclonal Antibody | inolimomab |
| Antibody | Monoclonal Antibody Conjugated | inotuzumab ozogamicin |
| Antibody | Monoclonal Antibody Conjugated | Iodine-131-Kab201 |
| Antibody | Monoclonal Antibody Conjugated | Iomab-B |
| Antibody | Monoclonal Antibody | IPH-5401 |
| Antibody | Monoclonal Antibody | ipilimumab |
| Antibody | Monoclonal Antibody | ipilimumab + nivolumab |
| Antibody | Monoclonal Antibody | isatuximab |
| Antibody | Bispecific Monoclonal Antibody | ISB-1302 |
| Antibody | Bispecific Monoclonal Antibody | ISB-1342 |
| Antibody | Monoclonal Antibody | ISB-830 |
| Antibody | Monoclonal Antibody | iscalimab |
| Antibody | Monoclonal Antibody | ISU-104 |
| Antibody | Monoclonal Antibody | itolizumab |
| Antibody | Monoclonal Antibody | ixekizumab |
| Antibody | Monoclonal Antibody | IXTM-200 |
| Antibody | Monoclonal Antibody | JMT-103 |
| Antibody | Monoclonal Antibody | JNJ-0839 |
| Antibody | Monoclonal Antibody | JNJ-3657 |
| Antibody | Monoclonal Antibody | JNJ-4500 |
| Antibody | Bispecific Monoclonal Antibody | JNJ-6372 |
| Antibody | Bispecific Monoclonal Antibody | JNJ-67571244 |
| Antibody | Bispecific Monoclonal Antibody | JNJ-7564 |
| Antibody | Bispecific Monoclonal Antibody | JNJ-7957 |
| Antibody | Bispecific Monoclonal Antibody | JNJ-9178 |
| Antibody | Monoclonal Antibody | JS-004 |
| Antibody | Monoclonal Antibody | JTX-4014 |
| Antibody | Monoclonal Antibody | JY-025 |
| Antibody | Monoclonal Antibody | K-170 |
| Antibody | Monoclonal Antibody | KHK-2823 |
| Antibody | Monoclonal Antibody | KHK-4083 |
| Antibody | Monoclonal Antibody | KHK-6640 |
| Antibody | Monoclonal Antibody Conjugated | Kid EDV |
| Antibody | Monoclonal Antibody | KLA-167 |
| Antibody | Bispecific Monoclonal Antibody | KN-026 |
| Antibody | Bispecific Monoclonal Antibody | KN-046 |
| Antibody | Monoclonal Antibody | KSI-301 |
| Antibody | Monoclonal Antibody | KY-1005 |
| Antibody | Monoclonal Antibody Conjugated | labetuzumab govitecan |
| Antibody | Monoclonal Antibody | lacnotuzumab |
| Antibody | Monoclonal Antibody | lacutamab |
| Antibody | Monoclonal Antibody Conjugated | ladiratuzumab vedotin |
| Antibody | Monoclonal Antibody | lanadelumab |
| Antibody | Monoclonal Antibody | LBL-007 |
| Antibody | Monoclonal Antibody Conjugated | LDOS-47 |
| Antibody | Monoclonal Antibody | lebrikizumab |
| Antibody | Monoclonal Antibody | lecanemab |
| Antibody | Monoclonal Antibody | Lemtrada |
| Antibody | Monoclonal Antibody | lenvervimab |
| Antibody | Monoclonal Antibody | lenzilumab |
| Antibody | Monoclonal Antibody | leronlimab |
| Antibody | Monoclonal Antibody | letolizumab |
| Antibody | Monoclonal Antibody | ligelizumab |
| Antibody | Monoclonal Antibody | lintuzumab |
| Antibody | Monoclonal Antibody; | liraglutide + NN-8828 |
| | Recombinant Peptide | |
| Antibody | Monoclonal Antibody | lirilumab |
| Antibody | Monoclonal Antibody | LKA-651 |
| Antibody | Monoclonal Antibody | LLG-783 |
| Antibody | Monoclonal Antibody | lodapolimab |
| Antibody | Monoclonal Antibody Conjugated | loncastuximab tesirine |
| Antibody | Monoclonal Antibody Conjugated | lorvotuzumab mertansine |
| Antibody | Monoclonal Antibody | LuAF-82422 |
| Antibody | Monoclonal Antibody | LuAF-87908 |
| Antibody | Monoclonal Antibody | lulizumab pegol |
| Antibody | Monoclonal Antibody | lumiliximab |
| Antibody | Monoclonal Antibody | LVGN-6051 |
| Antibody | Monoclonal Antibody | LY-3022855 |
| Antibody | Monoclonal Antibody | LY-3041658 |
| Antibody | Monoclonal Antibody | LY-3127804 |
| Antibody | Bispecific Monoclonal Antibody | LY-3434172 |
| Antibody | Antibody | LY-3435151 |
| Antibody | Antibody | LY-3454738 |
| Antibody | Monoclonal Antibody | LZM-009 |
| Antibody | Bispecific Monoclonal Antibody | M-1095 |
| Antibody | Antibody | M-254 |
| Antibody | Monoclonal Antibody | M-6495 |
| Antibody | Bispecific Monoclonal Antibody | M-802 |
| Antibody | Monoclonal Antibody | mAb-114 |
| Antibody | Monoclonal Antibody | magrolimab |
| Antibody | Monoclonal Antibody | margetuximab |
| Antibody | Monoclonal Antibody | marstacimab |
| Antibody | Monoclonal Antibody | MAU-868 |
| Antibody | Monoclonal Antibody | mavrilimumab |
| Antibody | Bispecific Monoclonal Antibody | MCLA-117 |
| Antibody | Bispecific Monoclonal Antibody | MCLA-145 |
| Antibody | Bispecific Monoclonal Antibody | MCLA-158 |
| Antibody | Monoclonal Antibody | MDX-1097 |
| Antibody | Monoclonal Antibody | MEDI-0618 |
| Antibody | Monoclonal Antibody | MEDI-1341 |
| Antibody | Monoclonal Antibody | MEDI-1814 |
| Antibody | Monoclonal Antibody | MEDI-3506 |
| Antibody | Monoclonal Antibody | MEDI-3617 + tremelimumab |
| Antibody | Monoclonal Antibody | MEDI-5117 |
| Antibody | Monoclonal Antibody Conjugated | MEDI-547 |
| Antibody | Monoclonal Antibody | MEDI-570 |
| Antibody | Bispecific Monoclonal Antibody | MEDI-5752 |
| Antibody | Bispecific Monoclonal Antibody | MEDI-7352 |
| Antibody | Monoclonal Antibody | melrilimab |
| Antibody | Monoclonal Antibody | MEN-1112 |
| Antibody | Monoclonal Antibody | mepolizumab |
| Antibody | Monoclonal Antibody | metelimumab |
| Antibody | Monoclonal Antibody | MG-1113A |
| Antibody | Monoclonal Antibody | MGA-012 |
| Antibody | Monoclonal Antibody | MGB-453 |
| Antibody | Monoclonal Antibody Conjugated | MGC-018 |
| Antibody | Bispecific Monoclonal Antibody | MGD-013 |
| Antibody | Monoclonal Antibody | MIL-62 |
| Antibody | Monoclonal Antibody | milatuzumab |
| Antibody | Monoclonal Antibody | mirikizumab |
| Antibody | Monoclonal Antibody Conjugated | mirvetuximab soravtansine |
| Antibody | Monoclonal Antibody | mitazalimab |
| Antibody | Monoclonal Antibody | MK-1308 |
| Antibody | Monoclonal Antibody | MK-1654 |
| Antibody | Monoclonal Antibody | MK-3655 |
| Antibody | Monoclonal Antibody | MK-4166 |
| Antibody | Monoclonal Antibody | MK-4280 |
| Antibody | Monoclonal Antibody | MK-5890 |
| Antibody | Monoclonal Antibody | mogamulizumab |
| Antibody | Monoclonal Antibody | monalizumab |
| Antibody | Monoclonal Antibody Conjugated | Monoclonal Antibody Conjugate to |
| | | Target CD20 for Leukemia and |
| | | Burkitt Lymphoma |
| Antibody | Monoclonal Antibody Conjugated | Monoclonal Antibody Conjugate to |
| | | Target CD45 for Oncology |
| Antibody | Monoclonal Antibody Conjugated | Monoclonal Antibody Conjugate to |
| | | Target CEA for Metastatic Liver, |
| | | Colorectal Cancer and Solid Tumor |
| Antibody | Monoclonal Antibody Conjugated | Monoclonal Antibody Conjugate to |
| | | Target CEACAM5 for Non Small |
| | | Cell Lung Cancer and Metastatic |
| | | Colorectal Cancer |
| Antibody | Monoclonal Antibody Conjugated | Monoclonal Antibody Conjugated to |
| | | Target EPCAM for Colorectal |
| | | Cancer |
| Antibody | Monoclonal Antibody Conjugated | Monoclonal Antibody Conjugated to |
| | | Target PSMA for Prostate Cancer |
| Antibody | Monoclonal Antibody | Monoclonal Antibody for |
| | | Coronavirus Disease 2019 (COVID- |
| | | 19) |
| Antibody | Monoclonal Antibody | Monoclonal Antibody for Dengue |
| Antibody | Monoclonal Antibody | Monoclonal Antibody to Antagonize |
| | | IL-2R Beta for Celiac Disease, |
| | | Oncology and Tropical Spastic |
| | | Paraparesis |
| Antibody | Monoclonal Antibody | Monoclonal Antibody to Inhibit |
| | | ANXA3 for Hepatocellular |
| | | Carcinoma |
| Antibody | Monoclonal Antibody | Monoclonal Antibody to Inhibit CD4 |
| | | for HIV-1 |
| Antibody | Monoclonal Antibody | Monoclonal Antibody to Inhibit GD2 |
| | | for Oncology |
| Antibody | Monoclonal Antibody | Monoclonal Antibody to Inhibit |
| | | Glycoprotein 120 for HIV-1 |
| | | infections |
| Antibody | Monoclonal Antibody | Monoclonal Antibody to Inhibit IL- |
| | | 17A and IL-17F for Unspecified |
| | | Indication |
| Antibody | Monoclonal Antibody | Monoclonal Antibody to Inhibit PD- |
| | | L1 for Solid Tumor |
| Antibody | Monoclonal Antibody | Monoclonal Antibody to Inhibit PD1 |
| | | for Solid Tumors |
| Antibody | Monoclonal Antibody | Monoclonal Antibody to Inhibit TNF- |
| | | Alpha for Dupuytren's contracture |
| Antibody | Monoclonal Antibody Conjugated | Monoclonal Antibody to Target |
| | | CD66b for Blood Cancer and |
| | | Metabolic Disorders |
| Antibody | Monoclonal Antibody | Monoclonal Antibody to Target |
| | | GP41 for HIV Infections |
| Antibody | Monoclonal Antibody | MOR-106 |
| Antibody | Monoclonal Antibody | MOR-202 |
| Antibody | Monoclonal Antibody Conjugated | MORAb-202 |
| Antibody | Bispecific Monoclonal Antibody | mosunetuzumab |
| Antibody | Monoclonal Antibody Conjugated | moxetumomab pasudotox |
| Antibody | Monoclonal Antibody | MSB-2311 |
| Antibody | Monoclonal Antibody | MSC-1 |
| Antibody | Monoclonal Antibody | MT-2990 |
| Antibody | Monoclonal Antibody | MT-3921 |
| Antibody | Monoclonal Antibody | murlentamab |
| Antibody | Monoclonal Antibody | muromonab-CD3 |
| Antibody | Monoclonal Antibody | MVT-5873 |
| Antibody | Monoclonal Antibody | namilumab |
| Antibody | Monoclonal Antibody Conjugated | naratuximab emtansine |
| Antibody | Monoclonal Antibody | narsoplimab |
| Antibody | Monoclonal Antibody | natalizumab |
| Antibody | Monoclonal Antibody | natalizumab biosimilar |
| Antibody | Bispecific Monoclonal Antibody | navicixizumab |
| Antibody | Monoclonal Antibody | naxitamab |
| Antibody | Monoclonal Antibody | NC-318 |
| Antibody | Monoclonal Antibody | nebacumab |
| Antibody | Monoclonal Antibody | necitumumab |
| Antibody | Monoclonal Antibody | nemolizumab |
| Antibody | Monoclonal Antibody | netakimab |
| Antibody | Monoclonal Antibody | NGM-120 |
| Antibody | Monoclonal Antibody | NI-006 |
| Antibody | Monoclonal Antibody | NI-0101 |
| Antibody | Monoclonal Antibody | nidanilimab |
| Antibody | Monoclonal Antibody | nimacimab |
| Antibody | Monoclonal Antibody | nimotuzumab |
| Antibody | Monoclonal Antibody | nimotuzumab biosimilar |
| Antibody | Monoclonal Antibody | nipocalimab |
| Antibody | Monoclonal Antibody | nirsevimab |
| Antibody | Monoclonal Antibody | NIS-793 |
| Antibody | Monoclonal Antibody | nivolumab |
| Antibody | Monoclonal Antibody Conjugated | NJH-395 |
| Antibody | Bispecific Monoclonal Antibody | NNC-03653769A |
| Antibody | Antibody | NP-024 |
| Antibody | Antibody | NP-025 |
| Antibody | Monoclonal Antibody | NP-137 |
| Antibody | Monoclonal Antibody | NPC-21 |
| Antibody | Bispecific Monoclonal Antibody | NXT-007 |
| Antibody | Monoclonal Antibody | NZV-930 |
| Antibody | Monoclonal Antibody | obexelimab |
| Antibody | Monoclonal Antibody | OBI-888 |
| Antibody | Monoclonal Antibody Conjugated | OBI-999 |
| Antibody | Monoclonal Antibody | obiltoxaximab |
| Antibody | Monoclonal Antibody | obinutuzumab |
| Antibody | Monoclonal Antibody Conjugated | OBT-076 |
| Antibody | Monoclonal Antibody | ocaratuzumab |
| Antibody | Monoclonal Antibody | ocrelizumab |
| Antibody | Bispecific Monoclonal Antibody | odronextamab |
| Antibody | Monoclonal Antibody | ofatumumab |
| Antibody | Monoclonal Antibody | olaratumab |
| Antibody | Monoclonal Antibody | oleclumab |
| Antibody | Monoclonal Antibody | olendalizumab |
| Antibody | Monoclonal Antibody | olinvacimab |
| Antibody | Monoclonal Antibody | olokizumab |
| Antibody | Monoclonal Antibody | omalizumab |
| Antibody | Monoclonal Antibody | omalizumab biosimilar |
| Antibody | Monoclonal Antibody Conjugated | omburtamab |
| Antibody | Monoclonal Antibody | omodenbamab |
| Antibody | Monoclonal Antibody | ONC-392 |
| Antibody | Monoclonal Antibody | ontamalimab |
| Antibody | Monoclonal Antibody | ontuxizumab |
| Antibody | Monoclonal Antibody | opicinumab |
| Antibody | Monoclonal Antibody | oregovomab |
| Antibody | Monoclonal Antibody | orilanolimab |
| Antibody | Monoclonal Antibody | orticumab |
| Antibody | Monoclonal Antibody | OS-2966 |
| Antibody | Monoclonal Antibody | OSE-127 |
| Antibody | Monoclonal Antibody | osocimab |
| Antibody | Monoclonal Antibody | otelixizumab |
| Antibody | Monoclonal Antibody | otilimab |
| Antibody | Monoclonal Antibody | otlertuzumab |
| Antibody | Monoclonal Antibody Conjugated | OTSA-101 |
| Antibody | Monoclonal Antibody Conjugated | OXS-1750 |
| Antibody | Monoclonal Antibody Conjugated | OXS-2050 |
| Antibody | Monoclonal Antibody | ozoralizumab |
| Antibody | Monoclonal Antibody | P-2G12 |
| Antibody | Monoclonal Antibody | pagibaximab |
| Antibody | Monoclonal Antibody | palivizumab |
| Antibody | Monoclonal Antibody | pamrevlumab |
| Antibody | Monoclonal Antibody | panitumumab |
| Antibody | Monoclonal Antibody | panobacumab |
| Antibody | Bispecific Monoclonal Antibody | pasotuxizumab |
| Antibody | Monoclonal Antibody | PAT-SC1 |
| Antibody | Monoclonal Antibody | patritumab |
| Antibody | Monoclonal Antibody | PC-mAb |
| Antibody | Monoclonal Antibody | PD-0360324 |
| Antibody | Monoclonal Antibody | pembrolizumab |
| Antibody | Monoclonal Antibody | pepinemab |
| Antibody | Monoclonal Antibody | pertuzumab |
| Antibody | Monoclonal Antibody | pertuzumab + trastuzumab |
| Antibody | Monoclonal Antibody | PF-04518600 |
| Antibody | Monoclonal Antibody | PF-06480605 |
| Antibody | Antibody | PF-06730512 |
| Antibody | Monoclonal Antibody | PF-06823859 |
| Antibody | Bispecific Monoclonal Antibody | PF-06863135 |
| Antibody | Monoclonal Antibody | pidilizumab |
| Antibody | Antibody | pit viper snake [Crotalidae] |
| | | (polyvalent) immunoglobulin F(ab′)2 |
| | | antivenom [equine] |
| Antibody | Bispecific Monoclonal Antibody | plamotamab |
| Antibody | Monoclonal Antibody | PNT-001 |
| Antibody | Monoclonal Antibody Conjugated | polatuzumab vedotin |
| Antibody | Antibody | PolyCAb |
| Antibody | Monoclonal Antibody | pozelimab |
| Antibody | Monoclonal Antibody | prasinezumab |
| Antibody | Monoclonal Antibody | pritumumab |
| Antibody | Monoclonal Antibody | PRL3-ZUMAB |
| Antibody | Monoclonal Antibody | prolgolimab |
| Antibody | Monoclonal Antibody | PRV-300 |
| Antibody | Bispecific Monoclonal Antibody | PRV-3279 |
| Antibody | Monoclonal Antibody | PRX-004 |
| Antibody | Bispecific Monoclonal Antibody | PSB-205 |
| Antibody | Monoclonal Antibody | PTX-35 |
| Antibody | Monoclonal Antibody | PTZ-329 |
| Antibody | Monoclonal Antibody | PTZ-522 |
| Antibody | Monoclonal Antibody | quetmolimab |
| Antibody | Monoclonal Antibody | QX-002N |
| Antibody | Monoclonal Antibody | R-1549 |
| Antibody | Monoclonal Antibody | rabies immune globulin (human) |
| Antibody | Monoclonal Antibody | racotumomab |
| Antibody | Monoclonal Antibody Conjugated | Radspherin |
| Antibody | Monoclonal Antibody | ramucirumab |
| Antibody | Monoclonal Antibody | ranibizumab |
| Antibody | Monoclonal Antibody | ranibizumab biosimilar |
| Antibody | Monoclonal Antibody | ranibizumab SR |
| Antibody | Monoclonal Antibody | ravagalimab |
| Antibody | Monoclonal Antibody | ravulizumab |
| Antibody | Monoclonal Antibody | ravulizumab next generation |
| Antibody | Monoclonal Antibody | raxibacumab |
| Antibody | Monoclonal Antibody Conjugated | RC-48 |
| Antibody | Monoclonal Antibody | |
| Antibody | Monoclonal Antibody | REGN-3048 |
| Antibody | Monoclonal Antibody | REGN-3051 |
| Antibody | Monoclonal Antibody | REGN-3500 |
| Antibody | Bispecific Monoclonal Antibody | REGN-4018 |
| Antibody | Monoclonal Antibody | REGN-4461 |
| Antibody | Antibody | REGN-5069 |
| Antibody | Bispecific Monoclonal Antibody | REGN-5458 |
| Antibody | Bispecific Monoclonal Antibody | REGN-5459 |
| Antibody | Bispecific Monoclonal Antibody | REGN-5678 |
| Antibody | Monoclonal Antibody | REGN-5713 |
| Antibody | Monoclonal Antibody | REGN-5714 |
| Antibody | Monoclonal Antibody | REGN-5715 |
| Antibody | Monoclonal Antibody | relatlimab |
| Antibody | Monoclonal Antibody | reslizumab |
| Antibody | Antibody | respiratory syncytial virus immune |
| | | globulin (human) |
| Antibody | Monoclonal Antibody | RG-6125 |
| Antibody | Bispecific Monoclonal Antibody | RG-6139 |
| Antibody | Monoclonal Antibody | RG-6149 |
| Antibody | Bispecific Monoclonal Antibody; | |
| | Monoclonal Antibody | RG-6160 |
| Antibody | Monoclonal Antibody | RG-6292 |
| Antibody | Antibody | RG-70240 |
| Antibody | Monoclonal Antibody Conjugated | RG-7861 |
| Antibody | Bispecific Monoclonal Antibody | RG-7992 |
| Antibody | Antibody | rho(D) immune globulin (human) |
| Antibody | Monoclonal Antibody | rilotumumab |
| Antibody | Monoclonal Antibody | risankizumab |
| Antibody | Monoclonal Antibody | rituximab |
| Antibody | Monoclonal Antibody | rituximab biosimilar |
| Antibody | Bispecific Monoclonal Antibody | RO-7082859 |
| Antibody | Bispecific Monoclonal Antibody | RO-7121661 |
| Antibody | Monoclonal Antibody | roledumab |
| Antibody | Bispecific Monoclonal Antibody | romilkimab |
| Antibody | Monoclonal Antibody | romosozumab |
| Antibody | Monoclonal Antibody Conjugated | rovalpituzumab tesirine |
| Antibody | Monoclonal Antibody | rozanolixizumab |
| Antibody | Monoclonal Antibody Conjugated | rozibafusp alfa |
| Antibody | Monoclonal Antibody | RZ-358 |
| Antibody | Antibody | SAB-301 |
| Antibody | Monoclonal Antibody Conjugated | sacituzumab govitecan |
| Antibody | Monoclonal Antibody | SAIT-301 |
| Antibody | Monoclonal Antibody Conjugated | SAR-408701 |
| Antibody | Monoclonal Antibody | SAR-439459 |
| Antibody | Bispecific Monoclonal Antibody | SAR-440234 |
| Antibody | Monoclonal Antibody | SAR-441236 |
| Antibody | Monoclonal Antibody | sarilumab |
| Antibody | Monoclonal Antibody | sasanlimab |
| Antibody | Monoclonal Antibody | satralizumab |
| Antibody | Monoclonal Antibody Conjugated | SC-003 |
| Antibody | Antibody | scorpion (polyvalent) |
| | | immunoglobulin F(ab′)2 antivenom |
| Antibody | Antibody | scorpion [centruroides] (polyvalent) |
| | | immunoglobulin F(ab′) 2 antivenom |
| | | [equine] |
| Antibody | Monoclonal Antibody | SCT-200 |
| Antibody | Monoclonal Antibody | SCT-630 |
| Antibody | Monoclonal Antibody | SEA-BCMA |
| Antibody | Monoclonal Antibody | SEA-CD40 |
| Antibody | Monoclonal Antibody | secukinumab |
| Antibody | Monoclonal Antibody | selicrelumab |
| Antibody | Monoclonal Antibody | semorinemab |
| Antibody | Monoclonal Antibody | setrusumab |
| Antibody | Monoclonal Antibody Conjugated | SGNCD-228A |
| Antibody | Monoclonal Antibody Conjugated | SGNCD-47M |
| Antibody | Antibody | SHR-1209 |
| Antibody | Monoclonal Antibody | SHR-1316 |
| Antibody | Monoclonal Antibody | siltuximab |
| Antibody | Monoclonal Antibody | Simponi Aria |
| Antibody | Monoclonal Antibody | sintilimab |
| Antibody | Monoclonal Antibody | siplizumab |
| Antibody | Monoclonal Antibody | sirukumab |
| Antibody | Monoclonal Antibody Conjugated | SKB-264 |
| Antibody | Monoclonal Antibody | solanezumab |
| Antibody | Monoclonal Antibody | spartalizumab |
| Antibody | Monoclonal Antibody | spesolimab |
| Antibody | Monoclonal Antibody | SRF-617 |
| Antibody | Monoclonal Antibody | SSS-07 |
| Antibody | Monoclonal Antibody | STIA-1014 |
| Antibody | Monoclonal Antibody Conjugated | STRO-001 |
| Antibody | Monoclonal Antibody Conjugated | STRO-002 |
| Antibody | Monoclonal Antibody | Sulituzumab |
| Antibody | Monoclonal Antibody | sutimlimab |
| Antibody | Monoclonal Antibody | suvratoxumab |
| Antibody | Monoclonal Antibody Conjugated | SYD-1875 |
| Antibody | Monoclonal Antibody | Sym-015 |
| Antibody | Monoclonal Antibody | Sym-021 |
| Antibody | Monoclonal Antibody | Sym-022 |
| Antibody | Monoclonal Antibody | Sym-023 |
| Antibody | Monoclonal Antibody | SYN-004 |
| Antibody | Monoclonal Antibody | SYN-023 |
| Antibody | Monoclonal Antibody | TAB-014 |
| Antibody | Monoclonal Antibody | TAB-08 |
| Antibody | Monoclonal Antibody | tafasitamab |
| Antibody | Antibody | taipan [Oxyuranus scutellatus] |
| | | antivenom [equine] |
| Antibody | Monoclonal Antibody | TAK-079 |
| Antibody | Monoclonal Antibody Conjugated | TAK-164 |
| Antibody | Monoclonal Antibody | talacotuzumab |
| Antibody | Monoclonal Antibody | tanezumab |
| Antibody | Monoclonal Antibody Conjugated | telisotuzumab vedotin |
| Antibody | Monoclonal Antibody | temelimab |
| Antibody | Monoclonal Antibody | teplizumab |
| Antibody | Monoclonal Antibody | teprotumumab |
| Antibody | Monoclonal Antibody | tesidolumab |
| Antibody | Antibody | tetanus immune globulin |
| Antibody | Monoclonal Antibody | tezepelumab |
| Antibody | Monoclonal Antibody Conjugated | TF-2 |
| Antibody | Bispecific Monoclonal Antibody | TG-1801 |
| Antibody | Monoclonal Antibody | THR-317 |
| Antibody | Bispecific Monoclonal Antibody | tibulizumab |
| Antibody | Monoclonal Antibody | tilavonemab |
| Antibody | Monoclonal Antibody | tildrakizumab |
| Antibody | Monoclonal Antibody | timigutuzumab |
| Antibody | Monoclonal Antibody | timolumab |
| Antibody | Monoclonal Antibody | tiragolumab |
| Antibody | Monoclonal Antibody | tislelizumab |
| Antibody | Monoclonal Antibody Conjugated | tisotumab vedotin |
| Antibody | Monoclonal Antibody | TJC-4 |
| Antibody | Monoclonal Antibody | TJD-5 |
| Antibody | Monoclonal Antibody | TJM-2 |
| Antibody | Monoclonal Antibody | TM-123 |
| Antibody | Bispecific Monoclonal Antibody | TMB-365 |
| Antibody | Bispecific Monoclonal Antibody | TNB-383B |
| Antibody | Monoclonal Antibody | tocilizumab |
| Antibody | Monoclonal Antibody | tocilizumab biosimilar |
| Antibody | Monoclonal Antibody | tomaralimab |
| Antibody | Monoclonal Antibody | tomuzotuximab |
| Antibody | Monoclonal Antibody | toripalimab |
| Antibody | Monoclonal Antibody | tosatoxumab |
| | | tositumomab + Iodine I 131 |
| Antibody | Monoclonal Antibody Conjugated | tositumomab |
| Antibody | Monoclonal Antibody | tralokinumab |
| Antibody | Monoclonal Antibody | trastuzumab |
| Antibody | Monoclonal Antibody | trastuzumab biosimilar |
| Antibody | Monoclonal Antibody Conjugated | trastuzumab deruxtecan |
| Antibody | Monoclonal Antibody Conjugated | trastuzumab duocarmazine |
| Antibody | Monoclonal Antibody Conjugated | trastuzumab emtansine |
| Antibody | Monoclonal Antibody | tremelimumab |
| Antibody | Monoclonal Antibody | trevogrumab |
| Antibody | Monoclonal Antibody | TRK-950 |
| Antibody | Monoclonal Antibody Conjugated | TRPH-222 |
| Antibody | Monoclonal Antibody | TTX-030 |
| Antibody | Monoclonal Antibody Conjugated | TX-250 |
| Antibody | Monoclonal Antibody Conjugated | U-31402 |
| Antibody | Monoclonal Antibody | U-31784 |
| Antibody | Monoclonal Antibody | UB-221 |
| Antibody | Monoclonal Antibody | UB-421 |
| Antibody | Monoclonal Antibody | UB-621 |
| Antibody | Monoclonal Antibody | ublituximab |
| Antibody | Monoclonal Antibody; Small | ublituximab + umbralisib tosylate |
| | Molecule | |
| Antibody | Monoclonal Antibody | UBP-1213 |
| Antibody | Monoclonal Antibody | UC-961 |
| Antibody | Monoclonal Antibody | UCB-0107 |
| Antibody | Monoclonal Antibody | UCB-6114 |
| Antibody | Monoclonal Antibody | UCB-7858 |
| Antibody | Monoclonal Antibody | ulocuplumab |
| Antibody | Monoclonal Antibody | urelumab |
| Antibody | Monoclonal Antibody | ustekinumab |
| Antibody | Monoclonal Antibody | ustekinumab biosimilar |
| Antibody | Monoclonal Antibody | utomilumab |
| Antibody | Monoclonal Antibody Conjugated | vadastuximab talirine |
| Antibody | Bispecific Monoclonal Antibody | vanucizumab |
| Antibody | Antibody | |
| Antibody | Monoclonal Antibody | varisacumab |
| Antibody | Monoclonal Antibody | varlilumab |
| Antibody | Monoclonal Antibody | vedolizumab |
| Antibody | Monoclonal Antibody | veltuzumab |
| Antibody | Monoclonal Antibody | VIR-2482 |
| Antibody | Monoclonal Antibody | VIS-410 |
| Antibody | Monoclonal Antibody | VIS-649 |
| Antibody | Monoclonal Antibody | vixarelimab |
| Antibody | Monoclonal Antibody Conjugated | VLS-101 |
| Antibody | Monoclonal Antibody | vobarilizumab |
| Antibody | Monoclonal Antibody | vofatamab |
| Antibody | Monoclonal Antibody | volagidemab |
| Antibody | Monoclonal Antibody | vopratelimab |
| Antibody | Monoclonal Antibody | VRC-01 |
| Antibody | Monoclonal Antibody | VRC-07523LS |
| Antibody | Monoclonal Antibody | vunakizumab |
| Antibody | Monoclonal Antibody Conjugated | W-0101 |
| Antibody | Monoclonal Antibody | WBP-297 |
| Antibody | Antibody | |
| Antibody | Antibody | Xembify |
| Antibody | Monoclonal Antibody | xentuzumab |
| Antibody | Monoclonal Antibody | Xgeva |
| Antibody | Bispecific Monoclonal Antibody | XmAb-14045 |
| Antibody | Bispecific Monoclonal Antibody | XmAb-22841 |
| Antibody | Bispecific Monoclonal Antibody | XmAb-23104 |
| Antibody | Monoclonal Antibody Conjugated | XMT-1536 |
| Antibody | Monoclonal Antibody | XOMA-213 |
| Antibody | Monoclonal Antibody | YS-110 |
| Antibody | Monoclonal Antibody | YYB-101 |
| Antibody | Monoclonal Antibody | zagotenemab |
| Antibody | Monoclonal Antibody | zalifrelimab |
| Antibody | Monoclonal Antibody | zanolimumab |
| Antibody | Bispecific Monoclonal Antibody | zenocutuzumab |
| Antibody | Monoclonal Antibody | zolbetuximab |
| Antibody | Bispecific Monoclonal Antibody | ZW-25 |
| Antibody/ | Antibody; Recombinant Enzyme | hyaluronidase (recombinant, |
| Enzyme | | human) + immune globulin (human) |
| Antibody/ | Fusion Protein; | durvalumab + oportuzumab |
| protein | Monoclonal Antibody | monatox |

TABLE 2
Peptides

| Broad class | Molecule Type | Drug Name |
|---|---|---|
| Peptide | Synthetic Peptide | A-10 + AS-21 |
| Peptide | Synthetic Peptide | A-6 |
| Peptide | Recombinant Peptide | AB-101 |
| Peptide | Recombinant Peptide | AB-102 |
| Peptide | Recombinant Peptide | AB-301 |
| Peptide | Synthetic Peptide | abaloparatide |
| Peptide | Synthetic Peptide | abarelix |
| Peptide | Synthetic Peptide | ABT-510 |
| Peptide | Recombinant Peptide | AC-2592 |
| Peptide | Synthetic Peptide | ACP-003 |
| Peptide | Synthetic Peptide | ACP-004 |
| Peptide | Synthetic Peptide | ACP-015 |
| Peptide | Synthetic Peptide | AcPepA |
| Peptide | Synthetic Peptide | ACX-107 |
| Peptide | Synthetic Peptide | Adipotide |
| Peptide | Recombinant Peptide | ADV-P2 |
| Peptide | Synthetic Peptide | AE-3763 |
| Peptide | Synthetic Peptide | AEM-28 |
| Peptide | Synthetic Peptide | afamelanotide acetate |
| Peptide | Synthetic Peptide | AFPep |
| Peptide | Synthetic Peptide | AGM-310 |
| Peptide | Recombinant Peptide | AI-401 |
| Peptide | Synthetic Peptide | AIM-102 |
| Peptide | Recombinant Peptide | AIM-DX |
| Peptide | Synthetic Peptide | AKL-0707 |
| Peptide | Recombinant Peptide | AKS-178 |
| Peptide | Synthetic Peptide | AL-242A1 |
| Peptide | Synthetic Peptide | AL-41A1 |
| Peptide | Synthetic Peptide | AL-78898A |
| Peptide | Synthetic Peptide | albenatide |
| Peptide | Synthetic Peptide | albuvirtide LAR |
| Peptide | Synthetic Peptide | alisporivir |
| Peptide | Synthetic Peptide | ALM-201 |
| Peptide | Synthetic Peptide | Alpha-1H |
| Peptide | Synthetic Peptide | Alpha-HGA |
| Peptide | Synthetic Peptide | ALRev-1 |
| Peptide | Synthetic Peptide | ALRN-5281 |
| Peptide | Synthetic Peptide | ALRN-6924 |
| Peptide | Synthetic Peptide | ALY-688 |
| Peptide | Synthetic Peptide | AMC-303 |
| Peptide | Synthetic Peptide | Ampion |
| Peptide | Synthetic Peptide | AMY-106 |
| Peptide | Synthetic Peptide | anaritide acetate |
| Peptide | Synthetic Peptide | angiotensin II acetate |
| Peptide | Recombinant Peptide | ANX-042 |
| Peptide | Synthetic Peptide | AP-138 |
| Peptide | Recombinant Peptide | APH-0907 |
| Peptide | Synthetic Peptide | APL-180 |
| Peptide | Synthetic Peptide | APL-9 |
| Peptide | Synthetic Peptide | APP-018 |
| Peptide | Synthetic Peptide | apraglutide |
| Peptide | Synthetic Peptide | ARG-301 |
| Peptide | Synthetic Peptide | argipressin |
| Peptide | Synthetic Peptide | ARI-1778 |
| Peptide | Synthetic Peptide | Artpep-2 |
| Peptide | Synthetic Peptide | ASP-5006 |
| Peptide | Recombinant Peptide | AT-247 |
| Peptide | Recombinant Peptide | AT-270 |
| Peptide | Synthetic Peptide | ATN-161 |
| Peptide | Synthetic Peptide | atosiban |
| Peptide | Synthetic Peptide | atosiban acetate |
| Peptide | Synthetic Peptide | Atrigel-GHRP-1 |
| Peptide | Recombinant Peptide | ATX-101 |
| Peptide | Synthetic Peptide | AVE-3247 |
| Peptide | Synthetic Peptide | avexitide acetate |
| Peptide | Synthetic Peptide | B27-PD |
| Peptide | Synthetic Peptide | bacitracin |
| Peptide | Synthetic Peptide | barusiban |
| Peptide | Synthetic Peptide | BBI-11008 |
| Peptide | Synthetic Peptide | BBI-21007 |
| Peptide | Synthetic Peptide | BDM-E |
| Peptide | Synthetic Peptide | BI-456906 |
| Peptide | Synthetic Peptide | BI-473494 |
| Peptide | Synthetic Peptide | bicalutamide + leuprolide acetate |
| Peptide | Recombinant Peptide | BIOD-105 |
| Peptide | Recombinant Peptide | BIOD-107 |
| Peptide | Recombinant Peptide | BIOD-123 |
| Peptide | Recombinant Peptide | BIOD-125 |
| Peptide | Recombinant Peptide | BIOD-238 |
| Peptide | Recombinant Peptide | BIOD-250 |
| Peptide | Recombinant Peptide | BIOD-531 |
| Peptide | Recombinant Peptide | BIOD-Adjustable Basal |
| Peptide | Synthetic Peptide | bivalirudin |
| Peptide | Synthetic Peptide | bivalirudin trifluoroacetate |
| Peptide | Peptide; Synthetic Peptide | BL-3020 |
| Peptide | Synthetic Peptide | BMS-686117 |
| Peptide | Synthetic Peptide | BMTP-11 |
| Peptide | Synthetic Peptide | BN-005 |
| Peptide | Synthetic Peptide | BN-006 |
| Peptide | Synthetic Peptide | BN-008 |
| Peptide | Synthetic Peptide | BN-054 |
| Peptide | Synthetic Peptide | BNZ-1 |
| Peptide | Recombinant Peptide | BNZ-2 |
| Peptide | Synthetic Peptide | BPI-3016 |
| Peptide | Synthetic Peptide | BQ-123 |
| Peptide | Synthetic Peptide | bremelanotide acetate |
| Peptide | Synthetic Peptide | brimapitide |
| Peptide | Synthetic Peptide | BRM-521 |
| Peptide | Synthetic Peptide | BT-5528 |
| Peptide | Synthetic Peptide | BTI-410 |
| Peptide | Synthetic Peptide | bulevirtide |
| Peptide | Synthetic Peptide | buserelin acetate |
| Peptide | Synthetic Peptide | buserelin acetate ER |
| Peptide | Synthetic Peptide | Bynfezia |
| Peptide | Synthetic Peptide | C-16G2 |
| Peptide | Synthetic Peptide | calcitonin |
| Peptide | Recombinant Peptide | calcitonin DR |
| Peptide | Recombinant Peptide | Capsulin IR |
| Peptide | Recombinant Peptide | Capsulin OAD |
| Peptide | Recombinant Peptide | CAR Peptide |
| Peptide | Synthetic Peptide | carbetocin |
| Peptide | Recombinant Peptide | Cardeva |
| Peptide | Recombinant Peptide | carperitide |
| Peptide | Synthetic Peptide | CBLB-612 |
| Peptide | Synthetic Peptide | CBP-501 |
| Peptide | Synthetic Peptide | CBX-129801 |
| Peptide | Recombinant Peptide | celmoleukin |
| Peptide | Recombinant Peptide | cenderitide |
| Peptide | Synthetic Peptide | cetrorelix |
| Peptide | Synthetic Peptide | cetrorelix acetate |
| Peptide | Synthetic Peptide | CGX-1007 |
| Peptide | Synthetic Peptide | CGX-1160 |
| Peptide | Synthetic Peptide | cibinetide |
| Peptide | Synthetic Peptide | CIGB-300 |
| Peptide | Recombinant Peptide | CIGB-370 |
| Peptide | Synthetic Peptide | CIGB-500 |
| Peptide | Synthetic Peptide | CIGB-552 |
| Peptide | Synthetic Peptide | CIGB-814 |
| Peptide | Synthetic Peptide | cilengitide |
| Peptide | Recombinant Peptide | CJC-1525 |
| Peptide | Synthetic Peptide | CMS-024 |
| Peptide | Synthetic Peptide | CN-105 |
| Peptide | Recombinant Peptide | CobOral Insulin |
| Peptide | Synthetic Peptide | COG-1410 |
| Peptide | Recombinant Peptide | Combulin |
| Peptide | Synthetic Peptide | corticorelin acetate |
| Peptide | Synthetic Peptide | corticotropin |
| Peptide | Synthetic Peptide | cosyntropin |
| Peptide | Synthetic Peptide | cosyntropin SR |
| Peptide | Synthetic Peptide | CPT-31 |
| Peptide | Synthetic Peptide | CTCE-9908 |
| Peptide | Recombinant Peptide | DACRA-042 |
| Peptide | Recombinant Peptide | DACRA-089 |
| Peptide | Synthetic Peptide | dalazatide |
| Peptide | Synthetic Peptide | danegaptide |
| Peptide | Synthetic Peptide | dasiglucagon |
| Peptide | Synthetic Peptide | DasKloster-0274-01 |
| Peptide | Synthetic Peptide | davunetide |
| Peptide | Synthetic Peptide | DD-04107 |
| Peptide | Synthetic Peptide | degarelix acetate |
| Peptide | Synthetic Peptide | delcasertib acetate |
| Peptide | Synthetic Peptide | delmitide acetate |
| Peptide | Synthetic Peptide | Dennexin |
| Peptide | Synthetic Peptide | Des-Asp Angiotensin 1 |
| Peptide | Recombinant Peptide | desirudin |
| Peptide | Synthetic Peptide | desmopressin |
| Peptide | Synthetic Peptide | desmopressin acetate |
| Peptide | Synthetic Peptide | desmopressin acetate ODT |
| Peptide | Synthetic Peptide | DiaPep-277 |
| Peptide | Synthetic Peptide | difelikefalin |
| Peptide | Synthetic Peptide | Dipep |
| Peptide | Synthetic Peptide | disitertide |
| Peptide | Synthetic Peptide | DMI-4983 |
| Peptide | Synthetic Peptide | dolcanatide |
| Peptide | Synthetic Peptide | DP-2018 |
| Peptide | Synthetic Peptide | DPC-016 |
| Peptide | Synthetic Peptide | DT-109 |
| Peptide | Synthetic Peptide | DT-110 |
| Peptide | Synthetic Peptide | DTI-100 |
| Peptide | Synthetic Peptide | DTI-117 |
| Peptide | Synthetic Peptide | dusquetide |
| Peptide | Synthetic Peptide | Dyofins |
| Peptide | Synthetic Peptide | E-21R |
| Peptide | Synthetic Peptide | EA-230 |
| Peptide | Recombinant Peptide | EB-613 |
| Peptide | Synthetic Peptide | Edotreotide Labeled Yttrium 90 |
| Peptide | Synthetic Peptide | edotreotide lutetium Lu-177 |
| Peptide | Synthetic Peptide | edratide |
| Peptide | Recombinant Peptide | efpeglenatide |
| Peptide | Recombinant Peptide; | efpeglenatide + HM-12470 |
| | Synthetic Peptide | |
| Peptide | Synthetic Peptide | elamipretide hydrochloride |
| Peptide | Synthetic Peptide | elcatonin |
| Peptide | Synthetic Peptide | ELIGO-3233 |
| Peptide | Synthetic Peptide | elsiglutide |
| Peptide | Recombinant Peptide | endostatin |
| Peptide | Synthetic Peptide | enfuvirtide |
| Peptide | Peptide; Synthetic Peptide | Engedi-1000 |
| Peptide | Synthetic Peptide | ENKASTIM-iv |
| Peptide | Synthetic Peptide | EP-100 |
| Peptide | Synthetic Peptide | EP-302 |
| Peptide | Synthetic Peptide | EP-342 |
| Peptide | Synthetic Peptide | EP-94 |
| Peptide | Synthetic Peptide | EPO-018B |
| Peptide | Synthetic Peptide | eptifibatide |
| Peptide | Recombinant Peptide | ES-135 |
| Peptide | Synthetic Peptide | etelcalcetide hydrochloride |
| Peptide | Synthetic Peptide | ETX-112 |
| Peptide | Synthetic Peptide | Evitar |
| Peptide | Synthetic Peptide | exenatide |
| Peptide | Synthetic Peptide | exenatide + Synthetic Peptide 1 |
| Peptide | Synthetic Peptide | exenatide + Synthetic Peptide 2 |
| Peptide | Synthetic Peptide | exenatide biobetter |
| Peptide | Synthetic Peptide | exenatide biosimilar |
| Peptide | Synthetic Peptide | exenatide CR |
| Peptide | Synthetic Peptide | exenatide ER |
| Peptide | Synthetic Peptide | exenatide Once Monthly |
| Peptide | Synthetic Peptide | exenatide SR |
| Peptide | Synthetic Peptide | exendin-(9-39) |
| Peptide | Synthetic Peptide | EXT-307 |
| Peptide | Synthetic Peptide | EXT-405 |
| Peptide | Synthetic Peptide | EXT-418 |
| Peptide | Synthetic Peptide | EXT-600 |
| Peptide | Synthetic Peptide | EXT-607 |
| Peptide | Synthetic Peptide | EXT-705 |
| Peptide | Recombinant Peptide | Extendin-Fc |
| Peptide | Synthetic Peptide | FE-204205 |
| Peptide | Synthetic Peptide | FF-3 |
| Peptide | Recombinant Peptide | Fiasp |
| Peptide | Synthetic Peptide | FM-19 |
| Peptide | Synthetic Peptide | FNS-007 |
| Peptide | Synthetic Peptide | forigerimod acetate |
| Peptide | Synthetic Peptide | Foxy-5 |
| Peptide | Synthetic Peptide | FP-001 |
| Peptide | Synthetic Peptide | FP-002 |
| Peptide | Synthetic Peptide | FP-005 |
| Peptide | Synthetic Peptide | FPP-003 |
| Peptide | Recombinant Peptide | FT-105 |
| Peptide | Synthetic Peptide | FX-06 |
| Peptide | Synthetic Peptide | G-3215 |
| Peptide | Synthetic Peptide | ganirelix acetate |
| Peptide | Synthetic Peptide | glatiramer acetate |
| Peptide | Synthetic Peptide | glatiramer acetate ER |
| Peptide | Synthetic Peptide | glatiramer biosimilar |
| Peptide | Synthetic Peptide | glepaglutide |
| Peptide | Recombinant Peptide | GLP-1 |
| Peptide | Recombinant Peptide | glucagon |
| Peptide | Recombinant Peptide | glucagon biosimilar |
| Peptide | Recombinant Peptide | Glucagon-Like Peptide-1 + insulin human |
| Peptide | Synthetic Peptide | glucosaminylmuramyl dipeptide |
| Peptide | Synthetic Peptide | GM-6 |
| Peptide | Synthetic Peptide | GO-2032c |
| Peptide | Synthetic Peptide | golotimod |
| Peptide | Synthetic Peptide | gonadorelin |
| Peptide | Synthetic Peptide | gonadorelin acetate |
| Peptide | Synthetic Peptide | goserelin |
| Peptide | Synthetic Peptide | goserelin acetate |
| Peptide | Synthetic Peptide | goserelin ER |
| Peptide | Synthetic Peptide | goserelin LA |
| Peptide | Synthetic Peptide | goserelin SR |
| Peptide | Recombinant Peptide | GP-40031 |
| Peptide | Synthetic Peptide | GSAO |
| Peptide | Synthetic Peptide | HaemoPlax |
| Peptide | Synthetic Peptide | hbEGF |
| Peptide | Recombinant Peptide | HDV-I |
| Peptide | Synthetic Peptide | hepcidin acetate |
| Peptide | Synthetic Peptide | histrelin |
| Peptide | Recombinant Peptide | HM-12460A |
| Peptide | Recombinant Peptide | HM-12470 |
| Peptide | Recombinant Peptide | HM-12480 |
| Peptide | Recombinant Peptide | HM-15136 |
| Peptide | Synthetic Peptide | HM-15211 |
| Peptide | Synthetic Peptide | Homspera |
| Peptide | Synthetic Peptide | HPI-1201 |
| Peptide | Synthetic Peptide | HPI-201 |
| Peptide | Synthetic Peptide | HPI-363 |
| Peptide | Synthetic Peptide | hPTH-137 |
| Peptide | Synthetic Peptide | HTD-4010 |
| Peptide | Synthetic Peptide | HTL-001 |
| Peptide | Recombinant Peptide | Humalog |
| Peptide | Synthetic Peptide | HXTC-901 |
| Peptide | Synthetic Peptide | Hydrogel Exenatide |
| Peptide | Synthetic Peptide | icatibant acetate |
| Peptide | Synthetic Peptide | IIIM-1 |
| Peptide | Synthetic Peptide | IMB-1007 |
| Peptide | Synthetic Peptide | ImmTher |
| Peptide | Recombinant Peptide | insulin |
| Peptide | Recombinant Peptide | insulin (bovine) |
| Peptide | Recombinant Peptide | insulin aspart |
| Peptide | Recombinant Peptide | insulin aspart 1 |
| Peptide | Recombinant Peptide | insulin aspart biosimilar |
| Peptide | Recombinant Peptide | insulin aspart injection |
| Peptide | Recombinant Peptide | insulin degludec |
| Peptide | Recombinant Peptide | insulin degludec LAR |
| Peptide | Recombinant Peptide | insulin detemir |
| Peptide | Recombinant Peptide | insulin glargine |
| Peptide | Recombinant Peptide | insulin glargine 1 |
| Peptide | Recombinant Peptide | insulin glargine biosimilar |
| Peptide | Recombinant Peptide | insulin glargine biosimilar 2 |
| Peptide | Recombinant Peptide | insulin glargine ER |
| Peptide | Recombinant Peptide | insulin glargine LA |
| Peptide | Recombinant Peptide | insulin glulisine |
| Peptide | Recombinant Peptide | insulin human |
| Peptide | Recombinant Peptide | insulin human (recombinant) |
| Peptide | Recombinant Peptide | insulin human 1 |
| Peptide | Recombinant Peptide | Insulin Human 30/70 Mix Marvel |
| Peptide | Recombinant Peptide | Insulin Human Long Marvel |
| Peptide | Recombinant Peptide | Insulin Human Rapid Marvel |
| Peptide | Recombinant Peptide | insulin human U100 |
| Peptide | Recombinant Peptide | insulin human zinc |
| Peptide | Recombinant Peptide | insulin I 131 |
| Peptide | Recombinant Peptide | insulin isophane |
| Peptide | Recombinant Peptide | insulin isophane human |
| Peptide | Recombinant Peptide | insulin lispro |
| Peptide | Recombinant Peptide | insulin lispro 2 |
| Peptide | Recombinant Peptide | insulin lispro U100 |
| Peptide | Recombinant Peptide | insulin lispro U200 |
| Peptide | Recombinant Peptide | insulin lispro U300 |
| Peptide | Recombinant Peptide | insulin neutral |
| Peptide | Recombinant Peptide | insulin peglispro |
| Peptide | Recombinant Peptide | insulin tregopil |
| Peptide | Recombinant Peptide | Insulin-PH20 |
| Peptide | Recombinant Peptide | Insulin-B12 Conjugate |
| Peptide | Recombinant Peptide | insulin, neutral |
| Peptide | Recombinant Peptide | Insuman |
| Peptide | Synthetic Peptide | IP-1510 |
| Peptide | Synthetic Peptide | IP-1510D |
| Peptide | Synthetic Peptide | ipamorelin |
| Peptide | Synthetic Peptide | IPL-344 |
| Peptide | Synthetic Peptide | IPP-102199 |
| Peptide | Synthetic Peptide | IPP-204106 |
| Peptide | Recombinant Peptide | Ir-CPI |
| Peptide | Synthetic Peptide | ISF-402 |
| Peptide | Recombinant Peptide | isophane protamine recombinant |
| | | human insulin |
| Peptide | Synthetic Peptide | ITCA-650 |
| Peptide | Synthetic Peptide | ITF-1697 |
| Peptide | Recombinant Peptide | ITF-2984 |
| Peptide | Recombinant Peptide | JDSCR-103 |
| Peptide | Synthetic Peptide | JMR-132 |
| Peptide | Synthetic Peptide | JNJ-26366821 |
| Peptide | Synthetic Peptide | JNJ-38488502 |
| Peptide | Synthetic Peptide | K-13 |
| Peptide | Synthetic Peptide | kahalalide F |
| Peptide | Synthetic Peptide | KAI-1678 |
| Peptide | Recombinant Peptide | KBP-088 |
| Peptide | Synthetic Peptide | KES-0001 |
| Peptide | Synthetic Peptide | Kisspeptin-10 |
| Peptide | Synthetic Peptide | KRX-0402 |
| Peptide | Synthetic Peptide | KSL-W |
| Peptide | Recombinant Peptide | KUR-112 |
| Peptide | Recombinant Peptide | KUR-113 |
| Peptide | Synthetic Peptide | L-1AD3 |
| Peptide | Recombinant Peptide | LAI-287 |
| Peptide | Recombinant Peptide | LAI-338 |
| Peptide | Synthetic Peptide | lanreotide acetate PR |
| Peptide | Synthetic Peptide | lanreotide SR |
| Peptide | Synthetic Peptide | larazotide acetate |
| Peptide | Synthetic Peptide | LAT-8881 |
| Peptide | Synthetic Peptide | LBT-1000 |
| Peptide | Synthetic Peptide | LBT-3627 |
| Peptide | Synthetic Peptide | LBT-5001 |
| Peptide | Synthetic Peptide | LBT-6030 |
| Peptide | Synthetic Peptide | LC-002 |
| Peptide | Synthetic Peptide | leconotide |
| Peptide | Synthetic Peptide | leuprolide |
| Peptide | Synthetic Peptide | leuprolide acetate |
| Peptide | Small Molecule; | leuprolide acetate + norethindrone |
| | Synthetic Peptide | |
| Peptide | Synthetic Peptide | leuprolide acetate ER |
| Peptide | Synthetic Peptide | leuprolide acetate PR |
| Peptide | Synthetic Peptide | leuprolide acetate SR |
| Peptide | Synthetic Peptide | leuprorelin acetate PR |
| Peptide | Synthetic Peptide | leuprorelin ER |
| Peptide | Synthetic Peptide | LH-021 |
| Peptide | Synthetic Peptide | LH-024 |
| Peptide | Synthetic Peptide | linaclotide |
| Peptide | Synthetic Peptide | linaclotide DR2 |
| Peptide | Recombinant Peptide | Linjeta |
| Peptide | Recombinant Peptide | liraglutide |
| Peptide | Synthetic Peptide | liraglutide biobetter |
| Peptide | Recombinant Peptide | liraglutide biosimilar |
| Peptide | Synthetic Peptide | livoletide |
| Peptide | Synthetic Peptide | lixisenatide |
| Peptide | Synthetic Peptide | lobradimil |
| Peptide | Synthetic Peptide | LP-003 |
| Peptide | Synthetic Peptide | LTX-315 |
| Peptide | Synthetic Peptide; | LTX-315 + tertomotide |
| | Vaccine | |
| Peptide | Synthetic Peptide | LTX-401 |
| Peptide | Synthetic Peptide | lutetium Lu 177 dotatate |
| Peptide | Synthetic Peptide | LY-2510924 |
| Peptide | Synthetic Peptide | LY-3143753 |
| Peptide | Synthetic Peptide | LY-3185643 |
| Peptide | Recombinant Peptide | LY-3209590 |
| Peptide | Synthetic Peptide | LY-3305677 |
| Peptide | Synthetic Peptide | LY-355703 |
| Peptide | Recombinant Peptide | LY-900027 |
| Peptide | Recombinant Peptide | Lyumjev |
| Peptide | Synthetic Peptide | M-012 |
| Peptide | Recombinant Peptide | Macrulin |
| Peptide | Synthetic Peptide | MALP-2S |
| Peptide | Synthetic Peptide | mannatide |
| Peptide | Synthetic Peptide | metenkefalin |
| Peptide | Synthetic Peptide | mibenratide |
| Peptide | Synthetic Peptide | mifamurtide |
| Peptide | Synthetic Peptide | mitolactol |
| Peptide | Recombinant Peptide | MOD-1001 |
| Peptide | Recombinant Peptide | MOD-1002 |
| Peptide | Recombinant Peptide | MOD-6030 |
| Peptide | Recombinant Peptide | MOD-6031 |
| Peptide | Synthetic Peptide | motixafortide |
| Peptide | Synthetic Peptide | Motrem |
| Peptide | Synthetic Peptide | MP-3167 |
| Peptide | Synthetic Peptide | MPE-002 |
| Peptide | Recombinant Peptide | MSTMB-103 |
| Peptide | Synthetic Peptide | MT-1002 |
| Peptide | Synthetic Peptide | MTX-1604 |
| Peptide | Synthetic Peptide | MVT-602 |
| Peptide | Synthetic Peptide | NAX-8102 |
| Peptide | Synthetic Peptide | NBI-6024 |
| Peptide | Synthetic Peptide | NBI-69734 |
| Peptide | Synthetic Peptide | NBP-14 |
| Peptide | Synthetic Peptide | nemifitide ditriflutate |
| Peptide | Synthetic Peptide | nepadutant |
| Peptide | Synthetic Peptide | Nephrilin |
| Peptide | Recombinant Peptide | nerinetide |
| Peptide | Synthetic Peptide | Nerofe |
| Peptide | Recombinant Peptide | nesiritide |
| Peptide | Recombinant Peptide | Neucardin |
| Peptide | Recombinant Peptide | NL-005 |
| Peptide | Synthetic Peptide | NLY-001 |
| Peptide | Recombinant Peptide | NN-1952 |
| Peptide | Recombinant Peptide | NN-1954 |
| Peptide | Recombinant Peptide | NN-1955 |
| Peptide | Recombinant Peptide | NN-1956 |
| Peptide | Recombinant Peptide | NN-1965 |
| Peptide | Synthetic Peptide | NN-9277 |
| Peptide | Synthetic Peptide | NN-9423 |
| Peptide | Recombinant Peptide | NN-9513 |
| Peptide | Synthetic Peptide | NN-9536 |
| Peptide | Synthetic Peptide | NN-9747 |
| Peptide | Synthetic Peptide | NN-9775 |
| Peptide | Synthetic Peptide | NN-9838 |
| Peptide | Synthetic Peptide | NN-9931 |
| Peptide | Synthetic Peptide | NNZ-2591 |
| Peptide | Synthetic Peptide | NOV-004 |
| Peptide | Synthetic Peptide | NRP-2945 |
| Peptide | Synthetic Peptide | NRX-1051 |
| Peptide | Recombinant Peptide | NsG-0501 |
| Peptide | Recombinant Peptide | NTRA-2112 |
| Peptide | Recombinant Peptide | NTRA-9620 |
| Peptide | Synthetic Peptide | NX-210 |
| Peptide | Recombinant Peptide | OA-150 |
| Peptide | Synthetic Peptide | OB-3 |
| Peptide | Synthetic Peptide | obinepitide |
| Peptide | Synthetic Peptide | octreotide |
| Peptide | Synthetic Peptide | octreotide acetate |
| Peptide | Synthetic Peptide | octreotide acetate CR |
| Peptide | Synthetic Peptide | octreotide acetate LA |
| Peptide | Synthetic Peptide | octreotide acetate LAR |
| Peptide | Synthetic Peptide | octreotide acetate MAR |
| Peptide | Synthetic Peptide | octreotide acetate microspheres |
| Peptide | Synthetic Peptide | octreotide acetate PR |
| Peptide | Synthetic Peptide | octreotide acetate SR |
| Peptide | Synthetic Peptide | octreotide LA |
| Peptide | Synthetic Peptide | OHR/AVR-118 |
| Peptide | Recombinant Peptide | OI-320GT |
| Peptide | Recombinant Peptide | OI-338GT |
| Peptide | Synthetic Peptide | OK-201 |
| Peptide | Synthetic Peptide | OKI-179 |
| Peptide | Synthetic Peptide | OKI-422 |
| Peptide | Recombinant Peptide | OMO-103 |
| Peptide | Recombinant Peptide | ONCase-PEG |
| Peptide | Synthetic Peptide | ONK-102 |
| Peptide | Synthetic Peptide | ONL-1204 |
| Peptide | Synthetic Peptide | Oratonin |
| Peptide | Synthetic Peptide | orilotimod potassium |
| Peptide | Synthetic Peptide | ornipressin |
| Peptide | Synthetic Peptide | ORTD-1 |
| Peptide | Synthetic Peptide | OXE-103 |
| Peptide | Recombinant Peptide | Oxymera |
| Peptide | Synthetic Peptide | oxyntomodulin |
| Peptide | Synthetic Peptide | oxytocin |
| Peptide | Synthetic Peptide | ozarelix |
| Peptide | Recombinant Peptide | Ozempic |
| Peptide | Synthetic Peptide | P-17 |
| Peptide | Synthetic Peptide | P-28 |
| Peptide | Synthetic Peptide | P-28R |
| Peptide | Synthetic Peptide | P-8 |
| Peptide | Recombinant Peptide | parathyroid hormone |
| Peptide | Synthetic Peptide | pasireotide |
| Peptide | Synthetic Peptide | pasireotide LAR |
| Peptide | Recombinant Peptide | PB-1023 |
| Peptide | Synthetic Peptide | PB-119 |
| Peptide | Synthetic Peptide | PCO-01 |
| Peptide | Synthetic Peptide | PCO-02 |
| Peptide | Synthetic Peptide | PDC-31 |
| Peptide | Recombinant Peptide | PE-0139 |
| Peptide | Synthetic Peptide | PEG Exenatide |
| Peptide | Synthetic Peptide | pegapamodutide |
| Peptide | Synthetic Peptide | pegcetacoplan |
| Peptide | Synthetic Peptide | peginesatide |
| Peptide | Synthetic Peptide | Pegylated Thymalfasin |
| Peptide | Recombinant Peptide | PEN-221 |
| Peptide | Peptide | |
| Peptide | Synthetic Peptide | Peptide T |
| Peptide | Peptide | Peptide to Inhibit Amyloid Beta Peptide for |
| | | Alzheimer's Disease |
| Peptide | Peptide | Peptide to Inhibit GRP-78 for Melanoma |
| Peptide | Synthetic Peptide | PHIN-1138 |
| Peptide | Synthetic Peptide | PHIN-837 |
| Peptide | Synthetic Peptide | PI-0824 |
| Peptide | Recombinant Peptide | PI-406 |
| Peptide | Synthetic Peptide | pidotimod |
| Peptide | Synthetic Peptide | PIN-201104 |
| Peptide | Synthetic Peptide | PL-3994 |
| Peptide | Synthetic Peptide | PL-8177 |
| Peptide | Synthetic Peptide | Plannexin |
| Peptide | Synthetic Peptide | plecanatide |
| Peptide | Synthetic Peptide | PLG-0206 |
| Peptide | Synthetic Peptide | plitidepsin |
| Peptide | Synthetic Peptide | PMZ-2123 |
| Peptide | Synthetic Peptide | PN-943 |
| Peptide | Synthetic Peptide | PNT-2002 |
| Peptide | Synthetic Peptide | polyethylene glycol loxenatide LAR |
| Peptide | Synthetic Peptide | PP-1420 |
| Peptide | Synthetic Peptide | pramlintide |
| Peptide | Synthetic Peptide | Preimplantation Factor |
| Peptide | Synthetic Peptide | PRI-002 |
| Peptide | Synthetic Peptide | PRI-003 |
| Peptide | Synthetic Peptide | PRI-004 |
| Peptide | Synthetic Peptide | protamine sulfate |
| Peptide | Recombinant Peptide | protamine zinc insulin |
| Peptide | Recombinant Peptide | Protaphane |
| Peptide | Synthetic Peptide | PT-302 |
| Peptide | Synthetic Peptide | PT-320 |
| Peptide | Synthetic Peptide | PT-330 |
| Peptide | Synthetic Peptide | PTG-200 |
| Peptide | Synthetic Peptide | PZ-128 |
| Peptide | Peptide | QUB-3164 |
| Peptide | Recombinant Peptide | rE-4 |
| Peptide | Synthetic Peptide | REC-0438 |
| Peptide | Recombinant Peptide | Recombinant Human Intestinal Trefoil |
| | | Factor |
| Peptide | Recombinant Peptide | Recombinant Peptide 1 to Agonize Insulin |
| | | Receptor for Type 1 and Type 2 Diabetes |
| Peptide | Recombinant Peptide | Recombinant Peptide to Agonize |
| | | Calcitonin Gene Related Peptide Receptor |
| | | for Osteoporosis and Hypertension |
| Peptide | Recombinant Peptide | Recombinant Peptide to Agonize GHRH |
| | | for Cardiovascular, Central Nervous |
| | | System, Musculoskeletal and Metabolic |
| | | Disorders |
| Peptide | Recombinant Peptide | Recombinant Peptide to Agonize GLP1R |
| | | for Type 2 Diabetes |
| Peptide | Recombinant Peptide | Recombinant Peptide to Agonize Insulin |
| | | receptor for Diabetes |
| Peptide | Recombinant Peptide | Recombinant Peptide to Agonize Insulin |
| | | Receptor for Type 1 and Type 2 Diabetes |
| Peptide | Recombinant Peptide | Recombinant Peptide to Agonize Insulin |
| | | Receptor for Type 1 Diabetes |
| Peptide | Recombinant Peptide | Recombinant Peptide to Agonize Insulin |
| | | Receptor for Type 2 Diabetes |
| Peptide | Recombinant Peptide | Recombinant Peptide to Agonize PTH-R |
| | | for Post Menopausal Osteoporosis |
| Peptide | Recombinant Peptide | Recombinant Peptide to Agonize PTH1R |
| | | for Bone Fracture |
| Peptide | Recombinant Peptide | Recombinant Peptide to Agonize PTH1R |
| | | for Hypoparathyroidism |
| Peptide | Recombinant Peptide | Recombinant Peptide to Inhibit TNF Alpha |
| | | for Crohn's Disease, Asthma And |
| | | Metabolic Syndrome |
| Peptide | Recombinant Peptide | Recombinant Peptide-1 to Activate GLP-1 |
| | | for Type 2 Diabetes |
| Peptide | Recombinant Peptide | Recombinant Peptides 6 to Agonize |
| | | Insulin Receptor for Type 1 and Type 2 |
| | | Diabetes |
| Peptide | Recombinant Peptide | Recombinant Peptides to Activate GLP-1 |
| | | for Type-2 Diabetes |
| Peptide | Recombinant Peptide | Recombinant Peptides to Agonize Insulin |
| | | Receptor for Type 1 and Type 2 Diabetes |
| Peptide | Recombinant Peptide | Recombinant Peptides to Agonize MFN2 |
| | | for Charcot Marie Tooth Disease Type IIA |
| | | and Hypertrophic Cardiomyopathy |
| Peptide | Synthetic Peptide | Reg-O3 |
| Peptide | Synthetic Peptide | relamorelin |
| Peptide | Synthetic Peptide | reltecimod sodium |
| Peptide | Recombinant Peptide | Rescue-G |
| Peptide | Synthetic Peptide | RGN-352 |
| Peptide | Recombinant Peptide | Rh-RGD-Hirudin |
| Peptide | Synthetic Peptide | risuteganib |
| Peptide | Synthetic Peptide | romidepsin |
| Peptide | Synthetic Peptide | RPI-78M |
| Peptide | Synthetic Peptide | RPI-MN |
| Peptide | Recombinant Peptide | RTP-025 |
| Peptide | Synthetic Peptide | rusalatide acetate |
| Peptide | Synthetic Peptide | Rybelsus |
| Peptide | Recombinant Peptide | SAR-161271 |
| Peptide | Synthetic Peptide | SAR-425899 |
| Peptide | Recombinant Peptide | Saxenda |
| Peptide | Synthetic Peptide | SBI-1301 |
| Peptide | Synthetic Peptide | SBT-20 |
| Peptide | Synthetic Peptide | SBT-272 |
| Peptide | Synthetic Peptide | SCO-094 |
| Peptide | Synthetic Peptide | SER-130 |
| Peptide | Synthetic Peptide | setmelanotide |
| Peptide | Synthetic Peptide | setmelanotide ER |
| Peptide | Synthetic Peptide | SGX-943 |
| Peptide | Recombinant Peptide | somatostatin |
| Peptide | Recombinant Peptide | somatrem |
| Peptide | Recombinant Peptide | somatrogon |
| Peptide | Synthetic Peptide | SORC-13 |
| Peptide | Synthetic Peptide | sovateltide |
| Peptide | Synthetic Peptide | SRI-31277 |
| Peptide | Synthetic Peptide | STR-324 |
| Peptide | Synthetic Peptide | Synthetic Peptide 1 to Inhibit PD-L1 for |
| | | Oncology |
| Peptide | Synthetic Peptide | Synthetic Peptide for Dengue |
| Peptide | Synthetic Peptide | Synthetic Peptide for Huntington Disease |
| Peptide | Synthetic Peptide | Synthetic Peptide for Oncology |
| Peptide | Synthetic Peptide | Synthetic Peptide for Zika Virus Infection |
| Peptide | Synthetic Peptide | Synthetic Peptide to Agonize GLP1R for |
| | | Type 2 Diabetes |
| Peptide | Synthetic Peptide | Synthetic Peptide to Agonize Insulin |
| | | Receptor for Type 2 Diabetes |
| Peptide | Synthetic Peptide | Synthetic Peptide to Inhibit Alpha |
| | | Synuclein for Parkinson's Disease |
| Peptide | Synthetic Peptide | Synthetic Peptide to Inhibit Connexin 43 |
| | | for Optic Neuropathy |
| Peptide | Synthetic Peptide | Synthetic Peptide to Inhibit ELK1 for |
| | | Central Nervous System Disorders |
| Peptide | Synthetic Peptide | Synthetic Peptide to Inhibit PCSK9 for |
| | | Hypercholesterolemia |
| Peptide | Synthetic Peptide | Synthetic Peptide to Inhibit SOD1 for |
| | | Amyotrophic Lateral Sclerosis |
| Peptide | Synthetic Peptide | Synthetic Peptide to Inhibit Tau for |
| | | Tauopathies |
| Peptide | Synthetic Peptide | Synthetic Peptide to Inhibit TNF-Alpha for |
| | | Rheumatoid Arthritis |
| Peptide | Synthetic Peptide | Synthetic Peptide to Inhibit VEGFD for |
| | | Oncology |
| Peptide | Synthetic Peptide | Synthetic Peptide to Modulate GHSR for |
| | | Chronic Kidney Disease |
| Peptide | Synthetic Peptide | Synthetic Peptide to Target CCKBR for |
| | | Medullary Thyroid Cancer |
| Peptide | Synthetic Peptide | Synthetic Peptide to Target Somatostatin |
| | | Receptor for Neuroendocrine |
| | | Gastroenteropancreatic Tumors |
| Peptide | Synthetic Peptide | Synthetic Peptide to Target Somatostatin |
| | | Receptor for Neuroendocrine Tumors |
| Peptide | Synthetic Peptide | Synthetic Peptides to Activate TMEM173 |
| | | for Oncology |
| Peptide | Synthetic Peptide | Synthetic Peptides to Agonize DOR1 and |
| | | MOR1 for Irritable Bowel Syndome with |
| | | Diarrhea |
| Peptide | Synthetic Peptide | Synthetic Peptides to Agonize GLP1R for |
| | | Type 2 Diabetes |
| Peptide | Synthetic Peptide | Synthetic Peptides to Agonize TLR for |
| | | Oncology |
| Peptide | Synthetic Peptide | Synthetic Peptides to Antagonize CXCR7 |
| | | for Oncology |
| Peptide | Synthetic Peptide | Synthetic Peptides to Inhibit Beta Catenin |
| | | for Oncology |
| Peptide | Synthetic Peptide | Synthetic Peptides to Inhibit Complement |
| | | C3 for Unspecified Indication |
| Peptide | Synthetic Peptide | Synthetic Peptides to Inhibit Cyclin E for |
| | | Oncology |
| Peptide | Synthetic Peptide | Synthetic Peptides to Inhibit |
| | | CyclinA/CDK2 for Oncology |
| Peptide | Synthetic Peptide | Synthetic Peptides to Inhibit DRB1 for |
| | | Multiple Sclerosis |
| Peptide | Synthetic Peptide | Synthetic Peptides to Inhibit E1 and E2 |
| | | Glycoprotein for HCV |
| Peptide | Synthetic Peptide | Synthetic Peptides to Inhibit Factor D for |
| | | Geographic Atrophy, Paroxysmal |
| | | Nocturnal Hemoglobinuria and Renal |
| | | Disease |
| Peptide | Synthetic Peptide | Synthetic Peptides to Inhibit Glycoprotein |
| | | VI for Thrombosis |
| Peptide | Synthetic Peptide | Synthetic Peptides to Inhibit MCL1 for |
| | | Oncology |
| Peptide | Synthetic Peptide | Synthetic Peptides to Inhibit SMURF2 for |
| | | Fibrosis and Oncology |
| Peptide | Synthetic Peptide | Synthetic Peptides to Inhibit TREM-1 for |
| | | Oncology, Sepsis, Rheumatoid Arthritis, |
| | | Retinopathy Of Prematurity and |
| | | Hemorrhagic Shock |
| Peptide | Recombinant Peptide | T-0005 |
| Peptide | Synthetic Peptide | T-20K |
| Peptide | Recombinant Peptide | TAC-201 |
| Peptide | Synthetic Peptide | Tatbeclin-1 |
| Peptide | Recombinant Peptide | TBR-760 |
| Peptide | Synthetic Peptide | TCANG-05 |
| Peptide | Synthetic Peptide | TCMCB-07 |
| Peptide | Recombinant Peptide | teduglutide |
| Peptide | Synthetic Peptide | teicoplanin |
| Peptide | Recombinant Peptide | teriparatide |
| Peptide | Recombinant Peptide | teriparatide acetate |
| Peptide | Recombinant Peptide | teriparatide biosimilar |
| Peptide | Synthetic Peptide | terlipressin |
| Peptide | Synthetic Peptide | tesamorelin acetate |
| Peptide | Synthetic Peptide | THR-149 |
| Peptide | Synthetic Peptide | thymalfasin |
| Peptide | Synthetic Peptide | |
| Peptide | Recombinant Peptide | tifacogin |
| Peptide | Synthetic Peptide | tirzepatide |
| Peptide | Synthetic Peptide | TPX-100 |
| Peptide | Synthetic Peptide | triptorelin |
| Peptide | Synthetic Peptide | triptorelin acetate |
| Peptide | Synthetic Peptide | triptorelin acetate ER |
| Peptide | Synthetic Peptide | triptorelin acetate SR |
| Peptide | Synthetic Peptide | triptorelin pamoate |
| Peptide | Synthetic Peptide | triptorelin pamoate ER |
| Peptide | Synthetic Peptide | triptorelin SR |
| Peptide | Synthetic Peptide | TXA-127 |
| Peptide | Synthetic Peptide | TXA-302 |
| Peptide | Recombinant Peptide | UGP-281 |
| Peptide | Recombinant Peptide | UGP-302 |
| Peptide | Recombinant Peptide | Ultratard |
| Peptide | Recombinant Peptide | Uni-E4 |
| Peptide | Synthetic Peptide | Upelior |
| Peptide | Synthetic Peptide | V-10 |
| Peptide | Synthetic Peptide | VAL-201 |
| Peptide | Synthetic Peptide | vapreotide acetate |
| Peptide | Synthetic Peptide | vasopressin |
| Peptide | Synthetic Peptide | veldoreotide ER |
| Peptide | Synthetic Peptide | veldoreotide IR |
| Peptide | Synthetic Peptide | VG-1177 |
| Peptide | Recombinant Peptide | VIAcal |
| Peptide | Recombinant Peptide | vosoritide |
| Peptide | Recombinant Peptide | VTCG-15 |
| Peptide | Peptide | XG-402 |
| Peptide | Peptide | XG-404 |
| Peptide | Synthetic Peptide | Y-14 |
| Peptide | Synthetic Peptide | YH-14618 |
| Peptide | Synthetic Peptide | ziconotide |
| Peptide | Synthetic Peptide | zilucoplan |
| Peptide | Recombinant Peptide | Znsulin |
| Peptide | Synthetic Peptide | ZP-10000 |
| Peptide | Synthetic Peptide | ZP-7570 |
| Peptide | Synthetic Peptide | ZT-01 |
| Peptide | Recombinant Peptide | ZT-031 |
| Peptide | Synthetic Peptide | ZYKR-1 |

TABLE 3
Enzymes

| Broad class | Molecule Type | Drug Name |
|---|---|---|
| Enzyme | Recombinant Enzyme | AB-002 |
| Enzyme | Recombinant Enzyme | ACN-00177 |
| Enzyme | Recombinant Enzyme | agalsidase alfa |
| Enzyme | Recombinant Enzyme | agalsidase beta |
| Enzyme | Recombinant Enzyme | albutrepenonacog alfa ER |
| Enzyme | Recombinant Enzyme | alglucerase |
| Enzyme | Recombinant Enzyme | alglucosidase alfa |
| Enzyme | Recombinant Enzyme | alteplase |
| Enzyme | Recombinant Enzyme | alteplase biosimilar |
| Enzyme | Enzyme | ancrod |
| Enzyme | Enzyme | anistreplase |
| Enzyme | Recombinant Enzyme | apadamtase alfa |
| Enzyme | Recombinant Enzyme | APN-01 |
| Enzyme | Recombinant Enzyme | asfotase alfa |
| Enzyme | Enzyme | asparaginase |
| Enzyme | Recombinant Enzyme | avalglucosidase alfa |
| Enzyme | Recombinant Enzyme | BCT-100 |
| Enzyme | Recombinant Enzyme | bRESCAP |
| Enzyme | Enzyme | bromelains |
| Enzyme | Recombinant Enzyme | calaspargase pegol |
| Enzyme | Recombinant Enzyme | cerliponase alfa |
| Enzyme | Enzyme | chymopapain |
| Enzyme | Enzyme | chymotrypsin |
| Enzyme | Recombinant Enzyme | coagulation factor IX (recombinant) |
| Enzyme | Recombinant Enzyme | coagulation factor IX (recombinant) biosimilar |
| Enzyme | Recombinant Enzyme | coagulation factor VIIa (recombinant) biosimilar |
| Enzyme | Recombinant Enzyme | coagulation factor XIII A-subunit (recombinant) |
| Enzyme | Enzyme | collagenase clostridium histolyticum |
| Enzyme | Recombinant Enzyme | condoliase |
| Enzyme | Recombinant Enzyme | CP-205 |
| Enzyme | Recombinant Enzyme | CUSA-081 |
| Enzyme | Recombinant Enzyme | dalcinonacog alfa |
| Enzyme | Recombinant Enzyme | elapegademase |
| Enzyme | Recombinant Enzyme | elosulfase alfa |
| Enzyme | Recombinant Enzyme | ERYGEN |
| Enzyme | Recombinant Enzyme | exebacase |
| Enzyme | Recombinant Enzyme | galsulfase |
| Enzyme | Recombinant Enzyme | glucarpidase |
| Enzyme | Enzyme | hemocoagulase |
| Enzyme | Recombinant Enzyme | HGT-1111 |
| Enzyme | Recombinant Enzyme | hRESCAP |
| Enzyme | Recombinant Enzyme | idursulfase |
| Enzyme | Recombinant Enzyme | idursulfase beta |
| Enzyme | Recombinant Enzyme | imiglucerase |
| Enzyme | Recombinant Enzyme | imiglucerase biosimilar |
| Enzyme | Recombinant Enzyme | imlifidase |
| Enzyme | Recombinant Enzyme | JR-141 |
| Enzyme | Recombinant Enzyme | JZP-458 |
| Enzyme | Recombinant Enzyme | KTP-001 |
| Enzyme | Recombinant Enzyme | laronidase |
| Enzyme | Recombinant Enzyme | lesinidase alfa |
| Enzyme | Recombinant Enzyme | Lumizyme |
| Enzyme | Recombinant Enzyme | marzeptacog alfa (activated) |
| Enzyme | Recombinant Enzyme | MEDI-6012 |
| Enzyme | Recombinant Enzyme | MOSS-AGAL |
| Enzyme | Recombinant Enzyme | ocriplasmin |
| Enzyme | Recombinant Enzyme | olipudase alfa |
| Enzyme | Recombinant Enzyme | OT-58 |
| Enzyme | Enzyme | pegademase bovine |
| Enzyme | Recombinant Enzyme | pegadricase |
| Enzyme | Recombinant Enzyme | pegargiminase |
| Enzyme | Recombinant Enzyme | pegaspargase |
| Enzyme | Recombinant Enzyme | pegaspargase biosimilar |
| Enzyme | Recombinant Enzyme | pegcrisantaspase |
| Enzyme | Recombinant Enzyme | pegloticase |
| Enzyme | Recombinant Enzyme | pegunigalsidase alfa |
| Enzyme | Recombinant Enzyme | pegvaliase |
| Enzyme | Recombinant Enzyme | pegvorhyaluronidase alfa |
| Enzyme | Recombinant Enzyme | pegzilarginase |
| Enzyme | Recombinant Enzyme | PF-05230907 |
| Enzyme | Enzyme | PRP |
| Enzyme | Recombinant Enzyme | PT-01 |
| Enzyme | Recombinant Enzyme | ranpirnase |
| Enzyme | Recombinant Enzyme | rasburicase |
| Enzyme | Recombinant Enzyme | |
| Enzyme | Recombinant Enzyme | Recombinant Glucosylceramidase Replacement for |
| | | Type I and Type III Gaucher's Disease |
| Enzyme | Recombinant Enzyme | Recombinant Human Alkaline Phosphatase |
| | | Replacement for Acute Renal Failure, |
| | | Hypophosphatasia, Sepsis and Ulcerative Colitis |
| Enzyme | Recombinant Enzyme | Recombinant Urate Oxidase Replacement for Acute |
| | | Hyperuricemia |
| Enzyme | Recombinant Enzyme | reteplase |
| Enzyme | Recombinant Enzyme | sebelipase alfa |
| Enzyme | Recombinant Enzyme | SHP-610 |
| Enzyme | Enzyme | SOBI-003 |
| Enzyme | Recombinant Enzyme | Spectrila |
| Enzyme | Recombinant Enzyme | staphylokinase |
| Enzyme | Enzyme | streptokinase |
| Enzyme | Recombinant Enzyme | TAK-611 |
| Enzyme | Recombinant Enzyme | taliglucerase alfa |
| Enzyme | Recombinant Enzyme | tenecteplase |
| Enzyme | Recombinant Enzyme | TNX-1300 |
| Enzyme | Recombinant Enzyme | tonabacase |
| Enzyme | Recombinant Enzyme | tralesinidase alfa |
| Enzyme | Enzyme | urokinase |
| Enzyme | Recombinant Enzyme | velaglucerase alfa |
| Enzyme | Recombinant Enzyme | velmanase alfa |
| Enzyme | Recombinant Enzyme | vestronidase alfa |
| Enzyme | Recombinant Enzyme | vonapanitase |
| Enzyme | Recombinant Enzyme | VX-210 |

TABLE 4
Proteins

| Broad Class | Molecule Type | Drug Name |
|---|---|---|
| Protein | Recombinant Protein | 3K3A-APC |
| Protein | Fusion Protein | abatacept |
| Protein | Recombinant Protein | abicipar pegol |
| Protein | Protein | abobotulinumtoxin A next generation |
| Protein | Protein | abobotulinumtoxinA |
| Protein | Recombinant Protein | ABY-035 |
| Protein | Recombinant Protein | ABY-039 |
| Protein | Protein | ACP-014 |
| Protein | Recombinant Protein | ACT-101 |
| Protein | Fusion Protein | AD-214 |
| Protein | Fusion Protein | aflibercept |
| Protein | Fusion Protein | aflibercept biosimilar |
| Protein | Fusion Protein | AGT-181 |
| Protein | Fusion Protein | AGT-182 |
| Protein | Fusion Protein | AKR-001 |
| Protein | Protein | Albicin |
| Protein | Recombinant Protein | albiglutide |
| Protein | Fusion Protein | albinterferon alfa-2b |
| Protein | Recombinant Protein | aldafermin |
| Protein | Recombinant Protein | aldesleukin |
| Protein | Fusion Protein | alefacept |
| Protein | Fusion Protein | ALKS-4230 |
| Protein | Fusion Protein | ALPN-101 |
| Protein | Fusion Protein | ALT-801 |
| Protein | Fusion Protein | ALTP-1 |
| Protein | Fusion Protein | ALX-148 |
| Protein | Recombinant Protein | AMRS-001 |
| Protein | Recombinant Protein | anakinra |
| Protein | Recombinant Protein | ancestim |
| Protein | Recombinant Protein | andexanet alfa |
| Protein | Recombinant Protein | antihemophilic factor (recombinant) |
| Protein | Recombinant Protein | antihemophilic factor (human) |
| Protein | Recombinant Protein | antihemophilic factor (recombinant) biosimilar |
| Protein | Fusion Protein | antihemophilic factor (recombinant), FcFusion |
| | | protein |
| Protein | Recombinant Protein | antihemophilic factor (recombinant), PEGylated |
| Protein | Recombinant Protein | antihemophilic factor (recombinant), |
| | | plasma/albumin free |
| Protein | Recombinant Protein | antihemophilic factor (recombinant), |
| | | plasma/albumin free method |
| Protein | Recombinant Protein | antihemophilic factor (recombinant), porcine |
| | | sequence |
| Protein | Recombinant Protein | antihemophilic factor (recombinant), single chain |
| Protein | Recombinant Protein | antithrombin (recombinant) |
| Protein | Fusion Protein | APN-301 |
| Protein | Fusion Protein | APO-010 |
| Protein | Fusion Protein | Aravive-S6 |
| Protein | Fusion Protein | asunercept |
| Protein | Fusion Protein | atacicept |
| Protein | Fusion Protein | ATYR-1923 |
| Protein | Recombinant Protein | ATYR-1940 |
| Protein | Recombinant Protein | AU-011 |
| Protein | Recombinant Protein | aviscumine |
| Protein | Recombinant Protein | avotermin |
| Protein | Fusion Protein | balugrastim |
| Protein | Recombinant Protein | batroxobin |
| Protein | Recombinant Protein | BBT-015 |
| Protein | Recombinant Protein | BCD-131 |
| Protein | Protein | bee venom |
| Protein | Fusion Protein | belatacept |
| Protein | Recombinant Protein | bempegaldesleukin |
| Protein | Protein | beractant |
| Protein | Recombinant Protein | BG-8962 |
| Protein | Fusion Protein | bintrafusp alfa |
| Protein | Recombinant Protein | BIO89-100 |
| Protein | Fusion Protein | BIVV-001 |
| Protein | Fusion Protein | blisibimod |
| Protein | Recombinant Protein; Small | boceprevir + peginterferon alfa-2b + ribavirin |
| | Molecule | |
| Protein | Protein | botulinum toxin type A |
| Protein | Protein | BXQ-350 |
| Protein | Protein | C1 esterase inhibitor (human) |
| Protein | Recombinant Protein | C1-esterase inhibitor |
| Protein | Protein | Cadisurf |
| Protein | Recombinant Protein | Cardiotrophin-1 |
| Protein | Protein | CB-24 |
| Protein | Fusion Protein | CD-24Fc |
| Protein | Recombinant Protein | CDX-301 |
| Protein | Recombinant Protein | cepeginterferon alfa-2b |
| Protein | Recombinant Protein | CER-001 |
| Protein | Recombinant Protein | CG-100 |
| Protein | Recombinant Protein | CG-367 |
| Protein | Recombinant Protein | choriogonadotropin alfa |
| Protein | Recombinant Protein | chorionic gonadotropin |
| Protein | Recombinant Protein | CIGB-128 |
| Protein | Protein | CIGB-845 |
| Protein | Recombinant Protein | cimaglermin alfa |
| Protein | Recombinant Protein | cintredekin besudotox |
| Protein | Fusion Protein | coagulation factor IX (recombinant), Fc fusion |
| | | protein |
| Protein | Recombinant Protein | coagulation factor IX (recombinant), glycopegylated |
| Protein | Recombinant Protein | coagulation Factor VIIa (Recombinant) |
| Protein | Recombinant Protein | coagulation factor VIII (recombinant) biosimilar |
| Protein | Fusion Protein | conbercept |
| Protein | Recombinant Protein | conestat alfa |
| Protein | Recombinant Protein | corifollitropin alfa |
| Protein | Fusion Protein | CSL-689 |
| Protein | Recombinant Protein | CSL-730 |
| Protein | Fusion Protein | CTI-1601 |
| Protein | Fusion Protein | CUE-101 |
| Protein | Recombinant Protein | CVBT-141A |
| Protein | Recombinant Protein | CVBT-141C |
| Protein | Recombinant Protein | CYT-6091 |
| Protein | Recombinant Protein | CYT-99007 |
| Protein | Recombinant Protein | Cyto-012 |
| Protein | Recombinant Protein | dapiclermin |
| Protein | Recombinant Protein | darbepoetin alfa |
| Protein | Recombinant Protein | darbepoetin alfa biosimilar LA |
| Protein | Recombinant Protein | darbepoetin alfa LA |
| Protein | Fusion Protein | darleukin |
| Protein | Fusion Protein | daromun |
| Protein | Fusion Protein | dazodalibep |
| Protein | Fusion Protein | Dekavil |
| Protein | Recombinant Protein | denenicokin |
| Protein | Fusion Protein | denileukin diftitox |
| Protein | Protein | Dextran-Hemoglobin |
| Protein | Fusion Protein | DI-Leu16-IL2 |
| Protein | Recombinant Protein | dianexin |
| Protein | Recombinant Protein | dibotermin alfa |
| Protein | Recombinant Protein | DM-199 |
| Protein | Fusion Protein | DMX-101 |
| Protein | Fusion Protein | DNL-310 |
| Protein | Recombinant Protein | drotrecogin alfa (activated) |
| Protein | Fusion Protein | DSP-107 |
| Protein | Fusion Protein | dulaglutide |
| Protein | Recombinant Protein | ecallantide |
| Protein | Recombinant Protein | ECI-301 |
| Protein | Recombinant Protein | edodekin alfa |
| Protein | Fusion Protein | efavaleukin alfa |
| Protein | Fusion Protein | efineptakin alfa |
| Protein | Recombinant Protein | efinopegdutide |
| Protein | Recombinant Protein | eflapegrastim |
| Protein | Recombinant Protein | efpegsomatropin |
| Protein | Fusion Protein | eftansomatropin alfa |
| Protein | Fusion Protein | eftilagimod alfa |
| Protein | Fusion Protein | eftozanermin alfa |
| Protein | Recombinant Protein | empegfilgrastim |
| Protein | Recombinant Protein | entolimod |
| Protein | Fusion Protein | envafolimab |
| Protein | Recombinant Protein | epidermal growth factor |
| Protein | Recombinant Protein | epoetin alfa |
| Protein | Recombinant Protein | epoetin alfa Long Acting |
| Protein | Recombinant Protein | epoetin beta |
| Protein | Recombinant Protein | epoetin delta |
| Protein | Recombinant Protein | epoetin theta |
| Protein | Recombinant Protein | epoetin zeta |
| Protein | Recombinant Protein | ErepoXen |
| Protein | Fusion Protein | etanercept |
| Protein | Fusion Protein | etanercept biosimilar |
| Protein | Protein | EYS-611 |
| Protein | Fusion Protein | F-627 |
| Protein | Fusion Protein | F-652 |
| Protein | Fusion Protein | F-899 |
| Protein | Recombinant Protein | Fertavid |
| Protein | Fusion Protein | fexapotide triflutate |
| Protein | Fusion Protein | fibromun |
| Protein | Recombinant Protein | filgrastim |
| Protein | Recombinant Protein | follicle stimulating hormone |
| Protein | Recombinant Protein | follitropin alfa |
| Protein | Recombinant Protein | |
| Protein | Recombinant Protein | follitropin beta |
| Protein | Recombinant Protein | follitropin delta |
| Protein | Recombinant Protein | FOV-2501 |
| Protein | Recombinant Protein | FSH-GEX |
| Protein | Fusion Protein | |
| Protein | Fusion Protein | Fusion Protein to Antagonize EGFR for |
| | | Glioblastoma Multiforme and Malignant Glioma |
| Protein | Fusion Protein | Fusion Protein to Inhibit CD25 for Oncology |
| Protein | Fusion Protein | Fusion Protein to Target Mesothelin for Oncology |
| Protein | Recombinant Protein | GEM-ONJ |
| Protein | Protein | gemibotulinumtoxin A |
| Protein | Recombinant Protein | GR-007 |
| Protein | | GT-0486 |
| Protein | Fusion Protein | GXG-3 |
| Protein | Fusion Protein | GXG-6 |
| Protein | Protein | Haegarda |
| Protein | Protein | haptoglobin (human) |
| Protein | Fusion Protein | HB-0021 |
| Protein | Protein | hemoglobin glutamer-250 (bovine) |
| Protein | Protein | hemoglobin raffimer |
| Protein | Recombinant Protein | HER-902 |
| Protein | Recombinant Protein | HM-15912 |
| Protein | Fusion Protein | HX-009 |
| Protein | Fusion Protein | IBI-302 |
| Protein | Fusion Protein | ICON-1 |
| Protein | Fusion Protein | IGN-002 |
| Protein | Fusion Protein | IMCF-106C |
| Protein | Fusion Protein | IMM-01 |
| Protein | Protein | INB-03 |
| Protein | Fusion Protein | inbakicept |
| Protein | Fusion Protein | INBRX-101 |
| Protein | Protein | incobotulinumtoxin A |
| Protein | Protein | INS-068 |
| Protein | Protein | interferon alfa |
| Protein | Recombinant Protein | interferon alfa-2a |
| Protein | Recombinant Protein | interferon alfa-2b |
| Protein | Recombinant Protein; Small | interferon alfa-2b + ribavirin |
| | Molecule | |
| Protein | Recombinant Protein | interferon alfa-n3 |
| Protein | Recombinant Protein | interferon alfacon-1 |
| Protein | Recombinant Protein | interferon alpha-n1 |
| Protein | Recombinant Protein | interferon beta-1a |
| Protein | Recombinant Protein | interferon beta-1b |
| Protein | Recombinant Protein | interferon gamma-1b |
| Protein | Recombinant Protein | IRL-201805 |
| Protein | Recombinant Protein | KAN-101 |
| Protein | Fusion Protein | KD-033 |
| Protein | Protein | KER-050 |
| Protein | Fusion Protein | KH-903 |
| Protein | Recombinant Protein | KMRC-011 |
| Protein | Recombinant Protein | Kovaltry |
| Protein | Recombinant Protein | KP-100IT |
| Protein | Recombinant Protein | lenograstim |
| Protein | Recombinant Protein | lepirudin |
| Protein | Fusion Protein | LEVI-04 |
| Protein | Recombinant Protein | liatermin |
| Protein | Fusion Protein | LIB-003 |
| Protein | Recombinant Protein | lipegfilgrastim |
| Protein | Fusion Protein | LMB-100 |
| Protein | Recombinant Protein | lonapegsomatropin |
| Protein | Protein | LTI-01 |
| Protein | Fusion Protein | luspatercept |
| Protein | Recombinant Protein | lusupultide |
| Protein | Recombinant Protein | lutropin alfa |
| Protein | Recombinant Protein | M-9241 |
| Protein | Fusion Protein | MDNA-55 |
| Protein | Recombinant Protein | mecasermin |
| Protein | Recombinant Protein | mecasermin rinfabate |
| Protein | Protein | Menopur |
| Protein | Protein | menotropins |
| Protein | Recombinant Protein | methoxy polyethylene glycol-epoetin beta |
| Protein | Recombinant Protein | metreleptin |
| Protein | Recombinant Protein | MG-29 |
| Protein | Recombinant Protein | molgramostim |
| Protein | Recombinant Protein | MP-0250 |
| Protein | Recombinant Protein | MP-0274 |
| Protein | Recombinant Protein | MP-0310 |
| Protein | Fusion Protein | MT-3724 |
| Protein | Recombinant Protein | Multiferon |
| Protein | Recombinant Protein | Multikine |
| Protein | Recombinant Protein | NA-704 |
| Protein | Fusion Protein | naptumomab estafenatox |
| Protein | Recombinant Protein | NE-180 |
| Protein | Recombinant Protein | nepidermina |
| Protein | Recombinant Protein | NGM-386 |
| Protein | Recombinant Protein | NGM-395 |
| Protein | Fusion Protein | NGR-hTNF |
| Protein | Protein | nivobotulinumtoxin A |
| Protein | Fusion Protein | NIZ-985 |
| Protein | Recombinant Protein | NKTR-255 |
| Protein | Recombinant Protein | NKTR-358 |
| Protein | Recombinant Protein | NL-201 |
| Protein | Recombinant Protein | NMIL-121 |
| Protein | Recombinant Protein | NN-7128 |
| Protein | Protein | NN-9215 |
| Protein | Recombinant Protein | NN-9499 |
| Protein | Recombinant Protein | novaferon |
| Protein | Fusion Protein | NPT-088 |
| Protein | Fusion Protein | NPT-189 |
| Protein | Protein | NStride APS |
| Protein | Fusion Protein | olamkicept |
| Protein | Protein | onabotulinumtoxin A |
| Protein | Protein | onabotulinumtoxinA biosimilar |
| Protein | Protein | onabotulinumtoxinA SR |
| Protein | Recombinant Protein | Oncolipin-IT |
| Protein | Recombinant Protein | OPK-88005 |
| Protein | Fusion Protein | oportuzumab monatox |
| Protein | Recombinant Protein | oprelvekin |
| Protein | Recombinant Protein | OPT-302 |
| Protein | Protein | OTO-413 |
| Protein | Fusion Protein | OXS-1550 |
| Protein | Fusion Protein | OXS-3550 |
| Protein | Recombinant Protein | palifermin |
| Protein | Fusion Protein | PB-1046 |
| Protein | Recombinant Protein | PBB-8-IN |
| Protein | Recombinant Protein | PD-1 Antagonist + ropeginterferon alfa-2b |
| Protein | Recombinant Protein | PEG-EPO |
| Protein | Recombinant Protein | pegbelfermin |
| Protein | Recombinant Protein | pegfilgrastim |
| Protein | Recombinant Protein | pegilodecakin |
| Protein | Recombinant Protein | peginterferon alfa-2a |
| Protein | Recombinant Protein; Small | peginterferon alfa-2a + ribavirin |
| | Molecule | |
| Protein | Recombinant Protein | peginterferon alfa-2b |
| Protein | Recombinant Protein; Small | peginterferon alfa-2b + ribavirin |
| | Molecule | |
| Protein | Recombinant Protein | peginterferon beta-1a |
| Protein | Recombinant Protein | peginterferon lambda-1a |
| Protein | Recombinant Protein | pegvisomant |
| Protein | Fusion Protein | PF-06755347 |
| Protein | Recombinant Protein | PIN-2 |
| Protein | Protein | plasminogen (human) |
| Protein | Protein | plasminogen (human) 1 |
| Protein | Fusion Protein | PR-15 |
| Protein | Protein | prabotulinumtoxin A biosimilar |
| Protein | Recombinant Protein | Prolanta |
| Protein | Recombinant Protein | PRS-080 |
| Protein | Fusion Protein | PRS-343 |
| Protein | Recombinant Protein | PRT-01 |
| Protein | Protein | PRTX-100 |
| Protein | Fusion Protein | PT-101 |
| Protein | Recombinant Protein | PTR-01 |
| Protein | Recombinant Protein | PTX-9908 |
| Protein | Fusion Protein | QL-1207 |
| Protein | Fusion Protein | RC-28 |
| Protein | Recombinant Protein | RecD-1 |
| Protein | Recombinant Protein | Recombinant Factor VIII Replacement for |
| | | Hemophilia A |
| Protein | Recombinant Protein | Recombinant Plasma Gelsolin Replacement for |
| | | Infectious Disease |
| Protein | Recombinant Protein | Recombinant Protein to Agonize BMPR1A, |
| | | BMPR1B and BMPR2 for Colorectal Cancer and |
| | | Glioblastoma Multiforme |
| Protein | Recombinant Protein | Recombinant Protein to Agonize IFNAR1 and |
| | | IFNAR2 for Oncology |
| Protein | Recombinant Protein | Recombinant Protein to Inhibit CD13 for |
| | | Lymphoma and Solid Tumor |
| Protein | Recombinant Protein | Recombinant Protein to Inhibit Coagulation Factor |
| | | XIV for Hemophilia A and Hemophilia B |
| Protein | Recombinant Protein | Recombinant Protein to Target FLT1 for Pre- |
| | | Eclampsia |
| Protein | Fusion Protein | reveglucosidase alfa |
| Protein | Fusion Protein | RG-6290 |
| Protein | Fusion Protein | RG-7461 |
| Protein | Fusion Protein | RG-7835 |
| Protein | Recombinant Protein | RG-7880 |
| Protein | Fusion Protein | rilonacept |
| Protein | Protein | rimabotulinumtoxin B |
| Protein | Recombinant Protein | RMC-035 |
| Protein | Fusion Protein | RO-7227166 |
| Protein | Fusion Protein | romiplostim |
| Protein | Fusion Protein | romiplostim biosimilar |
| Protein | Recombinant Protein | ropeginterferon alfa-2b |
| Protein | Recombinant Protein | RP-72 |
| Protein | Fusion Protein | RPH-104 |
| Protein | Fusion Protein | RPH-203 |
| Protein | Fusion Protein | RSLV-132 |
| Protein | Protein | RT-002 |
| Protein | Fusion Protein | SAL-016 |
| Protein | Recombinant Protein | Sanguinate |
| Protein | Fusion Protein | SAR-442085 |
| Protein | Recombinant Protein | sargramostim |
| Protein | Recombinant Protein | SC-0806 |
| Protein | Fusion Protein | SCB-313 |
| Protein | Recombinant Protein | serelaxin |
| Protein | Fusion Protein | SFR-9216 |
| Protein | Recombinant Protein | SHP-608 |
| Protein | Fusion Protein | SHR-1501 |
| Protein | Recombinant Protein | SIM-0710 |
| Protein | Fusion Protein | SL-279252 |
| Protein | Fusion Protein | SOC-101 |
| Protein | Recombinant Protein | somapacitan |
| Protein | Recombinant Protein | somatropin |
| Protein | Recombinant Protein | somatropin pegol |
| Protein | Recombinant Protein | somatropin PR |
| Protein | Recombinant Protein | somatropin SR |
| Protein | Recombinant Protein | somavaratan |
| Protein | Fusion Protein | sotatercept |
| Protein | Recombinant Protein | sprifermin |
| Protein | Recombinant Protein | SubQ-8 |
| Protein | Recombinant Protein | Sylatron |
| Protein | Fusion Protein | T-Guard |
| Protein | Recombinant Protein | TA-46 |
| Protein | Recombinant Protein | tadekinig alfa |
| Protein | Fusion Protein | tagraxofusp |
| Protein | Protein | TAK-101 |
| Protein | Fusion Protein | TAK-169 |
| Protein | Fusion Protein | TAK-573 |
| Protein | Fusion Protein | TAK-671 |
| Protein | Fusion Protein | talditercept alfa |
| Protein | Recombinant Protein | tasonermin |
| Protein | Recombinant Protein | TBI-302 |
| Protein | Recombinant Protein | tbo-filgrastim |
| Protein | Fusion Protein | tebentafusp |
| Protein | Fusion Protein | Teleukin |
| Protein | Fusion Protein | telitacicept |
| Protein | Fusion Protein | TG-103 |
| Protein | Recombinant Protein | THOR-707 |
| Protein | Recombinant Protein | thrombomodulin alfa |
| Protein | Recombinant Protein | thrombopoietin |
| Protein | Recombinant Protein | thyrotropin alfa |
| Protein | Recombinant Protein | tiprelestat |
| Protein | Recombinant Protein | topsalysin |
| Protein | Recombinant Protein | TransMID |
| Protein | Fusion Protein | trebananib |
| Protein | Fusion Protein | TTI-621 |
| Protein | Fusion Protein | TTI-622 |
| Protein | Fusion Protein | tucotuzumab celmoleukin |
| Protein | Recombinant Protein | TVN-102 |
| Protein | Fusion Protein | UCHT-1 |
| Protein | Fusion Protein | VAL-1221 |
| Protein | Fusion Protein | Vas-01 |
| Protein | Recombinant Protein | vatreptacog alfa (activated) |
| Protein | Fusion Protein | VB-4847 |
| Protein | Recombinant Protein | von willebrand factor (recombinant) |
| Protein | Fusion Protein | YSPSL |
| Protein | Fusion Protein | ziv-aflibercept |
| Protein | Protein | ZK-001 |
| Protein | Recombinant Protein | Zorbtive |

### B. Enzymes

The exogenous polypeptide may be an enzyme, e.g., an enzyme that catalyzes a biological reaction that is of use in the prevention or treatment of a condition or a disease, the prevention or treatment of a pathogen infection, the diagnosis of a disease, or the diagnosis of a disease or condition.

The enzyme may be a recombination enzyme, e.g., a Cre recombinase enzyme. In some aspects, the Cre recombinase enzyme is delivered by a PMP to a cell comprising a Cre reporter construct.

The enzyme may be an editing enzyme, e.g., a gene editing enzyme. In some aspects, the gene editing enzyme is a, e.g., a component of a CRISPR-Cas system (e.g., a Cas9 enzyme), a TALEN, or a zinc finger nuclease.

### C. Pathogen Control Agents

The exogenous polypeptide may be a pathogen control agent, e.g., a polypeptide that is an antibacterial, antifungal, insecticidal, nematicidal, antiparasitic, or virucidal. In some instances, the PMP or PMP composition described herein includes a polypeptide or functional fragments or derivative thereof, that targets pathways in the pathogen. A PMP composition including a polypeptide as described herein can be administered to a pathogen, a vector thereof, in an amount and for a time sufficient to: (a) reach a target level (e.g., a predetermined or threshold level) of polypeptide concentration; and (b) decrease or eliminate the pathogen. In some instances, a PMP composition including a polypeptide as described herein can be administered to an animal having or at risk of an infection by a pathogen in an amount and for a time sufficient to: (a) reach a target level (e.g., a predetermined or threshold level) of polypeptide concentration in the animal; and (b) decrease or eliminate the pathogen. The polypeptides described herein may be formulated in a PMP composition for any of the methods described herein, and in certain instances, may be associated with the PMP thereof.

Examples of polypeptides that can be used herein can include an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone.

The PMP described herein may include a bacteriocin. In some instances, the bacteriocin is naturally produced by Gram-positive bacteria, such as Pseudomonas, Streptomyces, Bacillus, Staphylococcus, or lactic acid bacteria (LAB, such as Lactococcus lactis). In some instances, the bacteriocin is naturally produced by Gram-negative bacteria, such as Hafnia alvei, Citrobacter freundii, Klebsiella oxytoca, Klebsiella pneumonia, Enterobacter cloacae, Serratia plymithicum, Xanthomonas campestris, Erwinia carotovora, Ralstonia solanacearum, or Escherichia coli. Exemplary bacteriocins include, but are not limited to, Class I-IV LAB antibiotics (such as lantibiotics), colicins, microcins, and pyocins.

The PMP described herein may include an antimicrobial peptide (AMP). Any AMP suitable for inhibiting a microorganism may be used. AMPs are a diverse group of molecules, which are divided into subgroups on the basis of their amino acid composition and structure. The AMP may be derived or produced from any organism that naturally produces AMPs, including AMPs derived from plants (e.g., copsin), insects (e.g., mastoparan, poneratoxin, cecropin, moricin, melittin), frogs (e.g., magainin, dermaseptin, aurein), and mammals (e.g., cathelicidins, defensins and protegrins).

### IV. Methods for Producing a PMP Comprising an Exogenous Polypeptide

In another aspect, the disclosure, in general, features a method of producing a PMP comprising an exogenous polypeptide. The method accordingly comprises (a) providing a solution comprising the exogenous polypeptide; and (b) loading the PMP with the exogenous polypeptide, wherein the loading causes the exogenous polypeptide to be encapsulated by the PMP.

The exogenous polypeptide may be placed in a solution, e.g., a phosphate-buffered saline (PBS) solution. The exogenous polypeptide may or may not be soluble in the solution. If the polypeptide is not soluble in the solution, the pH of the solution may be adjusted until the polypeptide is soluble in the solution. Insoluble polypeptides are also useful for loading.

Loading of the PMP with the exogenous polypeptide may comprise or consist of sonication of a solution comprising the exogenous polypeptide (e.g., a soluble or insoluble exogenous polypeptide) and a plurality of PMPs to induce poration of the PMPs and diffusion of the polypeptide into the PMPs, e.g., sonication according to the protocol described in Wang et al., Nature Comm., 4: 1867, 2013.

Alternatively, loading of the PMP with the exogenous polypeptide may comprise or consist of electroporation of a solution comprising the exogenous polypeptide (e.g., a soluble or insoluble exogenous polypeptide) and a plurality of PMPs, e.g., electroporation according to the protocol described in Wahlgren et al., Nucl. Acids. Res., 40(17), e130, 2012.

Alternatively, a small amount of a detergent (e.g., saponin) can be added to increase loading of the exogenous polypeptide into PMPs, e.g., as described in Fuhrmann et al., J Control Release., 205: 35-44, 2015.

Loading of the PMP with the exogenous polypeptide may comprise or consist of lipid extraction and lipid extrusion. Briefly, PMP lipids may be isolated by adding MeOH:CHCl₃ (e.g., 3.75 mL 2:1 (v/v) MeOH:CHCl₃) to PMPs in a PBS solution (e.g., 1 mL of PMPs in PBS) and vortexing the mixture. CHCl₃ (e.g., 1.25 mL) and ddH₂O (e.g., 1.25 mL) are then added sequentially and vortexed. The mixture is then centrifuged at 2,000 r.p.m. for 10 min at 22° C. in glass tubes to separate the mixture into two phases (aqueous phase and organic phase). The organic phase sample containing the PMP lipids is dried by heating under nitrogen (2 psi). To produce polypeptide-loaded PMPs, the isolated PMP lipids are mixed with the polypeptide solution and passed through a lipid extruder, e.g., according to the protocol from Haney et al., J Control Release, 207: 18-30, 2015.

PMP lipids may also be isolated using methods that isolate additional plant lipid classes, e.g., glycosylinositol phosphorylceramides (GIPCs), as described in Casas et al., Plant Physiology, 170: 367-384, 2016. Briefly, to extract PMP lipids including GIPCs, chloroform:methanol:HCl (e.g., 3.5 mL of chloroform:methanol:HCl (200:100:1, v/v/v)) plus butylated hydroxytoluene (e.g., 0.01% (w/v) of butylated hydroxytoluene) is added to and incubated with the PMPs. Next, NaCl (e.g., 2 mL of 0.9% (w/v) NaCl) is added and vortexed for 5 minutes. The sample is then centrifuged to induce the organic phase to aggregate at the bottom of the glass tube, and the organic phase is collected. The upper phase may undergo reextraction with chloroform (e.g., 4 mL of pure chloroform) to isolate lipids. The organic phases are combined and dried. After drying, the aqueous phase is resuspended in water (e.g., 1 mL of pure water) and GIPCs are back-extracted using butanol-1 (e.g., 1 mL of butanol-1) twice. To produce polypeptide-loaded PMPs, the isolated PMP lipid phases are mixed with the polypeptide solution and are passed through a lipid extruder according to the protocol from Haney et al., J Control Release, 207: 18-30, 2015. Alternatively, lipids may be extracted with methyl tertiary-butyl ether (MTBE):methanol:water plus butylated hydroxytoluene (BHT) or with propan-2-ol:hexane:water.

In some aspects, isolated GIPCs may be added to isolated PMP lipids.

In some aspects, loading of the PMP with the exogenous polypeptide comprises sonication and lipid extrusion, as described above.

In some aspects the exogenous polypeptide may be pre-complexed (e.g., using protamine sulfate), or a cationic lipid (e.g., DOTAP) may be added to facilitate encapsulation of negatively charged proteins.

Before use, the loaded PMPs may be purified, e.g., as described in Example 2, to remove polypeptides that are not bound to or encapsulated by the PMP. Loaded PMPs may be characterized as described in Example 3, and their stability may be tested as described in Example 4. Loading of the exogenous polypeptide may be quantified by methods known in the art for the quantification of proteins. For example, the Pierce Quantitative Colorimetric Peptide Assay may be used on a small sample of the loaded and unloaded PMPs, or a Western blot using specific antibodies may be used to detect the exogenous polypeptide. Alternatively, polypeptides may be fluorescently labeled, and fluorescence may be used to determine the labeled exogenous polypeptide concentration in loaded and unloaded PMPs.

### V. Therapeutic Methods

The PMPs and PMP compositions described herein are useful in a variety of therapeutic methods, particularly for the prevention or treatment of a condition or disease or for the prevention or treatment of pathogen infections in animals. The present methods involve delivering the PMP compositions described herein to an animal.

Provided herein are methods of administering to an animal a PMP composition disclosed herein. The methods can be useful for preventing or treating a condition or disease or for preventing a pathogen infection in an animal.

For example, provided herein is a method of treating an animal having a fungal infection, wherein the method includes administering to the animal an effective amount of a PMP composition including a plurality of PMPs, wherein the plurality of PMPs comprise an exogenous polypeptide that is a pathogen control agent, e.g., an antifungal agent. In some instances, the fungal infection is caused by Candida albicans. In some instances, the method decreases or substantially eliminates the fungal infection.

In another aspect, provided herein is a method of treating an animal having a bacterial infection, wherein the method includes administering to the animal an effective amount of a PMP composition including a plurality of PMPs. In some instances, the method includes administering to the animal an effective amount of a PMP composition including a plurality of PMPs, wherein the plurality of PMPs comprise an exogenous polypeptide that is a pathogen control agent, e.g., an antibacterial agent. In some instances, the bacterium is a Streptococcus spp., Pneumococcus spp., Pseudamonas spp., Shigella spp, Salmonella spp., Campylobacter spp., or an Escherichia spp. In some instances, the method decreases or substantially eliminates the bacterial infection. In some instances, the animal is a human, a veterinary animal, or a livestock animal.

The present methods are useful to treat an infection (e.g., as caused by an animal pathogen) in an animal, which refers to administering treatment to an animal already suffering from a disease to improve or stabilize the animal's condition. This may involve reducing colonization of a pathogen in, on, or around an animal by one or more pathogens (e.g., by about 1%, 2%, 5%, 10%, 20%, 30%, 40%, 50%, 60%, 70%, 80%, 90%, or 100%) relative to a starting amount and/or allow benefit to the individual (e.g., reducing colonization in an amount sufficient to resolve symptoms). In such instances, a treated infection may manifest as a decrease in symptoms (e.g., by about 1%, 2%, 5%, 10%, 20%, 30%, 40%, 50%, 60%, 70%, 80%, 90%, or 100%). In some instances, a treated infection is effective to increase the likelihood of survival of an individual (e.g., an increase in likelihood of survival by about 1%, 2%, 5%, 10%, 20%, 30%, 40%, 50%, 60%, 70%, 80%, 90%, or 100%) or increase the overall survival of a population (e.g., an increase in likelihood of survival by about 1%, 2%, 5%, 10%, 20%, 30%, 40%, 50%, 60%, 70%, 80%, 90%, or 100%). For example, the compositions and methods may be effective to “substantially eliminate” an infection, which refers to a decrease in the infection in an amount sufficient to sustainably resolve symptoms (e.g., for at least 1, 2, 3, 4, 5, 6, 7, 8, 9, 10, 11, or 12 months) in the animal.

The present methods are useful to prevent an infection (e.g., as caused by an animal pathogen), which refers to preventing an increase in colonization in, on, or around an animal by one or more pathogens (e.g., by about 1%, 2%, 5%, 10%, 20%, 30%, 40%, 50%, 60%, 70%, 80%, 90%, 100%, or more than 100% relative to an untreated animal) in an amount sufficient to maintain an initial pathogen population (e.g., approximately the amount found in a healthy individual), prevent the onset of an infection, and/or prevent symptoms or conditions associated with infection. For example, individuals may receive prophylaxis treatment to prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised individuals (e.g., individuals with cancer, with HIV/AIDS, or taking immunosuppressive agents), or in individuals undergoing long term antibiotic therapy.

The PMP composition can be formulated for administration or administered by any suitable method, including, for example, orally, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, topically, transdermally, intravitreally (e.g., by intravitreal injection), by eye drop, by inhalation (e.g., by a nebulizer), by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some instances, the PMP composition is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., orally or by injections, such as intravenous or subcutaneous injections, depending in part on whether the administration is brief or chronic. Various dosing schedules including but not limited to single or multiple administrations over various time-points, bolus administration, and pulse infusion are contemplated herein.

For the prevention or treatment of an infection described herein (when used alone or in combination with one or more other additional therapeutic agents) will depend on the type of disease to be treated, the severity and course of the disease, whether the is administered for preventive or therapeutic purposes, previous therapy, the patient's clinical history and response to the PMP composition. The PMP composition can be, e.g., administered to the patient at one time or over a series of treatments. For repeated administrations over several days or longer, depending on the condition, the treatment would generally be sustained until a desired suppression of disease symptoms occurs or the infection is no longer detectable. Such doses may be administered intermittently, e.g., every week or every two weeks (e.g., such that the patient receives, for example, from about two to about twenty, doses of the PMP composition. An initial higher loading dose, followed by one or more lower doses may be administered. However, other dosage regimens may be useful. The progress of this therapy is easily monitored by conventional techniques and assays.

In some instances, the amount of the PMP composition administered to individual (e.g., human) may be in the range of about 0.01 mg/kg to about 5 g/kg (e.g., about 0.01 mg/kg-0.1 mg/kg, about 0.1 mg/kg-1 mg/kg, about 1 mg/kg-10 mg/kg, about 10 mg/kg-100 mg/kg, about 100 mg/kg-1 g/kg, or about 1 g/kg-5 g/kg), of the individual's body weight. In some instances, the amount of the PMP composition administered to individual (e.g., human) is at least 0.01 mg/kg (e.g., at least 0.01 mg/kg, at least 0.1 mg/kg, at least 1 mg/kg, at least 10 mg/kg, at least 100 mg/kg, at least 1 g/kg, or at least 5 g/kg), of the individual's body weight. The dose may be administered as a single dose or as multiple doses (e.g., 2, 3, 4, 5, 6, 7, or more than 7 doses). In some instances, the PMP composition administered to the animal may be administered alone or in combination with an additional therapeutic agent or pathogen control agent. The dose of an antibody administered in a combination treatment may be reduced as compared to a single treatment. The progress of this therapy is easily monitored by conventional techniques.

In one aspect, the disclosure features a method for treating diabetes, the method comprising administering to a subject in need thereof an effective amount of a composition comprising a plurality of PMPs, wherein one or more exogenous polypeptides are encapsulated by the PMP. The administration of the plurality of PMPs may lower the blood sugar of the subject. In some aspects, the exogenous polypeptide is insulin.

### VI. Agricultural Methods

The PMP compositions described herein are useful in a variety of agricultural methods, particularly for the prevention or treatment of pathogen infections in animals and for the control of the spread of such pathogens, e.g., by pathogen vectors. The present methods involve delivering the PMP compositions described herein to a pathogen or a pathogen vector.

The compositions and related methods can be used to prevent infestation by or reduce the numbers of pathogens or pathogen vectors in any habitats in which they reside (e.g., outside of animals, e.g., on plants, plant parts (e.g., roots, fruits and seeds), in or on soil, water, or on another pathogen or pathogen vector habitat. Accordingly, the compositions and methods can reduce the damaging effect of pathogen vectors by for example, killing, injuring, or slowing the activity of the vector, and can thereby control the spread of the pathogen to animals. Compositions disclosed herein can be used to control, kill, injure, paralyze, or reduce the activity of one or more of any pathogens or pathogen vectors in any developmental stage, e.g., their egg, nymph, instar, larvae, adult, juvenile, or desiccated forms. The details of each of these methods are described further below.

A. Delivery to a Pathogen

Provided herein are methods of delivering a PMP composition to a pathogen, such as one disclosed herein, by contacting the pathogen with a PMP composition comprising an exogenous polypeptide, e.g., a pathogen control agent. The methods can be useful for decreasing the fitness of a pathogen, e.g., to prevent or treat a pathogen infection or control the spread of a pathogen as a consequence of delivery of the PMP composition. Examples of pathogens that can be targeted in accordance with the methods described herein include bacteria (e.g., Streptococcus spp., Pneumococcus spp., Pseudamonas spp., Shigella spp, Salmonella spp., Campylobacter spp., or an Escherichia spp), fungi (Saccharomyces spp. or a Candida spp), parasitic insects (e.g., Cimex spp), parasitic nematodes (e.g., Heligmosomoides spp), or parasitic protozoa (e.g., Trichomoniasis spp).

For example, provided herein is a method of decreasing the fitness of a pathogen, the method including delivering to the pathogen any of the compositions described herein, wherein the method decreases the fitness of the pathogen relative to an untreated pathogen. In some embodiments, the method includes delivering a PMP composition comprising an exogenous polypeptide, e.g., a pathogen control agent to at least one habitat where the pathogen grows, lives, reproduces, feeds, or infests. In some instances of the methods described herein, the composition is delivered as a pathogen comestible composition for ingestion by the pathogen. In some instances of the methods described herein, the composition is delivered (e.g., to a pathogen) as a liquid, a solid, an aerosol, a paste, a gel, or a gas.

Also provided herein is a method of decreasing the fitness of a parasitic insect, wherein the method includes delivering to the parasitic insect a PMP composition including a plurality of PMPs comprising an exogenous polypeptide, e.g., a pathogen control agent. For example, the parasitic insect may be a bedbug. Other non-limiting examples of parasitic insects are provided herein. In some instances, the method decreases the fitness of the parasitic insect relative to an untreated parasitic insect

Additionally provided herein is a method of decreasing the fitness of a parasitic nematode, wherein the method includes delivering to the parasitic nematode a PMP composition including a plurality of PMPs comprising an exogenous polypeptide, e.g., a pathogen control agent. For example, the parasitic nematode is Heligmosomoides polygyrus. Other non-limiting examples of parasitic nematodes are provided herein. In some instances, the method decreases the fitness of the parasitic nematode relative to an untreated parasitic nematode.

Further provided herein is a method of decreasing the fitness of a parasitic protozoan, wherein the method includes delivering to the parasitic protozoan a PMP composition including a plurality of PMPs comprising an exogenous polypeptide, e.g., a pathogen control agent. For example, the parasitic protozoan may be T. vaginalis. Other non-limiting examples of parasitic protozoans are provided herein. In some instances, the method decreases the fitness of the parasitic protozoan relative to an untreated parasitic protozoan.

A decrease in the fitness of the pathogen as a consequence of delivery of a PMP composition can manifest in a number of ways. In some instances, the decrease in fitness of the pathogen may manifest as a deterioration or decline in the physiology of the pathogen (e.g., reduced health or survival) as a consequence of delivery of the PMP composition. In some instances, the fitness of an organism may be measured by one or more parameters, including, but not limited to, reproductive rate, fertility, lifespan, viability, mobility, fecundity, pathogen development, body weight, metabolic rate or activity, or survival in comparison to a pathogen to which the PMP composition has not been administered. For example, the methods or compositions provided herein may be effective to decrease the overall health of the pathogen or to decrease the overall survival of the pathogen. In some instances, the decreased survival of the pathogen is about 2%, 5%, 10%, 20%, 30%, 40%, 50%, 60%, 70%, 80%, 90%, 100%, or greater than 100% greater relative to a reference level (e.g., a level found in a pathogen that does not receive a PMP composition comprising an exogenous polypeptide, e.g., a pathogen control agent. In some instances, the methods and compositions are effective to decrease pathogen reproduction (e.g., reproductive rate, fertility) in comparison to a pathogen to which the PMP composition has not been administered. In some instances, the methods and compositions are effective to decrease other physiological parameters, such as mobility, body weight, life span, fecundity, or metabolic rate, by about 2%, 5%, 10%, 20%, 30%, 40%, 50%, 60%, 70%, 80%, 90%, 100%, or greater than 100% relative to a reference level (e.g., a level found in a pathogen that does not receive a PMP composition).

In some instances, the decrease in pest fitness may manifest as an increase in the pathogen's sensitivity to an antipathogen agent and/or a decrease in the pathogen's resistance to an antipathogen agent in comparison to a pathogen to which the PMP composition has not been delivered. In some instances, the methods or compositions provided herein may be effective to increase the pathogen's sensitivity to a pesticidal agent by about 2%, 5%, 10%, 20%, 30%, 40%, 50%, 60%, 70%, 80%, 90%, 100%, or greater than 100% relative to a reference level (e.g., a level found in a pest that does not receive a PMP composition).

In some instances, the decrease in pathogen fitness may manifest as other fitness disadvantages, such as a decreased tolerance to certain environmental factors (e.g., a high or low temperature tolerance), a decreased ability to survive in certain habitats, or a decreased ability to sustain a certain diet in comparison to a pathogen to which the pathogen control (composition has not been delivered. In some instances, the methods or compositions provided herein may be effective to decrease pathogen fitness in any plurality of ways described herein. Further, the PMP composition may decrease pathogen fitness in any number of pathogen classes, orders, families, genera, or species (e.g., 1 pathogen species, 2, 3, 4, 5, 6, 7, 8, 9, 10, 15, 20, 30, 40, 50, 60, 70, 80, 90, 100, 150, 200, 200, 250, 500, or more pathogen species). In some instances, the PMP composition acts on a single pest class, order, family, genus, or species.

Pathogen fitness may be evaluated using any standard methods in the art. In some instances, pest fitness may be evaluated by assessing an individual pathogen. Alternatively, pest fitness may be evaluated by assessing a pathogen population. For example, a decrease in pathogen fitness may manifest as a decrease in successful competition against other pathogens, thereby leading to a decrease in the size of the pathogen population.

### VII. Methods for Treatment of Pathogens or Vectors Thereof

The PMP compositions and related methods described herein are useful to decrease the fitness of an animal pathogen and thereby treat or prevent infections in animals. Examples of animal pathogens, or vectors thereof, that can be treated with the present compositions or related methods are further described herein.

### A. Fungi

The PMP compositions and related methods can be useful for decreasing the fitness of a fungus, e.g., to prevent or treat a fungal infection in an animal. Included are methods for delivering a PMP composition to a fungus by contacting the fungus with the PMP composition. Additionally or alternatively, the methods include preventing or treating a fungal infection (e.g., caused by a fungus described herein) in an animal at risk of or in need thereof, by administering to the animal a PMP composition.

The PMP compositions and related methods are suitable for treatment or preventing of fungal infections in animals, including infections caused by fungi belonging to Ascomycota (Fusarium oxysporum, Pneumocystis jirovecii, Aspergillus spp., Coccidioides immitis/posadasii, Candida albicans), Basidiomycota (Filobasidiella neoformans, Trichosporon), Microsporidia (Encephalitozoon cuniculi, Enterocytozoon bieneusi), Mucoromycotina (Mucor circinelloides, Rhizopus oryzae, Lichtheimia corymbifera).

In some instances, the fungal infection is one caused by a belonging to the phylum Ascomycota, Basidomycota, Chytridiomycota, Microsporidia, or Zygomycota. The fungal infection or overgrowth can include one or more fungal species, e.g., Candida albicans, C. tropicalis, C. parapsilosis, C. glabrata, C. auris, C. krusei, Saccharomyces cerevisiae, Malassezia globose, M. restricta, or Debaryomyces hansenii, Gibberella moniliformis, Alternaria brassicicola, Cryptococcus neoformans, Pneumocystis carinii, P. jirovecii, P. murina, P. oryctolagi, P. wakefieldiae, and Aspergillus clavatus. The fungal species may be considered a pathogen or an opportunistic pathogen.

In some instances, the fungal infection is caused by a fungus in the genus Candida (i.e., a Candida infection). For example, a Candida infection can be caused by a fungus in the genus Candida that is selected from the group consisting of C. albicans, C. glabrata, C. dubliniensis, C. krusei, C. auris, C. parapsilosis, C. tropicalis, C. orthopsilosis, C. guiffiermondii, C. rugose, and C. lusitaniae. Candida infections that can be treated by the methods disclosed herein include, but are not limited to candidemia, oropharyngeal candidiasis, esophageal candidiasis, mucosal candidiasis, genital candidiasis, vulvovaginal candidiasis, rectal candidiasis, hepatic candidiasis, renal candidiasis, pulmonary candidiasis, splenic candidiasis, otomycosis, osteomyelitis, septic arthritis, cardiovascular candidiasis (e.g., endocarditis), and invasive candidiasis.

### B. Bacteria

The PMP compositions and related methods can be useful for decreasing the fitness of a bacterium, e.g., to prevent or treat a bacterial infection in an animal. Included are methods for administering a PMP composition to a bacterium by contacting the bacteria with the PMP composition. Additionally or alternatively, the methods include preventing or treating a bacterial infection (e.g., caused by a bacteria described herein) in an animal at risk of or in need thereof, by administering to the animal a PMP composition.

The PMP compositions and related methods are suitable for preventing or treating a bacterial infection in animals caused by any bacteria described further below. For example, the bacteria may be one belonging to Bacillales (B. anthracis, B. cereus, S. aureus, L. monocytogenes), Lactobacillales (S. pneumoniae, S. pyogenes), Clostridiales (C. botulinum, C. difficile, C. perfringens, C. tetani), Spirochaetales (Borrelia burgdorferi, Treponema pallidum), Chlamydiales (Chlamydia trachomatis, Chlamydophila psittaci), Actinomycetales (C. diphtheriae, Mycobacterium tuberculosis, M. avium), Rickettsiales (R. prowazekii, R. rickettsii, R. typhi, A. phagocytophilum, E. chaffeensis), Rhizobiales (Brucella melitensis), Burkholderiales (Bordetella pertussis, Burkholderia mallei, B. pseudomallei), Neisseriales (Neisseria gonorrhoeae, N. meningitidis), Campylobacterales (Campylobacter jejuni, Helicobacter pylon), Legionellales (Legionella pneumophila), Pseudomonadales (A. baumannii, Moraxella catarrhalis, P. aeruginosa), Aeromonadales (Aeromonas sp.), Vibrionales (Vibrio cholerae, V. parahaemolyticus), Thiotrichales, Pasteurellales (Haemophilus influenzae), Enterobacteriales (Klebsiella pneumoniae, Proteus mirabilis, Yersinia pestis, Y. enterocolitica, Shigella flexneri, Salmonella enterica, E. coli).

### EXAMPLES

The following are examples of the various methods of the invention. It is understood that various other embodiments may be practiced, given the general description provided above.

### Example 1: Crude Isolation of Plant Messenger Packs from Plants

This example describes the crude isolation of plant messenger packs (PMPs) from various plant sources, including the leaf apoplast, seed apoplast, root, fruit, vegetable, pollen, phloem, xylem sap and plant cell culture medium.

### Experimental Design:

a) PMP Isolation from the Apoplast of Arabidopsis thaliana Leaves

Arabidopsis (Arabidopsis thaliana Col-0) seeds are surface sterilized with 50% bleach and plated on 0.53 Murashige and Skoog medium containing 0.8% agar. The seeds are vernalized for 2 d at 4° C. before being moved to short-day conditions (9-h days, 22° C., 150 μEm⁻²). After 1 week, the seedlings are transferred to Pro-Mix PGX. Plants are grown for 4-6 weeks before harvest.

PMPs are isolated from the apoplastic wash of 4-6-week old Arabidopsis rosettes, as described by Rutter and Innes, Plant Physiol., 173(1): 728-741, 2017. Briefly, whole rosettes are harvested at the root and vacuum infiltrated with vesicle isolation buffer (20 mM MES, 2 mM CaCl₂), and 0.1 M NaCl, pH 6).

Infiltrated plants are carefully blotted to remove excess fluid, placed inside 30-mL syringes, and centrifuged in 50 mL conical tubes at 700 g for 20 min at 2° C. to collect the apoplast extracellular fluid containing PMPs. Next, the apoplast extracellular fluid is filtered through a 0.85 pm filter to remove large particles, and PMPs are purified as described in Example 2.

b) PMP Isolation from the Apoplast of Sunflower Seeds

Intact sunflower seeds (H. annuus L.) and are imbibed in water for 2 hours, peeled to remove the pericarp, and the apoplastic extracellular fluid is extracted by a modified vacuum infiltration-centrifugation procedure, adapted from Regente et al., FEBS Letters, 583: 3363-3366, 2009. Briefly, seeds are immersed in vesicle isolation buffer (20 mM MES, 2 mM CaCl₂), and 0.1 M NaCl, pH 6) and subjected to three vacuum pulses of 10s, separated by 30s intervals at a pressure of 45 kPa. The infiltrated seeds are recovered, dried on filter paper, placed in fritted glass filters, and centrifuged for 20 min at 400 g at 4° C. The apoplast extracellular fluid is recovered, filtered through a 0.85 pm filter to remove large particles, and PMPs are purified as described in Example 2.

c) PMP Isolation from Ginger Roots

Fresh ginger (Zingiber officinale) rhizomes are purchased from a local supplier and washed 3× with PBS. A total of 200 grams of washed roots is ground in a mixer (Osterizer 12-speed blender) at the highest speed for 10 min (pause 1 min for every 1 min of blending), and PMPs are isolated as described in Zhuang et al., J Extracellular Vesicles, 4(1): 28713, 2015. Briefly, ginger juice is sequentially centrifuged at 1,000 g for 10 min, 3,000 g for 20 min and 10,000 g for 40 min to remove large particles from the PMP-containing supernatant. PMPs are purified as described in Example 2.

d) PMP Isolation from Grapefruit Juice

Fresh grapefruits (Citrus×paradise) are purchased from a local supplier, the skins are removed, and the fruit is manually pressed, or ground in a mixer (Osterizer 12-speed blender) at the highest speed for 10 min (pause 1 min for every minute of blending) to collect the juice, as described by Wang et al., Molecular Therapy, 22(3): 522-534, 2014 with minor modifications. Briefly, juice/juice pulp is sequentially centrifuged at 1,000 g for 10 min, 3,000 g for 20 min, and 10,000 g for 40 min to remove large particles from the PMP-containing supernatant. PMPs are purified as described in Example 2.

e) PMP Isolation from a Broccoli Vegetable

Broccoli (Brassica oleracea var. italica) PMPs are isolated as previously described (Deng et al., Molecular Therapy, 25(7): 1641-1654, 2017). Briefly, fresh broccoli is purchased from a local supplier, washed three times with PBS, and ground in a mixer (Osterizer 12-speed blender) at the highest speed for 10 min (pause 1 min for every minute of blending). Broccoli juice is then sequentially centrifuged at 1,000 g for 10 min, 3,000 g for 20 min, and 10,000 g for 40 min to remove large particles from the PMP-containing supernatant. PMPs are purified as described in Example 2.

f) PMP Isolation from Olive Pollen

Olive (Olea europaea) pollen PMPs are isolated as previously described in Prado et al., Molecular Plant. 7(3):573-577, 2014. Briefly, olive pollen (0.1 g) is hydrated in a humid chamber at room temperature for 30 min before transferring to petri dishes (15 cm in diameter) containing 20 ml germination medium: 10% sucrose, 0.03% Ca(NO₃)₂, 0.01% KNO₃, 0.02% MgSO₄, and 0.03% H₃BO₃. Pollen is germinated at 30° C. in the dark for 16 h. Pollen grains are considered germinated only when the tube is longer than the diameter of the pollen grain. Cultured medium containing PMPs is collected and cleared of pollen debris by two successive filtrations on 0.85 um filters by centrifugation. PMPs are purified as described in Example 2.

g) PMP Isolation from Arabidopsis Phloem Sap

Arabidopsis (Arabidopsis thaliana Col-0) seeds are surface sterilized with 50% bleach and plated on 0.53 Murashige and Skoog medium containing 0.8% agar. The seeds are vernalized for 2 d at 4° C. before being moved to short-day conditions (9-h days, 22° C., 150 μEm⁻²). After 1 week, the seedlings are transferred to Pro-Mix PGX. Plants are grown for 4-6 weeks before harvest.

Phloem sap from 4-6-week old Arabidopsis rosette leaves is collected as described by Tetyuk et al., JoVE. 80, 2013. Briefly, leaves are cut at the base of the petiole, stacked, and placed in a reaction tube containing 20 mM K2-EDTA for one hour in the dark to prevent sealing of the wound. Leaves are gently removed from the container, washed thoroughly with distilled water to remove all EDTA, put in a clean tube, and phloem sap is collected for 5-8 hours in the dark. Leaves are discarded, phloem sap is filtered through a 0.85 pm filter to remove large particles, and PMPs are purified as described in Example 2.

h) PMP Isolation from Tomato Plant Xylem Sap

Tomato (Solanum lycopersicum) seeds are planted in a single pot in an organic-rich soil, such as Sunshine Mix (Sun Gro Horticulture, Agawam, Mass.) and maintained in a greenhouse between 22° C. and 28° C. About two weeks after germination, at the two true-leaf stage, the seedlings are transplanted individually into pots (10 cm diameter and 17 cm deep) filled with sterile sandy soil containing 90% sand and 10% organic mix. Plants are maintained in a greenhouse at 22-28° C. for four weeks.

Xylem sap from 4-week old tomato plants is collected as described by Kohlen et al., Plant Physiology. 155(2):721-734, 2011. Briefly, tomato plants are decapitated above the hypocotyl, and a plastic ring is placed around the stem. The accumulating xylem sap is collected for 90 min after decapitation. Xylem sap is filtered through a 0.85 pm filter to remove large particles, and PMPs are purified as described in Example 2.

i) PMP Isolation from Tobacco BY-2 Cell Culture Medium

Tobacco BY-2 (Nicotiana tabacum L cv. Bright Yellow 2) cells are cultured in the dark at 26° C., on a shaker at 180 rpm in MS (Murashige and Skoog, 1962) BY-2 cultivation medium (pH 5.8) comprising MS salts (Duchefa, Haarlem, Netherlands, at #M0221) supplemented with 30 g/L sucrose, 2.0 mg/L potassium dihydrogen phosphate, 0.1 g/L myo-inositol, 0.2 mg/L 2,4-dichlorophenoxyacetic acid, and 1 mg/L thiamine HCl. The BY-2 cells are subcultured weekly by transferring 5% (v/v) of a 7-day-old cell culture into 100 mL fresh liquid medium. After 72-96 hours, BY-2 cultured medium is collected and centrifuged at 300 g at 4° C. for 10 minutes to remove cells. The supernatant containing PMPs is collected and cleared of debris by filtration on 0.85 um filter. PMPs are purified as described in Example 2.

### Example 2: Production of Purified Plant Messenger Packs (PMPs)

This example describes the production of purified PMPs from crude PMP fractions as described in Example 1, using ultrafiltration combined with size-exclusion chromatography, a density gradient (iodixanol or sucrose), and the removal of aggregates by precipitation or size-exclusion chromatography.

### EXPERIMENTAL DESIGN

a) Production of Purified Grapefruit PMPs Using Ultrafiltration Combined with Size-Exclusion Chromatography

The crude grapefruit PMP fraction from Example 1a is concentrated using 100-kDA molecular weight cut-off (MWCO) Amicon spin filter (Merck Millipore). Subsequently, the concentrated crude PMP solution is loaded onto a PURE-EV size exclusion chromatography column (HansaBioMed Life Sciences Ltd) and isolated according to the manufacturer's instructions. The purified PMP-containing fractions are pooled after elution. Optionally, PMPs can be further concentrated using a 100-kDa MWCO Amicon spin filter, or by Tangential Flow Filtration (TFF). The purified PMPs are analyzed as described in Example 3.

b) Production of Purified Arabidopsis Apoplast PMPs Using an Iodixanol Gradient

Crude Arabidopsis leaf apoplast PMPs are isolated as described in Example 1a, and PMPs are produced by using an iodixanol gradient as described in Rutter and Innes, Plant Physiol. 173(1): 728-741, 2017. To prepare discontinuous iodixanol gradients (OptiPrep; Sigma-Aldrich), solutions of 40% (v/v), 20% (v/v), 10% (v/v), and 5% (v/v) iodixanol are created by diluting an aqueous 60% OptiPrep stock solution in vesicle isolation buffer (VIB; 20 mM MES, 2 mM CaCl₂), and 0.1 M NaCl, pH6). The gradient is formed by layering 3 ml of 40% solution, 3 mL of 20% solution, 3 mL of 10% solution, and 2 mL of 5% solution. The crude apoplast PMP solution from Example 1a is centrifuged at 40,000 g for 60 min at 4° C. The pellet is resuspended in 0.5 ml of VIB and layered on top of the gradient. Centrifugation is performed at 100,000 g for 17 h at 4° C. The first 4.5 ml at the top of the gradient is discarded, and subsequently 3 volumes of 0.7 ml that contain the apoplast PMPs are collected, brought up to 3.5 mL with VIB and centrifuged at 100,000 g for 60 min at 4° C. The pellets are washed with 3.5 ml of VIB and repelleted using the same centrifugation conditions. The purified PMP pellets are combined for subsequent analysis, as described in Example 3.

c) Production of Purified Grapefruit PMPs Using a Sucrose Gradient

Crude grapefruit juice PMPs are isolated as described in Example 1d, centrifuged at 150,000 g for 90 min, and the PMP-containing pellet is resuspended in 1 ml PBS as described in Mu et al., Molecular Nutrition & Food Research. 58(7):1561-1573, 2014. The resuspended pellet is transferred to a sucrose step gradient (8%/15%/30%/45%/60%) and centrifuged at 150,000 g for 120 min to produce purified PMPs. Purified grapefruit PMPs are harvested from the 30%/45% interface, and subsequently analyzed, as described in Example 3.

d) Removal of Aggregates from Grapefruit PMPs

In order to remove protein aggregates from produced grapefruit PMPs as described in Example 1d or purified PMPs from Example 2a-c, an additional purification step can be included. The produced PMP solution is taken through a range of pHs to precipitate protein aggregates in solution. The pH is adjusted to 3, 5, 7, 9, or 11 with the addition of sodium hydroxide or hydrochloric acid. pH is measured using a calibrated pH probe. Once the solution is at the specified pH, it is filtered to remove particulates. Alternatively, the isolated PMP solution can be flocculated using the addition of charged polymers, such as Polymin-P or Praestol 2640. Briefly, 2-5 g per L of Polymin-P or Praestol 2640 is added to the solution and mixed with an impeller. The solution is then filtered to remove particulates. Alternatively, aggregates are solubilized by increasing salt concentration. NaCl is added to the PMP solution until it is at 1 mol/L. The solution is then filtered to purify the PMPs. Alternatively, aggregates are solubilized by increasing the temperature. The isolated PMP mixture is heated under mixing until it has reached a uniform temperature of 50° C. for 5 minutes. The PMP mixture is then filtered to isolate the PMPs. Alternatively, soluble contaminants from PMP solutions are separated by size-exclusion chromatography column according to standard procedures, where PMPs elute in the first fractions, whereas proteins and ribonucleoproteins and some lipoproteins are eluted later. The efficiency of protein aggregate removal is determined by measuring and comparing the protein concentration before and after removal of protein aggregates via BCA/Bradford protein quantification. The produced PMPs are analyzed as described in Example 3.

### Example 3: Plant Messenger Pack Characterization

This example describes the characterization of PMPs produced as described in Example 1 or Example 2.

### Experimental Design

a) Determining PMP Concentration

PMP particle concentration is determined by Nanoparticle Tracking Analysis (NTA) using a Malvern NanoSight, nano flow cytometry using a NanoFCM, or by Tunable Resistive Pulse Sensing (TRPS) using an Spectradyne CS1, following the manufacturer's instructions. The protein concentration of purified PMPs is determined by using the DC Protein assay (Bio-Rad). The lipid concentration of purified PMPs is determined using a fluorescent lipophilic dye, such as DiOC6 (ICN Biomedicals) as described by Rutter and Innes, Plant Physiol. 173(1): 728-741, 2017. Briefly, purified PMP pellets from Example 2 are resuspended in 100 ml of 10 mM DiOC6 (ICN Biomedicals) diluted with MES buffer (20 mM MES, pH 6) plus 1% plant protease inhibitor cocktail (Sigma-Aldrich) and 2 mM 2,29-dipyridyl disulfide. The resuspended PMPs are incubated at 37° C. for 10 min, washed with 3 mL of MES buffer, repelleted (40,000 g, 60 min, at 4° C.), and resuspended in fresh MES buffer. DiOC6 fluorescence intensity is measured at 485 nm excitation and 535 nm emission.

b) Biophysical and Molecular Characterization of PMPs

PMPs are characterized by electron and cryo-electron microscopy on a JEOL 1010 transmission electron microscope, following the protocol from Wu et al., Analyst. 140(2):386-406, 2015. The size and zeta potential of the PMPs are also measured using a Malvern Zetasizer or iZon qNano, following the manufacturer's instructions. Lipids are isolated from PMPs using chloroform extraction and characterized with LC-MS/MS as demonstrated in Xiao et al. Plant Cell. 22(10): 3193-3205, 2010. Glycosyl inositol phosphorylceramides (GIPCs) lipids are extracted and purified as described by Cacas et al., Plant Physiology. 170: 367-384, 2016, and analyzed by LC-MS/MS as described above. Total RNA, DNA, and protein are characterized using Quant-It kits from Thermo Fisher according to instructions. Proteins on the PMPs are characterized by LC-MS/MS following the protocol in Rutter and Innes, Plant Physiol. 173(1): 728-741, 2017. RNA and DNA are extracted using Trizol, prepared into libraries with the TruSeq Total RNA with Ribo-Zero Plant kit and the Nextera Mate Pair Library Prep Kit from Illumina, and sequenced on an Illumina MiSeq following manufacturer's instructions.

### Example 4: Characterization of Plant Messenger Pack Stability

This example describes measuring the stability of PMPs under a wide variety of storage and physiological conditions.

### Experimental Design

PMPs produced as described in Examples 1 and 2 are subjected to various conditions. PMPs are suspended in water, 5% sucrose, or PBS and left for 1, 7, 30, and 180 days at −20° C., 4° C., 20° C., and 37° C. PMPs are also suspended in water and dried using a rotary evaporator system and left for 1, 7, and 30, and 180 days at 4° C., 20° C., and 37° C. PMPs are also suspended in water or 5% sucrose solution, flash-frozen in liquid nitrogen and lyophilized. After 1, 7, 30, and 180 days, dried and lyophilized PMPs are then resuspended in water. The previous three experiments with conditions at temperatures above 0° C. are also exposed to an artificial sunlight simulator in order to determine content stability in simulated outdoor UV conditions. PMPs are also subjected to temperatures of 37° C., 40° C., 45° C., 50° C., and 55° C. for 1, 6, and 24 hours in buffered solutions with a pH of 1, 3, 5, 7, and 9 with or without the addition of 1 unit of trypsin or in other simulated gastric fluids.

After each of these treatments, PMPs are bought back to 20° C., neutralized to pH 7.4, and characterized using some or all of the methods described in Example 3.

### Example 5. Loading PMPs with Polypeptide Cargo

This example describes methods of loading PMPs with polypeptides.

PMPs are produced as described in Example 1 and Example 2. To load polypeptides (e.g., proteins or peptides) into PMPs, PMPs are placed in solution with the polypeptide in phosphate-buffered saline (PBS). If the polypeptide is insoluble, the pH of the solution is adjusted until the polypeptide is soluble. If the polypeptide is still insoluble, the insoluble polypeptide is used. The solution is then sonicated to induce poration and diffusion into the PMPs according to the protocol from Wang et al., Nature Comm., 4: 1867, 2013. Alternatively, PMPs are electroporated according to the protocol from Wahlgren et al., Nucl. Acids. Res., 40(17), e130, 2012.

Alternatively, PMP lipids are isolated by adding 3.75 mL 2:1 (v/v) MeOH:CHCl₃ to 1 mL of PMPs in PBS and vortexing the mixture. CHCl₃ (1.25 mL) and ddH₂O (1.25 mL) are added sequentially and vortexed. The mixture is then centrifuged at 2,000 r.p.m. for 10 min at 22° C. in glass tubes to separate the mixture into two phases (aqueous phase and organic phase). The organic phase sample containing the PMP lipids is dried by heating under nitrogen (2 psi). To produce polypeptide-loaded PMPs, the isolated PMP lipids are mixed with the polypeptide solution and passed through a lipid extruder according to the protocol from Haney et al., J Control Release, 207: 18-30, 2015.

Alternatively, PMP lipids are isolated using methods that isolate additional plant lipid classes, including glycosylinositol phosphorylceramides (GIPCs), as described in Casas et al., Plant Physiology, 170: 367-384, 2016. Briefly, to extract PMP lipids including GIPCs, 3.5 mL of chloroform:methanol:HCl (200:100:1, v/v/v) plus 0.01% (w/v) of butylated hydroxytoluene, is added to and incubated with the PMPs. Next, 2 mL of 0.9% (w/v) NaCl is added and vortexed for 5 minutes. The sample is then centrifuged to induce the organic phase to aggregate at the bottom of the glass tube, and the organic phase is collected. The upper phase undergoes reextraction with 4 mL of pure chloroform to isolate lipids. The organic phases are combined and dried. After drying, the aqueous phase is resuspended with 1 mL of pure water and GIPCs are back-extracted using 1 mL of butanol-1 twice. To produce polypeptide-loaded PMPs, the isolated PMP lipid phases are mixed with the polypeptide solution and are passed through a lipid extruder according to the protocol from Haney et al., J Control Release, 207: 18-30, 2015.

Alternatively, 3.5 mL of methyl tertiary-butyl ether (MTBE):methanol:water (100:30:25, v/v/v) plus 0.01% (w/v) butylated hydroxytoluene (BHT) is added to and incubated with the PMPs. After incubation, 2 mL of 0.9% NaCl is added, is vortexed for 5 minutes, and is centrifuged. The organic phase (upper) is collected and the aqueous phase (lower) is subjected to reextraction with 4 mL of pure MTBE. The organic phases are combined and dried. After drying, the aqueous phase is resuspend with 1 mL of pure water and GIPCs are back-extracted using 1 mL of butanol-1 twice. To produce protein-loaded PMPs, the isolated PMP lipid phases are mixed with the protein solution and passed through a lipid extruder according to the protocol from Haney et al., J Control Release, 207: 18-30, 2015.

Alternatively, 3.5 mL of propan-2-ol:hexane:water (55:20:25, v/v/v) is incubated with the sample for 15 mins at 60° C. with occasional shaking. After incubation, samples are spun down at 500× g and the supernatant is transferred, and the process is repeated with 3.5 mL of the extraction solvent. Supernatants are combined and dried, followed by resuspension in 1 mL of pure water. GIPCs are then back-extracted with 1 mL of butanol-1 twice. GIPCs can be added to PMP lipids isolated via methods described in this example. To produce protein-loaded PMPs, the isolated PMP lipids are mixed with the protein solution and passed through a lipid extruder according to the protocol from Haney et al., J Control Release, 207: 18-30, 2015.

Before use, the loaded PMPs are purified using the methods as described in Example 2 to remove polypeptides that are not bound to or encapsulated by the PMP. Loaded PMPs are characterized as described in Example 3, and their stability is tested as described in Example 4. To measure loading of the protein or peptide, the Pierce Quantitative Colorimetric Peptide Assay is used on a small sample of the loaded and unloaded PMPs, or using Western blot detection using protein-specific antibodies. Alternatively, proteins can be fluorescently labeled, and fluorescence can be used to determine the labeled protein concentration in loaded and unloaded PMPs.

### Example 6: Treatment of Human Cells with Cre Recombinase Protein-Loaded PMPs

This example demonstrates loading of PMPs with a model protein with the purpose of delivering a functional protein into human cells. In this example, Cre recombinase is used as a model protein, and human embryonic kidney 293 cells (HEK293 cells) comprising a Cre reporter transgene (Hek293-LoxP-GFP-LoxP-RFP) (Puro; GenTarget, Inc.), are used as a model human cell line.

a) Production of Grapefruit PMPs Using TFF Combined with SEC

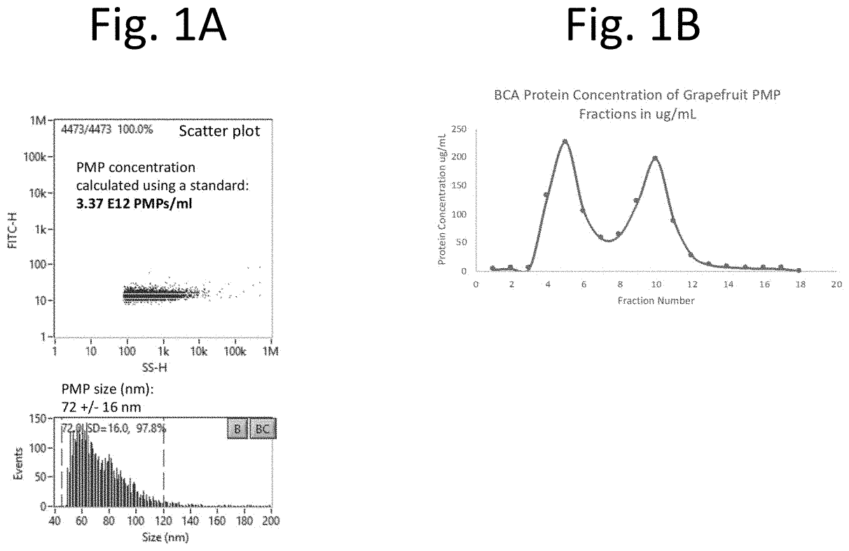

Red organic grapefruits were obtained from a local Whole Foods Market®. Two liters of grapefruit juice was collected using a juice press, and was subsequently centrifuged at 3000×g for 20 minutes, followed by 10,000×g for 40 minutes to remove large debris. PMPs were incubated in a final concentration of 50 mM EDTA (pH 7) for 30 minutes, and were subsequently passaged through a 1 pm and a 0.45 pm filter. Filtered juice was concentrated by tangential flow filtration (TFF) to 700 mL, washed with 500 mL of PBS, and concentrated to a final volume of 400 mL juice (total concentration 5×). Concentrated juice was dialyzed overnight in PBS using a 300 kDa dialysis membrane to remove contaminants. Subsequently, the dialyzed juice was further concentrated by TFF to a final concentration of 50 mL. Next, we used size exclusion chromatography to elute the PMP-containing fractions, and analyzed PMP size and concentration by nano-flow cytometry (NanoFCM) and protein concentration using a Pierce™ bicinchoninic acid (BCA) assay according to the manufacturer's instructions (FIGS. 1A and 1B). SEC fractions 8-12 contained contaminants. SEC fractions 4-6 contained purified PMPs and were pooled together, filter sterilized using 0.85 μm, 0.4 μm and 0.22 μm syringe filters, analyzed by NanoFCM (FIG. 1A) and used for loading Cre recombinase protein.

b) Loading of Cre Recombinase Protein into Grapefruit PMPs

Cre recombinase protein (ab134845) was obtained from Abcam, and was dissolved in UltraPure water to a final concentration of 0.5 mg/mL protein. Filter-sterilized PMPs were loaded with Cre recombinase protein by electroporation, using a protocol adapted from Rachael W. Sirianni and Bahareh Behkam (eds.), Targeted Drug Delivery: Methods and Protocols, Methods in Molecular Biology, vol. 1831. PMPs alone (PMP control), Cre recombinase protein alone (protein control), or PMP+Cre recombinase protein (protein-loaded PMPs) were mixed with 2× electroporation buffer (42% Optiprep™ (Sigma, D1556) in UltraPure water), see Table 5. Samples were transferred into a chilled cuvettes and electroporated at 0.400 kV, 125 μF (0.125 mF), resistance low 100 Ω-high 600Ω with two pulses (4-10 ms) using a Biorad GenePulser. The reaction was put on ice for 10 minutes, and transferred to a pre-ice chilled 1.5 ml ultracentrifuge tube. All samples containing PMPs were washed 3 times by adding 1.4 ml ultrapure water, followed by ultracentrifugation (100,000 g for 1.5 h at 4° C.). The final pellet was resuspended in a minimal volume of UltraPure water (30-50 μL) and kept at 4° C. until use. After electroporation, samples containing Cre protein only were diluted in UltraPure water (as indicated in Table 5), and stored at 4° C. until use.

TABLE 5
Cre recombinase protein loading into grapefruit PMPs.

| | | | | | | | | Cre | Cre |
|---|---|---|---|---|---|---|---|---|---|
| | | | | | | | | recom- | recom- |
| | | | | | | | | binase | binase |
| | | | | | | | | treatment | treatment |
| | | | (b) | | | | | dose: | dose: |
| | | | Loading: | | | | | Assuming | Assuming |
| | | | Cre | | | | | 100% | 10% |
| | | (a) | recom- | (c) | | | | loading | loading |
| | | PMP | binase | Loading: | | | | efficiency, | efficiency, |
| | | loading: | protein | Final | | | | maximum | maximum |
| | | PMPs | (0.5 | volume | PMP | | Treatment: | Cre | Cre |
| | Input | added to | mg/mL) | of PMP | concen- | Treatment: | PMP | recom- | recom- |
| | PMP | electro- | added to | formu- | tration | Amount | treatment | binase | binase |
| | concen- | poration | electro- | lation | after | of (c) | concen- | protein | protein |
| | tration | reaction | poration | after | loading | added | tration | concen- | concen- |
| | (PMPs/ | mixture | mixture | washing | (PMPs/ | to cells | (PMPs/ | tration | tration |
| | mL) | (μL) | (μL) | (μL) | mL) | (μL) | mL) | (μg/mL) | (μg/mL) |
| Cre- | 3.37 × 10¹² | 40 | 40 | 50 | 3.28 × 10¹¹ | 10 | 2.63 × 10¹⁰ | 40.00 | 4.00 |
| PMP | | | | | | | | | |
| electro- | | | | | | | | | |
| porated | | | | | | | | | |
| Cre- | 3.37 × 10¹² | 20 | 20 | 54 | 2.92 × 10¹¹ | 30 | 3.25 × 10¹⁰ | 55.56 | 5.56 |
| PMP not | | | | | | | | | |
| electro- | | | | | | | | | |
| porated | | | | | | | | | |
| (loading | | | | | | | | | |
| control) | | | | | | | | | |
| PMP | 3.37 × 10¹² | 10 | 0 | 48 | 5.49 × 10¹⁰ | 24 | 2.74 × 10⁹ | 0.00 | 0.00 |
| only | | | | | | | | | |
| electro- | | | | | | | | | |
| porated | | | | | | | | | |
| (PMP | | | | | | | | | |
| only | | | | | | | | | |
| control) | | | | | | | | | |
| Cre | 0.5 mg/mL | | 10 | 35 | | 6 | | 8.57 | |
| recom- | | | | | | | | | |
| binase | | | | | | | | | |
| electro- | | | | | | | | | |
| porated | | | | | | | | | |
| (protein | | | | | | | | | |
| only | | | | | | | | | |
| control) | | | | | | | | | |

c) Treatment of Hek293 LoxP-GFP-LoxP-RFP Cells with Cre-Recombinase-Loaded Grapefruit PMPs

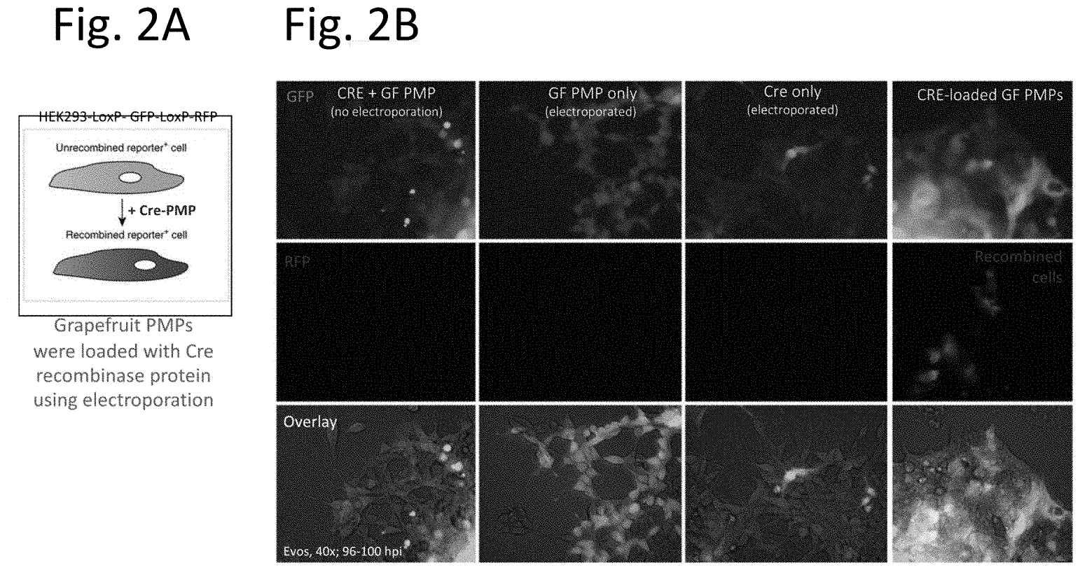

The Hek293 LoxP-GFP-LoxP-RFP (Puro) human Cre-reporter cell line was purchased from GenTarget, Inc., and was maintained according to the manufacturer's instructions without antibiotic selection. Cells were seeded into a 96 well plate and were treated for 24 hrs in complete medium with Cre-recombinase-loaded PMPs (electroporated PMPs+Cre recombinase protein; 2.63×10¹⁰ PMPs/mL), electroporated PMPs (PMP only control; 2.74×10⁹ PMPs/mL), electroporated Cre recombinase protein (protein only control; 8.57 μg/mL), or non-electroporated PMPs+Cre recombinase protein (loading control; 3.25×10¹⁰ PMPs/mL), as indicated in Table 5. After 24 hrs, cells were washed twice with Dulbecco's phosphate-buffered saline (DPBS), and fresh complete cell culture medium is added. 96-100 hrs post treatment, cells were imaged using an EVOS FL 2 fluorescence imaging system (Invitrogen). When Cre recombinase protein is functionally delivered into the cells and transported to the nucleus, GFP is recombined out, inducing a color switch in the cells from green to red (FIG. 2A). The presence of red fluorescent cells therefore indicates functional delivery of Cre recombinase protein by PMPs. FIG. 2B shows that recombined red fluorescent cells are observed only when cells are exposed to Cre-recombinase-loaded PMPs, while these are absent in the control treated Hek293 LoxP-GFP-LoxP-RFP cells. Our data shows that PMPs can be loaded with protein, and can functionally deliver protein cargo into human cells.

### Example 7: Treatment of Diabetic Mice with Insulin-Loaded PMPs

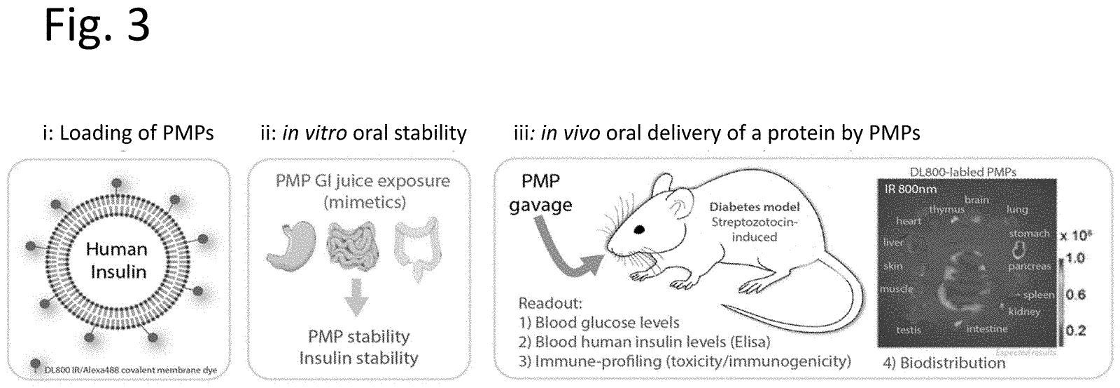

This example describes loading of PMPs with a protein with the purpose of delivering the protein in vivo via oral and systemic administration. In this example, insulin is used as a model protein, and streptozotocin-induced diabetic mice are used as an in vivo model (FIG. 3). This example further shows that PMPs are stable throughout the gastrointestinal (GI) tract and are able to protect protein cargo.

### Therapeutic Design:

The PMP solution is formulated to an effective insulin dose of 0, 0.001, 0.01, 0.1, 0.5, 1 mg/ml in PBS.

### Experimental Protocol:

a) Loading of Lemon PMPs with Insulin Protein

PMPs are produced from lemon juice and other plant sources according to Example 1-2. Human recombinant insulin (Gibco) and labeled insulin-FITC (Sigma Aldrich I3661) are solubilized at a concentration of 3 mg/ml in 10 mM HCl, pH 3. PMPs are placed in solution with the protein in PBS. If the protein is insoluble, pH is adjusted until it is soluble. If the protein is still insoluble, the insoluble protein is used. The solution is then sonicated to induce poration and diffusion into the PMP according to the protocol from Wang et al., Nature Comm., 4: 1867, 2013. Alternatively, the solution can be passed through a lipid extruder according to the protocol from Haney et al., J Control Release, 207: 18-30, 2015. Alternatively, PMPs can be electroporated according to the protocol from Wahlgren et al., Nucl. Acids. Res., 40(17), e130, 2012.

To produce protein-loaded PMPs, insulin or FITC-insulin can alternatively be loaded by mixing PMP-isolated lipids with the protein, and resealing using extrusion or sonication as described in Example 5. In brief, solubilized PMP lipids are mixed with a solution of insulin protein (pH 3, 10 mM HCl), sonicated for 20 minutes at 40° C., and extruded using polycarbonate membranes. Alternatively, insulin protein can be precomplexed prior to PMP lipid mixing with protamine sulfate (Sigma, P3369) in a 5:1 ratio, to facilitate encapsulation.

Insulin-loaded PMPs are purified by spinning down (100,000×g for 1 hour at 4° C.) and washing the pellet 2 times with acidic water (pH 4), followed by one wash with PBS (pH 7.4) to remove un-encapsulated protein in the supernatant. Alternatively, other purification methods can be used as described in Example 2. The final pellet is resuspended in a minimal volume of PBS (30-50 μL) and stored at 4° C. until use. Insulin-loaded PMPs are characterized as described in Example 3, and their stability is tested as described in Example 4.

Insulin encapsulation of PMPs is measured by HPLC, Western blot (anti-insulin antibody, Abcam ab181547) or by human insulin ELISA (Abcam, ab100578). FITC-insulin-loaded PMPs can alternatively be analyzed by fluorescence (Ex/Em 490/525). Pierce MicroBCA™ analysis (Thermo Scientific™) can be used to determine total protein concentration before and after loading. The Loading Efficacy (%) is determined by dividing the incorporated insulin (ug) by the total amount of insulin (ug) added to the reaction. PMP loading capacity is determined by dividing the amount of incorporated insulin (ug) by the number of labeled PMPs (in case of FITC-insulin) or PMPs (unlabeled insulin).

b) Gastro-Intestinal Stability of Insulin-FITC Loaded Lemon PMPs In Vitro

To determine the stability of PMPs in the GI tract, and the ability of PMPs to protect protein cargo from degradation, insulin-FITC-loaded PMPs are subjected to fasted and fed GI stomach and intestinal fluid mimetics purchased from Biorelevant (UK), which are prepared according to the manufacturer's instruction: FaSSIF (Fasted, small intestine, pH 6.5), FeSSIF (Fed, small intestine, pH 5, supplemented with pancreatin), FaSSGF (Fasted, stomach, pH 1.6), FaSSIF-V2 (Fasted, small intestine, pH 6.5), FeSSIF-V2 (Fed, small intestine, with digestive components, pH 5.8).

Twenty μl of insulin-FITC-loaded PMPs with an effective dose of 0 (PMP only control), 0.001, 0.01, 0.1, 0.5, 1 mg/ml Insulin-FITC, or free 0 (PBS control), 0.001, 0.01, 0.1, 0.5, 1 mg/ml Insulin-FITC are incubated with 1 mL of stomach, fed, and fasted intestinal juices (FaSSIF, F2SSIF, FaSSGF, FaSSIF-V2 and FeSSIF-V2), PMS (negative control), and PBS+0.1% SDS (PMP degradation control) for 1, 2, 3, 4, and 6 hours at 37° C. Alternatively, insulin-FITC-loaded PMPs or free protein are subsequently exposed to F2SSIF>FASSIF-V2 or F2SSIF>FESSIF-V2 for 1, 2, 3, 4, and 6 hours at 37° C. for each step. Next, Insulin-FITC-loaded PMPs are pelleted by ultracentrifugation at 100,000×g for 1 h at 4° C. Pellets are resuspended in 25-50 mM Tris pH 8.6, and analyzed for fluorescence intensity (Ex/Em 490/525), FITC⁺PMP concentration, PMP size, and insulin protein concentration. PMP supernatants after pelleting, and insulin-FITC protein only samples are analyzed by fluorescence intensity after adjusting the pH of the solutions to pH 8-9 (bicarbonate buffer), the presence of particles in the solution and their size is measured, and after precipitation, insulin protein concentration is determined by Western blot. To show that PMPs are stable throughout the GI tract and that their protein cargo is protected from degradation, total fluorescence (spectrophotometer), total insulin protein (Western), PMP size and fluorescent PMP concentration (NanoFCM) of Insulin-FITC-labeled PMPs and free Insulin-FITC protein are compared between the different GI juice mimetics and the PBS control. Insulin-FITC-labeled PMPs are stable when fluorescent PMPs and Insulin-FITC protein can be detected after GI juice exposure, compare to PBS incubation.

c) Treatment of Diabetic Mice with Insulin-Loaded PMPs Via Oral Administration

To show the ability of PMPs to deliver functional protein in vivo, PMPs are loaded with human recombinant insulin using the methods described in Example 7a. PMPs are labeled with DyLight-800 (DL800) infrared membrane dye (Invitrogen). Briefly, DyLight800 is dissolved in DMSO to a final concentration of 10 mg/mL and 200 μL of PMPs (1-3×10¹² PMPs/mL) are mixed with 5 μL dye and are incubated for 1 h at room temperature on a shaker. Labeled PMPs are washed 2-3 times by ultracentrifuge at 100,000×g for 1 hr at 4° C., and pellets are resuspended with 1.5 ml UltraPure water. The final DyLight800 labeled pellets are resuspended in a minimal amount of UltraPure PBS and are characterized using methods described herein.

Mouse experiments are performed at a contract research organization, using a well-established streptozotocin (STZ)-induced diabetic mouse model, and mice are treated and monitored according to standard procedures. In short, eight week old streptozotocin (STZ)-induced diabetic male C57BL/6J mice are orally gavaged with 300 μl insulin-loaded PMPs with an effective dose of 0 (PMP only control), 0.01, 0.1, 0.5, 1 mg/mL insulin, or free 0 (PBS control), 0.1, 0.5, 1 mg/mL insulin (5 mice per group). Blood glucose levels of the mice are monitored after 2, 4, 6, 12 and 24 hours, and at the end point, blood samples are collected for ELISA to determine human insulin levels in the mouse. PMPs can effectively deliver insulin orally when blood glucose levels are induced, when compared to free insulin, unloaded PMPs or PBS. The biodistribution of the PMPs is determined by isolating mouse organs and tissues at the experimental endpoint and measuring infrared fluorescence at 800 nm using a Licor Odyssey imager.

d) Treatment of Diabetic Mice with Insulin-Loaded PMPs Via IV Administration

To show the ability of PMPs to deliver functional protein in vivo, PMPs are loaded with human recombinant insulin using methods described in Example 7a. PMPs are labeled with DyLight-800 (DL800) infrared membrane dye (Invitrogen). Briefly, DyLight800 is dissolved in DMSO to a final concentration of 10 mg/mL and 200 μL of PMPs (1-3×10¹² PMPs/mL) are mixed with 5 μL dye and are incubated for 1 h at room temperature on a shaker. Labeled PMPs are washed 2-3 times by ultracentrifuge at 100,000×g for 1 hr at 4° C., and pellets are resuspended with 1.5 ml UltraPure water. The final DyLight800 labeled pellets are resuspended in a minimal amount of UltraPure PBS and are characterized using methods described herein.

Mouse experiments are performed at a contract research organization, using a well-established streptozotocin (STZ)-induced diabetic mouse model, and mice are treated and monitored according to standard procedures. In short, eight week old streptozotocin (STZ)-induced diabetic male C57BL/6J mice are systemically administered insulin-PMPs by tail vein injection with an effective dose of 0 (PMP only control), 0.01, 0.1, 0.5, 1 mg/ml Insulin, PBS (negative control), or 10-20 mg/kg free insulin (positive control) (5 mice per group). Blood glucose levels of the mice are monitored after 2, 4, 6, 12 and 24 hours, and at the end point, blood samples are collected for ELISA to determine human insulin levels in the mouse. PMPs can effectively deliver insulin systemically when blood glucose levels are induced, when compared unloaded PMPs and PBS. The biodistribution of the PMPs is determined by isolating mouse organs and tissues at the experimental endpoint, and measuring infrared fluorescence at 800 nm using a Licor Odyssey imager.

e) Treatment of Diabetic Mice with Insulin-Loaded PMPs Via IP Administration

To show the ability of PMPs to deliver functional protein in vivo, PMPs are loaded with human recombinant insulin using methods described in Example 7a. PMPs are labeled with DyLight-800 (DL800) infrared membrane dye (Invitrogen). Briefly, DyLight800 is dissolved in DMSO to a final concentration of 10 mg/mL and 200 μL of PMPs (1-3×10¹² PMPs/mL) are mixed with 5 μL dye and are incubated for 1 h at room temperature on a shaker. Labeled PMPs are washed 2-3 times by ultracentrifuge at 100,000×g for 1 hr at 4° C., and pellets are resuspended with 1.5 ml UltraPure water. The final DyLight800 labeled pellets are resuspended in a minimal amount of UltraPure PBS and are characterized using methods described herein.

Mouse experiments are performed at a contract research organization, using a well-established streptozotocin (STZ)-induced diabetic mouse model, and mice are treated and monitored according to standard procedures. In short, eight week old streptozotocin (STZ)-induced diabetic male C57BL/6J mice, are administered insulin-PMPs by intraperitoneal (IP) injection with an effective dose of 0 (PMP only control), 0.01, 0.1, 0.5, 1 mg/ml insulin, PBS (negative control), or 10-20 mg/kg free insulin (positive control) (5 mice per group). Blood glucose levels of the mice are monitored after 2, 4, 6, 12 and 24 hours, and at the end point, blood samples are collected for ELISA to determine human insulin levels in the mouse. PMPs can effectively deliver insulin systemically when blood glucose levels are induced, when compared unloaded PMPs and PBS. The biodistribution of the PMPs is determined by isolating mouse organs and tissues at the experimental endpoint and measuring infrared fluorescence at 800 nm, using a Licor Odyssey imager.

### Example 8: Treatment of Human, Bacterial, Fungal, Plant, and Nematode Cells with Protein-Loaded Plant Messenger Packs

A. Treatment of Human Cells with Protein-Loaded PMPs

This example describes loading of PMPs with a protein for the purpose of delivering a protein cargo to enhance or reduce fitness in mammalian cells. This example describes PMPs loaded with GFP that are taken up by human cells, and it further describes that protein-loaded PMPs are stable and retain their activity over a range of processing and environmental conditions. In this example, GFP is used as a model protein or polypeptide, and A549 lung cancer cells are used as model human cell line.

Therapeutic Dose:

PMPs loaded with GFP, formulated in water to a concentration that delivers 0 (unloaded PMP control), 0.01, 0.1, 1, 5, 10, or 100 μg/ml GFP protein-loaded in PMPs.

Experimental Protocol:

a) Loading of Lemon PMPs with GFP Protein

PMPs are produced from lemon juice and other plant sources according to Example 1. Green fluorescent protein is synthesized commercially (Abcam) and solubilized in PBS. PMPs are placed in solution with the protein in PBS. If the protein is insoluble, pH is adjusted until it is soluble. If the protein is still insoluble, the insoluble protein is used. The solution is then sonicated to induce poration and diffusion into the PMP according to the protocol from Wang et al., Nature Comm., 4: 1867, 2013. Alternatively, the solution can be passed through a lipid extruder according to the protocol from Haney et al., J Control Release, 207: 18-30, 2015. Alternatively, PMPs can be electroporated according to the protocol from Wahlgren et al., Nucl. Acids. Res., 40(17), e130, 2012.

To produce protein-loaded PMPs, GFP can alternatively be loaded by mixing PMP-isolated lipids with the protein, and resealing using extrusion or sonication as described in Example 5. In brief, solubilized PMP lipids are mixed with a solution of GFP protein (pH 5-6, in PBS), sonicated for 20 minutes at 40° C., and extruded using polycarbonate membranes. Alternatively, GFP protein can be precomplexed prior to PMP lipid mixing with protamine (Sigma) in a 10:1 ratio to facilitate encapsulation.

GFP-loaded PMPs are purified by spinning down (100,000×g for 1 hour at 4° C.) and washing the pellet three times to remove un-encapsulated protein in the supernatant, or by using other methods as described in Example 2. GFP-loaded PMPs are characterized as described in Example 3, and their stability is tested as described in Example 4. GFP encapsulation of PMPs is measured by Western blot or fluorescence.

b) Treatment of Human A549 Cells with GFP-Loaded Lemon PMPs

A549 lung cancer cells were purchased from the ATCC (CCL-185) and maintained in F12K medium supplemented with 10% FBS according to the manufacturer's instructions. To determine GFP-loaded PMP uptake by human cells, A549 cells are plated in a 48 well plate at a concentration of 1E5 cells/well, and cells are allowed to adhere for at least 6 hours at 37° C. or overnight. Next, medium is aspirated and cells are incubated with 0 (unloaded PMP control), 0.01, 0.1, 1, 5, 10, or 100 μg/ml GFP-loaded lemon-derived PMPs, or unloaded 0 (negative control), 0.01, 0.1, 1, 5, 10, or 100 μg/ml GFP protein in complete medium. After incubation of 2, 6, 12 and 24 hours at 37° C., the medium is aspirated and cells are gently washed 3 times for 5 minutes with DPBS or complete medium. Optionally, if tolerated, A549 cells are incubated with 0.5% triton X100 with/without ProtK (2 mg/mL) for 10 minutes at 37° C. to burst and degrade PMPs and protein that are not taken up by the cells. Next, images are acquired on a high-resolution fluorescence microscope. Uptake of GFP-loaded PMPs or GFP protein alone by A549 is demonstrated when the cytoplasm of the cell turns green. The percentage of GFP-loaded PMP treated cells with a green cytoplasm compared to control treatments with PBS and GFP only are recorded to determine uptake. In addition, GFP uptake by cells is measured by Western blot using an anti-GFP antibody (Abcam), after total protein isolation in treated and untreated cells, using standard methods. GFP protein levels are recorded and compared between cells treated with GFP-loaded PMPs, GFP protein alone, and untreated cells to determine uptake.

B. Treatment of Bacteria with Protein-Loaded PMPs

This example describes loading of PMPs with a protein for the purpose of delivering a protein cargo to enhance or reduce fitness in bacteria. This example describes PMPs loaded with GFP that are taken up by bacteria, and it further describes that protein-loaded PMPs are stable and retain their activity over a range of processing and environmental conditions. In this example, GFP is used as a model protein or peptide, and E coli are used as a model bacterium.

### Therapeutic Dose:

PMPs loaded with GFP are formulated as described in Example 8A.

### Experimental Protocol:

a) Loading of Lemon PMPs with GFP Protein

PMPs are produced as described in Example 8A.

b) Delivery of GFP-Loaded Lemon PMPs to E. coli

E. coli are acquired from ATCC (#25922) and grown on Trypticase Soy Agar/broth at 37° C. according to the manufacturer's instructions. To determine the GFP-loaded PMP uptake by E. coli, 10 uL of a 1 mL overnight bacterial suspension is incubated with 0 (unloaded PMP control), 0.01, 0.1, 1, 5, 10, 100 μg/mL GFP-loaded lemon-derived PMPs, or unloaded 0 (negative control), 0.01, 0.1, 1, 5, 10, 100 μg/mL GFP protein in liquid culture. After incubation of 5 min, 30 min and 1 h at room temperature, bacteria are washed 4 times with 0.5% triton X100, and optional ProtK treatment (2 mg/ml ProtK, 10 minutes at 37° C.; if tolerated by the bacteria) to burst and degrade PMPs and protein that are not taken up by the bacteria. Next, images are acquired on a high-resolution fluorescence microscope. Uptake of GFP-loaded PMPs or GFP protein alone by bacteria is demonstrated when the cytoplasm of the bacteria turns green. The percentage of GFP-loaded PMP treated bacteria with a green cytoplasm compared to control treatments with PBS and GFP only are recorded to determine uptake. In addition, GFP uptake by bacteria is measured by Western blot using an anti-GFP antibody (Abcam), after total protein isolation in treated and untreated bacteria, using standard methods. GFP protein levels are recorded and compared between bacteria treated with GFP-loaded PMPs, GFP protein alone, and untreated bacteria to determine uptake.

B. Treatment of Fungi with Protein-Loaded PMPs

This example describes loading of PMPs with a protein for the purpose of delivering a protein cargo to enhance or reduce fitness in fungi. This example describes PMPs loaded with GFP that are taken up by fungi (including yeast), and it further describes that protein-loaded PMPs are stable and retain their activity over a range of processing and environmental conditions. In this example, GFP is used as a model peptide and protein, and Saccharomyces cerevisiae is used as a model fungus.

### Therapeutic Dose:

PMPs loaded with GFP are formulated as described in Example 8A.

### Experimental Protocol:

a) Loading of Lemon PMPs with GFP Protein

PMPs are produced as described in Example 8A.

b) Delivery of GFP-Loaded Lemon PMPs to Saccharomyces cerevisiae

Saccharomyces cerevisiae is obtained from the ATCC (#9763) and maintained at 30° C. in yeast extract peptone dextrose broth (YPD) as indicated by the manufacturer. To determine the PMP uptake by S. cerevisiae, yeast cells are grown to an OD₆₀₀ of 0.4-0.6 in selection media, and incubated with 0 (unloaded PMP control), 0.01, 0.1, 1, 5, 10, 100 μg/ml GFP-loaded lemon-derived PMPs, or unloaded 0 (negative control), 0.01, 0.1, 1, 5, 10, 100 μg/ml GFP protein, in liquid culture. After incubation of 5 min, 30 min and 1 h at room temperature, yeast cells are washed 4 times with 0.5% triton X100, and optional ProtK treatment (2 mg/ml ProtK, 10 minutes at 37° C.; if tolerated by the cells) to burst and degrade PMPs and protein that are not taken up by the bacteria. Next, images are acquired on a high-resolution fluorescence microscope. Uptake of GFP-loaded PMPs or GFP protein alone by yeast is demonstrated when the cytoplasm of the yeast cell turns green. The percentage of GFP-loaded PMP treated yeast with a green cytoplasm compared to control treatments with PBS and GFP only are recorded to determine uptake. In addition, GFP uptake by yeast is measured by Western blot using an anti-GFP antibody (Abcam), after total protein isolation in treated and untreated yeast, using standard methods. GFP protein levels are recorded and compared between yeast treated with GFP-loaded PMPs, GFP protein alone, and untreated yeast to determine uptake.

C. Treatment of a Plant with Protein-Loaded PMPs

This example describes loading of PMPs with a protein for the purpose of delivering a protein cargo to enhance or reduce fitness in plants. This example describes PMPs loaded with GFP that are taken up by plants, and it further describes that protein-loaded PMPs are stable and retain their activity over a range of processing and environmental conditions. In this example, GFP is used as a model protein and peptide, and Arabidopsis thaliana seedlings are used as model plant.

### Therapeutic Dose:

PMPs loaded with GFP are formulated as described in Example 8A.

### Experimental Protocol:

a) Loading of Lemon PMPs with GFP Protein

PMPs are produced as described in Example 8A.

b) Delivery of GFP-Loaded PMPs to Arabidopsis thaliana Seedlings

Wild-type Columbia (Col)-1 ecotype Arabidopsis thaliana is obtained from the Arabidopisis Biological Resource Center (ABRC). Seeds are surface sterilized with a solution containing 70% (v/v) ethanol and 0.05% (v/v) Triton X-100, and are germinated on sterile plates in liquid medium containing half-strength Murashige and Skoog (MS), supplemented with 0.5% sucrose and 2.5 mM MES, pH 5.6. Three day old seedlings are treated with 0 (unloaded PMP control), 0.01, 0.1, 1, 5, 10, 100 μg/ml GFP-loaded lemon-derived PMPs, or unloaded 0 (negative control), 0.01, 0.1, 1, 5, 10, 100 μg/ml GFP protein, added to the MS medium for 6, 12, 24 and 48 hours. After treatment, seedlings are extensively washed in MS medium, optionally supplemented with 0.5% Triton X100, followed by ProtK treatment (2 mg/mL ProtK, 10 minutes at 37° C.; if tolerated by the seedlings) to burst and degrade PMPs and protein that are not taken up by the plant. Next, images are acquired on a high-resolution fluorescence microscope to detect GFP in the roots, leaves and other plant parts. GFP-loaded PMPs or GFP protein alone is taken up by seedlings when GFP protein localization can be detected in plant tissues. The number of seedlings with green fluorescence is compared between GFP-loaded PMPs and control treatments with PBS and GFP only to determine uptake. In addition, GFP uptake by seedlings can be quantified by Western blot using an anti-GFP antibody (Abcam), after total protein isolation in treated and untreated seedlings, using standard methods. GFP protein levels are recorded and compared between seedlings treated with GFP-loaded PMPs, GFP protein alone, and untreated seedlings to determine uptake.

D. Treatment of a Nematode with Protein-Loaded PMPs

This example describes loading of PMPs with a protein for the purpose of delivering a protein cargo to enhance or reduce fitness in nematodes. This example describes PMPs loaded with GFP that are taken up by nematodes, and it further describes that protein-loaded PMPs are stable and retain their activity over a range of processing and environmental conditions. In this example, GFP is used as a model peptide, and C. elegans is used as a model nematode.

### Therapeutic Dose:

PMPs loaded with GFP are formulated as described in Example 8A.

### Experimental PROTOCOL:

a) Loading of Lemon PMPs with GFP Protein

PMPs are produced as described in Example 8A.

b) Delivery of GFP-Loaded PMPs to C. elegans

C. elegans wild-type N2 Bristol strain (C. elegans Genomics Center) are maintained on an Escherichia coli (strain OP50) lawn on nematode growth medium (NGM) agar plates (3 WI NaCl, 17 WI agar, 2.5 g/l peptone, 5 mg/I cholesterol, 25 mM KH₂PO₄ (pH 6.0), 1 mM CaCl₂), 1 mM MgSO₄) at 20° C., from L1 until the L4 stage.

One-day old C. elegans are transferred to a new plate and are fed 0 (unloaded PMP control), 0.01, 0.1, 1, 5, 10, 100 μg/ml GFP-loaded lemon-derived PMPs, or unloaded 0 (negative control), 0.01, 0.1, 1, 5, 10, 100 μg/ml GFP protein in a liquid solution following the feeding protocol in Conte et al., Curr. Protoc. Mol. Bio., 109: 26.3.1-26.330, 2015. Worms are next examined for GFP-loaded PMP uptake in the digestive tract by using a fluorescent microscope for green fluorescence, compared to unloaded PMP-treatment, or GFP protein alone and a sterile water control. In addition, GFP uptake by C. elegans can be quantified by Western blot using an anti-GFP antibody (Abcam), after total protein isolation in treated and untreated nematodes, using standard methods. GFP protein levels are recorded and compared between nematodes treated with GFP-loaded PMPs, GFP protein alone, and untreated C. elegans to determine uptake.

E. In Vivo Delivery of Cre Recombinase to a Mouse

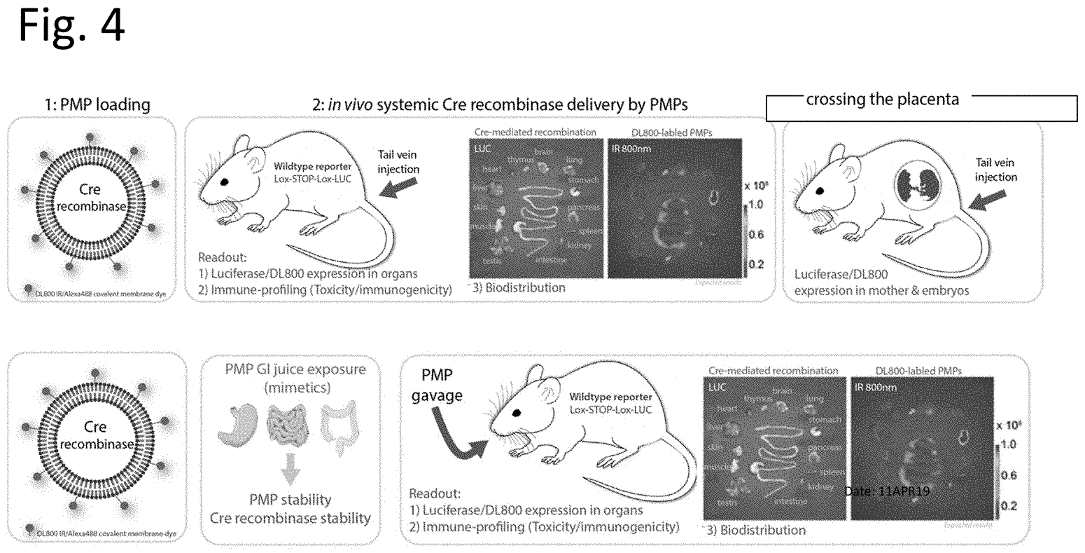

This example describes loading of PMPs with a protein with the purpose of delivering the protein in vivo via oral and systemic administration. In this example, Cre recombinase is used as a model protein, and mice having a luciferase Cre reporter construct (Lox-STOP-Lox-LUC) are used as an in vivo model (FIG. 4).

Delivery of a Cre recombinase to a mouse, as outlined in FIG. 4, may be performed using any of the methods described herein. Expression of luciferase in a mouse tissue indicates that Cre has been delivered by PMPs to the tissue.

### Example 9: PMP Production from Blended Fruit Juice Using Ultracentrifugation and Sucrose Gradient Purification

This example demonstrates that PMPs can be produced from fruit by blending the fruit and using a combination of sequential centrifugation to remove debris, ultracentrifugation to pellet crude PMPs, and using a sucrose density gradient to purify PMPs. In this example, grapefruit was used as a model fruit.

a) Production of Grapefruit PMPs by Ultracentrifugation and Sucrose Density Gradient Purification

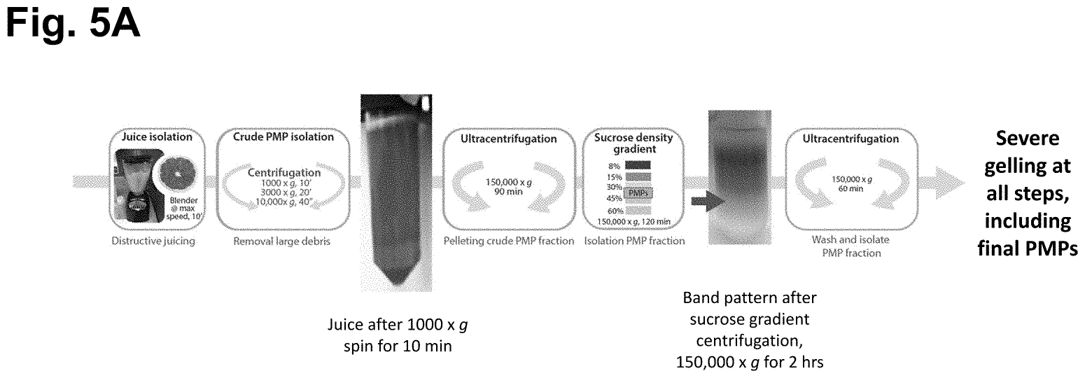

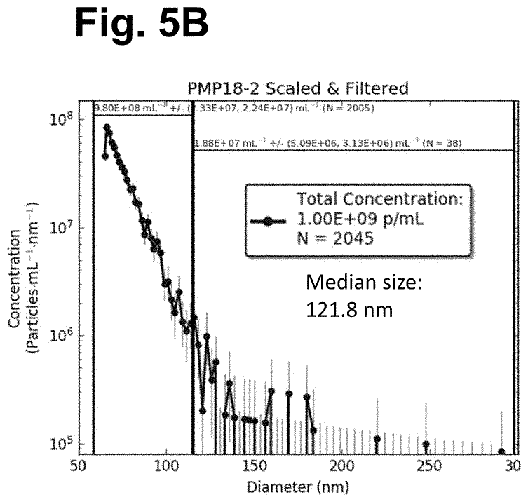

A workflow for grapefruit PMP production using a blender, ultracentrifugation and sucrose gradient purification is shown in FIG. 5A. One red grapefruit was purchased from a local Whole Foods Market®, and the albedo, flavedo, and segment membranes were removed to collect juice sacs, which were homogenized using a blender at maximum speed for 10 minutes. One hundred mL juice was diluted 5× with PBS, followed by subsequent centrifugation at 1000×g for 10 minutes, 3000× g for 20 minutes, and 10,000× g for 40 minutes to remove large debris. 28 mL of cleared juice was ultracentrifuged on a Sorvall™ MX 120 Plus Micro-Ultracentrifuge at 150,000× g for 90 minutes at 4° C. using a S50-ST (4×7 mL) swing bucket rotor to obtain a crude PMP pellet which was resuspended in PBS pH 7.4. Next, a sucrose gradient was prepared in Tris-HCL pH7.2, crude PMPs were layered on top of the sucrose gradient (from top to bottom: 8, 15, 30, 45 and 60% sucrose), and spun down by ultracentrifugation at 150,000×g for 120 minutes at 4° C. using a S50-ST (4×7 mL) swing bucket rotor. One mL fractions were collected and PMPs were isolated at the 30-45% interface. The fractions were washed with PBS by ultracentrifugation at 150,000×g for 120 minutes at 4° C. and pellets were dissolved in a minimal amount of PBS.

PMP concentration (1×10⁹ PMPs/mL) and median PMP size (121.8 nm) were determined using a Spectradyne nCS1™ particle analyzer, using a TS-400 cartridge (FIG. 5B). The zeta potential was determined using a Malvern Zetasizer Ultra and was −11.5+/−0.357 mV.

This example demonstrates that grapefruit PMPs can be isolated using ultracentrifugation combined with sucrose gradient purification methods. However, this method induced severe gelling of the samples at all PMP production steps and in the final PMP solution.

### Example 10: PMP Production from Mesh-Pressed Fruit Juice Using Ultracentrifugation and Sucrose Gradient Purification

This example demonstrates that cell wall and cell membrane contaminants can be reduced during the PMP production process by using a milder juicing process (mesh strainer). In this example, grapefruit was used as a model fruit.

a) Mild Juicing Reduces Gelling During PMP Production from Grapefruit PMPs

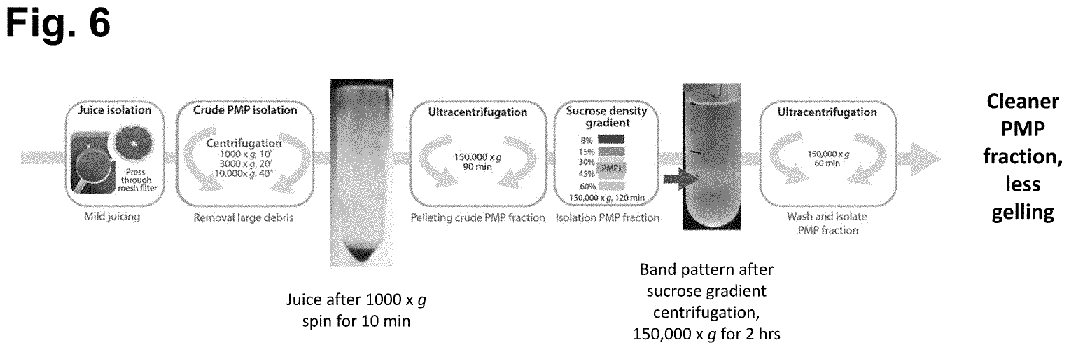

Juice sacs were isolated from a red grapefruit as described in Example 9. To reduce gelling during PMP production, instead of using a destructive blending method, juice sacs were gently pressed against a tea strainer mesh to collect the juice and to reduce cell wall and cell membrane contaminants. After differential centrifugation, the juice was more clear than after using a blender, and one clean PMP-containing sucrose band at the 30-45% intersection was observed after sucrose density gradient centrifugation (FIG. 6). There was overall less gelling during and after PMP production.

Our data shows that use of a mild juicing step reduces gelling caused by contaminants during PMP production when compared to a method comprising blending.

### Example 11: PMP Production Using Ultracentrifugation and Size Exclusion Chromatography

This example describes the production of PMPs from fruits by using Ultracentrifugation (UC) and Size Exclusion Chromatography (SEC). In this example, grapefruit is used as a model fruit.

a) Production of Grapefruit PMPs Using UC and SEC

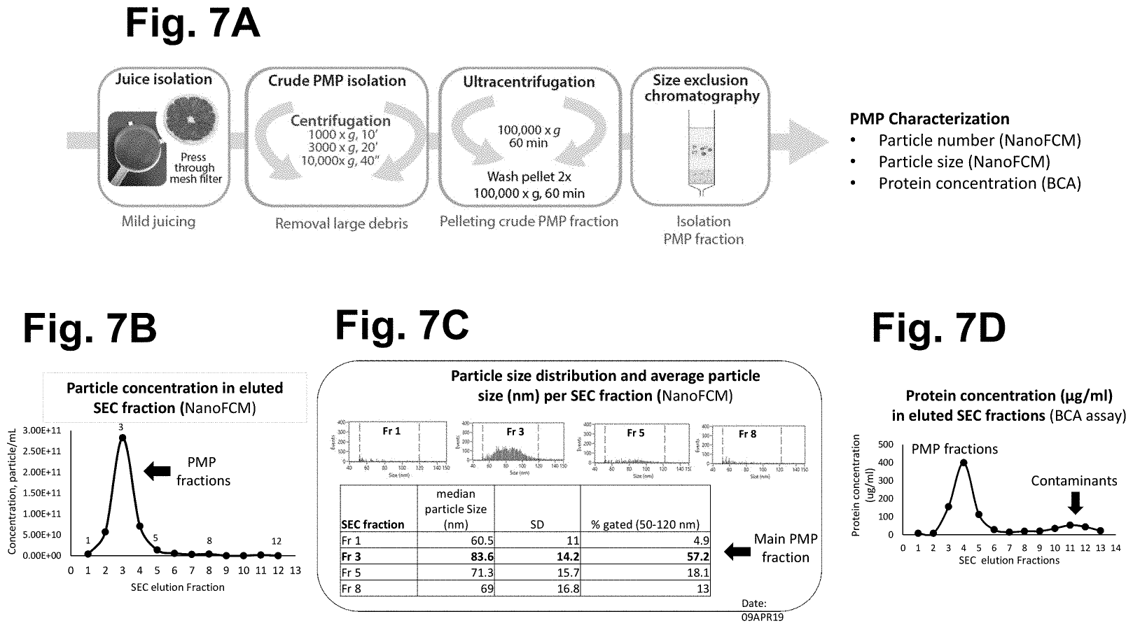

Juice sacs were isolated from a red grapefruit, as described in Example 9a, and were gently pressed against a tea strainer mesh to collect 28 ml juice. The workflow for grapefruit PMP production using UC and SEC is depicted in FIG. 7A. Briefly, juice was subjected to differential centrifugation at 1000×g for 10 minutes, 3000× g for 20 minutes, and 10,000× g for 40 minutes to remove large debris. 28 ml of cleared juice was ultracentrifuged on a Sorvall™ MX 120 Plus Micro-Ultracentrifuge at 100,000× g for 60 minutes at 4° C. using a S50-ST (4×7 mL) swing bucket rotor to obtain a crude PMP pellet which was resuspended in MES buffer (20 mM MES, NaCl, pH 6). After washing the pellets twice with MES buffer, the final pellet was resuspended in 1 ml PBS, pH 7.4. Next, we used size exclusion chromatography to elute the PMP-containing fractions. SEC elution fractions were analyzed by nano-flow cytometry using a NanoFCM to determine PMP size and concentration using concentration and size standards provided by the manufacturer. In addition, absorbance at 280 nm (SpectraMax®) and protein concentration (Pierce™ BCA assay, ThermoFisher) were determined on SEC fractions to identify in which fractions PMPs are eluted (FIGS. 7B-7D). SEC fractions 2-4 were identified as the PMP-containing fractions. Analysis of earlier- and later-eluting fractions indicated that SEC fraction 3 is the main PMP-containing fraction, with a concentration of 2.83×10¹¹ PMPs/mL (57.2% of all particles in the 50-120 nm size range), with a median size of 83.6 nm+/−14.2 nm (SD). While the late elution fractions 8-13 had a very low concentration of particles as shown by NanoFCM, protein contaminants were detected in these fractions by BCA analysis.

Our data shows that TFF and SEC can be used to isolate purified PMPs from late-eluting contaminants, and that a combination of the analysis methods used here can identify PMP fractions from late-eluting contaminants.

### Example 12: Scaled PMP Production Using Tangential Flow Filtration and Size Exclusion Chromatography Combined with EDTA/Dialysis to Reduce Contaminants

This example describes the scaled production of PMPs from fruits by using Tangential Flow Filtration (TFF) and Size Exclusion Chromatography (SEC), combined with an EDTA incubation to reduce the formation of pectin macromolecules, and overnight dialysis to reduce contaminants. In this example, grapefruit is used as a model fruit.

a) Production of Grapefruit PMPs Using TFF and SEC

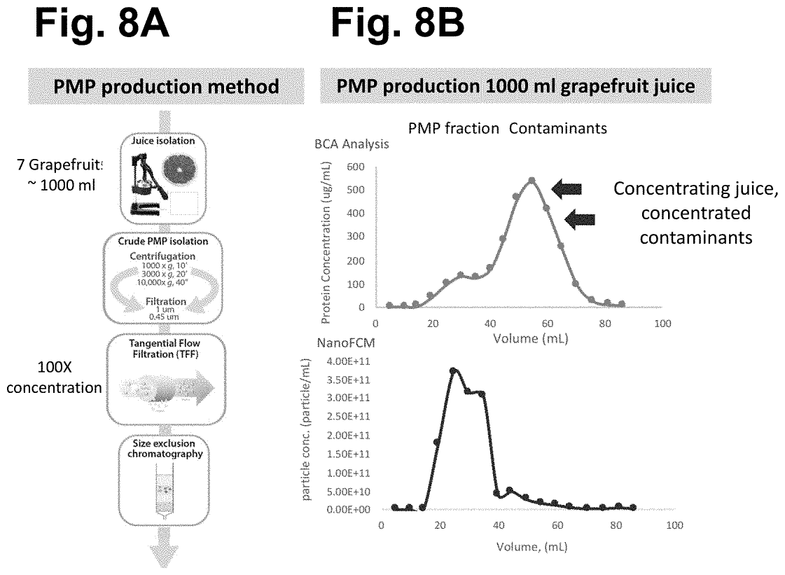

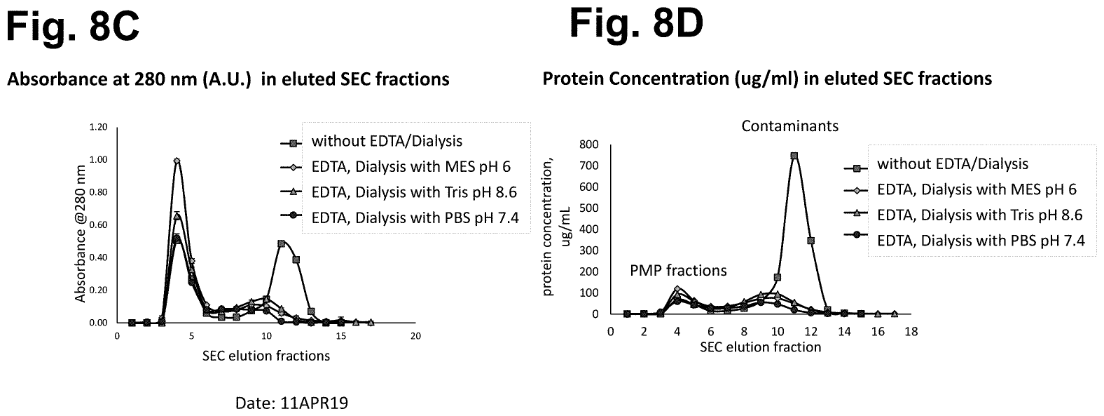

Red grapefruits were obtained from a local Whole Foods Market®, and 1000 ml juice was isolated using a juice press. The workflow for grapefruit PMP production using TFF and SEC is depicted in FIG. 8A. Juice was subjected to differential centrifugation at 1000×g for 10 minutes, 3000× g for 20 minutes, and 10,000× g for 40 minutes to remove large debris. Cleared grapefruit juice was concentrated and washed once using a TFF (5 nm pore size) to 2 mL (100×). Next, we used size exclusion chromatography to elute the PMP-containing fractions. SEC elution fractions were analyzed by nano-flow cytometry using a NanoFCM to determine PMP concentration using concentration and size standards provided by the manufacturer. In addition, protein concentration (Pierce™ BCA assay, ThermoFisher) was determined for SEC fractions to identify the fractions in which PMPs are eluted. The scaled production from 1 liter of juice (100× concentrated) also concentrated a high amount of contaminants in the late SEC fractions as can be detected by BCA assay (FIG. 8B, top panel). The overall total PMP yield (FIG. 8B, bottom panel) was lower in the scaled production when compared to single grapefruit isolations, which may indicate loss of PMPs.

b) Reducing Contaminants by EDTA Incubation and Dialysis

Red grapefruits were obtained from a local Whole Foods Market®, and 800 ml juice was isolated using a juice press. Juice was subjected to differential centrifugation at 1000×g for 10 minutes, 3000× g for 20 minutes, and 10,000× g for 40 minutes to remove large debris, and filtered through a 1 μm and 0.45 μm filter to remove large particles. Cleared grapefruit juice was split into 4 different treatment groups containing 125 ml juice each. Treatment Group 1 was processed as described in Example 4a, concentrated and washed (PBS) to a final concentration of 63×, and subjected to SEC. Prior to TFF, 475 ml juice was incubated with a final concentration of 50 mM EDTA, pH 7.15 for 1.5 hrs at RT to chelate iron and reduce the formation of pectin macromolecules. Afterwards, juice was split in three treatment groups that underwent TFF concentration with either a PBS (without calcium/magnesium) pH 7.4, MES pH 6, or Tris pH 8.6 wash to a final juice concentration of 63×. Next, samples were dialyzed in the same wash buffer overnight at 4° C. using a 300 kDa membrane and subjected to SEC. Compared to the high contaminant peak in the late elution fractions of the TFF only control, EDTA incubation followed by overnight dialysis strongly reduced contaminants, as shown by absorbance at 280 nm (FIG. 8C) and BCA protein analysis (FIG. 8D), which is sensitive to the presence of sugars and pectins. There was no difference in the dialysis buffers used (PBS without calcium/magnesium pH 7.4, MES pH 6, Tris pH 8.6). Our data indicates that incubation with EDTA followed by dialysis reduces the amount of co-purified contaminants, facilitating scaled PMP production.

### Example 13: PMP Production from Plant Cell Culture Medium

This example demonstrates that PMPs can be produced from plant cell culture. In this example, the Zea mays Black Mexican Sweet (BMS) cell line is used as a model plant cell line.

a) Production of Zea mays BMS Cell Line PMPs

The Zea mays Black Mexican sweet (BMS) cell line was purchased from the ABRC and was grown in Murashige and Skoog basal medium pH 5.8, containing 4.3 g/L Murashige and Skoog Basal Salt Mixture (Sigma M5524), 2% sucrose (S0389, Millipore Sigma), lx MS vitamin solution (M3900, Millipore Sigma), 2 mg/L 2,4-dichlorophenoxyacetic acid (D7299, Millipore Sigma) and 250 ug/L thiamine HCL (V-014, Millipore Sigma), at 24° C. with agitation (110 rpm), and was passaged 20% volume/volume every 7 days.

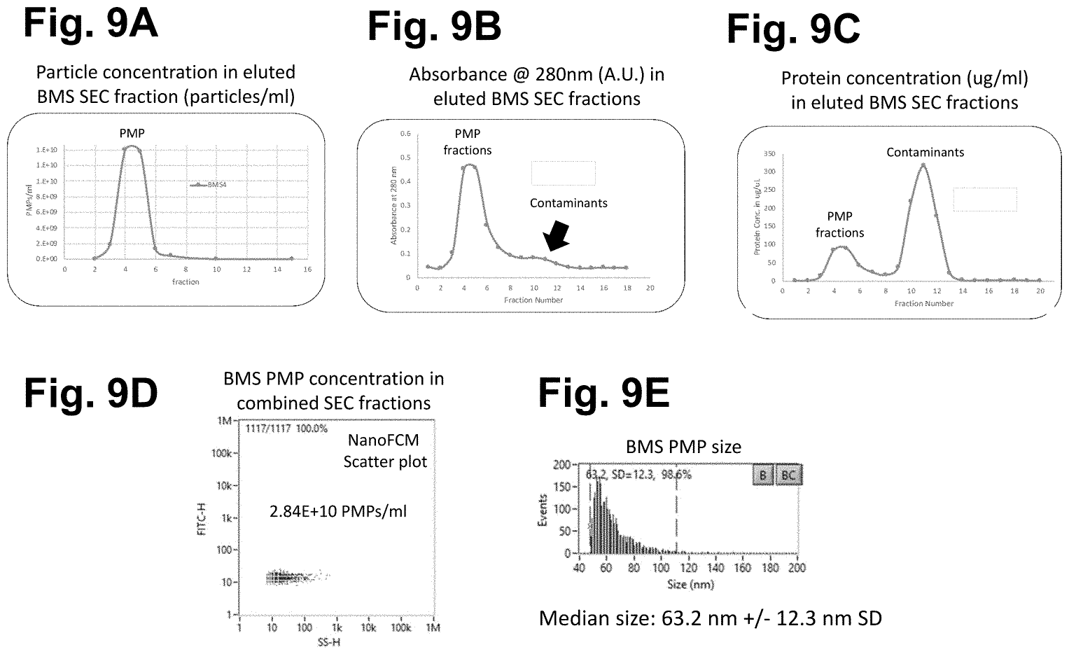

Three days after passaging, 160 ml BMS cells was collected and spun down at 500× g for 5 min to remove cells, and 10,000×g for 40 min to remove large debris. Medium was passed through a 0.45 μm filter to remove large particles, and filtered medium was concentrated and washed (100 ml MES buffer, 20 mM MES, 100 mM NaCL, pH 6) by TFF (5 nm pore size) to 4 mL (40×). Next, we used size exclusion chromatography to elute the PMP-containing fractions, which were analyzed by NanoFCM for PMP concentration, by absorbance at 280 nm (SpectraMax®), and by a protein concentration assay (Pierce™ BCA assay, ThermoFisher) to verify the PMP-containing fractions and late fractions containing contaminants (FIGS. 9A-9C). SEC fractions 4-6 contained purified PMPs (fractions 9-13 contained contaminants), and were pooled together. The final PMP concentration (2.84×10¹⁰ PMPs/ml) and median PMP size (63.2 nm+/−12.3 nm SD) in the combined PMP containing fractions were determined by NanoFCM, using concentration and size standards provided by the manufacturer (FIGS. 9D-9E).

These data show that PMPs can be isolated, purified, and concentrated from plant liquid culture media.

### Example 14: Treatment of a Microbe with Protein Loaded PMPs

This example demonstrates that PMPs can be exogenously loaded with a protein, PMPs can protect their cargo from degradation, and PMPs can deliver their functional cargo to an organism. In this example, grapefruit PMPs are used as model PMP, Pseudomonas aeruginosa bacteria is used as a model organism, and luciferase protein is used as a model protein.

While protein and peptide-based drugs have great potential to impact the fitness of a wide variety pathogenic bacteria and fungi that are resistant or hard to treat, their deployment has been unsuccessful due to their instability and formulation challenges.

a) Production of Grapefruit PMPs Using TFF Combined with SEC

Red organic grapefruits were obtained from a local Whole Foods Market®. Four liters of grapefruit juice were collected using a juice press, pH adjusted to pH4 with NaOH, incubated with 1 U/ml pectinase (Sigma, 17389) to remove pectin contaminants, and subsequently centrifuged at 3,000 g for 20 minutes, followed by 10,000 g for 40 minutes to remove large debris. Next, the processed juice was incubated with 500 mM EDTA pH8.6, to a final concentration of 50 mM EDTA, pH7.7 for 30 minutes to chelate calcium and prevent the formation of pectin macromolecules. Subsequently, the EDTA-treated juice was passaged through an 11 μm, 1 μm and 0.45 μm filter to remove large particles. Filtered juice was washed and concentrated by Tangential Flow Filtration (TFF) using a 300 kDa TFF. Juice was concentrated 5×, followed by a 6 volume exchange wash with PBS, and further filtrated to a final concentration 198 mL (20×). Next, we used size exclusion chromatography to elute the PMP-containing fractions, which were analyzed by absorbance at 280 nm (SpectraMax®) and protein concentration (Pierce™ BCA assay, ThermoFisher) to verify the PMP-containing fractions and late fractions containing contaminants. SEC fractions 3-7 contained purified PMPs (fractions 9-12 contained contaminants), were pooled together, were filter sterilized by sequential filtration using 0.8 μm, 0.45 μm and 0.22 μm syringe filters, and were concentrated further by pelleting PMPs for 1.5 hrs at 40,000× g and resuspending the pellet in 4 ml UltraPure™ DNase/RNase-Free Distilled Water (ThermoFisher, 10977023). Final PMP concentration (7.56×10¹² PMPs/ml) and average PMP size (70.3 nm+/−12.4 nm SD) were determined by NanoFCM, using concentration and size standards provided by the manufacturer.

b) Loading of Luciferase Protein into Grapefruit PMPs

Grapefruit PMPs were produced as described in Example 14a. Luciferase (Luc) protein was purchased from LSBio (cat. no. LS-G5533-150) and dissolved in PBS, pH7.4 to a final concentration of 300 μg/mL. Filter-sterilized PMPs were loaded with luciferase protein by electroporation, using a protocol adapted from Rachael W. Sirianni and Bahareh Behkam (eds.), Targeted Drug Delivery: Methods and Protocols, Methods in Molecular Biology, vol. 1831. PMPs alone (PMP control), luciferase protein alone (protein control), or PMP+luciferase protein (protein-loaded PMPs), were mixed with 4.8× electroporation buffer (100% Optiprep (Sigma, D1556) in UltraPure water) to have a final 21% Optiprep concentration in the reaction mix (see Table 6). Protein control was made by mixing luciferase protein with UltraPure water instead of Optiprep (protein control), as the final PMP-Luc pellet was diluted in water. Samples were transferred into chilled cuvettes and electroporated at 0.400 kV, 125 μF (0.125 mF), resistance low 100 Ω-high 600Ω with two pulses (4-10 ms) using a Biorad GenePulser®. The reaction was put on ice for 10 minutes, and transferred to a pre-ice chilled 1.5 ml ultracentrifuge tube. All samples containing PMPs were washed 3 times by adding 1.4 ml ultrapure water, followed by ultracentrifugation (100,000×g for 1.5 h at 4° C.). The final pellet was resuspended in a minimal volume of UltraPure water (50 μL) and kept at 4° C. until use. After electroporation, samples containing luciferase protein only were not washed by centrifugation and were stored at 4° C. until use.

To determine the PMP loading capacity, one microliter of Luciferase-loaded PMPs (PMP-Luc) and one microliter of unloaded PMPs were used. To determine the amount of Luciferase protein loaded in the PMPs, a Luciferase protein (LSBio, LS-G5533-150) standard curve was made (10, 30, 100, 300, and 1000 ng). Luciferase activity in all samples and standards was assayed using the ONE-Glo™ luciferase assay kit (Promega, E6110) and measuring luminescence using a SpectraMax® spectrophotometer. The amount of luciferase protein loaded in PMPs was determined using a standard curve of Luciferase protein (LSBio, LS-G5533-150) and normalized to the luminescence in the unloaded PMP sample. The loading capacity (ng luciferase protein per 1E+9 particles) was calculated as the luciferase protein concentration (ng) divided by the number of loaded PMPs (PMP-Luc). The PMP-Luc loading capacity was 2.76 ng Luciferase protein/1×10⁹ PMPs.

Our results indicate that PMPs can be loaded with a model protein that remains active after encapsulation.

TABLE 6
Luciferase protein loading strategy using electroporation.

| | Luciferase | Luciferase | PMP |
|---|---|---|---|
| | PMP (protein- | (protein | (PMP |
| | loaded PMPs) | control) | control) |
| Luciferase protein (300 | 25 | 25 | 0 |
| μg/mL (μL) | | | |
| Optiprep 100% (μL) | 14.7 | 0 | 14.7 |
| UltraPure water (μL) | 10.3 | 45 | 35.3 |
| PMP GF (PMP stock | 20 | 0 | 20 |
| concentration = 7.56 × 10¹² | | | |
| PMP/mL) | | | |
| Final volume | 70 | 70 | 70 |

Note:
25 μL luciferase is equivalent to 7.5 μg luciferase protein.

c) Treatment of Pseudomonas aeruginosa with Luciferase Protein-Loaded Grapefruit PMPs

Pseudomonas aeruginosa (ATCC) was grown overnight at 30° C. in tryptic soy broth supplemented with 50 ug/ml Rifampicin, according to the supplier's instructions. Pseudomonas aeruginosa cells (total volume of 5 ml) were collected by centrifugation at 3,000×g for 5 min. Cells were washed twice with 10 ml 10 mM MgCl₂ and resuspended in 5 ml 10 mM MgCl₂. The OD600 was measured and adjusted to 0.5.

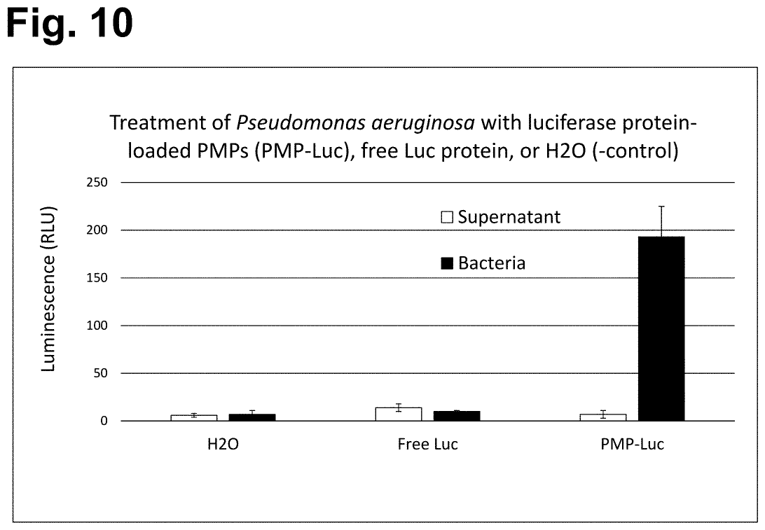

Treatments were performed in duplicate in 1.5 ml Eppendorf tubes, containing 50 μl of the resuspended Pseudomonas aeruginosa cells supplemented with either 3 ng of PMP-Luc (diluted in Ultrapure water), 3 ng free luciferase protein (protein only control; diluted in Ultrapure water), or Ultrapure water (negative control). Ultrapure water was added to 75 μl in all samples. Samples were mixed and incubated at room temperature for 2 h and covered with aluminum foil. Samples were next centrifuged at 6,000×g for 5 min, and 70 μl of the supernatant was collected and saved for luciferase detection. The bacterial pellet was subsequently washed three times with 500 μl 10 mM MgCl₂ containing 0.5% Triton X-100 to remove/burst PMPs that were not taken up. A final wash with 1 ml 10 mM MgCl₂ was performed to remove residual Triton X-100. 970 μl of the supernatant was removed (leaving the pellet in 30 ul wash buffer) and 20 μl 10 mM MgCl₂ and 25 μl Ultrapure water were added to resuspend the Pseudomonas aeruginosa pellets. Luciferase protein was measured by luminescence using the ONE-Glo™ luciferase assay kit (Promega, E6110), according to the manufacturer's instructions. Samples (bacterial pellet and supernatant samples) were incubated for 10 minutes, and luminescence was measured on a SpectraMax® spectrophotometer. Pseudomonas aeruginosa treated with Luciferase protein-loaded grapefruit PMPs had a 19.3 fold higher luciferase expression than treatment with free luciferase protein alone or the Ultrapure water control (negative control), indicating that PMPs are able to efficiently deliver their protein cargo into bacteria (FIG. 10). In addition, PMPs appear to protect luciferase protein from degradation, as free luciferase protein levels in both the supernatant and bacterial pellets are very low. Considering the treatment dose was 3 ng luciferase protein, based on the luciferase protein standard curve, free luciferase protein in supernatant or bacterial pellets after 2 hours of RT incubation in water corresponds to <0.1 ng luciferase protein, indicating protein degradation.

Our data shows that PMPs can deliver a protein cargo into organisms, and that PMPs can protect their cargo from degradation by the environment.

### Example 15: Insulin-Loaded PMPs Protect their Protein Cargo from Enzymatic Degradation

This example demonstrates that human insulin protein was loaded into lemon and grapefruit PMPs and that PMP-encapsulated insulin is protected from degradation by proteinase K and simulated gastrointestinal (GI) fluids. Compositions that can withstand degradation by GI fluids may be useful for oral delivery of compounds, e.g., proteins.

a) Production of PMPs

Lemons and grapefruits were obtained from a local grocery store. Fruits were washed with 1% Liquinox® (Alconox®) detergent and rinsed under warm water. Six liters each of lemon and grapefruit juice were collected using a juice press, depulped through a 1 mm mesh pore size metal strainer, and adjusted to pH 4.5 with 10 N sodium hydroxide before the addition of pectinase enzyme at a final concentration of 0.5 U/mL (Pectinase from Aspergillus niger, Sigma). The juice was incubated with the pectinase enzyme for 2 hours at 25° C. and subsequently centrifuged at 3,000×g for 20 minutes, followed by centrifugation at 10,000×g for 40 minutes to remove large debris. Next, EDTA was added to the processed juice to a final concentration of 50 mM, and pH was adjusted to 7.5. Juice clarification was performed by vacuum filtration through 11 μm filter paper (Whatman®), followed by 1 μM syringe-filtration (glass fiber, VWR®) and 0.45 μM vacuum filtration (PES, Celltreat® Scientific Products) to remove large particles.

Filtered juice was subsequently concentrated, washed, and concentrated again by tangential flow filtration (TFF) using a 300 kDa pore size hollow fiber filter. Juice was concentrated 8×, followed by diafiltration into 10 diavolumes of 1×PBS (pH 7.4), and further concentrated to a final concentration of 50× based on the initial juice volume. Next, we used size exclusion chromatography (SEC; maxiPURE-EVs size exclusion chromatography columns, HansaBioMed Life Sciences) to elute the PMP-containing fractions, which were analyzed by absorbance at 280 nm (SpectraMax® spectrophotometer) and protein concentration was determined by BCA assay (Pierce™ BCA Protein Assay Kit, Thermo Scientific) to verify the PMP-containing fractions and late fractions containing contaminants. Lemon SEC fractions 3-8 (early fractions) contained purified PMPs; fractions 9-14 contained contaminants. Grapefruit SEC fractions 3-7 (early fractions) contained purified PMPs; fractions 8-14 contained contaminants. The early fractions were combined and filter-sterilized by sequential filtration using 1 μm glass fiber syringe filters (Acrodisc®, Pall Corporation), 0.45 μm syringe filters (Whatman® PURADISC™), and 0.22 μm (Whatman® PURADISC™) syringe filters under aseptic conditions in a tissue culture hood. Then, PMPs were concentrated by ultracentrifugation for 1.5 hours at 40,000×g at 4° C. The PMP pellet was resuspended in 5.5 mL of sterile 1×PBS (pH 7.4). Final PMP concentration (7.59×10¹³ lemon PMPs/mL; 3.54×10¹³ grapefruit PMPs/mL) and PMP median size were determined by NanoFCM, using concentration and size standards provided by the manufacturer. Protein concentration of the final PMP suspension was determined by BCA (Pierce™ BCA Protein Assay Kit, Thermo Scientific) (lemon PMPs 1.1 mg/mL; grapefruit PMPs 4.4 mg/mL). 2 mL of the produced lemon PMPs and 2 mL of the produced grapefruit PMPs were ultracentrifuged (1.5 hours, 40,000×g, 4° C.) to replace the PBS buffer with UltraPure™ water (Invitrogen), and the concentration was remeasured by NanoFCM (8.42×10¹³ lemon PMPs/mL; 3.29×10¹³ grapefruit PMPs/mL). These PMP suspensions were used for lipid extraction as described in Example 15b.

b) Loading of PMPs with Insulin Protein

Total lipids from lemon and grapefruit PMPs were extracted using the Bligh-Dyer method (Bligh and Dyer, Can J Biochem Physiol, 37: 911-917, 1959). PMP pellets were prepared by ultracentrifugation at 40,000×g for 1.5 hours at 4° C. and resuspended in UltraPure™ water (Invitrogen). In a glass tube, a mixture of chloroform:methanol (CHCl₃:MeOH) at a 1:2 v/v ratio was prepared. For each 1 mL PMP sample, 3.75 mL of CHCl₃:MeOH was added and vortexed. Then, 1.25 mL CHCl₃ was added and vortexed. Finally, 1.25 mL UltraPure™ water (Invitrogen) was added and vortexed. This preparation was centrifuged at 210×g in table-top centrifuge for 5 minutes at room temperature to give a two-phase system (aqueous on top, organic at the bottom). The organic phase was recovered using a glass Pasteur pipette, taking care to avoid both the aqueous phase and the interphase. The organic phase was aliquoted into smaller volumes containing approximately 2-3 mg of lipids (1 L of citrus juice yields approximately 3-5×10¹³ PMPs, which corresponds to approximately 10 mg of lipids). Lipid aliquots were dried under nitrogen gas and stored at −20° C. until use.

Recombinant human insulin (Gibco, cat. no. A11382II) was dissolved in 10 mM hydrochloric acid at 10 mg/mL and diluted to 1 mg/mL in water. Insulin-loaded lipid reconstructed PMPs (recPMPs) were prepared from 3 mg dried lemon PMP lipids and 0.6 mg insulin (5:1 w/w ratio), which was added to the lipid film at a volume of 600 μL. Glass beads (˜7-8) were added, and the solution was agitated at room temperature for 1-2 hours. The samples were then sonicated in a water bath sonicator (Branson) for 5 minutes at room temperature, vortexed, and agitated again at room temperature for 1-2 hours. The formulations were then extruded using an Mini Extruder (Avanti® Polar Lipids) with sequential 800 nm, 400 nm, and 200 nm polycarbonate membranes. Subsequently, the formulation was purified using a Zeba™ Spin Desalting Column (40 kDa MWCO, Thermo Fisher Scientific), followed by ultracentrifugation at 100,000×g for 45 minutes, and washed once with UltraPure™ water. The pellet was resuspended in 1×PBS (pH 7.4) to a final concentration of 7.94×10¹¹ recPMPs/mL, measured using nanoFCM.

Insulin-loaded grapefruit recPMPs were similarly formulated, except that 2 mg of dried lipids was mixed with 0.4 mg insulin (maintaining the 5:1 w/w ratio). Samples were agitated at room temperature for 3.5 hours, sonicated for 5 minutes, vortexed, and again sonicated for 5 minutes, all at room temperature. Extrusion was performed as described above. Purification was done using Amicon® Ultra centrifugation filters (100K MWCO, Millipore) at 14,000×g for 5 minutes (repeated once), followed by Zeba™ Spin Desalting Column (40 kDa MWCO, Thermo Fisher Scientific) and ultracentrifugation as described above. The pellet was resuspended in 1×PBS to a final concentration of 1.19×10¹² recPMPs/mL, measured using nanoFCM.

To assess insulin loading into recPMPs and to test whether insulin-loaded recPMPs from lemon and grapefruit PMP lipids can protect human insulin protein, a proteinase K (ProtK) treatment followed by Western blot analysis was performed. To this end, insulin-loaded recPMP samples were incubated with 20 μg/mL ProtK (New England Biolabs® Inc.) in 50 mM Tris hydrochloride (pH 7.5) and 5 mM calcium chloride at 37° C. for 1 hour with agitation.

To assess insulin protein levels, samples (10 μL) were diluted with Laemmli sample buffer with Orange G (Sigma) substituted for bromophenol blue to eliminate signal interference during imaging. Samples were boiled for 10 minutes, cooled on ice, loaded onto Tris-glycine gels (TGX™, Bio-Rad). Subsequently, gels were transferred onto nitrocellulose membranes using an iBlot™ 2 system (Invitrogen) according to the manufacturer's instructions. Nitrocellulose membranes were briefly washed with 1×PBS (pH 7.4) and blocked with Odyssey blocking buffer (Li-COR) for 1 hour at room temperature. Membranes were then incubated with 1:1000 rabbit anti-insulin primary antibody (ab181547, Abcam), followed by 1:10,000 goat anti-rabbit IRDye® 800CW secondary antibody (Li-COR) for 2 hours each. Membranes were washed three times after each antibody incubation with 1×PBS with 0.1% Tween® 20 (Sigma) and a final rinse in 1×PBS. Membranes were imaged on an iBright™ 1500 FL (Invitrogen™). Lemon and grapefruit insulin-recPMP samples showed comparable levels of insulin protein with and without ProtK treatment, indicating that the insulin is encapsulated and protected within the PMPs. Quantification of the amount of loaded insulin based on free insulin protein standards and normalized for PMP concentration revealed loading of 21 ng of insulin per 10⁹ lemon recPMPs.

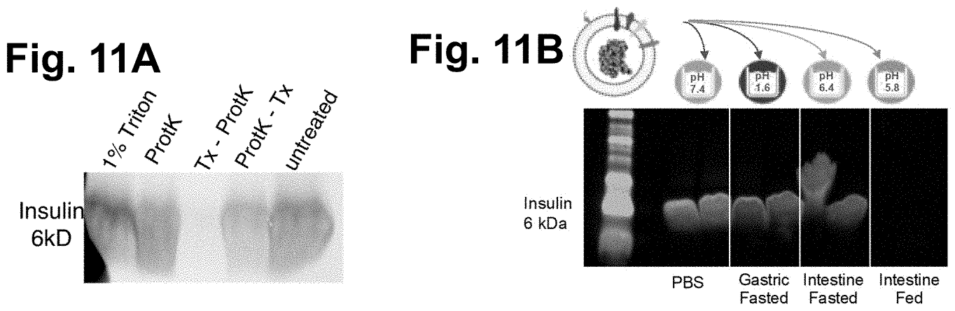

To determine whether lysing the PMP lipid membrane before or after proteinase K (ProtK) treatment affected insulin stability, grapefruit insulin-loaded recPMP samples were treated with (1) 1% TRITON™ X-100 for 30 minutes (lysing the lipid membranes and exposing the protein cargo); (2) 10 μg/mL ProtK treatment for 1 hour; (3) 1% TRITON™ X-100 for 30 minutes, followed by 10 μg/mL ProtK treatment for 1 hour, and inactivating the reaction by adding 10 mM PMSF; and (4) 10 μg/ml ProtK treatment for 1 hour, inactivating ProtK by adding 10 mM PMSF, followed by 1% TRITON™ X-100 for 30 minutes. All treatments were performed at 37° C. with agitation. A Western blot for insulin was performed for each sample as described above (FIG. 11A). Encapsulated insulin cargo was degraded only when PMP membranes were lysed by TRITON™ X-100 prior to ProtK digestion, demonstrating that insulin protein is encapsulated inside the PMPs and that PMPs protect protein cargo from enzymatic digestion by ProtK.

c) Stability of Insulin-Loaded PMPs in GI Fluids

To further assess the stability of encapsulated insulin, loaded PMPs prepared from lemon lipids were exposed to simulated GI fluids that contain relevant bile acids, digestive enzymes, and pH to mimic distinct gastrointestinal environments and conditions. Digestive buffers were purchased from Biorelevant and prepared according to the manufacturer's instructions. The following buffers were used: FaSSGF (fasted stomach, pH 1.6), FaSSIF (fasted small intestines, pH 6.4), and FeSSIF (fed small intestines, pH 5.8). 1×PBS (pH 7.4) was used as negative control. For each sample, 980 μL buffer was added to 20 μL insulin-loaded recPMPs (lemon; 7.94×10¹¹ recPMPs/mL) under low vortexing. Each treatment (buffer condition) was performed in duplicate. Insulin-loaded recPMPs were incubated in FaSSGF for 1 hour and in all other buffers for 4 hours to approximate the passage times in the human digestive system. All incubations were performed at 37° C. under slow rotation. Following incubation at 37° C., samples were placed on ice and centrifuged at 100,000×g for 50 minutes to pellet the insulin-loaded recPMPs. Samples were washed once by resuspension in UltraPure™ water (Invitrogen) and centrifuged again. Pellets were then resuspended in 10 μL UltraPure™ water and used for Western blot analysis to detect insulin protein as described above. Imaging of the GI buffer-treated samples (FIG. 11B) revealed that insulin-loaded recPMPs are stable in buffers simulating both fasted stomach (FaSSGF) and fasted small intestines (FaSSIF). In simulated fed small intestine (FeSSIF) buffer, however, insulin could not be detected (FIG. 11B), indicating that under these conditions insulin-loaded recPMPs vesicles were not able to protect insulin from degradation. Free insulin protein was stable only in 1×PBS, but unstable in all three GI buffers used (data not shown). Taken together, these experiments show that reconstructed PMPs from citrus lipids protect their protein payload from degradation by low pH (FaSSGF) and digestive enzymes/GI fluids (ProtK, FaSSIF).

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

Other embodiments are within the claims.

### APPENDIX

TABLE 7
Plant EV-Markers

| Example Species | Accession No. | Protein Name |
|---|---|---|
| Arabidopsis thaliana | C0LGG8 | Probable LRR receptor-like serine/threonine-protein kinase |
| | | At1g53430 (EC 2.7.11.1) |
| Arabidopsis thaliana | F4HQT8 | Uncharacterized protein |
| Arabidopsis thaliana | F4HWU0 | Protein kinase superfamily protein |
| Arabidopsis thaliana | F4I082 | Bifunctional inhibitor/lipid-transfer protein/seed storage 2S |
| | | albumin superfamily protein |
| Arabidopsis thaliana | F4I3M3 | Kinase with tetratricopeptide repeat domain-containing |
| | | protein |
| Arabidopsis thaliana | F4IB62 | Leucine-rich repeat protein kinase family protein |
| Arabidopsis thaliana | O03042 | Ribulose bisphosphate carboxylase large chain (RuBisCO |
| | | large subunit) (EC 4.1.1.39) |
| Arabidopsis thaliana | O03986 | Heat shock protein 90-4 (AtHSP90.4) (AtHsp90-4) (Heat |
| | | shock protein 81-4) (Hsp81-4) |
| Arabidopsis thaliana | O04023 | Protein SRC2 homolog (AtSRC2) |
| Arabidopsis thaliana | O04309 | Jacalin-related lectin 35 (JA-responsive protein 1) |
| | | (Myrosinase-binding protein-like At3g16470) |
| Arabidopsis thaliana | O04314 | PYK10-binding protein 1 (Jacalin-related lectin 30) (Jasmonic |
| | | acid-induced protein) |
| Arabidopsis thaliana | O04922 | Probable glutathione peroxidase 2 (EC 1.11.1.9) |
| Arabidopsis thaliana | O22126 | Fasciclin-like arabinogalactan protein 8 (AtAGP8) |
| Arabidopsis thaliana | O23179 | Patatin-like protein 1 (AtPLP1 (EC 3.1.1.—) (Patatin-related |
| | | phospholipase A IIgamma) (pPLAIIg) (Phospholipase A IVA) |
| | | (AtPLAIVA) |
| Arabidopsis thaliana | O23207 | Probable NAD(P)H dehydrogenase (quinone) FQR1-like 2 |
| | | (EC 1.6.5.2) |
| Arabidopsis thaliana | O23255 | Adenosylhomocysteinase 1 (AdoHcyase 1) (EC 3.3.1.1) |
| | | (Protein EMBRYO DEFECTIVE 1395) (Protein |
| | | HOMOLOGY-DEPENDENT GENE SILENCING 1) |
| | | (S-adenosyl-L-homocysteine hydrolase 1) |
| | | (SAH hydrolase 1) |
| Arabidopsis thaliana | O23482 | Oligopeptide transporter 3 (AtOPT3) |
| Arabidopsis thaliana | O23654 | V-type proton ATPase catalytic subunit A (V-ATPase subunit |
| | | A) (EC 3.6.3.14) (V-ATPase 69 kDa subunit) (Vacuolar H(+)-ATPase |
| | | subunit A) (Vacuolar proton pump subunit alpha) |
| Arabidopsis thaliana | O48788 | Probable inactive receptor kinase At2g26730 |
| Arabidopsis thaliana | O48963 | Phototropin-1 (EC 2.7.11.1) (Non-phototropic hypocotyl |
| | | protein 1) (Root phototropism protein 1) |
| Arabidopsis thaliana | O49195 | Vegetative storage protein 1 |
| Arabidopsis thaliana | O50008 | 5-methyltetrahydropteroyltriglutamate--homocysteine |
| | | methyltransferase 1 (EC 2.1.1.14) (Cobalamin-independent |
| | | methionine synthase 1) (AtMS1) (Vitamin-B12-independent |
| | | methionine synthase 1) |
| Arabidopsis thaliana | O64696 | Putative uncharacterized protein At2g34510 |
| Arabidopsis thaliana | O65572 | Carotenoid 9,10(9′,10′)-cleavage dioxygenase 1 (EC |
| | | 1.14.99.n4) (AtCCD1) (Neoxanthin cleavage enzyme NC1) |
| | | (AtNCED1) |
| Arabidopsis thaliana | O65660 | PLAT domain-containing protein 1 (AtPLAT1) (PLAT domain |
| | | protein 1) |
| Arabidopsis thaliana | O65719 | Heat shock 70 kDa protein 3 (Heat shock cognate 70 kDa |
| | | protein 3) (Heat shock cognate protein 70-3) (AtHsc70-3) |
| | | (Heat shock protein 70-3) (AtHsp70-3) |
| Arabidopsis thaliana | O80517 | Uclacyanin-2 (Blue copper-binding protein II) (BCB II) |
| | | (Phytocyanin 2) (Uclacyanin-II) |
| Arabidopsis thaliana | O80576 | At2g44060 (Late embryogenesis abundant protein, group 2) |
| | | (Similar to late embryogenesis abundant proteins) |
| Arabidopsis thaliana | O80725 | ABC transporter B family member 4 (ABC transporter |
| | | ABCB.4) (AtABCB4) (Multidrug resistance protein 4) |
| | | (P-glycoprotein 4) |
| Arabidopsis thaliana | O80837 | Remorin (DNA-binding protein) |
| Arabidopsis thaliana | O80852 | Glutathione S-transferase F9 (AtGSTF9) (EC 2.5.1.18) |
| | | (AtGSTF7) (GST class-phi member 9) |
| Arabidopsis thaliana | O80858 | Expressed protein (Putative uncharacterized protein |
| | | At2g30930) (Putative uncharacterized protein At2g30930; |
| | | F7F1.14) |
| Arabidopsis thaliana | O80939 | L-type lectin-domain containing receptor kinase IV.1 |
| | | (Arabidopsis thaliana lectin-receptor kinase e) (AthlecRK-e) |
| | | (LecRK-IV.1) (EC 2.7.11.1) (Lectin Receptor Kinase 1) |
| Arabidopsis thaliana | O80948 | Jacalin-related lectin 23 (Myrosinase-binding protein-like |
| | | At2g39330) |
| Arabidopsis thaliana | O82628 | V-type proton ATPase subunit G1 (V-ATPase subunit G1) |
| | | (Vacuolar H(+)-ATPase subunit G isoform 1) (Vacuolar |
| | | proton pump subunit G1) |
| Arabidopsis thaliana | P10795 | Ribulose bisphosphate carboxylase small chain 1A, |
| | | chloroplastic (RuBisCO small subunit 1A) (EC 4.1.1.39) |
| Arabidopsis thaliana | P10896 | Ribulose bisphosphate carboxylase/oxygenase activase, |
| | | chloroplastic (RA) (RuBisCO activase) |
| Arabidopsis thaliana | P17094 | 60S ribosomal protein L3-1 (Protein EMBRYO DEFECTIVE |
| | | 2207) |
| Arabidopsis thaliana | P19456 | ATPase 2, plasma membrane-type (EC 3.6.3.6) (Proton |
| | | pump 2) |
| Arabidopsis thaliana | P20649 | ATPase 1, plasma membrane-type (EC 3.6.3.6) (Proton |
| | | pump 1) |
| Arabidopsis thaliana | P22953 | Probable mediator of RNA polymerase II transcription subunit |
| | | 37e (Heat shock 70 kDa protein 1) (Heat shock cognate 70 |
| | | kDa protein 1) (Heat shock cognate protein 70-1) (AtHsc70-1) |
| | | (Heat shock protein 70-1) (AtHsp70-1) (Protein |
| | | EARLY-RESPONSIVE TO DEHYDRATION 2) |
| Arabidopsis thaliana | P23586 | Sugar transport protein 1 (Glucose transporter) (Hexose |
| | | transporter 1) |
| Arabidopsis thaliana | P24636 | Tubulin beta-4 chain (Beta-4-tubulin) |
| Arabidopsis thaliana | P25696 | Bifunctional enolase 2/transcriptional activator (EC 4.2.1.11) |
| | | (2-phospho-D-glycerate hydro-lyase 2) (2-phosphoglycerate |
| | | dehydratase 2) (LOW EXPRESSION OF OSMOTICALLY |
| | | RESPONSIVE GENES 1) |
| Arabidopsis thaliana | P25856 | Glyceraldehyde-3-phosphate dehydrogenase GAPA1, |
| | | chloroplastic (EC 1.2.1.13) (NADP-dependent |
| | | glyceraldehydephosphate dehydrogenase A subunit 1) |
| Arabidopsis thaliana | P28186 | Ras-related protein RABE1c (AtRABE1c) (Ras-related |
| | | protein Ara-3) (Ras-related protein Rab8A) (AtRab8A) |
| Arabidopsis thaliana | P30302 | Aquaporin PIP2-3 (Plasma membrane intrinsic protein 2-3) |
| | | (AtPIP2; 3) (Plasma membrane intrinsic protein 2c) (PIP2c) |
| | | (RD28-PIP) (TMP2C) (Water stress-induced tonoplast |
| | | intrinsic protein) (WSI-TIP) [Cleaved into: Aquaporin PIP2-3, |
| | | N-terminally processed] |
| Arabidopsis thaliana | P31414 | Pyrophosphate-energized vacuolar membrane proton pump |
| | | 1 (EC 3.6.1.1) (Pyrophosphate-energized inorganic |
| | | pyrophosphatase 1) (H(+)-PPase 1) (Vacuolar proton |
| | | pyrophosphatase 1) (Vacuolar proton pyrophosphatase 3) |
| Arabidopsis thaliana | P32961 | Nitrilase 1 (EC 3.5.5.1) |
| Arabidopsis thaliana | P38666 | 60S ribosomal protein L24-2 (Protein SHORT VALVE 1) |
| Arabidopsis thaliana | P39207 | Nucleoside diphosphate kinase 1 (EC 2.7.4.6) (Nucleoside |
| | | diphosphate kinase I) (NDK I) (NDP kinase I) (NDPK I) |
| Arabidopsis thaliana | P42643 | 14-3-3-like protein GF14 chi (General regulatory factor 1) |
| Arabidopsis thaliana | P42737 | Beta carbonic anhydrase 2, chloroplastic (AtbCA2) |
| | | (AtbetaCA2) (EC 4.2.1.1) (Beta carbonate dehydratase 2) |
| Arabidopsis thaliana | P42759 | Dehydrin ERD10 (Low-temperature-induced protein LTI45) |
| Arabidopsis thaliana | P42761 | Glutathione S-transferase F10 (AtGSTF10) (EC 2.5.1.18) |
| | | (AtGSTF4) (GST class-phi member 10) (Protein EARLY |
| | | RESPONSE TO DEHYDRATION 13) |
| Arabidopsis thaliana | P42763 | Dehydrin ERD14 |
| Arabidopsis thaliana | P42791 | 60S ribosomal protein L18-2 |
| Arabidopsis thaliana | P43286 | Aquaporin PIP2-1 (Plasma membrane intrinsic protein 2-1) |
| | | (AtPIP2; 1) (Plasma membrane intrinsic protein 2a) (PIP2a) |
| | | [Cleaved into: Aquaporin PIP2-1, N-terminally processed] |
| Arabidopsis thaliana | P46286 | 60S ribosomal protein L8-1 (60S ribosomal protein L2) |
| | | (Protein EMBRYO DEFECTIVE 2296) |
| Arabidopsis thaliana | P46422 | Glutathione S-transferase F2 (AtGSTF2) (EC 2.5.1.18) (24 |
| | | kDa auxin-binding protein) (AtPM24) (GST class-phi member 2) |
| Arabidopsis thaliana | P47998 | Cysteine synthase 1 (EC 2.5.1.47) (At.OAS.5-8) (Beta-substituted |
| | | Ala synthase 1; 1) (ARAth-Bsas1; 1) (CSase A) |
| | | (AtCS-A) (Cys-3A) (O-acetylserine (thiol)-lyase 1) (OAS-TL |
| | | A) (O-acetylserine sulfhydrylase) (Protein ONSET OF LEAF |
| | | DEATH 3) |
| Arabidopsis thaliana | P48347 | 14-3-3-like protein GF14 epsilon (General regulatory factor 10) |
| Arabidopsis thaliana | P48491 | Triosephosphate isomerase, cytosolic (TIM) (Triose-phosphate |
| | | isomerase) (EC 5.3.1.1) |
| Arabidopsis thaliana | P50318 | Phosphoglycerate kinase 2, chloroplastic (EC 2.7.2.3) |
| Arabidopsis thaliana | P53492 | Actin-7 (Actin-2) |
| Arabidopsis thaliana | P54144 | Ammonium transporter 1 member 1 (AtAMT1; 1) |
| Arabidopsis thaliana | P92963 | Ras-related protein RABB1c (AtRABB1c) (Ras-related |
| | | protein Rab2A) (AtRab2A) |
| Arabidopsis thaliana | P93004 | Aquaporin PIP2-7 (Plasma membrane intrinsic protein 2-7) |
| | | (AtPIP2; 7) (Plasma membrane intrinsic protein 3) (Salt |
| | | stress-induced major intrinsic protein) [Cleaved into: |
| | | Aquaporin PIP2-7, N-terminally processed] |
| Arabidopsis thaliana | P93025 | Phototropin-2 (EC 2.7.11.1) (Defective in chloroplast |
| | | avoidance protein 1) (Non-phototropic hypocotyl 1-like |
| | | protein 1) (AtKin7) (NPH1-like protein 1) |
| Arabidopsis thaliana | P93819 | Malate dehydrogenase 1, cytoplasmic (EC 1.1.1.37) |
| | | (Cytosolic NAD-dependent malate dehydrogenase 1) |
| | | (cNAD-MDH1) (Cytosolic malate dehydrogenase 1) |
| | | (Cytosolic MDH1) |
| Arabidopsis thaliana | Q03250 | Glycine-rich RNA-binding protein 7 (AtGR-RBP7) (AtRBG7) |
| | | (Glycine-rich protein 7) (AtGRP7) (Protein COLD, |
| | | CIRCADIAN RHYTHM, AND RNA BINDING 2) (Protein CCR2) |
| Arabidopsis thaliana | Q05431 | L-ascorbate peroxidase 1, cytosolic (AP) (AtAPx01) (EC |
| | | 1.11.1.11) |
| Arabidopsis thaliana | Q06611 | Aquaporin PIP1-2 (AtPIP1; 2) (Plasma membrane intrinsic |
| | | protein 1b) (PIP1b) (Transmembrane protein A) (AthH2) |
| | | (TMP-A) |
| Arabidopsis thaliana | Q07488 | Blue copper protein (Blue copper-binding protein) (AtBCB) |
| | | (Phytocyanin 1) (Stellacyanin) |
| Arabidopsis thaliana | Q0WLB5 | Clathrin heavy chain 2 |
| Arabidopsis thaliana | Q0WNJ6 | Clathrin heavy chain 1 |
| Arabidopsis thaliana | Q1ECE0 | Vesicle-associated protein 4-1 (Plant VAP homolog 4-1) |
| | | (AtPVA41) (Protein MEMBRANE-ASSOCIATED MANNITOL-INDUCED) |
| | | (AtMAMI) (VAMP-associated protein 4-1) |
| Arabidopsis thaliana | Q38882 | Phospholipase D alpha 1 (AtPLDalpha1) (PLD alpha 1) (EC |
| | | 3.1.4.4) (Choline phosphatase 1) (PLDalpha) |
| | | (Phosphatidylcholine-hydrolyzing phospholipase D 1) |
| Arabidopsis thaliana | Q38900 | Peptidyl-prolyl cis-trans isomerase CYP19-1 (PPIase CYP19-1) |
| | | (EC 5.2.1.8) (Cyclophilin of 19 kDa 1) (Rotamase |
| | | cyclophilin-3) |
| Arabidopsis thaliana | Q39033 | Phosphoinositide phospholipase C 2 (EC 3.1.4.11) |
| | | (Phosphoinositide phospholipase PLC2) (AtPLC2) (PI-PLC2) |
| Arabidopsis thaliana | Q39085 | Delta(24)-sterol reductase (EC 1.3.1.72) (Cell elongation |
| | | protein DIMINUTO) (Cell elongation protein Dwarf1) (Protein |
| | | CABBAGE1) (Protein ENHANCED VERY-LOW-FLUENCE |
| | | RESPONSE 1) |
| Arabidopsis thaliana | Q39228 | Sugar transport protein 4 (Hexose transporter 4) |
| Arabidopsis thaliana | Q39241 | Thioredoxin H5 (AtTrxh5) (Protein LOCUS OF |
| | | INSENSITIVITY TO VICTORIN 1) (Thioredoxin 5) (AtTRX5) |
| Arabidopsis thaliana | Q39258 | V-type proton ATPase subunit E1 (V-ATPase subunit E1) |
| | | (Protein EMBRYO DEFECTIVE 2448) (Vacuolar H(+)- |
| | | ATPase subunit E isoform 1) (Vacuolar proton pump subunit E1) |
| Arabidopsis thaliana | Q42112 | 60S acidic ribosomal protein P0-2 |
| Arabidopsis thaliana | Q42403 | Thioredoxin H3 (AtTrxh3) (Thioredoxin 3) (AtTRX3) |
| Arabidopsis thaliana | Q42479 | Calcium-dependent protein kinase 3 (EC 2.7.11.1) |
| | | (Calcium-dependent protein kinase isoform CDPK6) (AtCDPK6) |
| Arabidopsis thaliana | Q42547 | Catalase-3 (EC 1.11.1.6) |
| Arabidopsis thaliana | Q56WH1 | Tubulin alpha-3 chain |
| Arabidopsis thaliana | Q56WK6 | Patellin-1 |
| Arabidopsis thaliana | Q56X75 | CASP-like protein 4D2 (AtCASPL4D2) |
| Arabidopsis thaliana | Q56ZI2 | Patellin-2 |
| Arabidopsis thaliana | Q7Y208 | Glycerophosphodiester phosphodiesterase GDPDL1 (EC |
| | | 3.1.4.46) (Glycerophosphodiester phosphodiesterase-like 1) |
| | | (ATGDPDL1) (Glycerophosphodiesterase-like 3) (Protein |
| | | SHV3-LIKE 2) |
| Arabidopsis thaliana | Q84VZ5 | Uncharacterized GPI-anchored protein At5g19240 |
| Arabidopsis thaliana | Q84WU7 | Eukaryotic aspartyl protease family protein (Putative |
| | | uncharacterized protein At3g51330) |
| Arabidopsis thaliana | Q8GUL8 | Uncharacterized GPI-anchored protein At5g19230 |
| Arabidopsis thaliana | Q8GYA4 | Cysteine-rich receptor-like protein kinase 10 (Cysteine-rich |
| | | RLK10) (EC 2.7.11.—) (Receptor-like protein kinase 4) |
| Arabidopsis thaliana | Q8GYN5 | RPM1-interacting protein 4 |
| Arabidopsis thaliana | Q8GZ99 | At5g49760 (Leucine-rich repeat protein kinase family protein) |
| | | (Leucine-rich repeat receptor-like protein kinase) (Putative |
| | | receptor protein kinase) |
| Arabidopsis thaliana | Q8L636 | Sodium/calcium exchanger NCL (Na(+)/Ca(2+)-exchange |
| | | protein NCL) (Protein NCX-like) (AtNCL) |
| Arabidopsis thaliana | Q8L7S1 | At1g45200 (At1g45200/At1g45200) (Triacylglycerol |
| | | lipase-like 1) |
| Arabidopsis thaliana | Q8LAA6 | Probable aquaporin PIP1-5 (AtPIP1; 5) (Plasma membrane |
| | | intrinsic protein 1d) (PIP1d) |
| Arabidopsis thaliana | Q8LCP6 | Endoglucanase 10 (EC 3.2.1.4) (Endo-1,4-beta glucanase 10) |
| Arabidopsis thaliana | Q8RWV0 | Transketolase-1, chloroplastic (TK) (EC 2.2.1.1) |
| Arabidopsis thaliana | Q8S8Q6 | Tetraspanin-8 |
| Arabidopsis thaliana | Q8VZG8 | MDIS1-interacting receptor like kinase 2 (AtMIK2) (Probable |
| | | LRR receptor-like serine/threonine-protein kinase |
| | | At4g08850) (EC 2.7.11.1) |
| Arabidopsis thaliana | Q8VZU2 | Syntaxin-132 (AtSYP132) |
| Arabidopsis thaliana | Q8W4E2 | V-type proton ATPase subunit B3 (V-ATPase subunit B3) |
| | | (Vacuolar H(+)-ATPase subunit B isoform 3) (Vacuolar |
| | | proton pump subunit B3) |
| Arabidopsis thaliana | Q8W4S4 | V-type proton ATPase subunit a3 (V-ATPase subunit a3) |
| | | (V-type proton ATPase 95 kDa subunit a isoform 3) (V-ATPase |
| | | 95 kDa isoform a3) (Vacuolar H(+)-ATPase subunit a isoform |
| | | 3) (Vacuolar proton pump subunit a3) (Vacuolar proton |
| | | translocating ATPase 95 kDa subunit a isoform 3) |
| Arabidopsis thaliana | Q93VG5 | 40S ribosomal protein S8-1 |
| Arabidopsis thaliana | Q93XY5 | Tetraspanin-18 (TOM2A homologous protein 2) |
| Arabidopsis thaliana | Q93YS4 | ABC transporter G family member 22 (ABC transporter |
| | | ABCG.22) (AtABCG22) (White-brown complex homolog |
| | | protein 23) (AtWBC23) |
| Arabidopsis thaliana | Q93Z08 | Glucan endo-1,3-beta-glucosidase 6 (EC 3.2.1.39) |
| | | ((1->3)-beta-glucan endohydrolase 6) |
| | | ((1->3)-beta-glucanase 6) (Beta-1,3-endoglucanase |
| | | 6) (Beta-1,3-glucanase 6) |
| Arabidopsis thaliana | Q940M8 | 3-oxo-5-alpha-steroid 4-dehydrogenase (DUF1295) |
| | | (At1g73650/F25P22_7) |
| Arabidopsis thaliana | Q944A7 | Probable serine/threonine-protein kinase At4g35230 (EC |
| | | 2.7.11.1) |
| Arabidopsis thaliana | Q944G5 | Protein NRT1/PTR FAMILY 2.10 (AtNPF2.10) (Protein |
| | | GLUCOSINOLATE TRANSPORTER-1) |
| Arabidopsis thaliana | Q94AZ2 | Sugar transport protein 13 (Hexose transporter 13) |
| | | (Multicopy suppressor of snf4 deficiency protein 1) |
| Arabidopsis thaliana | Q94BT2 | Auxin-induced in root cultures protein 12 |
| Arabidopsis thaliana | Q94CE4 | Beta carbonic anhydrase 4 (AtbCA4) (AtbetaCA4) (EC |
| | | 4.2.1.1) (Beta carbonate dehydratase 4) |
| Arabidopsis thaliana | Q94KI8 | Two pore calcium channel protein 1 (Calcium channel protein |
| | | 1) (AtCCH1) (Fatty acid oxygenation up-regulated protein 2) |
| | | (Voltage-dependent calcium channel protein TPC1) (AtTPC1) |
| Arabidopsis thaliana | Q96262 | Plasma membrane-associated cation-binding protein 1 |
| | | (AtPCAP1) (Microtubule-destabilizing protein 25) |
| Arabidopsis thaliana | Q9C5Y0 | Phospholipase D delta (AtPLDdelta) (PLD delta) (EC 3.1.4.4) |
| Arabidopsis thaliana | Q9C7F7 | Non-specific lipid transfer protein GPI-anchored 1 |
| | | (AtLTPG-1) (Protein LTP-GPI-ANCHORED 1) |
| Arabidopsis thaliana | Q9C821 | Proline-rich receptor-like protein kinase PERK15 (EC |
| | | 2.7.11.1) (Proline-rich extensin-like receptor kinase 15) |
| | | (AtPERK15) |
| Arabidopsis thaliana | Q9C8G5 | CSC1-like protein ERD4 (Protein EARLY-RESPONSIVE TO |
| | | DEHYDRATION STRESS 4) |
| Arabidopsis thaliana | Q9C9C5 | 60S ribosomal protein L6-3 |
| Arabidopsis thaliana | Q9CAR7 | Hypersensitive-induced response protein 2 (AtHIR2) |
| Arabidopsis thaliana | Q9FFH6 | Fasciclin-like arabinogalactan protein 13 |
| Arabidopsis thaliana | Q9FGT8 | Temperature-induced lipocalin-1 (AtTIL1) |
| Arabidopsis thaliana | Q9FJ62 | Glycerophosphodiester phosphodiesterase GDPDL4 (EC |
| | | 3.1.4.46) (Glycerophosphodiester phosphodiesterase-like 4) |
| | | (ATGDPDL4) (Glycerophosphodiesterase-like 1) (Protein |
| | | SHV3-LIKE 1) |
| Arabidopsis thaliana | Q9FK68 | Ras-related protein RABA1c (AtRABA1c) |
| Arabidopsis thaliana | Q9FKS8 | Lysine histidine transporter 1 |
| Arabidopsis thaliana | Q9FM65 | Fasciclin-like arabinogalactan protein 1 |
| Arabidopsis thaliana | Q9FNH6 | NDR1/HIN1-like protein 3 |
| Arabidopsis thaliana | Q9FRL3 | Sugar transporter ERD6-like 6 |
| Arabidopsis thaliana | Q9FWR4 | Glutathione S-transferase DHAR1, mitochondrial (EC |
| | | 2.5.1.18) (Chloride intracellular channel homolog 1) (CLIC |
| | | homolog 1) (Glutathione-dependent dehydroascorbate |
| | | reductase 1) (AtDHAR1) (GSH-dependent dehydroascorbate |
| | | reductase 1) (mtDHAR) |
| Arabidopsis thaliana | Q9FX54 | Glyceraldehyde-3-phosphate dehydrogenase GAPC2, |
| | | cytosolic (EC 1.2.1.12) (NAD-dependent |
| | | glyceraldehydephosphate dehydrogenase C subunit 2) |
| Arabidopsis thaliana | Q9LE22 | Probable calcium-binding protein CML27 (Calmodulin-like |
| | | protein 27) |
| Arabidopsis thaliana | Q9LEX1 | At3g61050 (CaLB protein) (Calcium-dependent lipid-binding |
| | | (CaLB domain) family protein) |
| Arabidopsis thaliana | Q9LF79 | Calcium-transporting ATPase 8, plasma membrane-type (EC |
| | | 3.6.3.8) (Ca(2+)-ATPase isoform 8) |
| Arabidopsis thaliana | Q9LJG3 | GDSL esterase/lipase ESM1 (EC 3.1.1.—) (Extracellular lipase |
| | | ESM1) (Protein EPITHIOSPECIFIER MODIFIER 1) |
| | | (AtESM1) |
| Arabidopsis thaliana | Q9LJI5 | V-type proton ATPase subunit d1 (V-ATPase subunit d1) |
| | | (Vacuolar H(+)-ATPase subunit d isoform 1) (Vacuolar proton |
| | | pump subunit d1) |
| Arabidopsis thaliana | Q9LME4 | Probable protein phosphatase 2C 9 (AtPP2C09) (EC |
| | | 3.1.3.16) (Phytochrome-associated protein phosphatase 2C) |
| | | (PAPP2C) |
| Arabidopsis thaliana | Q9LNP3 | At1g17620/F11A6_23 (F1L3.32) (Late embryogenesis |
| | | abundant (LEA) hydroxyproline-rich glycoprotein family) |
| | | (Putative uncharacterized protein At1g17620) |
| Arabidopsis thaliana | Q9LNW1 | Ras-related protein RABA2b (AtRABA2b) |
| Arabidopsis thaliana | Q9LQU2 | Protein PLANT CADMIUM RESISTANCE 1 (AtPCR1) |
| Arabidopsis thaliana | Q9LQU4 | Protein PLANT CADMIUM RESISTANCE 2 (AtPCR2) |
| Arabidopsis thaliana | Q9LR30 | Glutamate--glyoxylate aminotransferase 1 (AtGGT2) (EC |
| | | 2.6.1.4) (Alanine aminotransferase GGT1) (EC 2.6.1.2) |
| | | (Alanine--glyoxylate aminotransferase GGT1) (EC 2.6.1.44) |
| | | (Alanine-2-oxoglutarate aminotransferase 1) (EC 2.6.1.—) |
| Arabidopsis thaliana | Q9LSI9 | Inactive LRR receptor-like serine/threonine-protein kinase |
| | | BIR2 (Protein BAK1-INTERACTING RECEPTOR-LIKE |
| | | KINASE 2) |
| Arabidopsis thaliana | Q9LSQ5 | NAD(P)H dehydrogenase (quinone) FQR1 (EC 1.6.5.2) |
| | | (Flavodoxin-like quinone reductase 1) |
| Arabidopsis thaliana | Q9LUT0 | Protein kinase superfamily protein (Putative uncharacterized |
| | | protein At3g17410) (Serine/threonine protein kinase-like |
| | | protein) |
| Arabidopsis thaliana | Q9LV48 | Proline-rich receptor-like protein kinase PERK1 (EC 2.7.11.1) |
| | | (Proline-rich extensin-like receptor kinase 1) (AtPERK1) |
| Arabidopsis thaliana | Q9LX65 | V-type proton ATPase subunit H (V-ATPase subunit H) |
| | | (Vacuolar H(+)-ATPase subunit H) (Vacuolar proton pump |
| | | subunit H) |
| Arabidopsis thaliana | Q9LYG3 | NADP-dependent malic enzyme 2 (AtNADP-ME2) |
| | | (NADP-malic enzyme 2) (EC 1.1.1.40) |
| Arabidopsis thaliana | Q9M088 | Glucan endo-1,3-beta-glucosidase 5 (EC 3.2.1.39) |
| | | ((1->3)-beta-glucan endohydrolase 5) |
| | | ((1->3)-beta-glucanase 5) (Beta-1,3-endoglucanase |
| | | 5) (Beta-1,3-glucanase 5) |
| Arabidopsis thaliana | Q9M2D8 | Uncharacterized protein At3g61260 |
| Arabidopsis thaliana | Q9M386 | Late embryogenesis abundant (LEA) hydroxyproline-rich |
| | | glycoprotein family (Putative uncharacterized protein |
| | | At3g54200) (Putative uncharacterized protein F24B22.160) |
| Arabidopsis thaliana | Q9M390 | Protein NRT1/PTR FAMILY 8.1 (AtNPF8.1) (Peptide |
| | | transporter PTR1) |
| Arabidopsis thaliana | Q9M5P2 | Secretory carrier-associated membrane protein 3 (AtSC3) |
| | | (Secretory carrier membrane protein 3) |
| Arabidopsis thaliana | Q9M8T0 | Probable inactive receptor kinase At3g02880 |
| Arabidopsis thaliana | Q9SDS7 | V-type proton ATPase subunit C (V-ATPase subunit C) |
| | | (Vacuolar H(+)-ATPase subunit C) (Vacuolar proton pump |
| | | subunit C) |
| Arabidopsis thaliana | Q9SEL6 | Vesicle transport v-SNARE 11 (AtVTI11) (Protein SHOOT |
| | | GRAVITROPISM 4) (Vesicle soluble NSF attachment protein |
| | | receptor VTI1a) (AtVTI1a) (Vesicle transport v-SNARE |
| | | protein VTI1a) |
| Arabidopsis thaliana | Q9SF29 | Syntaxin-71 (AtSYP71) |
| Arabidopsis thaliana | Q9SF85 | Adenosine kinase 1 (AK 1) (EC 2.7.1.20) (Adenosine |
| | | 5′-phosphotransferase 1) |
| Arabidopsis thaliana | Q9SIE7 | PLAT domain-containing protein 2 (AtPLAT2) (PLAT domain |
| | | protein 2) |
| Arabidopsis thaliana | Q9SIM4 | 60S ribosomal protein L14-1 |
| Arabidopsis thaliana | Q9SIU8 | Probable protein phosphatase 2C 20 (AtPP2C20) (EC |
| | | 3.1.3.16) (AtPPC3; 1.2) |
| Arabidopsis thaliana | Q9SJ81 | Fasciclin-like arabinogalactan protein 7 |
| Arabidopsis thaliana | Q9SKB2 | Leucine-rich repeat receptor-like serine/threonine/tyrosine-protein |
| | | kinase SOBIR1 (EC 2.7.10.1) (EC 2.7.11.1) (Protein |
| | | EVERSHED) (Protein SUPPRESSOR OF BIR1-1) |
| Arabidopsis thaliana | Q9SKR2 | Synaptotagmin-1 (NTMC2T1.1) (Synaptotagmin A) |
| Arabidopsis thaliana | Q9SLF7 | 60S acidic ribosomal protein P2-2 |
| Arabidopsis thaliana | Q9SPE6 | Alpha-soluble NSF attachment protein 2 (Alpha-SNAP2) |
| | | (N-ethylmaleimide-sensitive factor attachment protein alpha 2) |
| Arabidopsis thaliana | Q9SRH6 | Hypersensitive-induced response protein 3 (AtHIR3) |
| Arabidopsis thaliana | Q9SRY5 | Glutathione S-transferase F7 (EC 2.5.1.18) (AtGSTF8) (GST |
| | | class-phi member 7) (Glutathione S-transferase 11) |
| Arabidopsis thaliana | Q9SRZ6 | Cytosolic isocitrate dehydrogenase [NADP] (EC 1.1.1.42) |
| Arabidopsis thaliana | Q9SSK5 | MLP-like protein 43 |
| Arabidopsis thaliana | Q9SU13 | Fasciclin-like arabinogalactan protein 2 |
| Arabidopsis thaliana | Q9SU40 | Monocopper oxidase-like protein SKU5 (Skewed roots) |
| Arabidopsis thaliana | Q9SUR6 | Cystine lyase CORI3 (EC 4.4.1.35) (Protein CORONATINE |
| | | INDUCED 3) (Protein JASMONIC ACID RESPONSIVE 2) |
| | | (Tyrosine aminotransferase CORI3) |
| Arabidopsis thaliana | Q9SVC2 | Syntaxin-122 (AtSYP122) (Synt4) |
| Arabidopsis thaliana | Q9SVF0 | Putative uncharacterized protein AT4g38350 (Putative |
| | | uncharacterized protein F22I13.120) |
| Arabidopsis thaliana | Q9SW40 | Major facilitator superfamily protein (Putative uncharacterized |
| | | protein AT4g34950) (Putative uncharacterized protein |
| | | T11I11.190) |
| Arabidopsis thaliana | Q9SYT0 | Annexin D1 (AnnAt1) (Annexin A1) |
| Arabidopsis thaliana | Q9SZ11 | Glycerophosphodiester phosphodiesterase GDPDL3 (EC |
| | | 3.1.4.46) (Glycerophosphodiester phosphodiesterase-like 3) |
| | | (ATGDPDL3) (Glycerophosphodiesterase-like 2) (Protein |
| | | MUTANT ROOT HAIR 5) (Protein SHAVEN 3) |
| Arabidopsis thaliana | Q9SZN1 | V-type proton ATPase subunit B2 (V-ATPase subunit B2) |
| | | (Vacuolar H(+)-ATPase subunit B isoform 2) (Vacuolar |
| | | proton pump subunit B2) |
| Arabidopsis thaliana | Q9SZP6 | AT4g38690/F20M13_250 (PLC-like phosphodiesterases |
| | | superfamily protein) (Putative uncharacterized protein |
| | | AT4g38690) (Putative uncharacterized protein F20M13.250) |
| Arabidopsis thaliana | Q9SZR1 | Calcium-transporting ATPase 10, plasma membrane-type |
| | | (EC 3.6.3.8) (Ca(2+)-ATPase isoform 10) |
| Arabidopsis thaliana | Q9T053 | Phospholipase D gamma 1 (AtPLDgamma1) (PLD gamma 1) |
| | | (EC 3.1.4.4) (Choline phosphatase) (Lecithinase D) |
| | | (Lipophosphodiesterase II) |
| Arabidopsis thaliana | Q9T076 | Early nodulin-like protein 2 (Phytocyanin-like protein) |
| Arabidopsis thaliana | Q9T0A0 | Long chain acyl-CoA synthetase 4 (EC 6.2.1.3) |
| Arabidopsis thaliana | Q9T0G4 | Putative uncharacterized protein AT4g10060 (Putative |
| | | uncharacterized protein T5L19.190) |
| Arabidopsis thaliana | Q9XEE2 | Annexin D2 (AnnAt2) |
| Arabidopsis thaliana | Q9XGM1 | V-type proton ATPase subunit D (V-ATPase subunit D) |
| | | (Vacuolar H(+)-ATPase subunit D) (Vacuolar proton pump |
| | | subunit D) |
| Arabidopsis thaliana | Q9XI93 | At1g13930/F16A14.27 (F16A14.14) (F7A19.2 protein) |
| | | (Oleosin-B3-like protein) |
| Arabidopsis thaliana | Q9XIE2 | ABC transporter G family member 36 (ABC transporter |
| | | ABCG.36) (AtABCG36) (Pleiotropic drug resistance protein |
| | | 8) (Protein PENETRATION 3) |
| Arabidopsis thaliana | Q9ZPZ4 | Putative uncharacterized protein (Putative uncharacterized |
| | | protein At1g09310) (T31J12.3 protein) |
| Arabidopsis thaliana | Q9ZQX4 | V-type proton ATPase subunit F (V-ATPase subunit F) |
| | | (V-ATPase 14 kDa subunit) (Vacuolar H(+)-ATPase subunit F) |
| | | (Vacuolar proton pump subunit F) |
| Arabidopsis thaliana | Q9ZSA2 | Calcium-dependent protein kinase 21 (EC 2.7.11.1) |
| Arabidopsis thaliana | Q9ZSD4 | Syntaxin-121 (AtSYP121) (Syntaxin-related protein At-Syr1) |
| Arabidopsis thaliana | Q9ZV07 | Probable aquaporin PIP2-6 (Plasma membrane intrinsic |
| | | protein 2-6) (AtPIP2; 6) (Plasma membrane intrinsic protein |
| | | 2e) (PIP2e) [Cleaved into: Probable aquaporin PIP2-6, |
| | | N-terminally processed] |
| Arabidopsis thaliana | Q9ZVF3 | MLP-like protein 328 |
| Arabidopsis thaliana | Q9ZWA8 | Fasciclin-like arabinogalactan protein 9 |
| Arabidopsis thaliana | Q9ZSD4 | SYR1, Syntaxin Related Protein 1, also known as SYP121, |
| | | PENETRATION1/PEN1 (Protein PENETRATION 1) |
| Citrus lemon | A1ECK0 | Putative glutaredoxin |
| Citrus lemon | A9YVC9 | Pyrophosphate--fructose 6-phosphate 1-phosphotransferase |
| | | subunit beta (PFP) (EC 2.7.1.90) (6-phosphofructokinase, |
| | | pyrophosphate dependent) (PPi-PFK) |
| | | (Pyrophosphate-dependent 6-phosphofructose-1-kinase) |
| Citrus lemon | B2YGY1 | Glycosyltransferase (EC 2.4.1.—) |
| Citrus lemon | B6DZD3 | Glutathione S-transferase Tau2 (Glutathione transferase |
| | | Tau2) |
| Citrus lemon | C3VIC2 | Translation elongation factor |
| Citrus lemon | C8CPS0 | Importin subunit alpha |
| Citrus lemon | D3JWB5 | Flavanone 3-hydroxylase |
| Citrus lemon | E0ADY2 | Putative caffeic acid O-methyltransferase |
| Citrus lemon | E5DK62 | ATP synthase subunit alpha (Fragment) |
| Citrus lemon | E9M5S3 | Putative L-galactose-1-phosphate phosphatase |
| Citrus lemon | F1CGQ9 | Heat shock protein 90 |
| Citrus lemon | F8WL79 | Aminopeptidase (EC 3.4.11.—) |
| Citrus lemon | F8WL86 | Heat shock protein |
| Citrus lemon | K9JG59 | Abscisic acid stress ripening-related protein |
| Citrus lemon | Q000W4 | Fe(III)-chelate reductase |
| Citrus lemon | Q39538 | Heat shock protein (Fragment) |
| Citrus lemon | Q5UEN6 | Putative signal recognition particle protein |
| Citrus lemon | Q8GV08 | Dehydrin |
| Citrus lemon | Q8L893 | Cytosolic phosphoglucomutase (Fragment) |
| Citrus lemon | Q8S990 | Polygalacturonase-inhibiting protein |
| Citrus lemon | Q8W3U6 | Polygalacturonase-inhibitor protein |
| Citrus lemon | Q93XL8 | Dehydrin COR15 |
| Citrus lemon | Q941Q1 | Non-symbiotic hemoglobin class 1 |
| Citrus lemon | Q9MBF3 | Glycine-rich RNA-binding protein |
| Citrus lemon | Q9SP55 | V-type proton ATPase subunit G (V-ATPase subunit G) |
| | | (Vacuolar proton pump subunit G) |
| Citrus lemon | Q9THJ8 | Ribulose bisphosphate carboxylase large chain (EC 4.1.1.39) |
| | | (Fragment) |
| Citrus lemon | Q9ZST2 | Pyrophosphate--fructose 6-phosphate 1-phosphotransferase |
| | | subunit alpha (PFP) (6-phosphofructokinase, pyrophosphate |
| | | dependent) (PPi-PFK) (Pyrophosphate-dependent |
| | | 6-phosphofructose-1-kinase) |
| Citrus lemon | Q9ZWH6 | Polygalacturonase inhibitor |
| Citrus lemon | S5DXI9 | Nucleocapsid protein |
| Citrus lemon | S5NFC6 | GTP cyclohydrolase |
| Citrus lemon | V4RG42 | Uncharacterized protein |
| Citrus lemon | V4RGP4 | Uncharacterized protein |
| Citrus lemon | V4RHN8 | Uncharacterized protein |
| Citrus lemon | V4RJ07 | Uncharacterized protein |
| Citrus lemon | V4RJK9 | Adenosylhomocysteinase (EC 3.3.1.1) |
| Citrus lemon | V4RJM1 | Uncharacterized protein |
| Citrus lemon | V4RJX1 | 40S ribosomal protein S6 |
| Citrus lemon | V4RLB2 | Uncharacterized protein |
| Citrus lemon | V4RMX8 | Uncharacterized protein |
| Citrus lemon | V4RNA5 | Uncharacterized protein |
| Citrus lemon | V4RP81 | Glycosyltransferase (EC 2.4.1.—) |
| Citrus lemon | V4RPZ5 | Adenylyl cyclase-associated protein |
| Citrus lemon | V4RTN9 | Histone H4 |
| Citrus lemon | V4RUZ4 | Phosphoserine aminotransferase (EC 2.6.1.52) |
| Citrus lemon | V4RVF6 | Uncharacterized protein |
| Citrus lemon | V4RXD4 | Uncharacterized protein |
| Citrus lemon | V4RXG2 | Uncharacterized protein |
| Citrus lemon | V4RYA0 | Uncharacterized protein |
| Citrus lemon | V4RYE3 | Uncharacterized protein |
| Citrus lemon | V4RYH3 | Uncharacterized protein |
| Citrus lemon | V4RYX8 | Uncharacterized protein |
| Citrus lemon | V4RZ12 | Coatomer subunit beta′ |
| Citrus lemon | V4RZ89 | Uncharacterized protein |
| Citrus lemon | V4RZE3 | Uncharacterized protein |
| Citrus lemon | V4RZF3 | 1,2-dihydroxy-3-keto-5-methylthiopentene dioxygenase (EC |
| | | 1.13.11.54) (Acireductone dioxygenase (Fe(2+)-requiring)) |
| | | (ARD) (Fe-ARD) |
| Citrus lemon | V4RZM7 | Uncharacterized protein |
| Citrus lemon | V4RZX6 | Uncharacterized protein |
| Citrus lemon | V4S1V0 | Uncharacterized protein |
| Citrus lemon | V4S2B6 | Uncharacterized protein |
| Citrus lemon | V4S2N1 | Uncharacterized protein |
| Citrus lemon | V4S2S5 | Uncharacterized protein (Fragment) |
| Citrus lemon | V4S346 | Uncharacterized protein |
| Citrus lemon | V4S3T8 | Uncharacterized protein |
| Citrus lemon | V4S409 | Cyanate hydratase (Cyanase) (EC 4.2.1.104) (Cyanate |
| | | hydrolase) (Cyanate lyase) |
| Citrus lemon | V4S4E4 | Histone H2B |
| Citrus lemon | V4S4F6 | Flavin-containing monooxygenase (EC 1.—.—.—) |
| Citrus lemon | V4S4J1 | Uncharacterized protein |
| Citrus lemon | V4S4K9 | Uncharacterized protein |
| Citrus lemon | V4S535 | Proteasome subunit alpha type (EC 3.4.25.1) |
| Citrus lemon | V4S5A8 | Isocitrate dehydrogenase [NADP] (EC 1.1.1.42) |
| Citrus lemon | V4S5G8 | Uncharacterized protein |
| Citrus lemon | V4S5I6 | Uncharacterized protein |
| Citrus lemon | V4S5N4 | Uncharacterized protein (Fragment) |
| Citrus lemon | V4S5Q3 | Uncharacterized protein |
| Citrus lemon | V4S5X8 | Uncharacterized protein |
| Citrus lemon | V4S5Y1 | Uncharacterized protein |
| Citrus lemon | V4S6P4 | Calcium-transporting ATPase (EC 3.6.3.8) |
| Citrus lemon | V4S6W0 | Uncharacterized protein |
| Citrus lemon | V4S6W7 | Uncharacterized protein (Fragment) |
| Citrus lemon | V4S6Y4 | Uncharacterized protein |
| Citrus lemon | V4S773 | Ribosomal protein L19 |
| Citrus lemon | V4S7U0 | Uncharacterized protein |
| Citrus lemon | V4S7U5 | Uncharacterized protein |
| Citrus lemon | V4S7W4 | Pyruvate kinase (EC 2.7.1.40) |
| Citrus lemon | V4S885 | Uncharacterized protein |
| Citrus lemon | V4S8T3 | Peptidyl-prolyl cis-trans isomerase (PPIase) (EC 5.2.1.8) |
| Citrus lemon | V4S920 | Uncharacterized protein |
| Citrus lemon | V4S999 | Uncharacterized protein |
| Citrus lemon | V4S9G5 | Phosphoglycerate kinase (EC 2.7.2.3) |
| Citrus lemon | V4S9Q6 | Beta-amylase (EC 3.2.1.2) |
| Citrus lemon | V4SA44 | Serine/threonine-protein phosphatase (EC 3.1.3.16) |
| Citrus lemon | V4SAE0 | Alpha-1,4 glucan phosphorylase (EC 2.4.1.1) |
| Citrus lemon | V4SAF6 | Uncharacterized protein |
| Citrus lemon | V4SAI9 | Eukaryotic translation initiation factor 3 subunit M (eIF3m) |
| Citrus lemon | V4SAJ5 | Ribosomal protein |
| Citrus lemon | V4SAR3 | Uncharacterized protein |
| Citrus lemon | V4SB37 | Uncharacterized protein |
| Citrus lemon | V4SBI0 | Elongation factor 1-alpha |
| Citrus lemon | V4SBI8 | D-3-phosphoglycerate dehydrogenase (EC 1.1.1.95) |
| Citrus lemon | V4SBL9 | Polyadenylate-binding protein (PABP) |
| Citrus lemon | V4SBR1 | S-formylglutathione hydrolase (EC 3.1.2.12) |
| Citrus lemon | V4SBR6 | Uncharacterized protein |
| Citrus lemon | V4SCG7 | Uncharacterized protein |
| Citrus lemon | V4SCJ2 | Uncharacterized protein |
| Citrus lemon | V4SCQ6 | Peptidyl-prolyl cis-trans isomerase (PPIase) (EC 5.2.1.8) |
| Citrus lemon | V4SDJ8 | Uncharacterized protein |
| Citrus lemon | V4SE41 | Protein DETOXIFICATION (Multidrug and toxic compound |
| | | extrusion protein) |
| Citrus lemon | V4SE90 | Uncharacterized protein |
| Citrus lemon | V4SED1 | Succinate dehydrogenase [ubiquinone] flavoprotein subunit, |
| | | mitochondrial (EC 1.3.5.1) |
| Citrus lemon | V4SEI1 | Uncharacterized protein |
| Citrus lemon | V4SEN9 | Uncharacterized protein |
| Citrus lemon | V4SEX8 | Uncharacterized protein |
| Citrus lemon | V4SF31 | Uncharacterized protein |
| Citrus lemon | V4SF69 | 40S ribosomal protein S24 |
| Citrus lemon | V4SF76 | Cysteine synthase (EC 2.5.1.47) |
| Citrus lemon | V4SFK3 | Uncharacterized protein |
| Citrus lemon | V4SFL4 | Uncharacterized protein |
| Citrus lemon | V4SFW2 | Uncharacterized protein |
| Citrus lemon | V4SGC9 | Uncharacterized protein |
| Citrus lemon | V4SGJ4 | Uncharacterized protein |
| Citrus lemon | V4SGN4 | Uncharacterized protein |
| Citrus lemon | V4SGV6 | Uncharacterized protein |
| Citrus lemon | V4SGV7 | Uncharacterized protein |
| Citrus lemon | V4SHH1 | Plasma membrane ATPase (EC 3.6.3.6) (Fragment) |
| Citrus lemon | V4SHI2 | Uncharacterized protein |
| Citrus lemon | V4SHJ3 | Uncharacterized protein |
| Citrus lemon | V4SI86 | Uncharacterized protein |
| Citrus lemon | V4SI88 | Uncharacterized protein |
| Citrus lemon | V4SIA2 | Uncharacterized protein |
| Citrus lemon | V4SIC1 | Phospholipase D (EC 3.1.4.4) |
| Citrus lemon | V4SJ14 | Uncharacterized protein |
| Citrus lemon | V4SJ48 | Uncharacterized protein |
| Citrus lemon | V4SJ69 | Uncharacterized protein |
| Citrus lemon | V4SJD9 | Uncharacterized protein |
| Citrus lemon | V4SJS7 | Uncharacterized protein |
| Citrus lemon | V4SJT5 | Uncharacterized protein |
| Citrus lemon | V4SKA2 | Uncharacterized protein |
| Citrus lemon | V4SKG4 | Glucose-6-phosphate isomerase (EC 5.3.1.9) |
| Citrus lemon | V4SKJ1 | Uncharacterized protein |
| Citrus lemon | V4SL90 | Uncharacterized protein |
| Citrus lemon | V4SLC6 | Proteasome subunit beta type (EC 3.4.25.1) |
| Citrus lemon | V4SLI7 | Uncharacterized protein |
| Citrus lemon | V4SLQ6 | Uncharacterized protein |
| Citrus lemon | V4SMD8 | Uncharacterized protein |
| Citrus lemon | V4SMN7 | Uncharacterized protein |
| Citrus lemon | V4SMV5 | Uncharacterized protein |
| Citrus lemon | V4SN00 | Uncharacterized protein |
| Citrus lemon | V4SNA9 | Uncharacterized protein |
| Citrus lemon | V4SNC1 | Uncharacterized protein |
| Citrus lemon | V4SNC4 | Aconitate hydratase (Aconitase) (EC 4.2.1.3) |
| Citrus lemon | V4SNZ3 | Uncharacterized protein |
| Citrus lemon | V4SP86 | Uncharacterized protein |
| Citrus lemon | V4SPM1 | 40S ribosomal protein S12 |
| Citrus lemon | V4SPW4 | 40S ribosomal protein S4 |
| Citrus lemon | V4SQ71 | Uncharacterized protein |
| Citrus lemon | V4SQ89 | Uncharacterized protein |
| Citrus lemon | V4SQ92 | Uncharacterized protein |
| Citrus lemon | V4SQC7 | Peroxidase (EC 1.11.1.7) |
| Citrus lemon | V4SQG3 | Uncharacterized protein |
| Citrus lemon | V4SR15 | Uncharacterized protein |
| Citrus lemon | V4SRN3 | Transmembrane 9 superfamily member |
| Citrus lemon | V4SS09 | Uncharacterized protein |
| Citrus lemon | V4SS11 | Uncharacterized protein |
| Citrus lemon | V4SS50 | Uncharacterized protein |
| Citrus lemon | V4SSB6 | Uncharacterized protein |
| Citrus lemon | V4SSB8 | Proteasome subunit alpha type (EC 3.4.25.1) |
| Citrus lemon | V4SSL7 | Uncharacterized protein |
| Citrus lemon | V4SSQ1 | Uncharacterized protein |
| Citrus lemon | V4SST6 | Uncharacterized protein |
| Citrus lemon | V4SSW9 | Uncharacterized protein |
| Citrus lemon | V4SSX5 | Uncharacterized protein |
| Citrus lemon | V4SU82 | Uncharacterized protein |
| Citrus lemon | V4SUD3 | Uncharacterized protein |
| Citrus lemon | V4SUL7 | Uncharacterized protein |
| Citrus lemon | V4SUP3 | Uncharacterized protein |
| Citrus lemon | V4SUT4 | UDP-glucose 6-dehydrogenase (EC 1.1.1.22) |
| Citrus lemon | V4SUY5 | Uncharacterized protein |
| Citrus lemon | V4SV60 | Serine/threonine-protein phosphatase (EC 3.1.3.16) |
| Citrus lemon | V4SV61 | Uncharacterized protein |
| Citrus lemon | V4SVI5 | Proteasome subunit alpha type (EC 3.4.25.1) |
| Citrus lemon | V4SVI6 | Uncharacterized protein |
| Citrus lemon | V4SW04 | Uncharacterized protein (Fragment) |
| Citrus lemon | V4SWD9 | Uncharacterized protein |
| Citrus lemon | V4SWJ0 | 40S ribosomal protein S3a |
| Citrus lemon | V4SWQ9 | Uncharacterized protein |
| Citrus lemon | V4SWR9 | Uncharacterized protein |
| Citrus lemon | V4SWU9 | Fructose-bisphosphate aldolase (EC 4.1.2.13) |
| Citrus lemon | V4SX11 | Uncharacterized protein |
| Citrus lemon | V4SX99 | Uncharacterized protein |
| Citrus lemon | V4SXC7 | Proteasome subunit alpha type (EC 3.4.25.1) |
| Citrus lemon | V4SXQ5 | Uncharacterized protein |
| Citrus lemon | V4SXW1 | Beta-adaptin-like protein |
| Citrus lemon | V4SXY9 | Uncharacterized protein |
| Citrus lemon | V4SY74 | Uncharacterized protein |
| Citrus lemon | V4SY90 | Uncharacterized protein |
| Citrus lemon | V4SY93 | Uncharacterized protein |
| Citrus lemon | V4SYH9 | Uncharacterized protein |
| Citrus lemon | V4SYK6 | Uncharacterized protein |
| Citrus lemon | V4SZ03 | Uncharacterized protein |
| Citrus lemon | V4SZ73 | Uncharacterized protein |
| Citrus lemon | V4SZI9 | Uncharacterized protein |
| Citrus lemon | V4SZX7 | Uncharacterized protein |
| Citrus lemon | V4T057 | Ribosomal protein L15 |
| Citrus lemon | V4T0V5 | Eukaryotic translation initiation factor 3 subunit A (eIF3a) |
| | | (Eukaryotic translation initiation factor 3 subunit 10) |
| Citrus lemon | V4T0Y1 | Uncharacterized protein |
| Citrus lemon | V4T1Q6 | Uncharacterized protein |
| Citrus lemon | V4T1U7 | Uncharacterized protein |
| Citrus lemon | V4T2D9 | Uncharacterized protein |
| Citrus lemon | V4T2M6 | Tubulin beta chain |
| Citrus lemon | V4T3G2 | Uncharacterized protein |
| Citrus lemon | V4T3P3 | 6-phosphogluconate dehydrogenase, decarboxylating (EC |
| | | 1.1.1.44) |
| Citrus lemon | V4T3V9 | Uncharacterized protein |
| Citrus lemon | V4T3Y6 | Uncharacterized protein |
| Citrus lemon | V4T4H3 | Uncharacterized protein |
| Citrus lemon | V4T4I7 | Uncharacterized protein |
| Citrus lemon | V4T4M7 | Superoxide dismutase [Cu—Zn] (EC 1.15.1.1) |
| Citrus lemon | V4T539 | Uncharacterized protein |
| Citrus lemon | V4T541 | Uncharacterized protein |
| Citrus lemon | V4T576 | Uncharacterized protein |
| Citrus lemon | V4T5E1 | Uncharacterized protein |
| Citrus lemon | V4T5I3 | Uncharacterized protein |
| Citrus lemon | V4T5W7 | Uncharacterized protein |
| Citrus lemon | V4T6T5 | 60S acidic ribosomal protein P0 |
| Citrus lemon | V4T722 | Uncharacterized protein |
| Citrus lemon | V4T785 | Uncharacterized protein |
| Citrus lemon | V4T7E2 | Uncharacterized protein |
| Citrus lemon | V4T7I7 | Uncharacterized protein |
| Citrus lemon | V4T7N0 | Proteasome subunit beta type (EC 3.4.25.1) |
| Citrus lemon | V4T7N4 | Uncharacterized protein |
| Citrus lemon | V4T7T2 | Uncharacterized protein |
| Citrus lemon | V4T7W5 | Uncharacterized protein |
| Citrus lemon | V4T825 | Uncharacterized protein |
| Citrus lemon | V4T846 | Uncharacterized protein |
| Citrus lemon | V4T8E9 | S-acyltransferase (EC 2.3.1.225) (Palmitoyltransferase) |
| Citrus lemon | V4T8G2 | Uncharacterized protein |
| Citrus lemon | V4T8G9 | Chorismate synthase (EC 4.2.3.5) |
| Citrus lemon | V4T8Y6 | Uncharacterized protein |
| Citrus lemon | V4T8Y8 | Uncharacterized protein |
| Citrus lemon | V4T939 | Carboxypeptidase (EC 3.4.16.—) |
| Citrus lemon | V4T957 | Uncharacterized protein |
| Citrus lemon | V4T998 | Uncharacterized protein |
| Citrus lemon | V4T9B9 | Uncharacterized protein |
| Citrus lemon | V4T9Y7 | Uncharacterized protein |
| Citrus lemon | V4TA70 | Uncharacterized protein |
| Citrus lemon | V4TAF6 | Uncharacterized protein |
| Citrus lemon | V4TB09 | Uncharacterized protein |
| Citrus lemon | V4TB32 | Uncharacterized protein |
| Citrus lemon | V4TB89 | Uncharacterized protein |
| Citrus lemon | V4TBN7 | Phosphoinositide phospholipase C (EC 3.1.4.11) |
| Citrus lemon | V4TBQ3 | Uncharacterized protein |
| Citrus lemon | V4TBS4 | Uncharacterized protein |
| Citrus lemon | V4TBU3 | Uncharacterized protein |
| Citrus lemon | V4TCA6 | Uncharacterized protein |
| Citrus lemon | V4TCL3 | Uncharacterized protein |
| Citrus lemon | V4TCS5 | Pectate lyase (EC 4.2.2.2) |
| Citrus lemon | V4TD99 | Uncharacterized protein |
| Citrus lemon | V4TDB5 | Uncharacterized protein |
| Citrus lemon | V4TDI2 | Uncharacterized protein |
| Citrus lemon | V4TDY3 | Serine/threonine-protein kinase (EC 2.7.11.1) |
| Citrus lemon | V4TE72 | Uncharacterized protein |
| Citrus lemon | V4TE95 | Uncharacterized protein |
| Citrus lemon | V4TEC0 | Uncharacterized protein |
| Citrus lemon | V4TED8 | Uncharacterized protein |
| Citrus lemon | V4TES4 | Uncharacterized protein |
| Citrus lemon | V4TEY9 | Uncharacterized protein |
| Citrus lemon | V4TF24 | Proteasome subunit alpha type (EC 3.4.25.1) |
| Citrus lemon | V4TF52 | Uricase (EC 1.7.3.3) (Urate oxidase) |
| Citrus lemon | V4TFV8 | Catalase (EC 1.11.1.6) |
| Citrus lemon | V4TGU1 | Uncharacterized protein |
| Citrus lemon | V4TH28 | Uncharacterized protein |
| Citrus lemon | V4TH78 | Reticulon-like protein |
| Citrus lemon | V4THM9 | Uncharacterized protein |
| Citrus lemon | V4TIU2 | Ribulose-phosphate 3-epimerase (EC 5.1.3.1) |
| Citrus lemon | V4TIW6 | Uncharacterized protein |
| Citrus lemon | V4TIY6 | Uncharacterized protein |
| Citrus lemon | V4TIZ5 | Uncharacterized protein |
| Citrus lemon | V4TJ75 | Uncharacterized protein |
| Citrus lemon | V4TJC3 | Uncharacterized protein |
| Citrus lemon | V4TJQ9 | Uncharacterized protein |
| Citrus lemon | V4TK29 | NEDD8-activating enzyme E1 regulatory subunit |
| Citrus lemon | V4TL04 | Uncharacterized protein |
| Citrus lemon | V4TLL5 | Uncharacterized protein |
| Citrus lemon | V4TLP6 | Uncharacterized protein |
| Citrus lemon | V4TM00 | Uncharacterized protein |
| Citrus lemon | V4TM19 | Uncharacterized protein |
| Citrus lemon | V4TMB7 | Uncharacterized protein (Fragment) |
| Citrus lemon | V4TMD1 | Uncharacterized protein |
| Citrus lemon | V4TMD6 | Uncharacterized protein |
| Citrus lemon | V4TMV4 | Uncharacterized protein |
| Citrus lemon | V4TN30 | Uncharacterized protein |
| Citrus lemon | V4TN38 | Uncharacterized protein |
| Citrus lemon | V4TNY8 | Uncharacterized protein |
| Citrus lemon | V4TP87 | Carbonic anhydrase (EC 4.2.1.1) (Carbonate dehydratase) |
| Citrus lemon | V4TPM1 | Homoserine dehydrogenase (HDH) (EC 1.1.1.3) |
| Citrus lemon | V4TQB6 | Uncharacterized protein |
| Citrus lemon | V4TQM7 | Uncharacterized protein |
| Citrus lemon | V4TQR2 | Uncharacterized protein |
| Citrus lemon | V4TQV9 | Uncharacterized protein |
| Citrus lemon | V4TS21 | Proteasome subunit beta type (EC 3.4.25.1) |
| Citrus lemon | V4TS28 | Annexin |
| Citrus lemon | V4TSD8 | Uncharacterized protein (Fragment) |
| Citrus lemon | V4TSF8 | Uncharacterized protein |
| Citrus lemon | V4TSI9 | Uncharacterized protein |
| Citrus lemon | V4TT89 | Uncharacterized protein |
| Citrus lemon | V4TTA0 | Uncharacterized protein |
| Citrus lemon | V4TTR8 | Uncharacterized protein |
| Citrus lemon | V4TTV4 | Uncharacterized protein |
| Citrus lemon | V4TTZ7 | Uncharacterized protein |
| Citrus lemon | V4TU54 | Uncharacterized protein |
| Citrus lemon | V4TVB6 | Uncharacterized protein |
| Citrus lemon | V4TVG1 | Eukaryotic translation initiation factor 5A (eIF-5A) |
| Citrus lemon | V4TVJ4 | Profilin |
| Citrus lemon | V4TVM6 | Uncharacterized protein |
| Citrus lemon | V4TVM9 | Uncharacterized protein |
| Citrus lemon | V4TVP7 | Uncharacterized protein |
| Citrus lemon | V4TVT8 | Uncharacterized protein |
| Citrus lemon | V4TW14 | Uncharacterized protein |
| Citrus lemon | V4TWG9 | T-complex protein 1 subunit delta |
| Citrus lemon | V4TWU1 | Probable bifunctional methylthioribulose-1-phosphate |
| | | dehydratase/enolase-phosphatase E1 [Includes: Enolase-phosphatase |
| | | E1 (EC 3.1.3.77) (2,3-diketo-5-methylthio-1-phosphopentane |
| | | phosphatase); Methylthioribulose-1-phosphate dehydratase |
| | | (MTRu-1-P dehydratase) (EC 4.2.1.109)] |
| Citrus lemon | V4TWX8 | Uncharacterized protein |
| Citrus lemon | V4TXH0 | Glutamate decarboxylase (EC 4.1.1.15) |
| Citrus lemon | V4TXK9 | Uncharacterized protein |
| Citrus lemon | V4TXU9 | Thiamine thiazole synthase, chloroplastic (Thiazole |
| | | biosynthetic enzyme) |
| Citrus lemon | V4TY40 | Uncharacterized protein |
| Citrus lemon | V4TYJ6 | Uncharacterized protein |
| Citrus lemon | V4TYP5 | 60S ribosomal protein L13 |
| Citrus lemon | V4TYP6 | Uncharacterized protein |
| Citrus lemon | V4TYR6 | Uncharacterized protein |
| Citrus lemon | V4TYZ8 | Tubulin alpha chain |
| Citrus lemon | V4TZ91 | Guanosine nucleotide diphosphate dissociation inhibitor |
| Citrus lemon | V4TZA8 | Uncharacterized protein |
| Citrus lemon | V4TZJ1 | Uncharacterized protein |
| Citrus lemon | V4TZK5 | Uncharacterized protein |
| Citrus lemon | V4TZP2 | Uncharacterized protein |
| Citrus lemon | V4TZT8 | Uncharacterized protein |
| Citrus lemon | V4TZU3 | Mitogen-activated protein kinase (EC 2.7.11.24) |
| Citrus lemon | V4TZU5 | Dihydrolipoyl dehydrogenase (EC 1.8.1.4) |
| Citrus lemon | V4TZZ0 | Uncharacterized protein |
| Citrus lemon | V4U003 | Eukaryotic translation initiation factor 3 subunit K (eIF3k) |
| | | (eIF-3 p25) |
| Citrus lemon | V4U068 | Uncharacterized protein |
| Citrus lemon | V4U088 | Uncharacterized protein |
| Citrus lemon | V4U0J7 | Uncharacterized protein |
| Citrus lemon | V4U133 | Uncharacterized protein |
| Citrus lemon | V4U1A8 | Uncharacterized protein |
| Citrus lemon | V4U1K1 | Xylose isomerase (EC 5.3.1.5) |
| Citrus lemon | V4U1M1 | Uncharacterized protein |
| Citrus lemon | V4U1V0 | Uncharacterized protein |
| Citrus lemon | V4U1X7 | Uncharacterized protein |
| Citrus lemon | V4U1X9 | Proteasome subunit beta type (EC 3.4.25.1) |
| Citrus lemon | V4U251 | Uncharacterized protein |
| Citrus lemon | V4U283 | Uncharacterized protein |
| Citrus lemon | V4U2E4 | Uncharacterized protein |
| Citrus lemon | V4U2F7 | Uncharacterized protein |
| Citrus lemon | V4U2H8 | Uncharacterized protein |
| Citrus lemon | V4U2L0 | Malate dehydrogenase (EC 1.1.1.37) |
| Citrus lemon | V4U2L2 | Uncharacterized protein |
| Citrus lemon | V4U2W4 | V-type proton ATPase subunit C |
| Citrus lemon | V4U3L2 | Uncharacterized protein |
| Citrus lemon | V4U3W8 | Uncharacterized protein |
| Citrus lemon | V4U412 | Uncharacterized protein |
| Citrus lemon | V4U4K2 | Uncharacterized protein |
| Citrus lemon | V4U4M4 | Uncharacterized protein |
| Citrus lemon | V4U4N5 | Eukaryotic translation initiation factor 6 (eIF-6) |
| Citrus lemon | V4U4S9 | Uncharacterized protein |
| Citrus lemon | V4U4X3 | Serine hydroxymethyltransferase (EC 2.1.2.1) |
| Citrus lemon | V4U4Z9 | Uncharacterized protein |
| Citrus lemon | V4U500 | Uncharacterized protein |
| Citrus lemon | V4U5B0 | Eukaryotic translation initiation factor 3 subunit E (eIF3e) |
| | | (Eukaryotic translation initiation factor 3 subunit 6) |
| Citrus lemon | V4U5B8 | Glutathione peroxidase |
| Citrus lemon | V4U5R5 | Citrate synthase |
| Citrus lemon | V4U5Y8 | Uncharacterized protein |
| Citrus lemon | V4U6I5 | ATP synthase subunit beta (EC 3.6.3.14) |
| Citrus lemon | V4U6Q8 | Uncharacterized protein |
| Citrus lemon | V4U706 | Uncharacterized protein |
| Citrus lemon | V4U717 | Uncharacterized protein |
| Citrus lemon | V4U726 | Uncharacterized protein |
| Citrus lemon | V4U729 | Uncharacterized protein |
| Citrus lemon | V4U734 | Serine/threonine-protein phosphatase (EC 3.1.3.16) |
| Citrus lemon | V4U7G7 | Uncharacterized protein |
| Citrus lemon | V4U7H5 | Uncharacterized protein |
| Citrus lemon | V4U7R1 | Potassium transporter |
| Citrus lemon | V4U7R7 | Mitogen-activated protein kinase (EC 2.7.11.24) |
| Citrus lemon | V4U833 | Malic enzyme |
| Citrus lemon | V4U840 | Uncharacterized protein |
| Citrus lemon | V4U8C3 | Uncharacterized protein |
| Citrus lemon | V4U8J1 | 3-phosphoshikimate 1-carboxyvinyltransferase (EC 2.5.1.19) |
| Citrus lemon | V4U8J8 | T-complex protein 1 subunit gamma |
| Citrus lemon | V4U995 | Uncharacterized protein |
| Citrus lemon | V4U999 | Uncharacterized protein |
| Citrus lemon | V4U9C7 | Eukaryotic translation initiation factor 3 subunit D (eIF3d) |
| | | (Eukaryotic translation initiation factor 3 subunit 7) (eIF-3-zeta) |
| Citrus lemon | V4U9G8 | Proline iminopeptidase (EC 3.4.11.5) |
| Citrus lemon | V4U9L1 | Uncharacterized protein |
| Citrus lemon | V4UA63 | Phytochrome |
| Citrus lemon | V4UAC8 | Uncharacterized protein |
| Citrus lemon | V4UAR4 | Uncharacterized protein |
| Citrus lemon | V4UB30 | Uncharacterized protein |
| Citrus lemon | V4UBK8 | V-type proton ATPase subunit a |
| Citrus lemon | V4UBL3 | Coatomer subunit alpha |
| Citrus lemon | V4UBL5 | Uncharacterized protein (Fragment) |
| Citrus lemon | V4UBM0 | Uncharacterized protein |
| Citrus lemon | V4UBZ8 | Aspartate aminotransferase (EC 2.6.1.1) |
| Citrus lemon | V4UC72 | Uncharacterized protein |
| Citrus lemon | V4UC97 | Beta-glucosidase (EC 3.2.1.21) |
| Citrus lemon | V4UCE2 | Uncharacterized protein |
| Citrus lemon | V4UCT9 | Acetyl-coenzyme A synthetase (EC 6.2.1.1) |
| Citrus lemon | V4UCZ1 | Uncharacterized protein |
| Citrus lemon | V4UE34 | Uncharacterized protein |
| Citrus lemon | V4UE78 | Uncharacterized protein |
| Citrus lemon | V4UER3 | Uncharacterized protein |
| Citrus lemon | V4UET6 | Uncharacterized protein |
| Citrus lemon | V4UEZ6 | Uncharacterized protein |
| Citrus lemon | V4UFD0 | Uncharacterized protein |
| Citrus lemon | V4UFG8 | Uncharacterized protein |
| Citrus lemon | V4UFK1 | Uncharacterized protein |
| Citrus lemon | V4UG68 | Eukaryotic translation initiation factor 3 subunit I (eIF3i) |
| Citrus lemon | V4UGB0 | Uncharacterized protein |
| Citrus lemon | V4UGH4 | Uncharacterized protein |
| Citrus lemon | V4UGL9 | Uncharacterized protein |
| Citrus lemon | V4UGQ0 | Ubiquitinyl hydrolase 1 (EC 3.4.19.12) |
| Citrus lemon | V4UH00 | Uncharacterized protein |
| Citrus lemon | V4UH48 | Uncharacterized protein |
| Citrus lemon | V4UH77 | Proteasome subunit alpha type (EC 3.4.25.1) |
| Citrus lemon | V4UHD8 | Uncharacterized protein |
| Citrus lemon | V4UHD9 | Uncharacterized protein |
| Citrus lemon | V4UHF1 | Uncharacterized protein |
| Citrus lemon | V4UHZ5 | Uncharacterized protein |
| Citrus lemon | V4UI07 | 40S ribosomal protein S8 |
| Citrus lemon | V4UI34 | Eukaryotic translation initiation factor 3 subunit L (eIF3I) |
| Citrus lemon | V4UIF1 | Uncharacterized protein |
| Citrus lemon | V4UIN5 | Uncharacterized protein |
| Citrus lemon | V4UIX8 | Uncharacterized protein |
| Citrus lemon | V4UJ12 | Uncharacterized protein |
| Citrus lemon | V4UJ42 | Uncharacterized protein |
| Citrus lemon | V4UJ63 | Uncharacterized protein |
| Citrus lemon | V4UJB7 | Uncharacterized protein (Fragment) |
| Citrus lemon | V4UJC4 | Uncharacterized protein |
| Citrus lemon | V4UJX0 | Phosphotransferase (EC 2.7.1.—) |
| Citrus lemon | V4UJY5 | Uncharacterized protein |
| Citrus lemon | V4UK18 | Uncharacterized protein |
| Citrus lemon | V4UK52 | Uncharacterized protein |
| Citrus lemon | V4UKM9 | Uncharacterized protein |
| Citrus lemon | V4UKS4 | Uncharacterized protein |
| Citrus lemon | V4UKV6 | 40S ribosomal protein SA |
| Citrus lemon | V4UL30 | Pyrophosphate--fructose 6-phosphate 1-phosphotransferase |
| | | subunit beta (PFP) (EC 2.7.1.90) (6-phosphofructokinase, |
| | | pyrophosphate dependent) (PPi-PFK) |
| | | (Pyrophosphate-dependent 6-phosphofructose-1-kinase) |
| Citrus lemon | V4UL39 | Uncharacterized protein |
| Citrus lemon | V4ULH9 | Uncharacterized protein |
| Citrus lemon | V4ULL2 | Uncharacterized protein |
| Citrus lemon | V4ULS0 | Uncharacterized protein |
| Citrus lemon | V4UMU7 | Uncharacterized protein |
| Citrus lemon | V4UN36 | Uncharacterized protein |
| Citrus lemon | V4UNT5 | Uncharacterized protein |
| Citrus lemon | V4UNW1 | Uncharacterized protein |
| Citrus lemon | V4UP89 | Uncharacterized protein |
| Citrus lemon | V4UPE4 | Uncharacterized protein |
| Citrus lemon | V4UPF7 | Uncharacterized protein |
| Citrus lemon | V4UPK0 | Uncharacterized protein |
| Citrus lemon | V4UPX5 | Uncharacterized protein |
| Citrus lemon | V4UQ58 | Uncharacterized protein |
| Citrus lemon | V4UQF6 | Uncharacterized protein |
| Citrus lemon | V4UR21 | Uncharacterized protein |
| Citrus lemon | V4UR80 | Uncharacterized protein |
| Citrus lemon | V4URK3 | Uncharacterized protein |
| Citrus lemon | V4URT3 | Uncharacterized protein |
| Citrus lemon | V4US96 | Uncharacterized protein |
| Citrus lemon | V4USQ8 | Uncharacterized protein |
| Citrus lemon | V4UT16 | Uncharacterized protein |
| Citrus lemon | V4UTC6 | Uncharacterized protein |
| Citrus lemon | V4UTC8 | Uncharacterized protein |
| Citrus lemon | V4UTP6 | Uncharacterized protein |
| Citrus lemon | V4UTY0 | Proteasome subunit alpha type (EC 3.4.25.1) |
| Citrus lemon | V4UU96 | Uncharacterized protein |
| Citrus lemon | V4UUB6 | Uncharacterized protein |
| Citrus lemon | V4UUJ9 | Aminopeptidase (EC 3.4.11.—) |
| Citrus lemon | V4UUK6 | Uncharacterized protein |
| Citrus lemon | V4UV09 | Uncharacterized protein |
| Citrus lemon | V4UV83 | Lysine--tRNA ligase (EC 6.1.1.6) (Lysyl-tRNA synthetase) |
| Citrus lemon | V4UVJ5 | Diacylglycerol kinase (DAG kinase) (EC 2.7.1.107) |
| Citrus lemon | V4UW03 | Uncharacterized protein |
| Citrus lemon | V4UW04 | Uncharacterized protein |
| Citrus lemon | V4UWR1 | Uncharacterized protein |
| Citrus lemon | V4UWV8 | Uncharacterized protein |
| Citrus lemon | V4UX36 | Uncharacterized protein |
| Citrus lemon | V4V003 | Uncharacterized protein |
| Citrus lemon | V4V0J0 | 40S ribosomal protein S26 |
| Citrus lemon | V4V1P8 | Uncharacterized protein |
| Citrus lemon | V4V4V0 | Uncharacterized protein |
| Citrus lemon | V4V5T8 | Ubiquitin-fold modifier 1 |
| Citrus lemon | V4V600 | Uncharacterized protein |
| Citrus lemon | V4V622 | Aldehyde dehydrogenase |
| Citrus lemon | V4V6W1 | Uncharacterized protein |
| Citrus lemon | V4V6Z2 | Uncharacterized protein |
| Citrus lemon | V4V738 | Uncharacterized protein |
| Citrus lemon | V4V8H5 | Vacuolar protein sorting-associated protein 35 |
| Citrus lemon | V4V9P6 | Eukaryotic translation initiation factor 3 subunit F (eIF3f) |
| | | (eIF-3-epsilon) |
| Citrus lemon | V4V9V7 | Clathrin heavy chain |
| Citrus lemon | V4V9X3 | Uncharacterized protein |
| Citrus lemon | V4VAA3 | Superoxide dismutase (EC 1.15.1.1) |
| Citrus lemon | V4VAF3 | Uncharacterized protein |
| Citrus lemon | V4VBQ0 | Uncharacterized protein (Fragment) |
| Citrus lemon | V4VCL1 | Proteasome subunit beta type (EC 3.4.25.1) |
| Citrus lemon | V4VCZ9 | Uncharacterized protein |
| Citrus lemon | V4VDK1 | Peptidylprolyl isomerase (EC 5.2.1.8) |
| Citrus lemon | V4VEA1 | Uncharacterized protein |
| Citrus lemon | V4VEB3 | Alanine--tRNA ligase (EC 6.1.1.7) (Alanyl-tRNA synthetase) |
| | | (AlaRS) |
| Citrus lemon | V4VEE3 | Glutamine synthetase (EC 6.3.1.2) |
| Citrus lemon | V4VFM3 | Uncharacterized protein |
| Citrus lemon | V4VFN5 | Proteasome subunit beta type (EC 3.4.25.1) |
| Citrus lemon | V4VGD6 | Uncharacterized protein |
| Citrus lemon | V4VGL9 | Uncharacterized protein |
| Citrus lemon | V4VHI6 | Uncharacterized protein |
| Citrus lemon | V4VIP4 | Uncharacterized protein |
| Citrus lemon | V4VJT4 | Uncharacterized protein |
| Citrus lemon | V4VK14 | Uncharacterized protein |
| Citrus lemon | V4VKI5 | Protein-L-isoaspartate O-methyltransferase (EC 2.1.1.77) |
| Citrus lemon | V4VKP2 | Glyceraldehyde-3-phosphate dehydrogenase (EC 1.2.1.—) |
| Citrus lemon | V4VL73 | Acyl-coenzyme A oxidase |
| Citrus lemon | V4VLL7 | Uncharacterized protein |
| Citrus lemon | V4VN43 | Uncharacterized protein (Fragment) |
| Citrus lemon | V4VQH3 | Methylenetetrahydrofolate reductase (EC 1.5.1.20) |
| Citrus lemon | V4VTC9 | Uncharacterized protein (Fragment) |
| Citrus lemon | V4VTT4 | Uncharacterized protein |
| Citrus lemon | V4VTY7 | Uncharacterized protein |
| Citrus lemon | V4VU14 | Uncharacterized protein |
| Citrus lemon | V4VU32 | Uncharacterized protein |
| Citrus lemon | V4VUK6 | S-(hydroxymethyl)glutathione dehydrogenase (EC 1.1.1.284) |
| Citrus lemon | V4VVR8 | Uncharacterized protein |
| Citrus lemon | V4VXE2 | Uncharacterized protein |
| Citrus lemon | V4VY37 | Phosphomannomutase (EC 5.4.2.8) |
| Citrus lemon | V4VYC0 | Uncharacterized protein |
| Citrus lemon | V4VYV1 | Uncharacterized protein |
| Citrus lemon | V4VZ80 | Uncharacterized protein |
| Citrus lemon | V4VZJ7 | Uncharacterized protein |
| Citrus lemon | V4W2P2 | Alpha-mannosidase (EC 3.2.1.—) |
| Citrus lemon | V4W2Z9 | Chloride channel protein |
| Citrus lemon | V4W378 | Uncharacterized protein |
| Citrus lemon | V4W4G3 | Uncharacterized protein |
| Citrus lemon | V4W5F1 | Uncharacterized protein |
| Citrus lemon | V4W5N8 | Uncharacterized protein |
| Citrus lemon | V4W5U2 | Uncharacterized protein |
| Citrus lemon | V4W6G1 | Uncharacterized protein |
| Citrus lemon | V4W730 | Uncharacterized protein |
| Citrus lemon | V4W7J4 | Obg-like ATPase 1 |
| Citrus lemon | V4W7L5 | Uncharacterized protein |
| Citrus lemon | V4W8C5 | Uncharacterized protein |
| Citrus lemon | V4W8C9 | Uncharacterized protein |
| Citrus lemon | V4W8D3 | Uncharacterized protein |
| Citrus lemon | V4W951 | Uncharacterized protein |
| Citrus lemon | V4W9F6 | 60S ribosomal protein L18a |
| Citrus lemon | V4W9G2 | Uncharacterized protein (Fragment) |
| Citrus lemon | V4W9L3 | Uncharacterized protein |
| Citrus lemon | V4W9Y8 | Uncharacterized protein |
| Citrus lemon | V4WAP9 | Coatomer subunit beta (Beta-coat protein) |
| Citrus lemon | V4WBK6 | Cytochrome b-c1 complex subunit 7 |
| Citrus lemon | V4WC15 | Malic enzyme |
| Citrus lemon | V4WC19 | Uncharacterized protein |
| Citrus lemon | V4WC74 | Uncharacterized protein |
| Citrus lemon | V4WC86 | Serine/threonine-protein phosphatase 2A 55 kDa regulatory |
| | | subunit B |
| Citrus lemon | V4WCS4 | GTP-binding nuclear protein |
| Citrus lemon | V4WD80 | Aspartate aminotransferase (EC 2.6.1.1) |
| Citrus lemon | V4WDK0 | Uncharacterized protein |
| Citrus lemon | V4WDK3 | ATP-dependent 6-phosphofructokinase (ATP-PFK) |
| | | (Phosphofructokinase) (EC 2.7.1.11) (Phosphohexokinase) |
| Citrus lemon | V4WE00 | Uncharacterized protein |
| Citrus lemon | V4WEE3 | Uncharacterized protein |
| Citrus lemon | V4WEN2 | Uncharacterized protein |
| Citrus lemon | V4WG97 | Autophagy-related protein |
| Citrus lemon | V4WGV2 | Uncharacterized protein |
| Citrus lemon | V4WGW5 | Uridine kinase (EC 2.7.1.48) |
| Citrus lemon | V4WHD4 | Uncharacterized protein |
| Citrus lemon | V4WHF8 | Sucrose synthase (EC 2.4.1.13) |
| Citrus lemon | V4WHK2 | Pectinesterase (EC 3.1.1.11) |
| Citrus lemon | V4WHQ4 | Uncharacterized protein |
| Citrus lemon | V4WHT6 | Uncharacterized protein |
| Citrus lemon | V4WJ93 | Uncharacterized protein |
| Citrus lemon | V4WJA9 | Uncharacterized protein |
| Citrus lemon | V4WJB1 | Uncharacterized protein |
| Citrus lemon | V9HXG3 | Protein disulfide-isomerase (EC 5.3.4.1) |
| Citrus lemon | W8Q8K1 | Putative inorganic pyrophosphatase |
| Citrus lemon | W8QJL0 | Putative isopentenyl pyrophosphate isomerase |

| Grape | Accession Number | Identified Proteins |
|---|---|---|
| Grape | A5C5K3 (+2) | Adenosylhomocysteinase |
| Grape | Q9M6B5 | Alcohol dehydrogenase 6 |
| Grape | A3FA65 (+1) | Aquaporin PIP1; 3 |
| Grape | Q0MX13 (+2) | Aquaporin PIP2; 2 |
| Grape | A3FA69 (+4) | Aquaporin PIP2; 4 |
| Grape | A5AFS1 (+2) | Elongation factor 1-alpha |
| Grape | UPI0001985702 | elongation factor 2 |
| Grape | D7T227 | Enolase |
| Grape | D7TJ12 | Enolase |
| Grape | A5B118 (+1) | Fructose-bisphosphate aldolase |
| Grape | E0CQ39 | Glucose-6-phosphate isomerase |
| Grape | D7TW04 | Glutathione peroxidase |
| Grape | A1YW90 (+3) | Glutathione S-transferase |
| Grape | A5BEW0 | Histone H4 |
| Grape | UPI00015C9A6A | HSC70-1 (heat shock cognate 70 kDa protein 1); ATP |
| | | binding isoform 1 |
| Grape | D7FBC0 (+1) | Malate dehydrogenase |
| Grape | D7TBH4 | Malic enzyme |
| Grape | A5ATB7 (+1) | Methylenetetrahydrofolate reductase |
| Grape | A5JPK7 (+1) | Monodehydroascorbate reductase |
| Grape | A5AKD8 | Peptidyl-prolyl cis-trans isomerase |
| Grape | A5BQN6 | Peptidyl-prolyl cis-trans isomerase |
| Grape | A5CAF6 | Phosphoglycerate kinase |
| Grape | Q09VU3 (+1) | Phospholipase D |
| Grape | D7SK33 | Phosphorylase |
| Grape | A5AQ89 | Profilin |
| Grape | C5DB50 (+2) | Putative 2,3-bisphosphoglycerate-independent |
| | | phosphoglycerate mutase |
| Grape | D7TIZ5 | Pyruvate kinase |
| Grape | A5BV65 | Triosephosphate isomerase |
| Grapefruit | G8Z362 (+1) | (E)-beta-farnesene synthase |
| Grapefruit | Q5CD81 | (E)-beta-ocimene synthase |
| Grapefruit | D0UZK1 (+2) | 1,2 rhamnosyltransferase |
| Grapefruit | A7ISD3 | 1,6-rhamnosyltransferase |
| Grapefruit | Q80H98 | 280 kDa protein |
| Grapefruit | Q15GA4 (+2) | 286 kDa polyprotein |
| Grapefruit | D7NHW9 | 2-phospho-D-glycerate hydrolase |
| Grapefruit | D0EAL9 | 349 kDa polyprotein |
| Grapefruit | Q9DTG5 | 349-kDa polyprotein |
| Grapefruit | O22297 | Acidic cellulase |
| Grapefruit | Q8H986 | Acidic class I chitinase |
| Grapefruit | D3GQL0 | Aconitate hydratase 1 |
| Grapefruit | K7N8A0 | Actin |
| Grapefruit | A8W8Y0 | Alcohol acyl transferase |
| Grapefruit | Q84V85 | Allene oxide synthase |
| Grapefruit | F8WL79 | Aminopeptidase |
| Grapefruit | Q09MG5 | Apocytochrome f |
| Grapefruit | J7EIR8 | Ascorbate peroxidase |
| Grapefruit | B9VRH6 | Ascorbate peroxidase |
| Grapefruit | G9I820 | Auxin-response factor |
| Grapefruit | J7ICW8 | Beta-amylase |
| Grapefruit | Q8L5Q9 | Beta-galactosidase |
| Grapefruit | A7BG60 | Beta-pinene synthase |
| Grapefruit | C0KLD1 | Beta-tubulin |
| Grapefruit | Q91QZ1 | Capsid protein |
| Grapefruit | Q3SAK9 | Capsid protein |
| Grapefruit | D2U833 | Cation chloride cotransporter |
| Grapefruit | C3VPJ0 (+3) | Chaicone synthase |
| Grapefruit | D5LM39 | Chloride channel protein |
| Grapefruit | Q9M4U0 | Cinnamate 4-hydroxylase CYP73 |
| Grapefruit | Q39627 | Citrin |
| Grapefruit | G2XKD3 | Coat protein |
| Grapefruit | Q3L2I6 | Coat protein |
| Grapefruit | D5FV16 | CRT/DRE binding factor |
| Grapefruit | Q8H6S5 | CTV.2 |
| Grapefruit | Q8H6Q8 | CTV.20 |
| Grapefruit | Q8H6Q7 | CTV.22 |
| Grapefruit | Q1I1D7 | Cytochrome P450 |
| Grapefruit | Q7Y045 | Dehydrin |
| Grapefruit | F8WLD2 | DNA excision repair protein |
| Grapefruit | Q09MI8 | DNA-directed RNA polymerase subunit beta″ |
| Grapefruit | D2WKC9 | Ethylene response 1 |
| Grapefruit | D2WKD2 | Ethylene response sensor 1 |
| Grapefruit | D7PVG7 | Ethylene-insensitive 3-like 1 protein |
| Grapefruit | G3CHK8 | Eukaryotic translation initiation factor 3 subunit E |
| Grapefruit | A9NJG4 (+3) | Fatty acid hydroperoxide lyase |
| Grapefruit | B8Y9B5 | F-box family protein |
| Grapefruit | Q000W4 | Fe(III)-chelate reductase |
| Grapefruit | Q6Q3H4 | Fructokinase |
| Grapefruit | F8WL95 | Gag-pol polyprotein |
| Grapefruit | Q8L5K4 | Gamma-terpinene synthase, chloroplastic |
| Grapefruit | Q9SP43 | Glucose-1-phosphate adenylyltransferase |
| Grapefruit | Q3HM93 | Glutathione S-transferase |
| Grapefruit | D0VEW6 | GRAS family transcription factor |
| Grapefruit | F8WL87 | Heat shock protein |
| Grapefruit | H9NHK0 | Hsp90 |
| Grapefruit | Q8H6R4 | Jp18 |
| Grapefruit | G3CHK6 | Leucine-rich repeat family protein |
| Grapefruit | B2YGX9 (+1) | Limonoid UDP-glucosyltransferase |
| Grapefruit | Q05KK0 | MADS-box protein |
| Grapefruit | F8WLB4 | Mechanosensitive ion channel domain-containing protein |
| Grapefruit | Q5CD82 | Monoterpene synthase |
| Grapefruit | F8WLC4 | MYB transcription factor |
| Grapefruit | A5YWA9 | NAC domain protein |
| Grapefruit | Q09MC9 | NAD(P)H-quinone oxidoreductase subunit 5, chloroplastic |
| Grapefruit | Q8H6R9 | NBS-LRR type disease resistance protein |
| Grapefruit | Q8H6S0 | NBS-LRR type disease resistance protein |
| Grapefruit | Q8H6R6 | NBS-LRR type disease resistance protein |
| Grapefruit | J9WR93 | p1a |
| Grapefruit | Q1X8V8 | P23 |
| Grapefruit | E7DSS0 (+4) | P23 |
| Grapefruit | G0Z9I6 | p27 |
| Grapefruit | I3XHN0 | p33 |
| Grapefruit | B8YDL3 | p33 protein |
| Grapefruit | B9VB22 | p33 protein |
| Grapefruit | P87587 | P346 |
| Grapefruit | B9VB56 | p349 protein |
| Grapefruit | I3RWW7 | p349 protein |
| Grapefruit | B9VB20 | p349 protein |
| Grapefruit | Q9WID7 | p349 protein |
| Grapefruit | Q2XP16 | P353 |
| Grapefruit | O04886 (+1) | Pectinesterase 1 |
| Grapefruit | F8WL74 | Peptidyl-prolyl cis-trans isomerase |
| Grapefruit | Q0ZA67 | Peroxidase |
| Grapefruit | F1CT41 | Phosphoenolpyruvate carboxylase |
| Grapefruit | B1PBV7 (+2) | Phytoene synthase |
| Grapefruit | Q9ZWQ8 | Plastid-lipid-associated protein, chloroplastic |
| Grapefruit | Q94FM1 | Pol polyprotein |
| Grapefruit | Q94FM0 | Pol polyprotein |
| Grapefruit | G9I825 | Poly C-binding protein |
| Grapefruit | O64460 (+7) | Polygalacturonase inhibitor |
| Grapefruit | I3XHM8 | Polyprotein |
| Grapefruit | C0STR9 | Polyprotein |
| Grapefruit | H6U1F0 | Polyprotein |
| Grapefruit | B8QHP8 | Polyprotein |
| Grapefruit | I3V6C0 | Polyprotein |
| Grapefruit | C0STS0 | Polyprotein |
| Grapefruit | K0FGH5 | Polyprotein |
| Grapefruit | Q3HWZ1 | Polyprotein |
| Grapefruit | F8WLA5 | PPR containing protein |
| Grapefruit | Q06652 (+1) | Probable phospholipid hydroperoxide glutathione |
| | | peroxidase |
| Grapefruit | P84177 | Profilin |
| Grapefruit | Q09MB4 | Protein ycf2 |
| Grapefruit | A8C183 | PSI reaction center subunit II |
| Grapefruit | A5JVP6 | Putative 2b protein |
| Grapefruit | D0EFM2 | Putative eukaryotic translation initiation factor 1 |
| Grapefruit | Q18L98 | Putative gag-pol polyprotein |
| Grapefruit | B5AMI9 | Putative movement protein |
| Grapefruit | A1ECK5 | Putative multiple stress-responsive zinc-finger protein |
| Grapefruit | B5AMJ0 | Putative replicase polyprotein |
| Grapefruit | I7CYN5 | Putative RNA-dependent RNA polymerase |
| Grapefruit | Q8RVR2 | Putative terpene synthase |
| Grapefruit | B5TE89 | Putative uncharacterized protein |
| Grapefruit | Q8JVF3 | Putative uncharacterized protein |
| Grapefruit | F8WLB0 | Putative uncharacterized protein ORF43 |
| Grapefruit | A5JVP4 | Putative viral replicase |
| Grapefruit | M1JAW3 | Replicase |
| Grapefruit | H6VXK8 | Replicase polyprotein |
| Grapefruit | J9UF50 (+1) | Replicase protein 1a |
| Grapefruit | J9RV45 | Replicase protein 2a |
| Grapefruit | Q5EGG5 | Replicase-associated polyprotein |
| Grapefruit | G9I823 | RNA recognition motif protein 1 |
| Grapefruit | J7EPC0 | RNA-dependent RNA polymerase |
| Grapefruit | Q6DN67 | RNA-directed RNA polymerase L |
| Grapefruit | A9CQM4 | SEPALLATA1 homolog |
| Grapefruit | Q9SLS2 | Sucrose synthase |
| Grapefruit | Q9SLV8 (+1) | Sucrose synthase |
| Grapefruit | Q38JC1 | Temperature-induced lipocalin |
| Grapefruit | D0ELH6 | Tetratricopeptide domain-containing thioredoxin |
| Grapefruit | D2KU75 | Thaumatin-like protein |
| Grapefruit | C3VIC2 | Translation elongation factor |
| Grapefruit | D5LY07 | Ubiquitin/ribosomal fusion protein |
| Grapefruit | C6KI43 | UDP-glucosyltransferase family 1 protein |
| Grapefruit | A0FKR1 | Vacuolar citrate/H+ symporter |
| Grapefruit | Q944C8 | Vacuolar invertase |
| Grapefruit | Q9MB46 | V-type proton ATPase subunit E |
| Grapefruit | F8WL82 | WD-40 repeat family protein |
| Helianthuus annuus | HanXRQChr03g0080391 | Hsp90 |
| Helianthuus annuus | HanXRQChr13g0408351 | Hsp90 |
| Helianthuus annuus | HanXRQChr13g0408441 | Hsp90 |
| Helianthuus annuus | HanXRQChr14g0462551 | Hsp90 |
| Helianthuus annuus | HanXRQChr02g0044471 | Hsp70 |
| Helianthuus annuus | HanXRQChr02g0044481 | Hsp70 |
| Helianthuus annuus | HanXRQChr05g0132631 | Hsp70 |
| Helianthuus annuus | HanXRQChr05g0134631 | Hsp70 |
| Helianthuus annuus | HanXRQChr05g0134801 | Hsp70 |
| Helianthuus annuus | HanXRQChr10g0299441 | glutathione S-transferase |
| Helianthuus annuus | HanXRQChr16g0516291 | glutathione S-transferase |
| Helianthuus annuus | HanXRQChr03g0091431 | lactate/malate dehydrogenase |
| Helianthuus annuus | HanXRQChr13g0421951 | lactate/malate dehydrogenase |
| Helianthuus annuus | HanXRQChr10g0304821 | lactate/malate dehydrogenase |
| Helianthuus annuus | HanXRQChr12g0373491 | lactate/malate dehydrogenase |
| Helianthuus annuus | HanXRQChr01g0031071 | small GTPase superfamily, Rab type |
| Helianthuus annuus | HanXRQChr01g0031091 | small GTPase superfamily, Rab type |
| Helianthuus annuus | HanXRQChr02g0050791 | small GTPase superfamily, Rab type |
| Helianthuus annuus | HanXRQChr11g0353711 | small GTPase superfamily, Rab type |
| Helianthuus annuus | HanXRQChr13g0402771 | small GTPase superfamily, Rab type |
| Helianthuus annuus | HanXRQChr07g0190171 | isocitrate/isopropylmalate dehydrogenase |
| Helianthuus annuus | HanXRQChr16g0532251 | isocitrate/isopropylmalate dehydrogenase |
| Helianthuus annuus | HanXRQChr03g0079131 | phosphoenolpyruvate carboxylase |
| Helianthuus annuus | HanXRQChr15g0495261 | phosphoenolpyruvate carboxylase |
| Helianthuus annuus | HanXRQChr13g0388931 | phosphoenolpyruvate carboxylase |
| Helianthuus annuus | HanXRQChr14g0442731 | phosphoenolpyruvate carboxylase |
| Helianthuus annuus | HanXRQChr15g0482381 | UTP--glucose-1-phosphate uridylyltransferase |
| Helianthuus annuus | HanXRQChr16g0532261 | UTP--glucose-1-phosphate uridylyltransferase |
| Helianthuus annuus | HanXRQChr05g0135591 | tubulin |
| Helianthuus annuus | HanXRQChr06g0178921 | tubulin |
| Helianthuus annuus | HanXRQChr08g0237071 | tubulin |
| Helianthuus annuus | HanXRQChr11g0337991 | tubulin |
| Helianthuus annuus | HanXRQChr13g0407921 | tubulin |
| Helianthuus annuus | HanXRQChr05g0145191 | tubulin |
| Helianthuus annuus | HanXRQChr07g0187021 | tubulin |
| Helianthuus annuus | HanXRQChr07g0189811 | tubulin |
| Helianthuus annuus | HanXRQChr09g0253681 | tubulin |
| Helianthuus annuus | HanXRQChr10g0288911 | tubulin |
| Helianthuus annuus | HanXRQChr11g0322631 | tubulin |
| Helianthuus annuus | HanXRQChr12g0367231 | tubulin |
| Helianthuus annuus | HanXRQChr13g0386681 | tubulin |
| Helianthuus annuus | HanXRQChr13g0393261 | tubulin |
| Helianthuus annuus | HanXRQChr12g0371591 | ubiquitin |
| Helianthuus annuus | HanXRQChr12g0383641 | ubiquitin |
| Helianthuus annuus | HanXRQChr17g0569881 | ubiquitin |
| Helianthuus annuus | HanXRQChr06g0171511 | photosystem II HCF136, stability/assembly factor |
| Helianthuus annuus | HanXRQChr17g0544921 | photosystem II HCF136, stability/assembly factor |
| Helianthuus annuus | HanXRQChr16g0526461 | proteasome B-type subunit |
| Helianthuus annuus | HanXRQChr17g0565551 | proteasome B-type subunit |
| Helianthuus annuus | HanXRQChr05g0149801 | proteasome B-type subunit |
| Helianthuus annuus | HanXRQChr09g0241421 | proteasome B-type subunit |
| Helianthuus annuus | HanXRQChr11g0353161 | proteasome B-type subunit |
| Helianthuus annuus | HanXRQChr16g0506311 | proteinase inhibitor family I3 (Kunitz) |
| Helianthuus annuus | HanXRQChr16g0506331 | proteinase inhibitor family I3 (Kunitz) |
| Helianthuus annuus | HanXRQChr09g0265401 | metallopeptidase (M10 family) |
| Helianthuus annuus | HanXRQChr09g0265411 | metallopeptidase (M10 family) |
| Helianthuus annuus | HanXRQChr05g0154561 | ATPase, AAA-type |
| Helianthuus annuus | HanXRQChr08g0235061 | ATPase, AAA-type |
| Helianthuus annuus | HanXRQChr09g0273921 | ATPase, AAA-type |
| Helianthuus annuus | HanXRQChr16g0498881 | ATPase, AAA-type |
| Helianthuus annuus | HanXRQChr02g0058711 | oxoacid dehydrogenase acyltransferase |
| Helianthuus annuus | HanXRQChr08g0214191 | oxoacid dehydrogenase acyltransferase |
| Helianthuus annuus | HanXRQChr08g0208631 | small GTPase superfamily, SAR1-type |
| Helianthuus annuus | HanXRQChr11g0331441 | small GTPase superfamily, SAR1-type |
| Helianthuus annuus | HanXRQChr12g0371571 | small GTPase superfamily, SAR1-type |
| Helianthuus annuus | HanXRQChr12g0383571 | small GTPase superfamily, SAR1-type |
| Helianthuus annuus | HanXRQChr14g0446771 | small GTPase superfamily, SAR1-type |
| Helianthuus annuus | HanXRQChr17g0539461 | small GTPase superfamily, SAR1-type |
| Helianthuus annuus | HanXRQChr17g0548271 | small GTPase superfamily, SAR1-type |
| Helianthuus annuus | HanXRQChr17g0569871 | small GTPase superfamily, SAR1-type |
| Helianthuus annuus | HanXRQChr10g0311201 | ATPase, V1 complex, subunit A |
| Helianthuus annuus | HanXRQChr12g0359711 | ATPase, V1 complex, subunit A |
| Helianthuus annuus | HanXRQChr04g0124671 | fructose-1,6-bisphosphatase |
| Helianthuus annuus | HanXRQChr06g0176631 | fructose-1,6-bisphosphatase |
| Helianthuus annuus | HanXRQCPg0579861 | photosystem II PsbD/D2, reaction centre |
| Helianthuus annuus | HanXRQChr00c0439g0574731 | photosystem II PsbD/D2, reaction centre |
| Helianthuus annuus | HanXRQChr04g0099321 | photosystem II PsbD/D2, reaction centre |
| Helianthuus annuus | HanXRQChr08g0210231 | photosystem II PsbD/D2, reaction centre |
| Helianthuus annuus | HanXRQChr11g0326671 | photosystem II PsbD/D2, reaction centre |
| Helianthuus annuus | HanXRQChr17g0549121 | photosystem II PsbD/D2, reaction centre |
| Helianthuus annuus | HanXRQCPg0579731 | photosystem II protein D1 |
| Helianthuus annuus | HanXRQChr00c0126g0571821 | photosystem II protein D1 |
| Helianthuus annuus | HanXRQChr00c0165g0572191 | photosystem II protein D1 |
| Helianthuus annuus | HanXRQChr00c0368g0574171 | photosystem II protein D1 |
| Helianthuus annuus | HanXRQChr00c0454g0574931 | photosystem II protein D1 |
| Helianthuus annuus | HanXRQChr00c0524g0575441 | photosystem II protein D1 |
| Helianthuus annuus | HanXRQChr00c0572g0575941 | photosystem II protein D1 |
| Helianthuus annuus | HanXRQChr09g0257281 | photosystem II protein D1 |
| Helianthuus annuus | HanXRQChr11g0326571 | photosystem II protein D1 |
| Helianthuus annuus | HanXRQChr11g0327051 | photosystem II protein D1 |
| Helianthuus annuus | HanXRQChr16g0503941 | photosystem II protein D1 |
| Helianthuus annuus | HanXRQCPg0580061 | photosystem II cytochrome b559 |
| Helianthuus annuus | HanXRQChr01g0020331 | photosystem II cytochrome b559 |
| Helianthuus annuus | HanXRQChr10g0283581 | photosystem II cytochrome b559 |
| Helianthuus annuus | HanXRQChr10g0284271 | photosystem II cytochrome b559 |
| Helianthuus annuus | HanXRQChr10g0289291 | photosystem II cytochrome b559 |
| Helianthuus annuus | HanXRQChr10g0318171 | photosystem II cytochrome b559 |
| Helianthuus annuus | HanXRQChr11g0326851 | photosystem II cytochrome b559 |
| Helianthuus annuus | HanXRQChr16g0529011 | photosystem II cytochrome b559 |
| Helianthuus annuus | HanXRQChr08g0219051 | chlorophyll A-B binding protein |
| Helianthuus annuus | HanXRQChr12g0370841 | chlorophyll A-B binding protein |
| Helianthuus annuus | HanXRQChr02g0053151 | chlorophyll A-B binding protein |
| Helianthuus annuus | HanXRQChr02g0053161 | chlorophyll A-B binding protein |
| Helianthuus annuus | HanXRQCPg0580051 | cytochrome f |
| Helianthuus annuus | HanXRQChr01g0020341 | cytochrome f |
| Helianthuus annuus | HanXRQChr10g0283571 | cytochrome f |
| Helianthuus annuus | HanXRQChr10g0284261 | cytochrome f |
| Helianthuus annuus | HanXRQChr10g0289281 | cytochrome f |
| Helianthuus annuus | HanXRQChr10g0318181 | cytochrome f |
| Helianthuus annuus | HanXRQChr11g0326841 | cytochrome f |
| Helianthuus annuus | HanXRQChr15g0497521 | cytochrome f |
| Helianthuus annuus | HanXRQChr06g0163851 | ribosomal protein |
| Helianthuus annuus | HanXRQChr09g0252071 | ribosomal protein |
| Helianthuus annuus | HanXRQChr12g0374041 | ribosomal protein |
| Helianthuus annuus | HanXRQChr04g0128141 | ribosomal protein |
| Helianthuus annuus | HanXRQChr05g0163131 | ribosomal protein |
| Helianthuus annuus | HanXRQChr03g0076971 | ribosomal protein |
| Helianthuus annuus | HanXRQChr05g0159851 | ribosomal protein |
| Helianthuus annuus | HanXRQChr05g0159971 | ribosomal protein |
| Helianthuus annuus | HanXRQChr11g0324631 | ribosomal protein |
| Helianthuus annuus | HanXRQChr13g0408051 | ribosomal protein |
| Helianthuus annuus | HanXRQChr03g0089331 | ribosomal protein |
| Helianthuus annuus | HanXRQChr13g0419951 | ribosomal protein |
| Helianthuus annuus | HanXRQChr15g0497041 | ribosomal protein |
| Helianthuus annuus | HanXRQChr16g0499761 | ribosomal protein |
| Helianthuus annuus | HanXRQChr04g0106961 | ribosomal protein |
| Helianthuus annuus | HanXRQChr06g0175811 | ribosomal protein |
| Helianthuus annuus | HanXRQChr04g0122771 | ribosomal protein |
| Helianthuus annuus | HanXRQChr09g0245691 | ribosomal protein |
| Helianthuus annuus | HanXRQChr16g0520021 | ribosomal protein |
| Helianthuus annuus | HanXRQChr03g0060471 | ribosomal protein |
| Helianthuus annuus | HanXRQChr14g0429531 | ribosomal protein |
| Helianthuus annuus | HanXRQChr06g0171911 | ribosomal protein |
| Helianthuus annuus | HanXRQChr15g0479091 | ribosomal protein |
| Helianthuus annuus | HanXRQChr15g0479101 | ribosomal protein |
| Helianthuus annuus | HanXRQChr17g0543641 | ribosomal protein |
| Helianthuus annuus | HanXRQChr17g0543661 | ribosomal protein |
| Helianthuus annuus | HanXRQChr04g0105831 | ribosomal protein |
| Helianthuus annuus | HanXRQChr09g0258341 | ribosomal protein |
| Helianthuus annuus | HanXRQChr10g0287141 | ribosomal protein |
| Helianthuus annuus | HanXRQChr15g0463911 | ribosomal protein |
| Helianthuus annuus | HanXRQChr03g0076171 | ribosomal protein |
| Helianthuus annuus | HanXRQChr05g0159291 | ribosomal protein |
| Helianthuus annuus | HanXRQChr13g0407551 | ribosomal protein |
| Helianthuus annuus | HanXRQChr12g0380701 | ribosomal protein |
| Helianthuus annuus | HanXRQChr15g0477271 | ribosomal protein |
| Helianthuus annuus | HanXRQChr17g0545211 | ribosomal protein |
| Helianthuus annuus | HanXRQChr17g0570741 | ribosomal protein |
| Helianthuus annuus | HanXRQChr17g0570761 | ribosomal protein |
| Helianthuus annuus | HanXRQChr02g0044021 | ribosomal protein |
| Helianthuus annuus | HanXRQChr05g0152871 | ribosomal protein |
| Helianthuus annuus | HanXRQChr01g0012781 | ribosomal protein |
| Helianthuus annuus | HanXRQChr08g0230861 | ribosomal protein |
| Helianthuus annuus | HanXRQChr13g0391831 | ribosomal protein |
| Helianthuus annuus | HanXRQChr11g0337791 | bifunctional trypsin/alpha-amylase inhibitor |
| Helianthuus annuus | HanXRQChr10g0312371 | 2-oxoacid dehydrogenase acyltransferase |
| Helianthuus annuus | HanXRQChr09g0276191 | acid phosphatase (class B) |
| Helianthuus annuus | HanXRQChr05g0142271 | aldose-1-epimerase |
| Helianthuus annuus | HanXRQChr14g0439791 | alpha-D-phosphohexomutase |
| Helianthuus annuus | HanXRQChr09g0251071 | alpha-L-fucosidase |
| Helianthuus annuus | HanXRQChr05g0147371 | annexin |
| Helianthuus annuus | HanXRQChr09g0247561 | Asp protease (Peptidase family A1) |
| Helianthuus annuus | HanXRQChr13g0409681 | berberine-bridge enzyme (S)-reticulin: oxygen oxido-reductase |
| Helianthuus annuus | HanXRQChr10g0295971 | beta-hydroxyacyl-(acyl-carrier-protein) dehydratase |
| Helianthuus annuus | HanXRQChr13g0412571 | carbohydrate esterase family 13 - CE13 (pectin acylesterase - PAE) |
| Helianthuus annuus | HanXRQChr12g0360101 | carbohydrate esterase family 8 - CE8 (pectin methylesterase - PME) |
| Helianthuus annuus | HanXRQChr01g0019231 | carbonic anhydrase |
| Helianthuus annuus | HanXRQChr02g0036611 | cellular retinaldehyde binding/alpha-tocopherol transport |
| Helianthuus annuus | HanXRQChr10g0313581 | chaperonin Cpn60 |
| Helianthuus annuus | HanXRQChr09g0251791 | chlathrin |
| Helianthuus annuus | HanXRQChr11g0329811 | chlorophyll A-B binding protein |
| Helianthuus annuus | HanXRQChr13g0398861 | cobalamin (vitamin B12)-independent methionine synthase |
| Helianthuus annuus | HanXRQChr10g0298981 | cyclophilin |
| Helianthuus annuus | HanXRQChr04g0103281 | Cys protease (papain family) |
| Helianthuus annuus | HanXRQChr09g0268361 | cytochrome P450 |
| Helianthuus annuus | HanXRQChr17g0535591 | dirigent protein |
| Helianthuus annuus | HanXRQChr03g0065901 | expansin |
| Helianthuus annuus | HanXRQChr11g0336761 | expressed protein (cupin domain, seed storage protein domain) |
| Helianthuus annuus | HanXRQChr10g0280931 | expressed protein (cupin domain, seed storage protein domain) |
| Helianthuus annuus | HanXRQChr10g0288971 | expressed protein (cupin domain, seed storage protein domain) |
| Helianthuus annuus | HanXRQChr12g0380361 | expressed protein (cupin domain, seed storage protein domain) |
| Helianthuus annuus | HanXRQChr09g0254381 | expressed protein (cupin domain, seed storage protein domain) |
| Helianthuus annuus | HanXRQChr04g0112711 | expressed protein (cupin domain, seed storage protein domain) |
| Helianthuus annuus | HanXRQChr07g0196131 | expressed protein (cupin domain, seed storage protein domain) |
| Helianthuus annuus | HanXRQChr10g0301281 | expressed protein (cupin domain, seed storage protein domain) |
| Helianthuus annuus | HanXRQChr10g0301931 | expressed protein (cupin domain, seed storage protein domain) |
| Helianthuus annuus | HanXRQChr13g0404461 | expressed protein (cupin domain) |
| Helianthuus annuus | HanXRQChr01g0015821 | expressed protein (DUF642) |
| Helianthuus annuus | HanXRQChr03g0065301 | expressed protein (Gnk2-homologous domain, antifungal |
| | | protein of Ginkgo seeds) |
| Helianthuus annuus | HanXRQChr03g0068311 | expressed protein (LRR domains) |
| Helianthuus annuus | HanXRQChr10g0291371 | expressed protein (LRR domains) |
| Helianthuus annuus | HanXRQChr03g0075061 | fasciclin-like arabinogalactan protein (FLA) |
| Helianthuus annuus | HanXRQChr08g0221961 | ferritin |
| Helianthuus annuus | HanXRQChr09g0257521 | FMN-dependent dehydrogenase |
| Helianthuus annuus | HanXRQChr14g0441641 | fructose-bisphosphate aldolase |
| Helianthuus annuus | HanXRQChr10g0312621 | germin |
| Helianthuus annuus | HanXRQChr09g0244271 | glucose-methanol-choline oxidoreductase |
| Helianthuus annuus | HanXRQChr03g0061571 | glutamate synthase |
| Helianthuus annuus | HanXRQChr05g0144801 | glyceraldehyde 3-phosphate dehydrogenase |
| Helianthuus annuus | HanXRQChr17g0550211 | glycerophosphoryl diester phosphodiesterase |
| Helianthuus annuus | HanXRQChr06g0175391 | glycoside hydrolase family 16 - GH16 (endoxyloglucan |
| | | transferase) |
| Helianthuus annuus | HanXRQChr11g0351571 | glycoside hydrolase family 17 - GH17 (beta-1,3-glucosidase) |
| Helianthuus annuus | HanXRQChr05g0141461 | glycoside hydrolase family 18 - GH18 |
| Helianthuus annuus | HanXRQChr09g0276721 | glycoside hydrolase family 19 - GH19 |
| Helianthuus annuus | HanXRQChr02g0046191 | glycoside hydrolase family 2 - GH2 |
| Helianthuus annuus | HanXRQChr16g0524981 | glycoside hydrolase family 20 - GH20 |
| | | (N-acetyl-beta-glucosaminidase) |
| Helianthuus annuus | HanXRQChr11g0322851 | glycoside hydrolase family 27 - GH27 |
| | | (alpha-galactosidase/melibiase) |
| Helianthuus annuus | HanXRQChr10g0293191 | glycoside hydrolase family 3 - GH3 |
| Helianthuus annuus | HanXRQChr16g0511881 | glycoside hydrolase family 31 - GH31 (alpha-xylosidase) |
| Helianthuus annuus | HanXRQChr14g0461441 | glycoside hydrolase family 32 - GH32 (vacuolar invertase) |
| Helianthuus annuus | HanXRQChr13g0423671 | glycoside hydrolase family 35 - GH35 (beta-galactosidase) |
| Helianthuus annuus | HanXRQChr10g0319301 | glycoside hydrolase family 35 - GH35 (beta-galactosidase) |
| Helianthuus annuus | HanXRQChr09g0256531 | glycoside hydrolase family 38 - GH38 (alpha-mannosidase) |
| Helianthuus annuus | HanXRQChr11g0320901 | glycoside hydrolase family 5 - GH5 (glucan-1,3-beta |
| | | glucosidase) |
| Helianthuus annuus | HanXRQChr05g0130491 | glycoside hydrolase family 51 - GH51 |
| | | (alpha-arabinofuranosidase) |
| Helianthuus annuus | HanXRQChr10g0314191 | glycoside hydrolase family 79 - GH79 |
| | | (endo-beta-glucuronidase/heparanase |
| Helianthuus annuus | HanXRQChr13g0397411 | homologous to A. thaliana PMR5 (Powdery Mildew |
| | | Resistant) (carbohydrate acylation) |
| Helianthuus annuus | HanXRQChr14g0444681 | inhibitor family I3 (Kunitz-P family) |
| Helianthuus annuus | HanXRQChr14g0445181 | lactate/malate dehydrogenase |
| Helianthuus annuus | HanXRQChr17g0564111 | lectin (D-mannose) |
| Helianthuus annuus | HanXRQChr17g0558861 | lectin (PAN-2 domain) |
| Helianthuus annuus | HanXRQChr02g0039251 | lipase acylhydrolase (GDSL family) |
| Helianthuus annuus | HanXRQChr01g0000161 | lipid transfer protein/trypsin-alpha amylase inhibitor |
| Helianthuus annuus | HanXRQChr02g0047121 | mannose-binding lectin |
| Helianthuus annuus | HanXRQChr10g0303361 | mitochondrial carrier protein |
| Helianthuus annuus | HanXRQChr15g0489551 | multicopper oxidase |
| Helianthuus annuus | HanXRQChr05g0135581 | neutral/alkaline nonlysosomal ceramidase |
| Helianthuus annuus | HanXRQChr01g0017621 | nucleoside diphosphate kinase |
| Helianthuus annuus | HanXRQChr10g0295991 | peroxidase |
| Helianthuus annuus | HanXRQChr13g0398251 | peroxiredoxin |
| Helianthuus annuus | HanXRQChr11g0333171 | phosphate-induced (phi) protein 1 |
| Helianthuus annuus | HanXRQChr03g0060421 | phosphodiesterase/nucleotide pyrophosphatase/phosphate |
| | | transferase |
| Helianthuus annuus | HanXRQChr03g0078011 | phosphofructokinase |
| Helianthuus annuus | HanXRQChr13g0408831 | phosphoglycerate kinase |
| Helianthuus annuus | HanXRQChr10g0286701 | phosphoglycerate mutase |
| Helianthuus annuus | HanXRQChr06g0171591 | photosystem II PsbP, oxygen evolving complex |
| Helianthuus annuus | HanXRQChr14g0434951 | plastid lipid-associated protein/fibrillin conserved domain |
| Helianthuus annuus | HanXRQChr05g0146621 | plastocyanin (blue copper binding protein) |
| Helianthuus annuus | HanXRQChr11g0330251 | polyphenol oxidase |
| Helianthuus annuus | HanXRQChr04g0094541 | proteasome A-type subunit |
| Helianthuus annuus | HanXRQChr03g0081271 | proteasome B-type subunit |
| Helianthuus annuus | HanXRQChr12g0356851 | purple acid phosphatase |
| Helianthuus annuus | HanXRQChr15g0485781 | pyridoxal phosphate-dependent transferase |
| Helianthuus annuus | HanXRQChr11g0336791 | ribosomal protein |
| Helianthuus annuus | HanXRQChr11g0330521 | ribosomal protein |
| Helianthuus annuus | HanXRQChr11g0326801 | ribulose bisphosphate carboxylase, large subunit |
| Helianthuus annuus | HanXRQChr16g0523951 | ribulose-1,5-bisphosphate carboxylase small subunit |
| Helianthuus annuus | HanXRQChr01g0022151 | S-adenosyl-L-homocysteine hydrolase |
| Helianthuus annuus | HanXRQChr14g0454811 | S-adenosylmethionine synthetase |
| Helianthuus annuus | HanXRQChr04g0109991 | SCP-like extracellular protein (PR-1) |
| Helianthuus annuus | HanXRQChr03g0072241 | Ser carboxypeptidase (Peptidase family S10) |
| Helianthuus annuus | HanXRQChr12g0377221 | Ser protease (subtilisin) (Peptidase family S8) |
| Helianthuus annuus | HanXRQChr02g0055581 | superoxide dismutase |
| Helianthuus annuus | HanXRQChr15g0493261 | thaumatin (PR5) |
| Helianthuus annuus | HanXRQChr16g0532531 | transketolase |
| Helianthuus annuus | HanXRQChr07g0197421 | translation elongation factor EFTu/EF1A |
| Helianthuus annuus | HanXRQChr06g0173951 | translationally controlled tumour protein |

What is claimed is:

1. A plant messenger pack (PMP) comprising one or more exogenous polypeptides, wherein the one or more exogenous polypeptides are mammalian therapeutic agents and are encapsulated by the PMP, and wherein the exogenous polypeptides are not pathogen control agents.

2. The PMP of claim 1, wherein the mammalian therapeutic agent is an enzyme; an antibody or an antibody fragment; an Fc fusion protein; a hormone; a peptide; a receptor agonist, or a receptor antagonist.

3. The PMP of claim 2, wherein the enzyme is a recombination enzyme or an editing enzyme.

4. The PMP of claim 2, wherein the mammalian therapeutic agent is insulin.

5. The PMP of claim 1, wherein the mammalian therapeutic agent has a size of less than 100 kD.

6. The PMP of claim 5, wherein the mammalian therapeutic agent has a size of less than 50 kD.

7. The PMP of claim 1, wherein the mammalian therapeutic agent has an overall charge that is neutral.

8. The PMP of claim 7, wherein the mammalian therapeutic agent has been modified to have a charge that is neutral.

9. The PMP of claim 1, wherein the mammalian therapeutic agent has an overall charge that is positive.

10. The PMP of claim 1, wherein the mammalian therapeutic agent has an overall charge that is negative.

11. The PMP of claim 1, wherein the exogenous polypeptide is released from the PMP in a target cell with which the PMP is contacted.

12. The PMP of claim 11, wherein the exogenous polypeptide exerts activity in the cytoplasm of the target cell.

13. The PMP of claim 11, wherein the exogenous polypeptide is translocated to the nucleus of the target cell.

14. The PMP of claim 13, wherein the exogenous polypeptide exerts activity in the nucleus of the target cell.

15. The PMP of claim 1, wherein uptake by a cell of the exogenous polypeptide encapsulated by the PMP is increased relative to uptake of the exogenous polypeptide not encapsulated by a PMP.

16. The PMP of claim 1, wherein the effectiveness of the exogenous polypeptide encapsulated by the PMP is increased relative to the effectiveness of the exogenous polypeptide not encapsulated by a PMP.

17. The PMP of claim 1, wherein the exogenous polypeptide comprises at least 50 amino acid residues.

18. The PMP of claim 1, wherein the PMP comprises a purified plant extracellular vesicle (EV), or a segment or extract thereof.

19. The PMP of claim 23, wherein the EV or segment or extract thereof is obtained from a citrus fruit.

20. The PMP of claim 24, wherein the citrus fruit is a grapefruit or a lemon.

21. A composition comprising a plurality of PMPs, wherein each of the PMPs is a plant EV, or a segment or extract thereof, wherein each of the plurality of PMPs encapsulate an exogenous polypeptide, wherein the exogenous polypeptide is a mammalian therapeutic agent, the exogenous polypeptide is not a pathogen control agent, and the composition is formulated for delivery to an animal.

22. The composition of claim 21, wherein the PMPs in the composition are at a concentration effective to increase the fitness of a mammal.

23. The composition of claim 21, wherein the exogenous polypeptide is at a concentration of at least 0.01, 0.1, 0.2, 0.3, 0.4, 0.5, or 1 μg polypeptide/mL.

24. The composition of claim 21, wherein the composition is formulated for administration to a mammal and/or formulated for administration to a mammalian cell.

25. The composition of claim 21, further comprising a pharmaceutically acceptable vehicle, carrier, or excipient.

26. A method of producing a PMP comprising an exogenous polypeptide, wherein the exogenous polypeptide is a mammalian therapeutic agent, and wherein the exogenous polypeptide is not a pathogen control agent, the method comprising:
(a) providing a solution comprising the exogenous polypeptide; and
(b) loading the PMP with the exogenous polypeptide, wherein the loading causes the exogenous polypeptide to be encapsulated by the PMP.

27. The method of claim 26, wherein the exogenous polypeptide is soluble in the solution.

28. The method of claim 26, wherein the loading comprises one or more of sonication, electroporation, and lipid extrusion.

29. The method of claim 28, wherein PMP lipids are isolated prior to lipid extrusion.

30. The method of claim 29, wherein the isolated PMP lipids comprise glycosylinositol phosphorylceramides (GIPCs).
